(19) United States
(12) Patent Application Publication
(10) Pub. No.: US 2017/0330045 A1
(43) Pub. Date: Nov. 16, 2017

(54) METHOD AND APPARATUS TO BUILD NEW ELECTRONIC INSTRUMENTS AND DEVICES: THE 3D-FLOW OPRA TO SOLVE APPLICATIONS OF FAST, REAL-TIME, MULTI-DIMENSION OBJECT PATTERN RECOGNITION ALGORITHMS (OPRA) AND THE 3D-CBS (3-D COMPLETE BODY SCREENING) TO ACCURATELY MEASURE MINIMUM ABNORMAL BIOLOGICAL PROCESSES OF DISEASES AT AN EARLY CURABLE STAGE SUCH AS CANCER IMPROVING DIAGNOSIS AND PROGNOSES FOR MAXIMIZING REDUCTION OF PREMATURE DEATHS AND MINIMIZE THE COST PER EACH LIFE SAVED

(71) Applicant: Dario B. Crosetto, DeSoto, TX (US)

(72) Inventor: Dario B. Crosetto, DeSoto, TX (US)

(21) Appl. No.: 15/338,256

(22) Filed: Oct. 28, 2016

Related U.S. Application Data
(60) Provisional application No. 62/386,876, filed on Dec. 13, 2015.
(60) Provisional application No. 62/285,388, filed on Oct. 28, 2015.

(51) Int. Cl. G06K 9/00; A61B 6/00; G06F 19/00

(52) U.S. Cl. CPC G06K 9/00973; A61B 6/5217; G06F 19/321

(57) ABSTRACT

The present 3D-Flow OPRA is a revolutionary electronic instrument for multiple applications: advancing science, saving lives, finding and tracking fast moving objects, etc. It allows to build a flexible, scalable, technology-independent, cost-effective powerful tool to uncover the unknown and to confirm or exclude the existence of a subatomic particle predicted by theoretical physicists. When used for Medical Imaging applications the 3D-Flow OPRA allows to accurately measure minimum abnormal biological processes of diseases at an early curable stage such as cancer in a 3D-CBS (3-D Complete Body Screening), improving diagnosis and prognosis to maximize reduction of premature deaths and minimize cost per each life saved. It is capable of executing programmable pattern recognition algorithms in real-time of multidimensional objects by analyzing in a single crate of electronics of 36 cubic cm, ALL data arriving at ultra-high speed from a matrix of thousands of transducers at over 20 TB/sec with zero dead-time. Both instruments, the 3D-Flow OPRA and the 3D-CBS can benefit from the additional ER/DSU invention also described in this non-provisional patent application, which allows to record real data from detectors and replay them to the 3D-Flow OPRA and 3D-CBS systems in a controlled environment to facilitate testing, debugging and measuring the efficiency.

### CROSS REFERENCE TO RELATED APPLICATIONS (IF ANY). (RELATED APPLICATIONS MAY BE LISTED ON AN APPLICATION DATA SHEET, EITHER INSTEAD OF OR TOGETHER WITH BEING LISTED IN THE SPECIFICATION.)

This application claim prior provisional application filed Oct. 28, 2015, No. 62/285,388, entitled FLEXIBLE, SCALABLE, TECHNOLOGY-INDEPENDENT, VERIFIABLE 3D-FLOW IMPLEMENTATION TO BUILD A COST-EFFECTIVE POWERFUL TOOL TO DISCOVER NEW PARTICLES AND TO ACCURATELY MEASURE MINIMUM ABNORMAL BIOLOGICAL PROCESSES SUCH AS CANCER IN A 3-D COMPLETE BODY SCREENING (3D-CBS) SYSTEM FOR EARLY DIAGNOSIS AND ACCURATE PROGNOSES TO MAXIMIZE REDUCTION OF PREMATURE DEATHS AND MINIMIZE THE COST PER EACH LIFE SAVED, the disclosure of which is incorporated herein in its entirety by reference thereto.

This application claim prior provisional application filed Dec. 13, 2015, No. 62/386,876, entitled 3D-FLOW OPRA—A NEW ELECTRONIC INSTRUMENT AND DEVICE TO SOLVE TARGETED APPLICATIONS OF FAST, REAL-TIME, MULTI-DIMENSION OBJECT PATTERN RECOGNITION ALGORITHMS (OPRA) ON DATA ARRIVING IN PARALLEL FROM A MATRIX OF THOUSANDS OF TRANSDUCERS AT A VERY HIGH SPEED, the disclosure of which is incorporated herein in its entirety by reference thereto.

### STATEMENT OF FEDERALLY SPONSORED RESEARCH OR DEVELOPMENT (IF ANY)

No Federally sponsored research for this application.

### THE NAMES OF THE PARTIES TO A JOINT RESEARCH AGREEMENT IF THE CLAIMED INVENTION WAS MADE AS A RESULT OF ACTIVITIES WITHIN THE SCOPE OF A JOINT RESEARCH AGREEMENT

This claimed invention was not made as a result of activities within the scope of a joint research agreement, it is solely the invention of the author Dario B. Crosetto.

### REFERENCE TO A “SEQUENCE LISTING,” A TABLE, OR A COMPUTER PROGRAM LISTING APPENDIX SUBMITTED ON A COMPACT DISC AND AN INCORPORATION BY REFERENCE OF THE MATERIAL ON THE COMPACT DISC. THE TOTAL NUMBER OF COMPACT DISC INCLUDING DUPLICATES AND THE FILES ON EACH COMPACT DISC SHALL BE SPECIFIED.

No “Sequential Listing”, table, or computer listing appendix on a compact discs is submitted with this application.

### BACKGROUND OF THE INVENTION

The innovative 3D-Flow parallel-processing architechture, and fault-tolerant system, recognized valuable by major, public, scientific reviews, proven feasible and functional in hardware, breaks the speed barrier in real-time applications such as in High Energy Physics (HEP) and Medical Imaging. It is flexible, scalable, programmable, modular, and technology-independent since it is able to migrate to the most advanced and cost-effective technology.

It creates a revolution in the field because it provides experimenters with a new instrument to test the efficacy of their theory and apparatus just as an oscilloscope or a Logic State Analyzer tests the theory of a circuit design and that its implementation is working properly.

The 3D-Flow instrumentation for fast, real-time Object Pattern Recognition (OPRA) on data arriving in parallel from thousands of sensors at a very high speed with its accessories such as LHC TER/DSU, 3D-CBS/DSU, RAU, ATCA-PRAI, etc. is like an Oscilloscope or Logic State Analyzer with their accessories such as a Trimode differential probe, a DDR3 SODIMM Interposer, etc.

To understand the difference between the new 3D-Flow OPRA, an oscilloscope and a Logic State Analyzer, instrumentations existing for many years, I provide here a short description of each one—what is it and what it is used for.

What is an oscilloscope? It is a type of electronic instrument that allows observation of constantly varying signal voltages, usually as a two-dimensional plot of one or more signals as a function of time. Modern digital instruments may calculate and display these properties directly. However, oscilloscopes are somewhat limited with only two or four input channels to correlate a small number of digital, analog and serial signals.

Which applications can benefit from an oscilloscope? It is used to visualize and measure the characteristic of signals and to troubleshoot malfunctioning equipment. The usefulness of an oscilloscope is not limited to the world of electronics. With the proper transducer, an oscilloscope can measure all kinds of phenomena. A transducer is a device that creates an electrical signal in response to physical stimuli, such as sound (microphone), mechanical stress, pressure, light, or heat.

What is a Logic State Analyzer? A logic analyzer is an electronic instrument that captures and displays multiple signals from a digital system or digital circuit. It may convert the captured data into timing diagrams, protocol decodes, state machine traces, assembly language, or may correlate assembly with source-level software. Logic Analyzers have advanced triggering capabilities, and are useful when a user needs to see the timing relationships between many signals in a digital system. A logic analyzer can be triggered on a complicated sequence of digital events, then capture a large amount of digital data from the system under test.

Which applications can benefit from a Logic State Analyzer? Logic analyzers provide an ideal tool to verify and debug complex designs for electrical engineers. Logic analyzers are useful when multiple signals must be observed simultaneously, as well as when you need to look at a system's signals in the same way its hardware does. The biggest difference from the oscilloscope is the extra input channels it offers.

The implementation of the verifiable 3D-Flow system with the capability of extracting ALL valuable information from radiation, which can save taxpayers billions of dollars by providing a very powerful tool to discover new particles and benefit humanity with an effective early cancer detection potentially saving millions of lives. Unlike the traditional Level-1 Trigger algorithm implementation in “cabled-logic” which provides the capability to execute one algorithm, or the CERN-CMS approach of executing 128 algorithms, or the costly lower performance FPGA approach, the 3D-Flow architecture allows the execution of trillions of different algorithms like a generic processor, but has the advantage over any Pentium, ARM, SPARC, Hypercube, etc., processor/architecture in that it can execute specialized instructions (or “OPRA steps” for an optimized Object Pattern Recognition Algorithm) to identify particles with the capability to execute at each “step” up to 26 operations such as add, subtract, compare with 24 values, etc., in less than 3 nanoseconds. The 3D-Flow performance is further increased by its bypass switch and NEWS communication channels with neighbors.
1. Sixteen Hours of Movie Praising “Guesswork” Costing Trillions of Dollars to Control Millions of Variables that No One Still Fully Understands and Ignoring the “Certainty of Results” that Can Be Obtained with a Few Controllable Variables

Recently U.S. National Public Television (PBS, Channel 13 in Dallas) broadcasted a series of three episodes by the title: “The Emperor of all Maladies” providing an overview of the war on cancer for more than a century with many promises and apparent victories followed by many failures that still leave most of the victims of cancer dead, with the prospective by the World Health Organization in agreement with other major Government agencies and Cancer organizations that the number of cancer deaths will double in the next 20 years.

In 2008, PBS broadcasted “The Truth About Cancer” 116 minutes, Linda Garmon, available on DVD $7.99 and “Health Matters: Cancer, Heart disease, etc.” 532 minutes available in 5 DVDs for $29.99.

Facts & data show that we are losing the war on cancer. In spite of its global economic cost of over $1.4 Trillion per year, the cancer mortality rate during the past 50 years was reduced by only 5%, while for stroke it was reduced by 74%, 64% for heart disease and a 58% reduction of flu and pneumonia and there is no significant difference in the mortality rate between countries spending over $800 per person per year on cancer and those countries that do not.

However, despite the fact that we are losing the war on cancer, the recent program “The Emperor of all Maladies” reported making good progress on cancer e.g. by devoting the first 2-hour episode almost exclusively to childhood leukemia which according to them now has a 90% survival rate. In so doing, it gives viewers the impression that cancer is being defeated. However, leukemia accounts for only 4.1% of the total deaths from cancer, costing $500,000 per treatment and the Surveillance Epidemiology End Results (NIH-SEER) reports a 57.2% survival rate, while lung cancer, that accounts for 27.2% of the total cancer deaths, has a survival rate of only 16.8% (see http://seer.cancer.gov/statfacts/html/leuks.html).

The following are sentences that I extracted from the first episode “The Emperor of all Maladies” that you can watch in streaming at http://video.pbs.org/program/story-cancer-emperor-all-maladies/ (or on DVD for $29.99): “Dec. 28, 1947, . . . Robert [a child] was injected with poison . . . no one knew what was going to happen, not even the doctors. . . . radiation is powerless when cancer is spread, . . . for hundreds of years doctors have searched for a chemical mixture to cure cancer, . . . chemicals were a little more than a guess, . . . 1948 Robert recovers, . . . during the following months all children succumbed to cancer, Robert died on Apr. 2, 1949, . . . Dr. Sydney Faber [one of the most influential scientists on cancer, past President of the American Cancer Society] in 1952 offered a hope for the cure of cancer, Faber engaging in hope and faith, . . . the new immune system turned against itself, . . . 1950 Faber was making a little progress, . . . in the end his child always died, he needed an ally, Mary Lasker, a philanthropist became his ally, . . . Mary Lasker was very vocal but she lost her battle at home on May 30, 1942 when her husband Albert Lasker [owner of the cigarette Company Lucky Strike] died of colon cancer, . . . Mary needed a scientist to validate her evangelical belief to cure cancer, this person was Dr. Sidney Faber, . . . 1955, tens of thousands of cancer drugs entered the FDA pipeline, . . . as the drug seemed to work, cancer will develop a defense, . . . the problem was, resistance, . . . 1962 VAMP trials with four drugs, . . . they were just experimenting, . . . Luca died at 6 years old . . . caused by the drug, . . . 1969, the partnership Government-Science conquered space [man landing on the moon] why should not conquer cancer? . . . President Nixon in 1971 signed the Healthcare Act to conquer cancer, $1.6 billion to NCI to create an entity based on a promise, . . . cancer crusade, . . . President Nixon: “It will not fail because of lack of money, if $100 million for this year will not be enough, we will provide more money.” . . . Elusive, . . . the cancer cell, that its true nature no one still fully understood, until that mystery is sorted, victory could never be won”.

The global strategy in fighting cancer for more than a century is very disheartening. Trillions of dollars have been spent on research where “no one knew what was going to happen, not even doctors . . . chemicals were a little more than a guess, . . . engaging in hope and faith, . . . ten thousands of drugs entered the FDA pipeline, they were just experimenting, . . . create an entity based on a promise [not on knowledge, on sound scientific reasoning that can be confirmed experimentally to significantly reduce cancer deaths], . . . cancer crusade . . . ” without funding research based on precise calculations, logical reasoning in understanding the laws of nature of physics which are more deterministic, easy to predict and prove.

Certainty of results can be obtained by accurately measuring a few controllable variables, but funding innovations that can accurately measure these variables is not provided and the dissemination of these technological breakthrough are even boycotted, oppressed, suppressed.

We have known for over 60 years (confirmed by experimental results) that when cancer is detected at an early stage it can be successfully cured and save 50% of all cancer deaths. However, we do not have effective drugs to cure the majority of late stage cancers (these very expensive drugs on average prolong the patient's life for a few months when cancer is detected at an advanced stage).

What is needed to save many lives, therefore, is an effective early cancer detection because we have already the means (surgery, radiation therapy, drugs, etc.) to cure cancer if it is detected at an early stage.

The logical investigation is to look at the best signals provided by the mutation of the very first normal cells into cancerous cells and accurately detect those signals to obtain an effective early detection.

Among changes in odor, temperature, tissue conductivity, density, fluorescence, etc., caused by the start of the development of cancer, the most reliable is a change in metabolism because cancer cells are growing faster than normal cells and consume from 5 to 70 times more nutrient than cancer cells. Associating a radioisotope emitting 511 keV photons in opposite directions to a molecule of nutrient (Oxygen-15, Ammonia, C-11, Glucose FDG, Rubidium-82, etc.) and accurately measuring the impact point of the photons in the detector (particle detection), gives us the possibility to track the minimum abnormal consumption of nutrient from body cells in different organs and parts of the body.

Measuring a few of these variables (changes in tissue density, fluorescence, etc.) can further improve the certainty of results, and for this reason we have multimodality instrumentations (PET/CT, PET/MRI, PET/Ultrasound, etc.), blood tests and ultimately the biopsy to look at the cells' structures through a microscope.

Because the most important variables that need to be measured in a non-invasive test that can be extended to a large population at a low radiation dose and low examination cost depend on accurately extracting and measuring all the relevant information for specific modality (511 keV for PET, 140 keV for SPECT, 60 keV for CT, etc.) from the many signals generated from radiation, it is necessary to address improvements and techniques in particle detection.

The measurements of the few characteristics (or variables) of these photons are controllable. They are: photon's total energy, arrival time, coordinates of the impact point in the detector, high signal-to-noise ratio and the capability to capture as many signals as possible at a very high input data rate and filtering them from the many other signals from radiation.

Inventions (the 3D-CBS, “3-D Complete Body Screening” and the MR/3D-CBS) and new techniques can improve measurements of each of the above variables. Calculations and logical reasoning can predict the advantages from each new technique. I trust that a scientist will recognize and explain research in physics where the variables are limited, and calculations and logical reasoning can predict with high accuracy the expected results of an invention designed to solve technical problems. Whereas in the field of medicine, because our body is very complex, what was stated at the end of the movie that “the true nature of a body cell no one fully understood” is true, as the variables in the body are thousands or even millions, difficult if not impossible to control; this allows scientists to make only a “guess” rather than estimate results with any accuracy.
A. Background material relative to these inventions related to the project's research plan

There has been no similar case in history where the Director of one of the most prestigious world research laboratories requested and gathered top scientists and experts from all over the world to determine whether an invention could be a turning point in the discovery of new particles.

Usually, new ideas and projects are presented at conferences and published in peer-review scientific journals and Crosetto did this; however, after receiving many letters of support from top scientists and leaders in the field who realized the exceptional value and potential of Crosetto's invention, the Director of the Super Conducting Super Collider (SSC) decided to call an international summit to evaluate Crosetto's invention.

The challenge in High Energy Physics experiments is to identify good events at the lowest cost per each good event captured from data relative to over a billion collisions per second arriving from the detector at a rate higher than 80 million events per second.

As analogy, it would be like watching a movie from a projector showing high resolution frames, each containing billions of bacteria (or a live event) sped up to 40, 80, or over 100 million frames per second instead of the usual 24 frames per second, and trying to identify and extract all frames containing a rare bacteria with a specific shape occurring on average once every 10 billion frames, without being able to slow the movie down or store the data for later examination.

Typically a few micrometers in length, bacteria have a number of shapes, ranging from spheres to rods and spirals.

The 3D-Flow system is basically a “decision box” or “Trigger” having the task to process accurately and at great speed each high resolution image and decide whether to keep it or trash it without the possibility to store all of them because in one day the data would fill all the hard drives on the planet.

Finding the rare Higgs boson-like particle or any new particle among billions generated each fraction of a second that experimenters would like to find when the data received satisfy their Object Pattern Real-Time Recognition Algorithm is like finding a rare bacterium among billions of bacteria shown at very high speed.

In Medical Imaging application the challenge is to extract all valuable information from radiation (emitting hundred millions signals per second) related to tumor markers (or other biological processes) in order to reduce the radiation dosage to the patient and identify the smallest irregular biological process at the lowest cost per valid signal captured from these tumor markers. This would provide an effective early cancer detection (or detection of anomalies in other biological processes) at a very low radiation dosage and at an affordable examination cost.

The challenge for both applications is to design a “decision box” called Trigger that can analyze each frame in real-time and can find the rare object or new particle in physics (or anomalous biological process in medicine).

Crosetto's inventions break the speed barrier in real-time applications and could not have been envisioned with current technology before his inventions. They provide a very powerful tool to discover new subatomic particles while lowering the cost of High Energy Physics experiments, and can provide an effective, low radiation, low cost, early cancer detection.

Crosetto's basic invention is the 3D-Flow architecture capable of executing experimenters' desired programmable complex Object Pattern Real-Time Recognition Algorithms (OPRA), while sustaining an input data rate of over 80 million events per second from over a billion collisions per second, with zero dead-time, and a lower cost per each event captured than other approaches.

For Medical Imaging applications, his basic 3D-Flow invention is combined with other inventions he conceived after the year 2000. They allow all valuable information to be extracted from the tumor markers (linked to radiation) at the lowest possible cost.

Crosetto has explained his basic invention in one page. (See FIGS. 29a, 29b and 29c) and used a practical analogy to explain his inventive concept to middle school students in a book [36] and to high school students in a video &&].

He most recently gave an overview of its technological advantages and the benefits it can bring humanity in a presentation at the ISECM2015 Conference “Energy Challenges and Mechanics—toward the big picture” on Jul. 7-9, 2015 where he was invited as Session keynote speaker. Here you can find Crosetto's presentation:

link to the video https://www.youtube.com/watch?v=Q9bUg3ZsiUk&feature=youtube_gdata;

link to the slides https://drive.google.com/file/d/0BxWfo2ViJ6r5UXpqVHc2NWFnWm8/view?usp=sharing;

link to the abstract http://nscj.co.uk/ecm3/sessions/306_DarioCrosetto.pdf;

link to the bio sketch; http://nscj.co.uk/ecm3/sessions/DarioCrosetto.pdf

Besides contributing to the creation of new powerful tools for the discovery of new particles, Crosetto's goal is to reduce cancer deaths and cost.

One way to increase accountability and achieve this goal would be to demand that
1. funding agencies which use taxpayer money to fund projects request a plan to demonstrate the efficacy of the proposed project that should be tested on a test-bench like is submitting this proposal in FIG. [symbol] , FIG. [symbol] , FIG. [symbol] and FIG. [symbol] , where the “Hardware Detector Simulator” on the left of the figure send at the desired speed from 1 to 80 MHz 8, 192, 16-bit words to the proposed system and the “Results Analyzer Unit” on the right of the figure verifies if ALL “rare” events embedded in the most difficult noisy environment of pileup, and ambiguous pattern to be recognized are found in real-time by the proposed project
2. funding agencies which use taxpayer and donation money to fight cancer, whether through a new drug, vaccine, medical imaging device, or healthy lifestyle promotion, etc., estimate the reduction of cancer deaths and cost they expect to attain with their project (or combined with other existing techniques) and present a plan to test it on a sample population. For example, test the plan on 10,000 people ages 55-74 taken from a location where the mortality rate has been constant for the past 20 years. A difference or no difference in mortality rate will quantify the success or failure of the proposed solution.

Crosetto presented his 3D-CBS invention targeted to early cancer detection which makes use of the 3D-Flow system at the 2000 IEEE-NSS-MIC conference in Lyon, France, in two articles and one book that he distributed in 200 copies free of charge to the leaders and experts in the field at the conference.
1. Crosetto, D.: “A modular VME or IBM PC based data acquisition system for multi-modality PET/CT scanners of different sizes and detector types.” Presented at the IEEE Nuclear Science Symposium and Medical Imaging Conference, Lyon, France, 2000, IEEE-2000-563, [38].
2. Crosetto, D.: “Real-time, programmable, digital signal-processing electronics for extracting the information from a detector module for multi-modality PET/SPECT/CT scanners.” Presented at the IEEE Nuclear Science Symposium and Medical Imaging Conference, Lyon, France, 2000, IEEE-2000-567, [39].
3. Crosetto, D.: “400⁺ times improved PET efficiency for lower-dose radiation, low-cost cancer screening.” Book: ISBN 0-9702897-0-7. 2000. Available at Amazon.com, [40].
   1. Benefits of PI's inventions in the fields of HEP particle detection and Medical Imaging with a single modular industrialized board with 68×3D-Flow processors.

The 3D-Flow DAQ IBM PC Board for Particle Detection and Photon Detection in PET and PET/CT

In December 2002 Crosetto designed and in March 2003 built and tested in hardware a modular board system that would guarantee a difference between any two clock signals among hundreds of thousands of channels of less than 40 picoseconds.

Crosetto provided the schematics [41] and PCB layout so that anyone can verify that these results can be achieved. The schematics and the layout of the board were provided at the following website

https://drive.google.com/file/d/0BxWfo2ViJ6r5WI82SG0xSC1hakU/view?usp=sharing.

This design in 2003 would have reduced CERN OPERA's experiment uncertainty announced in Sep. 23, 2011 by several thousands of picoseconds in measuring the arrival time. (It could also be used at the start time when measuring the path of the muon ‘sister particle’).

Note the traces of equal length (in green in the photo) that guarantee a minimum difference in time among signals within the board, while the use of the programmable delay line MC100EP195 keeps the difference in time among signal in the entire system within 40 picoseconds.

The article [42] title “3D-Flow DAQ IBM PC board for Photon Detection in PET and PET/CT” was presented at the IEEE-NSS-MIC Conference in 2003 and is available at the Conference Record. M3-130.

http://www.crosettofoundation.com/uploads/105.pdf
2. Additional recognition of the scientific merits of Crosetto's inventions after winning the Leonardo da Vinci competition on the most efficient solution in particle detection for early cancer diagnosis which publicly compared the scientific merits of different approaches
A. PI's inventions remain valuable today because the potential benefits of creating powerful tools for the discovery of new particles, for an effective early cancer detection and for benefitting many other fields are enormous
1. One-page summary of the background material in particle detection benefitting several fields presented at the 2013 IEEE-NSS-MIC-RTSD conference in Seoul, South Korea

Abstract of the article [43] presented at the 2013 IEEE-NSS-MIC-RTSD Conference along with 1,679 articles

www.UnitedToEndCancer.org/doc/366.pdf

### Breaking the Speed Barrier in Real-Time Applications to Make Advances in Particle Detection, Medical Imaging and Astrophysics Dario B. Crosetto

Abstract—This paper addresses the approach I took “Beyond Imagination of Future Science” that breaks the speed barrier in real-time applications, which I presented at 3 international conferences within a month in 1992. That same year it was published in scientific peer-review journals. It was recognized valuable by a major scientific review requested by the SSC Director in 1993 (http://links.u2ec.net/doc2/300.pdf) and in letters from several leaders in the field. A study of the scientific literature before and after my invention shows that the effort to develop fast, expensive, high power consumption Ga—As, ECL, etc., circuits for level-1 trigger has become obsolete because my invention can use cost-effective technology, sustain a high input data rate, while at the same time executing complex real-time uninterruptable algorithms for a time period longer than the interval between two consecutive input data. However, as occurs with many breakthroughs, it takes time to fully uncover and develop all the benefits that I integrated with other inventions in detector assembly, segmentation, and coupling of detectors to the electronics that go beyond the original 3D-Flow parallel-processing architecture. The basic 3D-Flow invention, together with other inventions I developed after the year 2000 it can greatly benefit medical imaging applications, thus making effective early cancer detection possible while lowering radiation exposure and the examination cost. This paper presents the proof of concept of my invention as demonstrated in the hardware modular system I built. It addresses what has been understood, what still needs to be understood and what needs to be implemented. This novel decision unit (trigger) will provide a more powerful tool to accurately capture and measure the characteristics of new particles, helping to rule out or confirm expectations. More importantly, it will provide a significant leap in reducing cancer deaths and cost through effective early cancer detection.
2. Summary of the background material specific to Medical Imaging applications presented in 2009 in Houston, 2010 in Dallas, submitted at the 2013 IEEE Life Science Grand Challenges Conference in Singapore and presented in 2014—at the Mondino Institute in Pavia, Italy

Presented at the Italian Researcher Conference in Houston 2009

### Funding 3D-CBS: A Breakthrough Technology, Safe for Screening and Efficacious for Early Cancer Detection—Dario B. Crosetto

The role of Positron Emission Technology (PET) should be changed with use of the 3D-CBS (Three Dimensional Complete Body Screening) for maximizing the capture of signals that will detect minimum abnormal metabolism (or other biological processes), achievable by capturing simultaneously and accurately as many signals as possible from the tumor markers from all organs of the body in order to identify the smallest anomaly, at the lowest cost per signal captured and requiring the minimum radiation to the patient. This paper provides scientific arguments for setting new parameters for industry to establish the correct relation between the goal of obtaining substantial reduction in cancer deaths and the implementation of innovations and technology that will provide the expected results through early cancer detection

Presented at the Life-Science event in Dallas on November 2010 and at the Italian Researcher Conference in Houston in December 2010

### How to Solve the Problem of Cancer and Reduce its Economical Burden: Fund Only Research Projects with Real Potential to Reduce Premature Cancer Deaths. Dario Crosetto.

Abstract: To achieve as soon as possible the goal of maximizing the reduction in premature cancer deaths while minimizing the cost per life saved, and at the same time keeping the door open to progress through the development of basic research (long-term development) it is necessary for every researcher who submits a cancer research project (and each DECISION MAKER who plans a service related to cancer) to provide an estimate, supported by scientific arguments, of the percentage of reduction of premature cancer deaths and a fair estimate of the percentage of reduction in cost for each life saved that their project should attain.

According to the World Health Organization (WHO), by 2030 there will be more than 13 million deaths from cancer worldwide and nearly 21 million cases diagnosed annually [44] (see www.crosettofoundation.org/uploads/383.html).

Over the past 50 years, reduction in cancer deaths has been recorded as a mere 5%, while for heart disease the reduction was 64%, although smaller investments were allocated to the latter [17]. (see www.crosettofoundation.org/uploads/382.html)

These facts tell us that there is the need for a change in cancer research, which can be summarized as follows:
1. The need to equate a reduction in cancer deaths as the principal measurement for assessing the progress in the fight against cancer is accepted in all fields as reported in The New York Times, Apr. 24, 2009 [17]. Therefore, it follows logically that the researchers should provide a quantitative estimate of what their research will achieve in terms of reduction of cancer deaths supported by scientific arguments, and a plan describing the procedure to measure their results (for example, a measurement plan with a nonhazardous, safe test for the patient performed on a representative sample of 10,000 people ages 50-75, selected in a location with a constant cancer death rate of 50 deaths per year recorded over the previous 20 years). Finally they should follow up with the data of their experimental results (measuring on a sample population under age 75 the percentage of reduction of cancer death attained by their project and not limited to showing achievements in tumor shrinkage, as in many cases the patients still dies after a few months because the original tumor metastasizes).
2. The need to implement the DIALOGUE, involving, physics, medicine, etc., as requested by CERN Director General, Rolf Heuer, during his opening speech at the first workshop of PHYSICS FOR HEALTH held at CERN, Geneva, in 2010.

The “Cancer Research Projects Comparison Table”, reporting so far 124,737 cancer research projects already funded for a total of $37 Billion, translates into practice the above points 1 and 2 and when “implemented consistently” becomes the tool that could lead to a substantial reduction of premature cancer deaths and cost for each life saved. (www.crosettofoundation.org/table.php?lang=en). The projects with the highest potential to reduce cancer deaths and the ones that are a waste of money will stand out first using the powerful tools of the table and then through the dialogue among their Principal Investigators who would be required to support their claims with scientific arguments.

For the first objective the table allows searching and sorting data on: 1. Projects that provide the highest estimate, supported by scientific arguments, of cancer deaths, 2. Active projects that have received conspicuous funding without providing an estimate of results as far as cancer death reduction, 3. Projects ended without providing results in reduction of cancer deaths, and other information.

For the second objective, since it is impossible to set up a live discussion among all 124,000 projects, it makes sense to compare any project under consideration with one which received awards, high funding or which claims (supported by solid scientific arguments) the highest reduction in cancer deaths. A practical example of an ongoing Public, Open “Scientific Procedure” is the one being implemented for some time, which involves emails to experts, workshops at CERN, meetings at BNL, as well as at the University and Polyclinic S. Matteo of Pavia to make the scientific truth prevail on projects with highest potential to reduce cancer deaths and cost [46] (www.crosettofoundation.org/uploads/408.pdf). After several meetings broadcast worldwide over several years on the subject of evaluating in depth the innovative 3D-CBS technology for early cancer detection by the author Dario Crosetto, on Oct. 28, 2010 a worldwide meeting (connected via EVO Caltec system to U.S., Canada, CERN, etc.) was organized at the University of Pavia (Italy) to compare the 3D-CBS project that has been waiting for funding for more than a decade with the Axial-PET project by Christian Joram that won the first prize at the Workshop “Physics for Health” at CERN on Feb. 3, 2010. Surprising and shocking facts are emerging from this scientific procedure, some of which are summarized with testimonials in the YouTube video available at: http://www.youtube.com/watch?v=65MI5_ddlvU. This scientific procedure is core to the implementation of the comparison table and should not be limited to these two projects but by using this table, any cancer research project that can claim higher results in reducing cancer deaths and costs can be compared with those that received awards.

Poster Submitted to the IEEE Life Science Grand Challenges Conference—Dec. 2-3, 2013—NUS—National University of Singapore

### BRIEF SUMMARY OF THE INVENTION

What is the 3D-Flow OPRA? The 3D-Flow OPRA is a new electronic instrument and device to solve target application problems of fast, real-time multi-dimension Object Pattern Recognition (OPRA) on data arriving in parallel from a matrix of thousands of sensors (or transducers) at a very high speed that are sent to an equivalent matrix of thousands of 3D-Flow processors.

The 3D-Flow architecture provides data exchange (2×2, 3×3, 4×4, 5×5, . . . ) with neighboring processors, while its bypass switch allows the execution of uninterruptable algorithms for a time longer than the interval between two consecutive input data sets. Each 3D-Flow processor can execute several different programmable “OPRA steps,” called OPRAS, each consisting of up to 26 operations such as adding, subtracting, comparing to 24 values, etc., in less than 3 nanoseconds.

The result is that it can execute users' desired programmable complex Object Pattern Real-Time Recognition Algorithms (OPRA) comparing the desired object (shape and detailed characteristics) with billions of objects per second, while sustaining an input data rate of several million frames per seconds, with zero dead-time.

It can measure all kinds of phenomena and identify all kinds of objects in 3-D that create an electrical signal in response to physical stimuli.

For example, a shape of different colors, a shape of different levels of heat, a shape of different levels of sound volume and frequencies, a shape of different energies, a shape of different mechanical stress, a shape of different pressure, a shape of different light, the characteristics of a specific subatomic particle measured from signals generated by CCD, APD, PMT, SiPM, PADs, silicon strip detectors, wire-chambers, drift-chambers, etc.

It can find an object that can match several of these properties combined. For example:
a) by combining on the same channel to the 3D-Flow processor array the information from a movie camera in the visible light with another in the infrared light, with a radar, a lidar, etc. Using thousands of movie cameras, each covering a tiny field of view and sending the images to a 3D-Flow OPRA system in a VME crate with up to 5,120 channels or to a VXI 3D-Flow OPRA crate with up to 10,240 channels. For example, each channel carries the information of 8-14 bit color, 8-14 bit heat (infrared light), other bits for the radar and Lidar information, etc.
b) by combining on the same channel to the 3D-Flow processor array the information from PMT, SiPM, Pad transducers on the calorimeter Trigger Tower 108, from a wire chamber, drift chamber, silicon strip detector, etc. in the same view angle. Using a detector with 2,304 detector arrays in a PET/3D-CBS device or 8,192 Trigger Towers 108 in HEP applications. For example, each channel carries 8-bit energy information, a few bits of time information and spatial resolution, etc.

Data from a matrix of different types of sensors or transducers are transferred to an equivalent matrix of 3D-Flow processors at the maximum rate of 1.28 Gbps per channel. The maximum transfer rate for a 5,120 channel VME crate 3D-Flow OPRA system is 6.5 Tbps and for a 10,240 channels VXI 3D-Flow OPRA system is 13.1 Tbps.

The 3D-Flow OPRA has advanced triggering capabilities, and are useful when a user needs to find a specific object or multiple objects with a specific timing relationship between them, or needs to see the timing relationship between several objects in a digital system. It can trigger on a specific multi-dimensional object which has been identified by measuring different kinds of phenomena that create an electrical signal. It can trigger on a complicated sequence of digital events.

When trigger conditions are met, it can save the data of the event(s) with the rare object(s) and time-stamp for subsequent visualization by the user.

It can correlate and trigger on two identified objects located far apart in the array but within a specific relation in time; for example, it can identify back-to-back photons in the annihilation of a positron with an electron in the 3D-CBS Medical Imaging application.

Which applications can benefit from the 3D-Flow OPRA?

In HEP, this 3D-Flow OPRA system or instrumentation can be used for Level-1 Trigger to extract all valuable information from radiation to capture the new desired particle theorized by physicists.

In Medical Imaging, this 3D-Flow OPRA system or instrumentation can be used to extract all valuable information from radiation to enable the detection of cancer and many other diseases at an early curable stage, as well as allowing a significant reduction in the radiation dose given to the patient. Its effectiveness will save many lives [¹] and the low examination cost will reduce healthcare costs.

In a multi-lens movie camera application, the 3D-Flow OPRA system or instrumentation can be used to recognize objects from data arriving from thousands of movie cameras each looking at tiny details with a very small field of view.

And it can benefit many other real-time applications.

Features & Implementation Types and Costs

Because the main objective is fast object pattern recognition on data arriving at a very high speed which needs to be exchanged between neighboring processors, one of the most important requirements is to keep the length of the cables between adjacent 3D-Flow processor boards as short as possible and the entire system in a small compact box. Neighboring processors in different chips or boards will require the longest time to transfer data (e.g. a 30 mm wire requires more than 100 picoseconds).

Although the 3D-Flow architecture that I invented in 1992 can satisfy experimenters' requirements to execute programmable complex algorithms at the highest LHC bunch-crossing rate with zero dead-time, using longer cables will increase the algorithm's execution time and the number of 3D-Flow processor layers; this in turn will increase the power consumption of the system, making the system bigger as it needs to dissipate more power which will further increase the cable length in a cycle that will increase the cost of the system until an optimized geometry balances all these parameters. (The number of layers of 3D-Flow processors needed is calculated by dividing the time needed to execute an algorithm by the time interval between two consecutive input data and rounding the result up to the next integer). This explains why keeping the cable length between adjacent processors in different chips, boards and crates as short as possible and keeping the system as compact as possible will maximize performance at the lowest cost.

In 1994, I proposed the implementation of a 1280 channel 3D-Flow system in a cylindrical geometry 1 m in diameter×1.8 m tall (reflecting the cylindrical geometry of the detector: calorimeter in HEP and PET in Medical Imaging) with the longest cable between adjacent 3D-Flow processors boards only 13 cm. The same cylindrical geometry remains today the most cost-effective solution to achieve highest performance at the lowest cost, and with today's technology the cylindrical dimensions for an 8,192 channel 3D-Flow system for Level-1 Trigger of a large experiment at LHC would be 40 cm in diameter and 80 cm tall with the longest cable between 3D-Flow processor boards only 8 cm.

However, for practicality, one can give up some cost-efficiency and use the most convenient form factor which can be VXI, VME, ATCA, Micro-ATCA, VPX, etc.

In this proposal, I have selected VXI 490, 495 for the large boards and VME 300 for the smaller boards; however, if there is a specific requirement from CERN or another user, they can be implemented in any form factor because the backplane is custom designed. Among the considerations made in selecting VXI and VME was the fact that they are more economical per volume of electronic circuits implemented, that there are already many of these crates at CERN that can be reused from dismissed electronics, and that the 43,008×3D-Flow processors for a 8,192 channel 3D-Flow system fit into a compact VXI volume 36 cm×36 cm×24 cm, and 25,600×3D-Flow processors for 4,096 channels fit into a compact VME cube 16 cm×16 cm×16 cm minimizing the distance to exchange data between neighboring processors in different chips and boards.

After proving feasibility and functionality of the 3D-Flow invention in hardware in two modular boards each with 68×3D-flow processors implemented in large FPGAs, the major components to build a 3D-Flow system have been quoted by several companies with a reputable record of working products (some have a catalogue of products they build and commercialize). Some components of the 3D-Flow system have been quoted by two or three companies in competition showing feasibility.

The following report is a comparison between the cost of this new instrument—the 3D-Flow OPRA system with some of its accessories and the price of Oscilloscopes and Logic State Analyzers with some of their accessories. Note that the price per channel of the mainframe unit and cables or probes of the accessories of the 3D-Flow OPRA system is very competitive with the price of the mainframe and probes of the oscilloscopes and Logic State Analyzers.

The budgetary quote of the mainframe 3D-Flow system in a VXI and VME form factor and its accessories reported below when ordered with this R&D proposal is based on the estimates received in the quotes provided in the budget justification of this proposal. When these products become available by a manufacturing company that includes them in their catalogue, the price will be determined by the manufacturing company according to the market value.

TABLE 1
Price comparison for Mainframe Instrumentation

| | | Cost |
|---|---|---|
| Item | Unit cost | per channel |
| 4 ch. Oscilloscope 33 GHz (Tektronix | $479,000 | S119,750 |
| DPS77004SX) | | |
| 4 ch. Oscilloscope 23 GHz (Tektronix | $203,000 | S50,750 |
| MSO72304DX) | | |
| 4 ch. Oscilloscope 4 GHz (Tektronix | $44,900 | S11,225 |
| MSO070404C) | | |
| 136 ch., Logic State Analyzer (Tektronix | $99,600 | $732 |
| TLA7BB4 + TLA7012) | | |
| 2,304 ch., 3D-Flow OPRA System, VME | $46,500$ | $20 |
| version with 14,400 × 3D-Flow processors | | |
| for 3D-CBS Medical Imaging for detection | | |
| of cancer and many other diseases at an | | |
| early curable stage, for accurate diagnoses, | | |
| prognoses and efficiently monitoring of | | |
| treatments. (Crosetto-Patent Pending- | | |
| 2304 ch-20 MHz-64 bit-120-OPRAS) | | |
| 4,096 ch., 3D-Flow OPRA System, VME | $78,500 | $19 |
| version with 26,500 × 3D-Flow processors | | |
| for a programmable, zero dead-time Level-1 | | |
| trigger for LHC experiments. (Crosetto- | | |
| Patent Pending-4096 ch-80 MHz-16 bit-30- | | |
| OPRAS) | | |
| 8,192 ch., 3D-Flow OPRA System, VXI 490 | $98,000 | $12 |
| version with 43,008 × 3D-Flow processors | | |
| (see FIG. 69, FIG. 70, FIG. 71 for a | | |
| programmable, zero dead-time Level-1 | | |
| Trigger for LHC experiments for the | | |
| discovery of new particles. (Crosetto-Patent | | |
| Pending-8192 ch-80 MHz-16 bit-20-0PRAS) | | |

TABLE 2
Price Comparison for Probes

| | | Cost |
|---|---|---|
| Item | Unit cost | per channel |
| 4 Oscilloscope Probe-23 GHz-4 × 23 GHz | $86,000 | $21,500 |
| TriMode Differential Probe (Tektronix | | |
| P7520A). | | |
| 4 Oscilloscope probe-4G Hz-4 × 4 GHz | $23,600 | $5,900 |
| Probe (Tektronix P7504) | | |
| 136 ch. Logic State Analyzer Probe 4 × 34 | $37,600 | $276 |
| Probes (Tektronix P6910) | | |
| 2,304 ch. 3D-Flow OPRA Probe-18 × | $2,700 | $1.17 |
| 128 × 10 Gbps per Twinax 0.5 m cable and | | |
| 2 × 400-pin connectors (Crosetto-Patent | | |
| Pending) | | |
| 4,096 ch. 3D-Flow OPRA Prob-32 × 128 × | $4,800 | $1.17 |
| 10 Gbps per Twinax 0.5 m cable and 2 × | | |
| 400-pin connectors (Crosetto-Patent | | |
| Pending) | | |
| 8,192 ch. 3D-Flow OPRA Probe-10 Gbps | $10,880 | $1.32 |
| per Twinax 1 m cable and 2 × 400-pin | | |
| connectors (Crosetto-Patent Pending) | | |

TABLE 3
Price Comparison for Pattern Generators

| | | Cost |
|---|---|---|
| Item | Unit cost | per channel |
| 2 ch. Arbitrary Waveform Generator | $47,300 | $23,650 |
| 1.2 Gsps, 14-bit per channel (Tektronix | | |
| AWG5012C) | | |
| 64 ch. Pattern Generator 300 MHz | $22,600 | $353.12 |
| (Tektronix PG3A ) | | |
| 181 ch. DDR3 SODIMM interposer | $57,700 | $318.78 |
| (Nexus Technologies NEX- | | |
| SODDR3INTR-XL) | | |
| 2,304 ch. Pattern Generator & Event | $23,000 | $9.98 |
| Recorder @ 320 MHz (640 Mbps) per | | |
| channel, 3D-CBS ER/DSU for testing the | | |
| 3D-Flow OPRA for the 3D-CBS Medical | | |
| Imaging applications. (Crosetto-Patent | | |
| Pending) | | |
| 8,192 ch. Pattern Generator & Event | $40,000 | $4.88 |
| Recorder @ 320 MHz (640 Mbps) per | | |
| channel, LHC TER/DSU for testing the | | |
| 3D-Flow OPRA for a programmable, | | |
| zero dead-time Level-1 Trigger for LHC | | |
| experiments. (Crosetto-Patent Pending) | | |
| 4,096 ch. Pattern Generator & Event | $60,000 | $14.64 |
| Recorder @ 640 MHz (1280 Mbps) per | | |
| channel, LHC TER/DSU for testing the | | |
| 3D-Flow OPRA for a programmable, | | |
| zero dead-time Level-1 Trigger for LHC | | |
| experiments. (Crosetto-Patent Pending) | | |

B. VERIFIABLE INVENTION: This proposal provides a complete set of tools able to verify that the 3D-Flow invention delivers what it promises: the capability of discovering new particles, and a reduce cancer deaths and costs when tested on a sample population

FIG. 9a, 9b and 9c illustrates the invention process flow from concept, to simulation, to the design of details, to the verification in hardware of the different parts, to the testing on a sample population.

VERIFICATION: The 3D-Flow System has been proven feasible and functional in hardware in two modular boards. Over ten companies with a record of products showing competence have provided quotes to build all parts showing feasibility. Efficacy of the 3D-Flow system in extracting ALL information from radiation can be verified on a test bench with the DSU.

FINAL VERIFICATION IN SAVING LIVES AND REDUCING HEALTH CARE COSTS: Fund the NRE, fund the 3D-CBS device, test it on 10,000 people ages 55-74 taken from a location where the mortality rate has been constant for the past 20 years.

The expectation is that it will reduce cancer deaths by 33% in 6 years and 50% in ten years and cut at least in half the cost per each life saved, thus significantly reducing healthcare costs. Why an invention shown feasible and beneficial has not been funded?
A. The 3D-Flow invention advances science by providing a very powerful tool for discovering new particles

The proposed implementation of the 3D-Flow system is verifiable for any implementation selected. The right section of FIG. 10, FIG. 12 and FIG. 13 shows the Detector Simulator Unit (DSU) 300 accommodating 16×DSU boards 310 that could replace the billion dollar LHC experimental setup with a unit costing approximately $40,000 generating 15 million (or 60 million) events (each with a size 8,000 channels×16-bit) stored in 256 GB (or 1 TB) RAM memory sending them out at the current 40 MHz LHC bunch-crossing rate with the possibility to double this rate to verify that this proposed 3D-Flow system implemented in one crate will also work ten years plus from now when the LHC will a much higher luminosity and it will be necessary to acquire 32-bit from each of the 8,192 trigger channels carrying information from additional sub-detectors.
3. A 1.3 TB/sec 3D-Flow system for 8,192 channels implemented in one VXI crate having the capability to execute up to 20 OPRA steps/event/channel @80 MHz, 16-bit/channel

The left column of FIG. 10 shows the LHC TER-Trigger Event-Recording and Simulator 200 (generically known as: DSU—Detector Simulator Unit):
This would provide the opportunity for any university or research center to have on the test bench of their laboratory a box of electronics costing approximately $40,000 providing the exact functionality of a multi-billion dollar LHC detector and collider except that it would be in their lab generating up to 16-bit/channel events at the current LHC 40 MHz bunch-crossing rate, or 32/bit/channel events from future experiments, and a rough environment of very high luminosity planned to be available 10 to 15 years from now.
   This way, “remote” experimenters at their home university will not only be able to test the current Level-1 Trigger with real raw trigger data and prepare a working system for 10 to 15 years from now, but they will also be able to manually edit any raw data of the 15-120 million events stored in the memory of the DSU 210, OR 220, creating the most difficult patterns, with pileup events and other very noisy conditions to make it more difficult for the 3D-Flow System to extract all valuable information from the most unexpected environments. This would thoroughly test the performance and capability of the 3D-Flow System to analyze up to 32-bit/data per channel carrying the information from billions of collisions per second arriving in parallel from 8,192 channels at 40 million events/sec and filtering the good events.
This would provide the opportunity for CERN and other laboratories with particle accelerators and detectors that run million-dollar or billion-dollar experiments, to record for 0.2 seconds 256 GB of real trigger row data (or for 1.6 seconds, recording 2 TB of data, if the 128×2 GB SODIMM memory modules are replaced with 128×16 GB SODIMM memory modules) from all the detectors (calorimeter, tracking, etc.) connected through the PRAI boards 110 (Patch Panel Regrouping Associates Ideas) to the DSU crate. The 256 GB (or 2 TB) raw-data file can then be sent to the Scientific Associates at universities in different parts of the world who are participating in the experiments and have designed parts of the electronics. This 256 GB (or 2 T) of raw-data recorded at CERN or other accelerator sites by a TER/DSU unit (Trigger Event Recorder, the same DSU unit working in acquisition mode rather than generating signals to be sent to the 3D-Flow System in Simulator mode) can be loaded into the DSU System on the test bench at the remote laboratory of a university participating to the experiment. This precious data will provide the Scientific Associates at the remote university the means to not only test the functionality and performance of the 3D-Flow System in real-time, but to also check that their electronics installed on the detector at CERN (or other site) are working properly.
   At the remote laboratory of the university, several tests can be performed on these 256 GB (or 2 TB) of trigger raw data. For example, the data can be analyzed to check that the electronics of all 8,192 channels are generating expected data or to identify any dead channels. If an electronic channel or module is shown to be defective, the experimenters can efficiently plan a trip from their university to CERN or other accelerator site with a working module to replace the defective one.
Another advantage of having a TER/DSU unit at CERN (or another site performing similar experiments), is to use the TER/DSU unit at the accelerator-detector site to record 256 GB (or 2T) of trigger raw data with an LHC beam at different known energies (or the detectors can be stimulated with LED light or other known sources that will generate an electrical signal from the detector of a known energy and duration in time). This recorded trigger raw data from different stimuli to the detectors at known energy values will be very useful to the scientific associates at different universities to determine the pedestal values to be subtracted for each channel and the gain they have to store in the lookup tables of each of the 8,192 channels of their electronics. This will be essential for the calibration of all parts of the instruments (CMS, Atlas, etc.) to avoid discrepancies in data such as the Higgs boson-like particle whose energy was recorded as 125.3 GeV and 126.5 GeV in different experiments.

The center column of FIG. 10 shows the 3D-Flow OPRA system for 8,192 channels capable of extracting from 8-64 million events (32-bit×8,192) arriving at a rate of 1.3 TB/sec, ALL valuable information from radiation using up to 40 steps of Object Pattern Real-Time Recognition Algorithms executed in parallel on each of the 32,768×3D-Flow processors @$1 each (10,240×3D-Flow processors out of the total 43,008 processors in the system are used by the 3D-Flow pyramid to funnel data to a single output channel).

The right column of FIG. 10 shows the Results Analyzer Unit (RAU) which verifies that the 3D-Flow system has extracted all valuable information from radiation. (The rare particles found by the 3D-Flow system satisfying experimenters' Level-1 Trigger algorithm). See FIG. 10.
B. The 3D-Flow invention used in the 3D-CBS (3-D Complete Body Screening) benefits humanity with the potential to save millions of lives and reduce healthcare costs when used in Medical Imaging as an early detection tool on asymptomatic people and to accurately prognoses and efficiently monitor the treatment of many diseases
4. A 368 GB/sec 3D-Flow system for 2,304 channels implemented in one VME crate with the capability to execute up to 120 OPRA steps/event/channel @20 MHz, 64-bit/channel

The left column of FIG. [symbol] shows the 3D-CBS ER—Event Recording—and Simulator (generically known as: DSU—Detector Simulator Unit):

It provides the opportunity for any laboratory developing and improving the 3D-CBS or other PET detectors at a university or research center to have on the test bench of their laboratory a box of electronics costing approximately $25,000 providing the exact functionality of million dollar PET detector. See FIG. 11.
C. The 3D-Flow invention breaks the speed barrier in real-time applications; it is flexible, scalable, programmable, modular, technology-independent; it can be built in different platforms (e.g. VME, VXI, VPX, ATCA, μTCA, etc.); it can extract all valuable information from the most noisy and rough radiation environment
5. A 1.3 TB/sec 3D-Flow system for 8,192 channels implemented in two VME crates having the capability to execute up to 30 OPRA steps/event/channel @80 MHz, 16-bit/channel

See FIG. 12 and FIG. 14.
6. the capability to execute up to 35 OPRA steps/event/channel @80 MHz, 16-bit/channel

See FIG. 13.

### BRIEF DESCRIPTION OF THE SEVERAL VIEWS OF THE DRAWING (IF ANY)

FIG. 1—Breakthrough invention. 3D-Flow OPRA—a revolutionary electronic instrument for multiple applications: advancing science, saving lives, fighting terrorism, . . . The figure illustrates 3D-Flow OPRA electronic instrument that can be implemented in a 36 cm cube of electronics, which is capable of executing pattern recognition algorithms in real-time of multidimensional objects (different ideas, or algorithms are represented as a light bulb) by analyzing all data arriving at ultra-high speed from a matrix of thousands of tranducers at over 20 TB/seconds with zero dead time. It provides three examples of possible applications: a) discovering new particles (Level-1 Trigger); b) saving millions of lives and reducing healthcare costs with the 3D-CBS (3-D Complete Body Screening); and c) fighting terrorism (identifying potential threats, find a needle in a haystack)

FIG. 2—Layout of the 3D-Flow programmable system satisfying the requirements for the Level-1 Trigger of current large HEP experiments and their future upgrades. The PRAI-ATCA crate 180 in the center of the figure receives trigger raw data events from the detectors 105, 110, 115, 120 on different connectors, speeds, protocols and formats on electronic board PRAI-B 130, synchronizing them, formatting each event into 8,192 channels×16-bits and sending it via a dual backplane 135, using the board PRAI-B 140 every 25 ns, or 12.5 ns @1.3 TB/sec through 64×128 channels to the 3D-Flow system 9 U×400 mm boards 410, or 420 housed in crate 490.

FIG. 3—Many crates of electronics in HEP experiments would be replaced by a single VXI 3D-Flow crate 490 providing a much more powerful tool to uncover the unknown and to confirm or exclude the existence of a subatomic particle predicted by theoretical physicists. The one crate 3D-Flow system has the capability of executing experimenters' desired programmable complex Object Pattern Recognition Algorithm (OPRA) for the Level-1 Trigger, while sustaining an input data rate over 80 million events per second from over a billion collisions per second, with zero dead-time, at a lower cost (compared to current approaches) per each good event captured.

FIG. 4—Details of the VXI implementation of 8,192 channels 3D-Flow OPRA system for Level-1 Trigger. The system extracts all valuable information using Object Pattern Real-Time Recognition Algorithms (OPRA) from 80 million events/second (radiation) at 1.3 TB/second transfer rate from over a billion collisions/second, using 43,008×3D-Flow processors @$1 each. Data are received at the front end of the 3D-Flow OPRA boards 410 or 420 inserted in crate 490 via 512×16 Twinax (see FIG. 96a and FIG 96b) ribbon cables 145 which are soldered on a small board 645 at the receiving end of crate 490 and are soldered on a small board 646 (see FIG. 106) at the sending crate 180.

FIG. 5a—Details of the VME implementation of the 2,304 channels, 14,400×3D-Flow processors System for the 3D-CBS. The 3D-CBS has the capability to extract ALL valuable information from radiation, reduces considerably the amount of radiation required to be administered to each patient, and enables for the first time an effective early detection of cancer and other diseases in a single examination covering all organs of the body in just four minutes. Because it gives doctors very precise information, they are better equipped to make accurate diagnoses, prognoses and efficiently monitor treatment. Additional benefits are a reduction in the cost of screening examinations and the cost of healthcare. Data are received at the front end of the 3D-Flow OPRA boards 310 or 320 inserted in upper crate 300 via 72×16 Twinax (see FIG. 96a and FIG. 96b) ribbon cables 145 which are soldered on a small board 545 at the receiving end of the upper crate 300 and are soldered on a small board 546 (see FIG. 105) at the sending crate 180.

FIG. 5b—the 3D-Flow architecture from concept (left) to implementation in two FPGA 715 (Field Programmable Gate Array) to board 700 with 68×3D-Flow processors implemented in FPGA, to an ASIC 750 with 64×3D-Flow processors in a chip, to a VME board 310 with 1,600×3D-Flow processors, to the electronic system for the 3D-CBS with 14,400×3D-Flow processors in a crate 300.

FIG. 6—Experimental data over half century show that we are not winning the war on cancer with a reduction of mortality rate of less than 5%, while for the heart disease for the same period was over 50%, while the cost of cancer has increased over 100 fold. The figure illustrated the path to identify the most deadly and costly calamity in the world, and the approach to take to solve the problem.

FIG. 7—Illustration why it is important to extract ALL valuable information from radiation which is related to visualizing abnormal biological processes enabling early cancer detection.

FIG. 8—Illustration of the Positron Emission Technology

FIG. 9—Illustrates the invention process flow from concept, to simulation, to the design of details, to the verification in hardware of the different parts, to the testing on a sample population to reduce cancer deaths and cost. It is composed of FIG. 9a, FIG. 9b and FIG. 9c which have been separated in different sheets to maintain the size of the character legible on a letter size page 8½″×11″, however for better understanding the flow from concept to simulation, to design details and verification the three figures should be placed one next to the other on a vertical layout with FIG. 9a at the top and FIG. 9c at the bottom.

FIG. 9a—Illustrates the conceptual interrelation between components of the 3D-Flow system for application in medical imaging and in physics experiments (input data from different detectors, possibility to execute different algorithms, and generation of different results) and the flow of the data in the system.

FIG. 9b—Same as FIG. 31, providing the logical layout of the 3D-CBS system, where each column is related to the same column in FIG. 9a on top and FIG. 9c on the bottom.

FIG. 9c—Illustrates the physical layout of the different components from the DSU unit 205 generating radiation data recorded from a PET detector 102 in the left column, the 3D-Flow OPRA under test (crate 300 housing data processing boards 310 and the coincidence board 360) with the task to extract all valuable information from the radiation data (tumor markers) in the center column and the RAU unit 240 to the right, analyzing the results found and measuring the efficiency of the system. The lower layer from left to right shows the components related to the application of improving medical imaging.

FIG. 10—3D-FLOW VERIFIABLE SYSTEM for 8,192 Channels 20 OPRA steps/16-bit-channel @80 MHz. Detector Simulator (see crate 200 housing boards 210 or 220 using SODIMM 205. See FIG. 41, FIG. 42, FIG. 43, FIG. 41, FIG. 44, FIG. 48, FIG. 49, FIG. 50, FIG. 51, FIG. 52) with the capability to generate up to 131,072-bit/event sent at 1.3 TB/sec transfer rate to the 3D-Flow System. 3D-Flow System crate 410 (center) housing data processing boards 410 or 420 and the channel reduction board 460 extracts all valuable information from radiation events arriving every 12.5 ns from 8,192 detector channels, 16-bit/channel, executing max 20 OPRA steps/event. Result Analyzer unit 215 in crate 240 (right) verifying all events containing valuable information have been extracted from radiation by the 3D -Flow System.

FIG. 11—3D-FLOW VERIFIABLE SYSTEM for 2,304 Channels, 120 OPRA steps/64-bit-channel @20 MHz. Detector Simulator (see crate 200 housing boards 210 or 220 using SODIMM 205. See FIG. 41, FIG. 42, FIG. 43, FIG. 41, FIG. 44, FIG. 48, FIG. 49, FIG. 50, FIG. 51, FIG. 52) with the capability to generate up to 32,544-bit/event sent at 368 GB/sec transfer rate to the 3D-Flow System. 3D-Flow System crate 300 (center) housing data processing boards 310 or 320 and the channel reduction board 360 extracts all valuable information from radiation events arriving every 50 ns from 2,304 detector channels, 64-bit/channel, executing max 120 OPRA steps/event. Result Analyzer unit 218 in crate 240 (right) verifying all events containing valuable information have been extracted from radiation by the 3D -Flow System.

FIG. 12—3D-FLOW VERIFIABLE SYSTEM for 8,192 Channels, 30 OPRA steps/16-bit-channel @80 MHz. Detector Simulator (left) generating 131,072-bit/event sent at 1.3 TB/sec transfer rate to the 3D-Flow System. 3D-Flow System (center) extracts all valuable information from radiation events arriving every 12.5 ns from 8,192 detector channels, 16-bit/channel, executing max 30 OPRA steps/event. Result Analyzer unit (right) verifying all events containing valuable information have been extracted from radiation by the 3D-Flow System.

FIG. 13—3D-FLOW VERIFIABLE SYSTEM for 8,192 Channels, 35 OPRA steps/16-bit-channel @80 MHz. Detector Simulator (see crate 200 housing boards 210 or 220 using SODIMM 205. See FIG. 41, FIG. 42, FIG. 43, FIG. 41, FIG. 44, FIG. 48, FIG. 49, FIG. 50, FIG. 51, FIG. 52) with the capability to generate up to 131,072-bit/event sent at 1.3 TB/sec transfer rate to the 3D-Flow System. 3D-Flow System crates 495 (center)) housing data processing boards 430 and the channel reduction board 460 extracts all valuable information from radiation events arriving every 12.5 ns from 8,192 detector channels, 16-bit/channel, executing max 35 OPRA steps/event. Result Analyzer unit 215 in crate 240 (right) verifying all events containing valuable information have been extracted from radiation by the 3D -Flow System.

FIG. 14 show the details of the layout of the two VME crates 300, each housing 16×3D-Flow, 256 channels boards 310 or 320 connected to eight ATCA blades, each with 1024 channels received from detectors such as Atlas CMS, etc. To show the path of eight 2×16-Twinax equal length ribbon cables on each of the 64 connectors, I have used the rainbow colors to facilitate following their path. Data are received at the front end of the 3D-Flow OPRA boards 310 or 320 inserted in two VME crates 300 via 2×256×16 Twinax (see FIG. 96a and FIG. 96b) ribbon cables 145 which are soldered on a small board 545 at the receiving end of crate 300 and are soldered on a small board 546 (see FIG. 105) at the sending crate 180. FIG. 15—Photo of the 3D-Flow DAQ-DSP IBM PC modular board with 68×3D-Flow processors suitable to build 3D-Flow systems for detector of any size and proving feasibility and functionality.

FIG. 16. The 3D-CBS on the right provides precise information on the minimum abnormal biological process with a number (top of the fraction) measured versus a number (bottom of the fraction) considered normal of the metabolic activity (or any biological process useful to the physician to identify abnormalities leading to degenerative diseases such as cancer). On the left of the figure is shown the information provided to the physicians from current PET.

FIG. 17—The 3D-Flow system in a cylindrical assembly reflecting the geometry of the calorimeter detector.

FIG. 18—The same 3D-Flow system in an open configuration for flat detectors such as LHCb experiment or in a convenient layout of the cylindrical assembly during construction and maintenance.

FIG. 19—Mini-crate of the 3D-Flow system to optimize short cable length in a cylindrical geometry

FIG. 20—Details of the Mini-crate showing the 90° interconnection between the DAQ board and the mother board interfacing to the stack of daughter boards of the 3D-Flow system to optimize short cable length in a cylindrical geometry.

FIG. 21—Details of the DAQ boards of the Mini-crate showing the 90° interconnection between the DAQ board and the mother board.

FIG. 22—Details of the Mini-crate showing the front view of the motherboard of the 3D-Flow system to optimize short cable length in a cylindrical geometry.

FIG. 23—Details of the Mini-crate showing the rear view of the motherboard of the 3D-Flow system to optimize short cable length in a cylindrical geometry.

FIG. 24—Details of the daughter board without Flex cables of the 3D-Flow system to optimize short cable length in a cylindrical geometry.

FIG. 25—Details of the daughter board with Flex cables of the 3D-Flow system to optimize short cable length in a cylindrical geometry.

FIG. 26—Details of the daughter board with cables, showing the details of the cables-connectors.

FIG. 27—Details of the daughter board with cables, showing the maximum length of 13 cm of the 3D-Flow system to optimize short cable length in a cylindrical geometry.

FIG. 28—3D-Flow software tools: Simulator for a 3D-Flow System with thousands of processors.

FIG. 29—Description of one of 3D-Flow innovative concepts that enables acquiring data at a very high input rate while simultaneously allowing necessary time to accurately analyze the information. It is composed of FIG. 29a, FIG. 29b and FIG. 29c which have been separated in different sheets to maintain the size of the character legible on a letter size page 8½″×11″, however for better understanding the flow of the data through the 3D-Flow system the three figures should be placed one next to the other on a vertical layout with FIG. 29a at the top and FIG. 29c at the bottom.

FIG. 29a—Description of the first 4 steps of a 12 steps sequence of the 3D-Flow parallel-processing architecture for one input/output channel of a stack of 3D-Flow processors as shown in FIG. 30. On the right of the figure there is a graphic representation of the flow of the data for the first 4 steps. On the left of the figure is shown in a table the 12 steps of the flow of input data, processing time in different processors and output results.

FIG. 29b—Illustration of the 12 steps sequence of the flow of the input data and output results in the 3D-Flow parallel-processing architecture for one input/output channel of a stack of 3D-Flow processors as shown in FIG. 30.

FIG. 29c—Description of the last 4 steps of a 12 steps sequence of the 3D-Flow parallel-processing architecture for one input/output channel of a stack of 3D-Flow processors as shown in FIG. 30. On the right of the figure there is a graphic representation of the flow of the data for the last 4 steps. On the left of the figure is shown in a table the 12 steps of the flow of input data, processing time in different processors and output results.

FIG. 30—The 3D-Flow Logical Unit 710 assembled in layers and stack 720 architecture for a pipeline process of frames, each frame entirely processed in one processor

FIG. 31—3D-CBS Logical Design with its functions split for engineering them into hardware.

FIG. 32—3D-CBS Physical layout and electronics crate

FIG. 33—Technological advantages of the 3D-CBS compared to current PET

FIG. 34—3D-CBS for measuring anatomical and functional parameters

FIG. 35—Specification logical drawing VME LHC TER/DSU board

FIG. 36—Specification and tentative layout of the components on the PCB for the LHC TER/DSU board (front of the board).

FIG. 37—Specification and tentative layout of the components on the PCB for the LHC TER/DSU board (back of the board).

FIG. 38—Excel spreadsheet moved to these specification of the non-provisional patent.

FIG. 39—Excel spreadsheet moved to these specification of the non-provisional patent.

FIG. 40—Excel spreadsheet moved to these specification of the non-provisional patent.

FIG. 41—Specification logical drawing VME ER/DSU board using Altera FPGA for a 320 MHz, 512-bit long word, DDR: 640 Mbps×512=40.96 GB/sec Transfer rate.

FIG. 42—Specification logical drawing VME ER/DSU board using Altera Xilinx for a 320 MHz, 512-bit long word, DDR: 640 Mbps×512=40.96 GB/sec Transfer rate.

FIG. 43—Specification and tentative layout of the components on the PCB for the 320 MHz VME ER/DSU board (front of the board).

FIG. 44—Specification and tentative layout of the Altera FPGA components on the PCB for the VME ER/DSU board (back of the board).

FIG. 45—Deleted.

FIG. 46—Deleted.

FIG. 47—Deleted.

FIG. 48—Specification and tentative layout of the Xilinx FPGA components on the PCB for the VME ER/DSU board (back of the board).

FIG. 49—Specification logical drawing VME ER/DSU board using Altera FPGA for a 640 MHz, 512-bit long word, DDR: 1280 Mbps×512=81.92 GB/sec Transfer rate.

FIG. 50—Specification logical drawing VME ER/DSU board using Xilinx FPGA for a 640 MHz, 512-bit long word, DDR: 1280 Mbps×512=81.92 GB/sec Transfer rate.

FIG. 51—Specification and tentative layout of the components on the PCB for the 640 MHz VME ER/DSU board (front of the board).

FIG. 52—Specification and tentative layout of the Altera FPGA components on the PCB for the VME ER/DSU board (back of the board).

FIG. 53—Excel spreadsheet moved to these specification of the non-provisional patent.

FIG. 54—Excel spreadsheet moved to these specification of the non-provisional patent.

FIG. 55—Excel spreadsheet moved to these specification of the non-provisional patent.

FIG. 56—VME 3D-Flow single board, components on both sides, 256 channels, 1600×3D-Flow processors

FIG. 57—VME 3D-Flow single board, tentative components layout on the front of the board for a 256 channels, 1600×3D-Flow processors.

FIG. 58—VME 3D-Flow single board, tentative components layout on the back of the board for a 256 channels, 1600×3D-Flow processors.

FIG. 59—Excel spreadsheet moved to these specification of the non-provisional patent.

FIG. 60—Excel spreadsheet moved to these specification of the non-provisional patent.

FIG. 61—Excel spreadsheet moved to these specification of the non-provisional patent.

FIG. 62—VME 3D-Flow mother board, 256 channels, 832×3D-Flow processors

FIG. 63—VME 3D-Flow daughter board, 256 channels, 768×3D-Flow processors

FIG. 64—VME 3D-Flow mother board, tentative components layout for a 256 channels, 832×3D-Flow processors.

FIG. 65—VME 3D-Flow daughter board, tentative components layout for a 256 channels, 768×3D-Flow processors.

FIG. 66—Excel spreadsheet moved to these specification of the non-provisional patent.

FIG. 67—Excel spreadsheet moved to these specification of the non-provisional patent.

FIG. 68—Excel spreadsheet moved to these specification of the non-provisional patent.

FIG. 69—VXI 3D-Flow board, 84 ASICs, 1,024 channels, 5,376×3D-Flow processors.

FIG. 70—VXI 3D-Flow board, 40 ASICs, tentative components layout on the front of the board for a 1,024 channels, 2,560×3D-Flow processors.

FIG. 71—VXI 3D-Flow board, 44 ASICs, tentative components layout on the back of the board for a 1,024 channels, 2,816×3D-Flow processors.

FIG. 72—Excel spreadsheet moved to these specification of the non-provisional patent.

FIG. 73—Excel spreadsheet moved to these specification of the non-provisional patent.

FIG. 74—Excel spreadsheet moved to these specification of the non-provisional patent.

FIG. 75—VXI 3D-Flow board, 68 ASICs, 1,024 channels, 4,352×3D-Flow processors.

FIG. 76—VXI 3D-Flow board 430, 66 ASICs, 512 channels, 4,224×3D-Flow processors.

FIG. 77—VXI 3D-Flow board 430, 56 ASICs, tentative components layout on the front of the board for a 512 channels, 3,584×3D-Flow processors.

FIG. 78—VXI 3D-Flow board 430, 10 ASICs, tentative components layout on the back of the board for a 512 channels, 640×3D-Flow processors.

FIG. 79—Excel spreadsheet moved to these specification of the non-provisional patent.

FIG. 80—Excel spreadsheet moved to these specification of the non-provisional patent.

FIG. 81—Excel spreadsheet moved to these specification of the non-provisional patent.

FIG. 82—layout of the backplane carrying the connections between neighboring processors in the 3D-Flow array located in different boards. This implementation is similar for VME and VXI crates.

FIG. 83—Eye of the 781 picoseconds signal that is the basic element to refer to for creating the specifications of all instrumentation needed to build a test bench for current and future LHC Trigger requirements. Current LHC Trigger requirements transfer 10-bit data per channel every 25 ns, which requires a signal with an eye of 2.5 ns; transferring 16-bit data per channel would require an eye of 1.56 ns; foreseeing an increase to 32-bit data per channel at 40 MHz LHC bunch crossing when more detectors are added at the Level-1 Trigger, the signal will generate an eye of 781 picoseconds.

FIG. 84—Overview of the instruments needed to be able to test the Level-1 Trigger implemented in a VXI form factor on a test bench of a remote laboratory from CERN-LHC experiments. The Figure illustrates how to build such a test bench system for 2 to 4 channels using commercially available instrumentation that costs over $50,000 per channel. The goal is to provide universities and research laboratories an alternative, 1/3,300th the cost instrument with 8,192 channel test bench system to generate 871 picosecond signals (data) and/or to store them into a memory (or any number of channels using a modular VME form factor approach) at a cost <$15/channel.

FIG. 85—Overview of the instruments needed to be able to test the Level-1 Trigger implemented in a VME form factor on a test bench of a remote laboratory from CERN-LHC experiments. The Figure illustrates how to build such a test bench system for 2 to 4 channels using commercially available instrumentation that costs over $50,000 per channel. The goal is to provide universities and research laboratories an alternative, 1/3,300th the cost instrument with 8,192 channel test bench system to generate 871 picoseconds signals (data) and/or to store them into a memory (or any number of channels using a modular VME form factor approach) at a cost <$15/channel

FIG. 86—Use the Arbitrary Waveform Generator as stimuli to generate signals similar to those generated by the detectors (CMS, Atlas, etc.) at LHC to test the DSU board. Oscilloscope and LSA check for signal integrity.

FIG. 87—Use the previously tested DSU board as a stimuli to generate signals similar to those generated by the detectors (CMS, Atlas, etc.) at LHC to test the TER board. Oscilloscope and LSA check for signal integrity & crosstalk.

FIG. 88—Develop a DSU 8,192 channels instrument using 16×512 channels VME DSU boards plugged into a VME crate and interface them to the host computer to be able to read/write data into the DSU boards memories and to start/stop sending data out from the front panel of each board.

FIG. 89—Testing VME boards with an extender board to facilitate accessing signals with Oscilloscopes and Logic State analyzer's probes.

FIG. 90—Demonstration of the working hardware 3D-Flow to the members of the review panel on Jul. 1, 2003. The inventor of the pocket calculator, Mr. Jerry Merriman was among the members of the review panel. On the left is the demonstration on the test bench of my lab and on the right the gantry for the 3D-CBS that I built in the garage and that can lift over 3 ton of crystal detectors. (On the background: Jerry Merryman the co-inventor with Jack Kilby of the pocket calculator and Paul Bartholdi expert in photos from the Observatory of Geneva, Switzerland).

FIG. 91—Develop a TER 8,192 channels instrument using 16×512 channels VME TER boards plugged into a VME crate and interface them to the host computer to be able to read/write data into the TER boards memories and to start/stop receiving data from the front panel of each board.

FIG. 92—Use the 8,192 channels DSU crate as a stimuli to generate signals similar to those generated by the detectors (CMS, Atlas, etc.) at LHC to test the TER crate. Oscilloscope and LSA check for signal integrity & crosstalk

FIG. 93—Swap the 8,192 channels crate previously used as DSU to work as TER and vice versa for the other crate. Perform full test. Oscilloscope and LSA check for signal integrity & crosstalk

FIG. 94—Pin assignment for SEAM connectors at 36 Gbps LVDS signals

FIG. 95—SamTec SEAF 1.27 mm pitch connectors specifications.

FIG. 96a—Micro Twinax 2-16 ribbon ribbon cable 145 specifications.

FIG. 96b—Micro Twinax ribbon cable 145 performance data and insertion loss & return loss graphs.

FIG. 97—Pin assignment of the LVDS signals to SamTec connector SEAM-40-03.5-S-10-2-A.

FIG. 98—Assembly details of the eight Micro Twinax 16×2 ribbon cables to the small boards at both ends of the ribbon, which house SamTec SEAF-40-03.5-S-10-2-A, 1.27 mm pitch connector on each board. The small board is 59.62 mm wide and 70 mm long. It has pads on one side, at one end of the small PCB to accommodate the 400-pin connector. At the other end of the small PCB on both sides has two columns and two rows of 16×2 pads to solder four Micro Twinax 16×2 ribbons. The connector is secured with four screws to the larger application PCB.

FIG. 99—SamTec SEAF8, 0.80 mm pitch connectors specifications

FIG. 100—Details of how the eight Micro Twinax 16×2 ribbon cables are assembled to the small boards at both ends of the ribbon which house SamTec SEAF8-40-05.0-S-10-2-K, 0.80 mm pitch connector on each board. The small board is 37.84 mm wide and 75 mm long. At one end of the small PCB there are pads on one side to accommodate the 400-pin connector. At the other end of the small PCB on both sides are four columns of 16×2 pads to solder four Micro Twinax 16×2 ribbons. The connector is secured with four screws to the larger application PCB. The eight 16×2 ribbons and the small PCB are tightened together with a strain reliever and this assembly is tightened to the larger application PCB with a second strain reliever. Connectors SEAM8-40-S02.0-S-10-2-K mating with SEAF8-40-05.0-S-10-2-K have a stacking height of 6.9 mm, mounted 80 mm from the edge of the larger application PCB board.

FIG. 101—Details of how the eight Micro Twinax 16×2 ribbon cables to the small boards at both ends of the ribbon, which house SamTec SEAF8-40-05.0-S-10-2-K, 0.80 mm pitch connector on each board. The small board is 37.84 mm wide and 75 mm long. At one end of the small PCB there are pads on one side to accommodate the 400-pin connector. At the other end of the small PCB on both sides there are four columns of 16×2 pads to solder four Micro Twinax 16×2 ribbons. The connector is secured with four screws to the larger application PCB. The eight 16×2 ribbons and the small PCB are tightened together with a strain reliever. Connectors SEAM8-40-S05.0-S-10-2-K mating with SEAF8-40-05.0-S-10-2-K have a stacking height of 10 mm. The connectors are mounted at the edge of the larger application PCB board.

FIG. 102—Details of how the eight Micro Twinax 16×2 ribbon cables are assembled to the small 90° shaped boards at both ends of the ribbon which house SamTec SEAF8-40-05.0-S-10-2-K, 0.80 mm pitch connector on each board. The small 90° shaped board is 37.84 mm wide and 100 mm long along the connector end and 62 mm along the ribbon cable end. At one end of the small PCB there are pads on one side to accommodate the 400-pin connector. At the other end at 90° with respect to the connector, on both sides there are four columns of 16×2 pads to solder four Micro Twinax 16×2 ribbons. The connector is secured with four screws to the larger application PCB. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 103—Details of how the eight Micro Twinax 16×2 ribbon cables are assembled to the small boards at both ends of the ribbon which house a SamTec SEAF8-40-05.0-S-10-2-K, 0.80 mm pitch connector on each board. The small board is 38 mm wide and 128 mm long. At one end of the small PCB there are pads on one side to accommodate the 400-pin connector. At the other end of the small PCB on both sides at 90° with respect to the connector there are two columns of two rows of 16×2 pads to solder four Micro Twinax 16×2 ribbons at 90° with respect to the connector. The connector is secured with four screws to the larger application PCB. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 104—Details of how the eight Micro Twinax 16×2 ribbon cables are assembled to the small boards at both ends of the ribbon which house SamTec SEAF8-40-05.0-S-10-2-K 0.80 mm pitch connector on each board. The small board is 38 mm wide and 180 mm long. At one end of the PCB there are pads on one side to accommodate the 400-pin connector. At the other end of the small PCB, on both sides at 90° with respect to the connector is one column of four rows of 16×2 pads to solder four Micro Twinax 16×2 ribbons at 90° with respect to the connector. The connector is secured with four screws to the larger application PCB. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 105—Details of how the eight Micro Twinax 145 16×2 ribbon cables are assembled to the small boards at both ends of the ribbon which house SamTec SEAF8-40-05.0-S-10-2-K 0.80 mm pitch connector on each board. The small board 545 and 546 is 38 mm wide and 210 mm long. It has a cut on one side of the PCB board as long as the width of the four ribbon cables to create a window on adjacent PCBs assemblies where the four ribbon cables can cross from one side of the PCBs to the other side. At one end of the small PCB there are pads on one side to accommodate the 400-pin connector. At the other end of the PCB, on both sides at 90° with respect to the connector is one column of four rows of 16×2 pads to solder four Micro Twinax 16×2 ribbons at 90° with respect to the connector. The connector is secured with four screws to the larger application PCB. The side of the small PCB board opposite the connector has a hole at the corner in order to secure the board to a frame relieving any mechanical strain on the connector, on the four screws tightening the connector to the board, and to hold the weight of the ribbon cables. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 106—Details of how the eight Micro Twinax 145 16×2 ribbon cables are assembled to the small boards at both ends of the ribbons which house SamTec SEAF8-40-05.0-S-10-2-K 0.80 mm pitch connector on each board. The small board 645 and 646 is 38 mm wide and 300 mm long. It has a cut on one side of the PCB board as long as the width of the four ribbon cables to create a window on adjacent PCBs assemblies where the eight ribbon cables can cross from one side of the PCBs to the other side. At one end of the small PCB there are pads to accommodate the 400-pin connector. At the other end of the PCB at 90° with respect to the connector there is one column of eight rows of 16×2 pads to solder eight Micro Twinax 16×2 ribbons at 90° with respect to the connector. In one small PCB the eight ribbons are soldered on the same side of the connector, while on the other board they are soldered on the side opposite the connector. The connector is secured with four screws to the larger application PCB. The side of the small PCB board opposite the connector has a hole at the corner in order to secure the board to a frame to relieve any mechanical strain on the connector, on the four screws tightening the connector to the board and to hold the weight of the ribbon cables. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 107—Details of how the eight Micro Twinax 16×2 ribbon cables are assembled to the small boards at both ends of the ribbon which house SamTec SEAF8-40-05.0-S-10-2-K, 0.80 mm pitch connector on each board. The small board is 38 mm wide and 300 mm long. It has a cut on one side of the PCB board as long as the width of the four ribbon cables to create a window on adjacent PCBs assemblies where the eight ribbon cables can cross from one side of the PCBs to the other side. At one end of the small PCB there are pads to accommodate the 400-pin connector. At the other end of the PCB at 90° with respect to the connector is one column of eight rows of 16×2 pads to solder eight Micro Twinax 16×2 ribbons at 90° with respect to the connector. In one small PCB the eight ribbons are soldered on the same side of the connector, while on the other board they are soldered on the side opposite the connector. The connector is secured with four screws to the larger application PCB. The side of the small PCB board opposite the connector has a hole at the corner in order to secure the board to a frame to relieve any mechanical strain on the connector, on the four screws tightening the connector to the board, and to hold the weight of the ribbon cables. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

### DETAILED DESCRIPTION OF THE INVENTION

D. The revolutionary scientific advantages of the 3D-Flow invention which extracts ALL valuable information from radiation when applied to HEP experiments

The 3D-Flow System provides a very powerful tool to the HEP community because it extracts all valuable information from radiation.

In HEP applications, the 3D-Flow architecture provides data exchange with neighboring processors while its bypass switch allows the execution of uninterruptable algorithms for a time longer than the interval between two consecutive input data. The result is it can execute experimenters' desired programmable complex Object Pattern Recognition Level-1 Trigger Algorithms (OPRA), while sustaining an input data rate of over 80 million events per second from over a billion collisions per second, with zero dead-time, at a lower cost per each good event captured than other approaches.

Many physics experiments would have already benefitted from this powerful tool to discover new particles and save a great amount of money if DOE had provided the NRE cost in 1995 to build the 3D-Flow chip after funding its study. After receiving $906,000 from the DOE in 1995 and paying Synopsys, one of the best companies designing integrated circuits that generated the RTL files for the silicon foundry to build 4×3D-Flow processors in 350 nanometer technology in a chip, funding went to more costly projects with lower performance. My 3D-Flow invention was recognized valuable by many scientists who wrote letters of appreciation after 1992. In 1993, it underwent a formal scientific review at FERMILab gaining further recognition. In 1994, it was recognized by DOE as a breakthrough invention benefitting science and published on page 216 in the DOE Technology Transfer book DOE/LM-0002 DE94005148. In 2001 it was proven feasible and functional in hardware in 2×FPGAs (Field Programmable Gate Array), each containing 4×3D-Flow processors, interconnected in a cube 2×2×2 structure. In 2003 it was proven feasible and functional in two modular industrialized boards, each with 68×3D-Flow processors in 17 FPGA, interconnected within the board in a (4×4×4) cube structure +1 FPGA of 4 processors for channel reduction. The successful testing of the communication between the two modular boards proved that 3D-Flow systems can be built for detectors of any size.

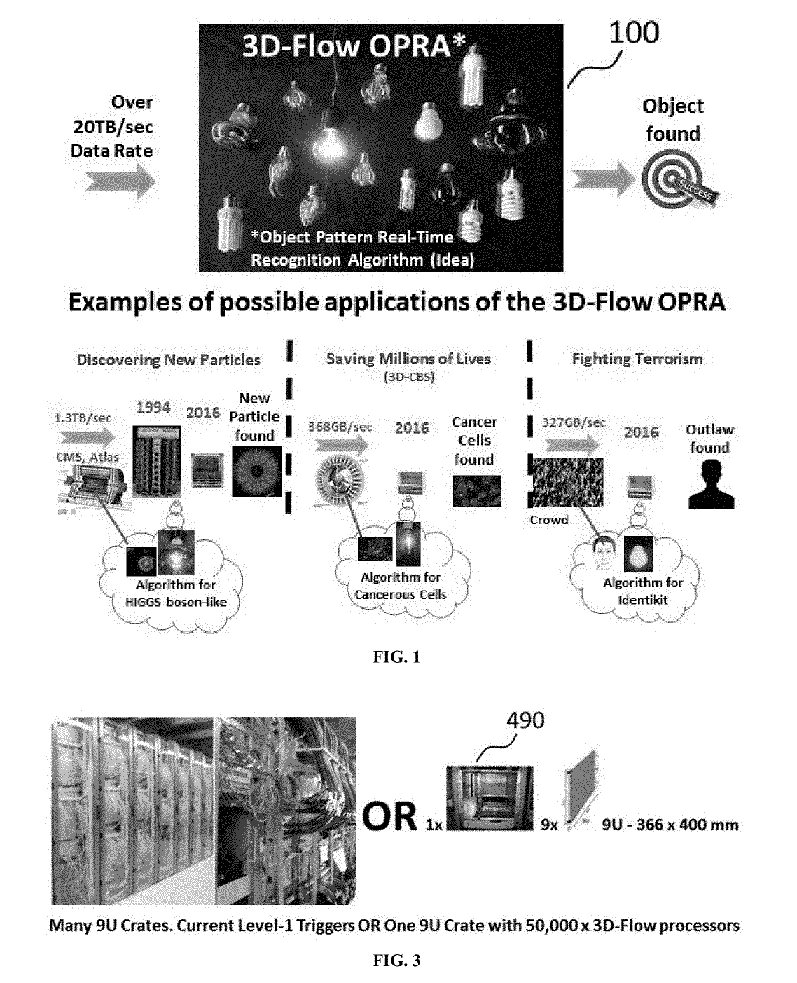

The entire Level-1 programmable system of over 40,000×3D-Flow processors at $1 each can fit into a single crate. A patch panel PRAI-ATCA (see FIG. 1—Breakthrough invention. 3D-Flow OPRA—a revolutionary electronic instrument for multiple applications: advancing science, saving lives, fighting terrorism, . . . The figure illustrates 3D-Flow OPRA electronic instrument that can be implemented in a 36 cm cube of electronics, which is capable of executing pattern recognition algorithms in real-time of multidimensional objects (different ideas, or algorithms are represented as a light bulb) by analyzing all data arriving at ultra-high speed from a matrix of thousands of tranducers at over 20 TB/seconds with zero dead time. It provides three examples of possible applications: a) discovering new particles (Level-1 Trigger); b) saving millions of lives and reducing healthcare costs with the 3D-CBS (3-D Complete Body Screening); and c) fighting terrorism (identifying potential threats, find a needle in a haystack)

FIG. [symbol] ) receives events from the detectors, synchronizes and formats each event into 8,192 channels×16-bit and sends them to the 3D-Flow system—one every 12.5 ns (or 1 every 25 ns with a longer 32-bit word).

Because the LHC collider and experiments cost billions of dollars and the Level-1 Trigger has the most important and challenging task of capturing rare events (one out of 10 billion in the case of the Higgs boson-like particle), if they are missed all the work of analyzing data by thousands of people will be meaningless and the entire money and effort of building for more than 20 years the LHC collider and detectors would be wasted. It would therefore be prudent to thoroughly test the efficacy of the Level-1 Trigger and give the opportunity to more than one group of experimenters/scientists to test their ideas.

In order to allow more than one group of scientists within an experiment to implement and test their Level-1 Trigger algorithms and hardware, a suggestion would be for scientific committees which approve experiments and funding agencies in Europe, U.S. and other countries participating at CERN experiments as member states, associates or observers to create opportunities for open PUBLIC fair scientific reviews where scientists and inventors can submit their ideas for funding, implementation and testing a new Level-1 Trigger on LHC experiments.

Level-1 Triggers of the large experiments at LHC: CMS Atlas, Alice and LHCb do not provide zero dead-time; CMS and Atlas have found only 40 Higgs boson-like events during analysis of data captured randomly instead of intelligently, by the Level-1 Trigger; their leaders recognize they must trash the current Level-1 Trigger electronics because they do not have the capability to execute Object Pattern Recognition Algorithms.

What we should learn from these past errors is that an investment of over $50 billion [²] and 25 years of work by 10,000 people should not be left to a single Level-1 Trigger group in CMS and another in Atlas who did not compete on merit scientific ideas in a fair PUBLIC scientific review with different projects. If such a merit review had been conducted, the 3D-Flow with OPRA capabilities and zero dead-time recognized by a formal public scientific review at FERMILab in 1993 should have been adopted by the LHC experiments.

I am not going to elaborate in this document with examples of how to give the opportunity for innovative ideas requiring support from scientific committees and funding agencies; instead, I am addressing the technical aspect of how a single experiment can provide the hardware environment to test different Level-1 Trigger implementations on the LHC beam.

To facilitate the explanation, I am using the analogy of an oscilloscope made by HP/Agilent, LeCroy or Tektronix to implement the Level-1 Trigger. If all scientists and all Level-1 Trigger collaboration groups have supporting evidence that their real-time Object Pattern Recognition Level-1 Trigger algorithm will work and agree to use HP/Agilent instrument (oscilloscope in the analogy or in this case a new instrument called “HP/Agilent Level-1 Trigger System”), then a single version of the hardware can be installed in both experiments, and universities and trigger groups at different laboratories may purchase the same oscilloscope (or in this case the same “HP/Agilent Level-1 Trigger System”) and can learn how to use their instrument, test that their algorithm works on the test bench of their lab. Then, they can download the same instrument setup and algorithm at the CMS and Atlas experimental site at CERN assigning a different LHC beam time slot to each group.

In the event that the two groups within the same experiment choose a different hardware system, e.g. one HP/Agilent and the other LeCroy, then it will be necessary to provide a platform at CERN that would allow both hardware to be installed. A solution would be to build a patch panel similar to the ATCA-PRAI as suggested in FIG. 1—Breakthrough invention. 3D-Flow OPRA—a revolutionary electronic instrument for multiple applications: advancing science, saving lives, fighting terrorism, . . . The figure illustrates 3D-Flow OPRA electronic instrument that can be implemented in a 36 cm cube of electronics, which is capable of executing pattern recognition algorithms in real-time of multidimensional objects (different ideas, or algorithms are represented as a light bulb) by analyzing all data arriving at ultra-high speed from a matrix of thousands of tranducers at over 20 TB/seconds with zero dead time. It provides three examples of possible applications: a) discovering new particles (Level-1 Trigger); b) saving millions of lives and reducing healthcare costs with the 3D-CBS (3-D Complete Body Screening); and c) fighting terrorism (identifying potential threats, find a needle in a haystack)

FIG. [symbol] of this document to which is attached the HP/Agilent system for a period of LHC beam run time and the LeCroy system for a different LHC beam run time.

If during the design phase of placing both Level-1 Trigger systems working in parallel at the experiment location there is evidence that signal integrity would be compromised, then the system selected as first priority by the scientific committee will receive trigger raw data signals directly from the detector and a copy of the same signals will be provided at an output connector for the alternative, challenger Level-1 Trigger system that should be faster in order to make up for the delay in buffering the raw trigger data signals.

With these hardware arrangements, two groups or individuals having solid scientific grounds to design and implement a new hardware and Object Pattern Recognition Algorithm will have a chance to test the efficiency of their idea with some LHC beam run time.

The $200,000 approximate total cost of a 8,192 channels 3D-Flow OPRA Level-1 Trigger System, as reported by the quotes from reputable companies building similar electronics and detailed in the budget justification of this proposal, is largely justified because it will reduce the risk of a huge investment in money and labor on a different system which would likely be trashed in another 15 years when the level-1 Trigger again does not work.

Because the current Level-1 Trigger has not provided accurate measurements about the unknown particle predicted in theory, helping to rule out or confirm theoretical predictions, CMS and Atlas will continue to claim that more data needs to be acquired to fully understand the nature of the new found particle. However, if this rare event occurs one time in 10 billion events and the Level-1 Trigger does not work effectively, casually capturing some of them amounts to pure luck not experimental science, takes a long time, and is like waiting to win the lottery.

Instead, it will be worth having four or more 3D-Flow OPRA systems, one installed in the experiment at CERN and three on test benches at remote universities and other laboratories to test experimenters' desired real-time algorithm on a real 3D-Flow OPRA system.

The cost of $98,000 for the main frame 3D-Flow system, $40,000 for the DSU (Detector Simulator Unit sending LHC recorded data and difficult to recognize, special events with pileup signals edited manually to the 3D-Flow System) and $10,000 for 8,192 Twinax cables and connectors as detailed in the design below and in the quote #34a) for a total cost of approximately $200,000×4 systems (one at CERN and 3 installed at remote universities)=$800,000 will be well justified to reduce the risk of wasting an investment of over $50 billion and 25 years work by over 10,000 people, by failing to capture data satisfying experimenters' desired complex algorithm.

Its faster, powerful OPRA filtering capability with 43,008×3D-Flow processors compared to FPGA can thoroughly analyze all data within the 2 to 3 microseconds allocated for the Level-1 Trigger and make up for the lost time buffering all data from the official Level-1 Trigger system selected by the scientific committee.
7. THE BIG PICTURE: Assessing the FUTURE need of instrumentation in High Energy Physics

The increase in power at the LHC Collider poses a challenging task in assessing how performant the instrumentation should be, in particular Level-1 Trigger, responsible for making the first very important decision of which events among the trillions of events generated by the LHC, have important information valuable enough to capture. If Level-1 Trigger does not have the capability to capture these valuable events, then thousands of scientists will analyze garbage data and billions of dollars and many years of work will be wasted.

The requirements in 1994 for the Level-1 Trigger were defined in 8,000 channels, each transferring 10-bit of information every 25 ns (see page 124, last paragraph of the documents). This was confirmed recently on page 79, Section 7.2.1 of the CMS 2013 upgrade.

This proposal is providing instrumentation satisfying those requirements and going beyond them to satisfy requirements for future LHC upgrades.

I noticed in several official documents that CERN is moving toward the standardization of 10 Gbps links. I will be glad to comply with this standardization all the way from the CMS, Atlas, Alice and LHCb detectors to the instrumentation analyzing this data with the 3D-Flow OPRA system. It is just a matter of cost, but as I have cost-effectively designed the cables and connectors at $1.17 per channel listed in item 60 on page 152 of the Budget Justification satisfying 10 Gbps speed, I could design the other parts of the system complying with 10 Gbps.

However, it is not the transfer speed which is the real issue. If we need to transfer 10 Tbps Trigger data, whether we use 8,192 cables at 1.28 Gbps, 1,048 cables at 10 Gbps, or 105 cables at 100 Gbps is determined by the cost of each approach. The current components (cables, connectors, SERDES) may offer a more cost-effective solution now at one speed, but tomorrow's market may offer other components.

What is important to address instead is the amount of trigger data generated by each detector that needs to be processed by the Trigger instrument (3D-Flow OPRA or FPGA). From now until 2018 it is the number of bits generated by each of the 8,000 (or 8,192) Trigger Towers 108 every 25 ns which is 10-bits. This generates 3.2 Tbps of data.

Whether the granularity of the Level-1 Trigger is increasing or the LHC bunch-crossings or detectors are added to the Level-1 trigger such as the Inner tracker, the amount of data to be processed at the Level-1 Trigger will increase; however, it is unlikely that the LHC bunch-crossings will increase over 40 MHz until the beam heating problem is solved.

If the Trigger Tower 108 granularity is increased from 8,192 channels to 32,768, one could use 4× crates, each with 8,192 channels and 43,000×3D-Flow processors; however, the OPRA algorithm will increase in complexity (will be longer) because each processor will have to handle more neighboring data from the impact of the particle in the detector, affecting more elements in a smaller granularity.

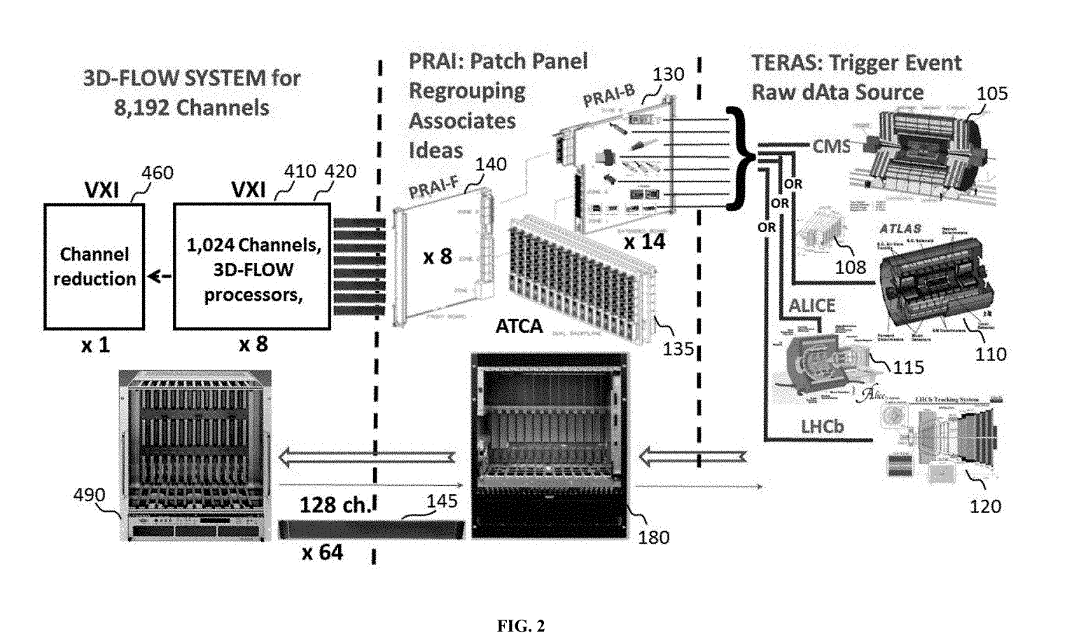

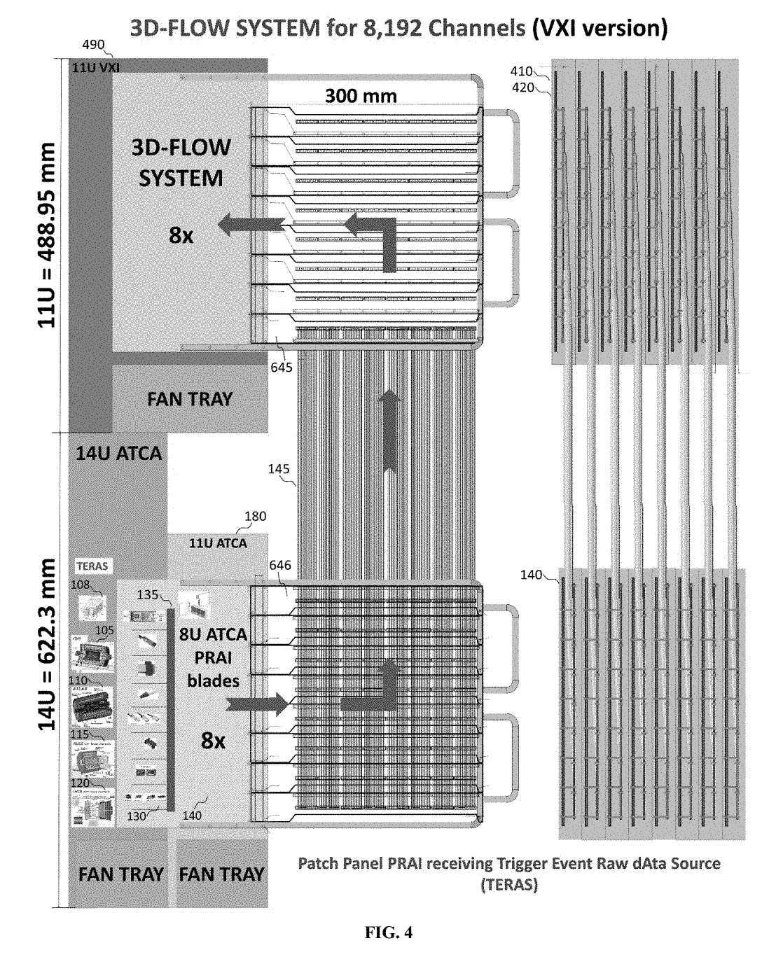

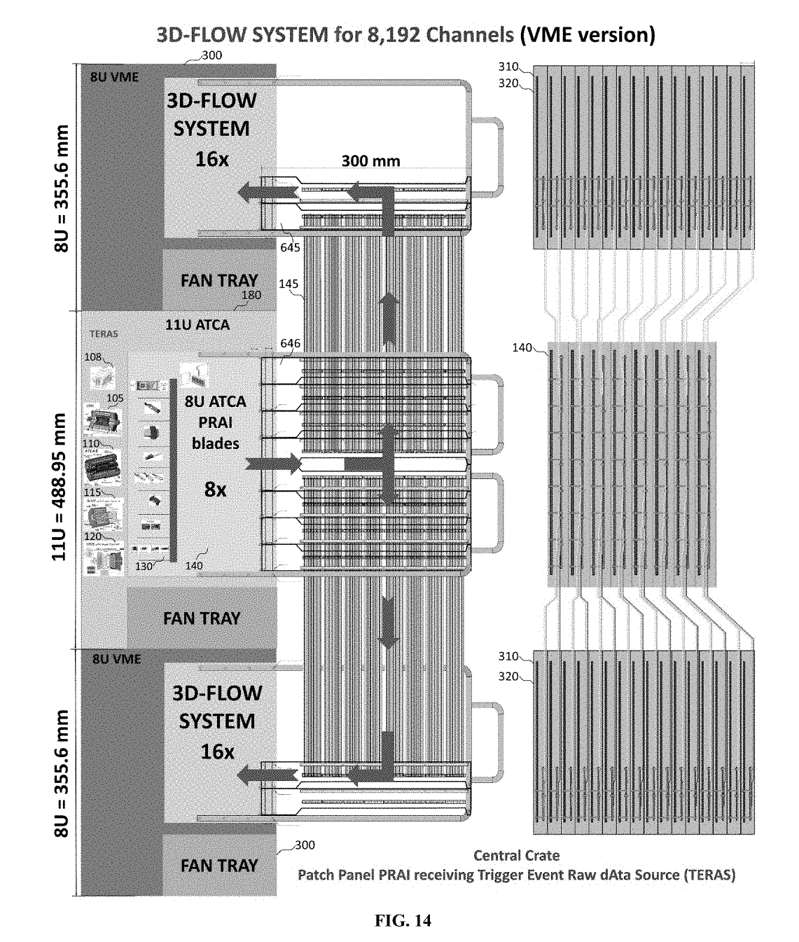

If the number of detectors is increased and instead of generating 10-bit every 25 ns, the Trigger Towers 108 in FIG. 2, FIG. 4 and FIG. 14 generates 16-bits, 32-bits, 64-bits, . . . then the Level-1 trigger should have the capability to process more data.

To simplify this study, let's suppose that the LHC bunch-crossing will not increase over 40 MHz and the 10-bit per channel increases to cope with higher luminosity and more detector information at Level-1 Trigger to:
1. Level A “16-bit per Trigger Tower every 25 ns” until 2018 (already an improvement over the current 10-bit information from each Trigger Tower)
2. Level B “32-bit per Trigger Tower every 25 ns” beginning 2018
3. Level C “250-bit per Trigger Tower every 25 ns” to comply with CERN 10 Gbps standard

As I mentioned before, the 3D-Flow OPRA new instrument can satisfy any additional requirements in a very cost-effective manner. It is important to assess the future needs of instrumentation in HEP in order to optimize the cost-performance.

The following guide through the Budget Justification spreadsheet from pages 149 to 152 will help funding agencies decide which level is most cost-effective.

The proposed project and budget to build the 8,192 channel 3D-Flow OPRA system described in FIG. [symbol] will achieve Level A and Level B.

To facilitate the understanding of cost-performance, I have divided the cost into four main categories:
a) cost of “instrumentation”
b) cost of “cables+connectors”
c) cost of the “3D-Flow OPRA”
d) cost of the “LHC TER/DSU”

To save money until 2018, groups of experimenters who have a small budget and need to have good instrumentation to test the Level-1 Trigger on the test-bench of their lab can add their order to this proposal for a 640 Mbps LHC TER/DSU for about $40,000 as reported in item 58 of the Budget Justification and supported by two quotes on pages 140 and 142. This item complies with Level A.

To upgrade the performance of the entire Level-1 trigger system to Level B, it will only be necessary to have the 1.28 Gbps LHC TER/DSU costing about $120,000 as reported in item 56 on page 151 of the Budget Justification and supported by two quotes on pages 109 and 141 of the same document.

The 8,192 cables+connectors of this proposal in item 60 of the Budget Justification, supported by two quotes on pages 133 and 140, for a total cost of $10,000 to transfer up to 82 Tbps complies with Level C.

To bring the four items listed earlier into compliance with Level C will require the following:
a) “instrumentation” Item 6 for about $500K and item 8 for about $100K of the Budget Justification spreadsheet should be purchased instead at the current oscilloscope price of $64k of item 7.
b) “cable+connectors” will remain the same with a total cost $10,000
c) “3D-Flow OPRA” will need to change to a technology supporting SERDES at 10 Gbps input and output. This should be addressed in depth with ASIC designers. During an informal conversation I was told that 10 Gbps was certainly feasible but the cost of the NRE to the foundry could quadruple.
d) “LHC TER/DSU” should increase its speed from 1.28 Gbps per channel to 10 Gbps per channel. This will also increase the cost and is the reason why I have designed a version at 640 Mbps and, one at 1.28 Gbps; a higher speed version is justified only if required by the other instruments.

Each 3D-Flow processor can execute programmable, complex Object Pattern Real-Time Recognition Algorithms by exchanging data with neighboring processors and manipulating any field or group of bits of the 16-bit, 32-bit, 64-bit . . . or 250-bit word with OPRA steps (OPRAS), each with the capability to execute up to 26 operations such as, addition, subtraction, comparison with 24 values, etc. in less than 3 nanoseconds.

Although the 3D-Flow architecture can satisfy experimenters' requirements to execute longer algorithms when the input word is increased from 16-bit to many more bits by adding the number of 3D-Flow processor layers, do we really need 10 Gbps to carry 250-bit information from each Trigger Tower or is a compromise between 32-bit of the proposed 3D-Flow processor with 1.28 Gbps input/output and 10 Gbps acceptable? Perhaps a 2.56 Gbps offering 64-bit word input from each Trigger Tower would be sufficient and the 3D-Flow processor NRE at the Silicon Foundry will not quadruple.

This proposal offers a set of instruments that are cost-effective, flexible and scalable to satisfy increasing requirements. It will be important to address the issue of whether the information from the Trigger Towers in 2020, after additional detectors are added to cope with increased luminosity, will exceed 32-bits every 25 nanoseconds, rather than to impose a standard to transfer 10 Tbps.

Whether 10 Tbps are transferred with 8,192 cables at 1.2 Gbps, 1048 cables at 10 Gbps or 105 cables at 100 Gbps should be determined by the most cost-effective solution commercially available, but this will have no effect on its ability to find a new particle—one solution just costs more or less than another.
8. SAVING TAXPAYER MONEY: The 3D-Flow System can replace many crates of electronics in HEP experiments with a single crate, providing a much more powerful tool to discover new particles or to disprove a theory

The most difficult task that I have to face for two decades is to make leaders of large experiments understand the importance of creating a tool capable of extracting ALL valuable information from radiation with zero dead-time. This means to thoroughly analyze at the Level-1 Trigger decision, ALL events' raw data received directly from the detector using programmable complex Object Pattern Real-Time Recognition Algorithms (OPRA) for a time longer than the interval between two consecutive events.

Although the 3D-Flow invention having these capabilities was recognized valuable by academia, industry and the world's most prestigious research centers in a formal scientific review, leaders of large experiments failed to recognize its benefits, funding instead less efficient and more costly approaches that could not thoroughly analyze each event without zero dead-time.

These other approaches are documented in at least 12 volumes of proceedings, about 600 pages each, generated during 20 years of “Workshops on Electronics for LHC and Future experiments” (e.g. CERN-2007-001, CERN-LHCC-2007-006, LHCC-G-125, Jan. 15, 2007, the “12^{th} Workshop on Electronics for LHC . . . ”), and in several volumes, some 600 pages each, generated by the Trigger groups during 23 years of large experiments at CERN, such as the 600-page “The Trigger and Data Acquisition project, Volume 1, The Level-1 Trigger, Technical Design Report” CERN/LHC 2000-038, CMS TDR 6.1, Dec. 15, 2000, their upgrades in 2013, and the proceedings from several conferences including the 23 annual IEEE-NSS-MIC conferences, one of which (2013) generated over 10,000 pages of proceedings as reported at this link to its abstracts. The electronics described in these documents costing hundreds of millions of dollars was the result of over 10,000 scientists costing additional billions of dollars in salaries and equipment being lead in the wrong direction because the Level-1 trigger did not have the capability to extract ALL valuable information from radiation. It did not have the capability to capture ALL rare events satisfying experimenters' desired characteristics of new particles.

CERN documents state that in 2011-2012, the Large Hadron Collider (LHC) at CERN generated 1,000 trillion (10¹⁵) events, 100,000 of which were estimated to be Higgs boson-like. On Jul. 4, 2012, CERN announced they had found 40 such Higgs boson-like events at each experiment, Atlas and CMS, by analyzing several terabytes of acquired data and noticing an unusual energy at 125.3 GeV in about 40 events by CMS and at 126.5 GeV for the same number of events by Atlas. These 40 Higgs boson-like events out of the estimated 100,000 indicate a casual recording of those events rather than an informed decision made by the Level-1 Trigger matching desired conditions.

The level-1 Trigger of CMS and Atlas have many shortfalls including not having the capability to capture ALL rare events satisfying experiments' desired characteristic of the new particle. Here are some facts and observations that support this statement:
1. Their current Level-1 Triggers are not dead-time free, nor are their designs for future upgrades of CMS and Atlas Level-1 Triggers. This is not only evident by examining the schematics in the previously mentioned documentation, and in the articles and documentation for future upgrade of the electronics of the Level-1 Triggers of CMS and Atlas, published and presented at conferences, but was also confirmed when I asked this question directly to the presenters of these documents at the Trigger Session of the 2013 IEEE-NSS-MIC-RTSD. A deeper investigation will show that they have very limited programmability, cannot execute complex real-time trigger Object Pattern Recognition Algorithms, and cannot extract ALL valuable information from radiation satisfying experimenters' desire to capture new particles with specific characteristics.
2. This proves that even if the hardware is working correctly, they do not have the capability to find and capture new particle with the desired characteristics satisfying experimenters' complex algorithm.
3. A further proof would be to load the trigger raw data of those 40 Higgs boson-like events with other spurious events in the LHC TER/Simulator (DSU) hardware unit. Then connect this crate to the CMS and Atlas Level-1 Trigger electronics to verify if it fires a Level-1 Trigger only for those 40 events and not for the other events.
4. The fact that the upgrade documents for CMS and Atlas state that the electronics for their Level-1 Trigger must be completely replaced is confirmation that the current system does not work.

Chapter “Lost Opportunities” provides references to publications, documents, even photos of the cylinder structure housing the 3D-Flow system that was one meter in diameter and 1.8 meters high; it included acquisition boards, mother boards, daughter boards, flexible printed circuits, simulators, software tools, etc., that had demonstrated the capability to execute experimenters' programmable desired complex OPRA Level-1 Trigger algorithm with zero dead-time; however, the NRE to build the 3D-Flow ASIC was never funded and so it was never completed.

The 3D-Flow invention was published in several scientific journals and presented at conferences in 1992. Since then it has been acclaimed in several letters by world experts in the field, and was recognized valuable by a formal scientific review in 1993, after which I received $150,000 beyond the termination date of the Superconducting Super Collider to bring the project to a stage where it could be continued with additional funding.

In 1994, I developed all the parts of the 3D-Flow system, the racks, the mini-crates, the acquisition boards, the mother boards, the daughter boards, etc. The technology then did not offer Gbps serial links as we have today, so I solved the interconnection problem and showed its feasibility with an ingenious combination of mechanics, geometry, stacking connectors, and short flexible printed circuits carrying much data in parallel from each processor to its neighbors, which can be seen in the photos of the system. The calculations of different parameters (desired speed, power dissipation, etc.) showed its functionality. I had many considerations when designing the system to make it practical and feasible. I designed the cylinder structure so that it could be easily assembled and accessed in the event there needed to be repairs made by providing an opening along a line wall accessible on both sides. Its cylindrical structure reflected the cylinder structure of the calorimeter and tracking detectors would have given maximum efficiency by allowing every detector element to communicate within a short distance of five inches to its neighbors in the adjacent mini-crates.

My 3D-Flow invention was also published in the DOE Technology Transfer book DOE/LM-002, DE94005148 in 1994. In 1995, I was awarded a grant from DOE of $906,000 to develop the simulator in C++ at the system level of several thousands of 3D-Flow processors, down to the gate level in VHLD. The funding covered the cost to pay Synopsys, a major company renowned for designing ASIC, to generate the tape-out files to be sent to the foundry to make a 3D-Flow chip with 4 processors in 350 nanometer technology; however, the NRE to produce the chips was never provided. Therefore, at my own expense, I built the 3D-Flow system in hardware using FPGAs in two modular boards, each with 68×3D-Flow processors that demonstrated the feasibility and functionality of building a 3D-Flow system for detectors of any size.

The evolution of technology since 1993 now allows the 1,280 channels 3D-Flow cylinder 1 m×1.8 m to shrink to a 19 inch×19 inch×28 inch box of electronics for 8,192 channels with 43,008×3D-Flow processors consuming less power and having greater overall performance.

Even so, the 3D-Flow system in the cylindrical structure design of 1994 not only had the capability to execute experimenters' programmable desired complex OPRA Level-1 Trigger algorithm with zero dead-time to satisfy today's needs, but could have replaced economically the thousands of electronic boards and many crates of electronics. It even shows superiority when compared to the design of the future upgrades of the trigger with FPGA as described in the 2013 documents of CMS and Atlas experiments.

A detailed analysis of the implementation of the 3D-Flow system with current more advanced nanometer technology, paying attention to the feasibility of the chip, fitting over 40,000×3D-Flow processors @$1 each in one crate made of eight boards (replacing the many crates of other trigger designs) and paying attention to the design details of each board, their interconnection in the crate and interface to the raw-data received from the detector through a Patch-Panel PRAI-ATCA, will show its enormous advantages in performance, power consumption, and cost to any FPGA design for other Level-1 Trigger projects planned by Atlas and CMS.

Recently, I had the opportunity to study the state-of-the-art FPGA technology by the two major companies and their projection of the new components to be delivered next year. Without diminishing the value of FPGA technology, which has great advantages when solving problems in many fields, in this specific application its advantages do not measure up or even come close to the advantages of the 3D-Flow architecture and invention. The 3D-Flow has the capability to execute on each of the 40,000 plus processors in parallel a different programmable sequence of steps/instructions with a high-speed, short latency and low power consumption data exchange capability between adjacent processors and executing up to 26 operations such as add, subtract, compare with 24 values, etc. in less than 3 nanoseconds.

A 8,192 channels Level-1 Trigger system built using FPGA would not fit in a crate, the power consumption and cost would be exorbitant, and it would never come close to having the capability to execute Object Pattern Recognition Algorithms with the same complexity as the 3D-Flow system, directly on raw trigger data received at high speed at each of the thousands of processor nodes.

This can be proven by having funding agencies organize a meeting between myself and the designers of the other Level-1 trigger system built with FPGA where each of us provides calculations and references to technology supporting expected results to prove the global overall performance of the Level-1 Trigger system feasible.

It will be impossible for a Level-1 Trigger system built with FPGA to provide the same performance as the 3D-Flow to the experimenters' and the consequence will be that they will be limited in executing object pattern recognition algorithms, that will not be efficient in extracting ALL information from radiation, missing the rare particle and not having the capability to filter increase noise and separate pileup events at a higher LHC luminosity.

This will continue to limit experimenters' discovery of new existing particles because the Level-1 trigger cannot extract all their characteristics and likewise will not be able to disprove a theory with certainty because of the same limitations. There will continue to be inconclusive results as occurred after the announcement of the discovery of the Higgs boson-like particle on Jul. 4, 2012, when scientists stated the need to acquire more data to understand the real nature of the new particle found.

Unfortunately, instead of following scientific procedures, implementing a dialogue among scientists, showing transparency in science and responding to legitimate scientific questions, since the year 2000 the scientific community of the major conferences in the field has rejected all the papers I submitted where I have explained the importance of extracting ALL valuable information from radiation at the lowest cost per valid event captured; they have also refused a public dialogue where young scientists, PhD students and senior scientists could present their ideas and question each other publicly in two workshops that I proposed at the same conference in 2014, and continued to reject my papers this year as detailed at the Section “Recent Communications” with reviewers giving non-scientific reasons: <<The abstract and summary appear to be a rehashing of work done 23 years ago. Can't tell if there is anything new or interesting. I am unaware of any experiment that has used the 3D-Flow-OPRT technology since then>>, or this other non-scientific reason <<This is one of those topics “that won't go away”. I would like to hear a discussion about this to “once and for all” settle the question raised—is this approach any good? . . . I think it would be up to the moderators of the session whether they want to take this topic on, or just tell him (once more) to go away>>.

After recognizing the validity of my 3D-Flow invention in a formal scientific review in 1993, after it was acclaimed in many letters by scientists and experts in the field, after it was proven feasible and functional in hardware, after the 3D-Flow system was proven superior to any other approach yet funding went to less efficient and more costly Level-1 trigger systems, after having the proof that thousands of electronic boards and hundreds of crates of electronics of the other funded approaches failed to provide a system with the capability to extract ALL valuable information from radiation wasting taxpayers' money and the time of over 10,000 scientists who were provided with data that led to inconclusive results, after the refusal for 25 years to fund the NRE of the 3D-Flow chip and to complete the construction of a full 3D-Flow system as recognized valuable and started its funding in 1994 ($150,000) and 1995 ($906,000), after the scientific community refused to implement transparency in science and follow scientific procedures, after having prevented the presentations of my research work at the IEEE-NSS-MIC conferences since the year 2000 with the exception of when Ralph James was General Chairman in 2003, after leaders of the same conference prevented a dialogue with students, young scientists and senior scientists in two workshops [³] where they could question each other publicly, after this last rejection of my papers with the clear desire by one of the IEEE reviewers “. . . I would like to hear a discussion about this to “once and for all” settle the question raised—is this approach any good?”, and because the scientific community spending taxpayers' money to build the over $50 billion LHC experiments has been incapable for 23 years to implement this discussion, it would be the call and responsibility of the funding agencies that are handling taxpayers' money to organize a fair PUBLIC merit review that will make the scientific truth for the benefit of mankind emerge.

Ultimately, the final judge in science should be left to the results of an experiment but the funding agencies should ensure that tools required to measure the performance of different trigger systems are funded and built, such as the two proposed LHC TER/DSU (LHC Trigger Event Recorder and Detector Simulator Unit) and the RAU (Results Analyzer Unit) that will provide impartial measurable results among the proposed Trigger Systems. If political, personal or power-related interests cannot find a more cost-effective solution to advance science for the benefit of humanity then it would be justified to build these two Units: DSU and RAU costing less than $50,000, which would prevent $50 billion dollars and years of work by over 10,000 scientists from being wasted.
E. The revolutionary benefits to humanity of extracting ALL valuable information from radiation provided by the 3D-Flow invention when applied to Medical Imaging

One of the most difficult tasks that I have had to face for over a decade is to make reviewers of medical research funding agencies realize the importance of creating a Medical Imaging device capable of cost-effectively extracting ALL valuable information from radiation because this is key to making giant leaps in technological improvements in spatial resolution, sensitivity, lower examination cost, and lower radiation dose, that will provide staggering benefits in saving lives through an effective early detection of cancer and many other diseases when detected at a curable stage, and will help with diagnoses, prognoses, and monitoring the treatment of many diseases while reducing healthcare costs.

Researchers are led by the misconception that the most important feature of all medical devices is spatial resolution [⁴] because they receive such requests from doctors, hospitals, pharmaceutical companies, cancer organizations and leaders in the field who want to measure the smallest tumor size and the minimum tumor shrinkage as a consequence of administering a drug. Consequently, they do not focus on extracting ALL valuable information from radiation but, instead throw away over 90% of the useful data from radiation using a poor geometry design that at the same time misses the signals that would provide them better information on spatial resolution.

What did the most disservice to taxpayers was when I submitted more than ten proposals for over ten years for the development of medical imaging devices having the capability to extract ALL valuable information from radiation that could have already saved many lives and reduced healthcare costs, and reviewers rejected [⁵] these proposals, requesting instead that I modify my proposal for the development of PET detector modules focusing primarily on improving spatial resolution to the detriment of sensitivity.

They suggested I use the traditional economical geometry of the detector (16 cm short detector Field of View) using expensive crystals missing most of the valuable radiation rather than approving my geometry of the detector covering most of the patient's body with economical crystals to extract ALL possible valuable information from radiation (radioisotopes) at the lowest cost per valid signal captured. They still do not understand that my 3D-CBS innovative technology using the 3D-Flow architecture not only extracts all valuable information from the radiation but also maximizes the spatial resolution together with all other parameters providing great improvements in the features of medical imaging devices.

In Medical Imaging applications (see Error! Reference source not found.), the 3D-Flow, one of the basic inventions of the 3D-CBS (3-D Complete Body Screening), together with other inventions I conceived after the year 2000, offers a powerful, cost-effective very low radiation diagnostic tool capable of extracting all valuable information from radiation (radioisotope) associated with biological processes, and provides an unprecedented means to effectively detect anomalies such as cancer and many other diseases in those biological processes at an early curable stage. This has the potential to save millions of lives and significantly reduce Healthcare costs. If the current DOE HEP budget does not allow the funding of large generic R&D projects like the 64×3D-Flow chip, boards and systems, because our duty is to serve our leaders and together serve humanity, separate funding should be sought to implement innovations beneficial to humanity that are supported by calculations and scientific evidence in order not to miss again this opportunity to save money, lives, and to create a powerful tool to advance HEP research.

By using the 3D-Flow architecture in the 3D-CBS (3-D Complete Body Screening) to extract ALL valuable information from radiation by thoroughly analyzing all events' raw data (radiation) received directly from the detector, filtering all spurious events and carrying information regarding different tissue density in X-ray, CT and from the tracer (radioisotope) in the 3D-CBS or in PET, medical imaging will be greatly improved because the extracted characteristics of the radiation can optimize measurements of all parameters at the same time (spatial resolution, sensitivity, energy of the detected particle, etc.)
9. SAVING LIVES: The 3D-Flow System when used in the 3D-CBS (3-D Complete Body Screening) for Medical Imaging will have a significant impact on cancer in both numbers and healthcare costs, causing a sharp downturn in the number of cancer deaths.

The most difficult task that I had to face for over a decade is to make people aware of the benefits to humanity of a Medical Imaging device capable of cost-effectively extracting ALL valuable information from radiation that could have already saved many lives and reduced healthcare costs. The people include political leaders, health care organizations, doctors, hospital administrators, cancer organizations, funding agencies, philanthropists, foundations supporting humanitarian causes, cultural groups and anyone who cares to defeat the most deadly and costly calamity, cancer, and who cares to advance health care with better diagnostic devices that give less radiation to the patient and provide more accurate information to doctors to helping them diagnose, prognoses and monitor treatment.

I believe I would be failing in my duty if I did not draw your attention to the adversities and obstacles that I have faced this past decade that have delayed implementation of my invention capable of extracting ALL valuable information from radiation, and the resulting burden to taxpayers this inaction has brought, not only monetarily but in avoidable deaths and suffering that will continue to occur unless immediate action is taken and the NRE (Non-Recurring Engineering) of the 3D-Flow and 3D-CBS projects are funded.

I hope that newspaper, radio, television and online media journalists as well as social networks feel a responsibility toward those who suffer and die needlessly and will reprint and disseminate this information and the link to this proposal so that everyone, including funding agencies handling taxpayer money or donations, will request a fair PUBLIC merit review that will reveal the scientific truth to benefit humanity.

Philanthropists and/or investors who are interested in making a difference in the world by funding this project and are willing to sign an NDA to verify the specific details of its implementation should contact Crosetto@att.net.

See reference [⁶], [⁷], [⁸], [⁹], [¹⁰], [¹¹], [¹²], [¹³], [¹⁴], [¹⁵], [¹⁶], [¹⁷], [¹⁸], [¹⁹], [²⁰], [²¹], [²²], [²³], [²⁴]

Why is Extracting ALL Valuable Information from Radiation important to improve Early Cancer Detection?

Radiation is related to biological processes, therefore by accurately extracting all valuable information from radiation (on spatial resolution, time resolution, energy and sensitivity) it allows a reduction in the radiation dose to the patient, reduces costs and provides valuable information to doctors on anomalies in morphological changes and in biological processes, enabling improved diagnoses, prognoses and monitoring of the treatment of many diseases, while reducing healthcare costs.

How is ALL Valuable Information from Radiation Extracted?

By implementing a harmonious combination of several features ALL concurring to optimize the execution of Object Pattern Real-Time Recognition Algorithms (OPRA) on thousands of radiation data arriving in parallel from the detector at a very high speed. The 3D-Flow architecture is capable of executing uninterruptable complex algorithms for a time longer than the time between two consecutive input data sets by adding layers of 3D-Flow processors communicating through a bypass switch assuring zero dead-time. I then minimized the time required to exchange data between neighboring elements necessary for the execution of typical 3×3, 4×4, 5×5, . . . Object Pattern Real-Time Recognition Algorithms by implementing in 1994 a cylindrical assembly geometry of the electronics (1 m. in diameter by 1.8 m. tall, using cables with a maximum length of 13 cm) reflecting the shape of the detector. The number of layers of 3D-Flow processors needed is calculated by dividing the time to execute an algorithm by the time interval between two consecutive input data and rounding the result up to the next integer. Because longer cables increase the algorithm's execution time (e.g. exchanging data between processors on different PCB boards connected through a 30 cm cable adds more than 1,000 picoseconds), this proposed system of 14,000×3D-Flow processors is confined to a 16 cm×16 cm×16 cm cube which keeps the number of layers of processors low and consequently lowers the power consumption of the system. The performance of this 3D-Flow system in recognizing objects by analyzing data arriving at a very high speed is far superior to any alternative system built with FPGA (Field Programmable Gate Array). This is because in FPGA a lot of silicon area with electronic circuits which are not optimized for OPRA consume power, requiring a larger system and longer cables, thus never able to achieve the performance and lower cost of the 3D-Flow.

How much does the 3D-Flow OPRA system cost to extract all valuable information from radiation to make a revolution in an effective early detection that could save many lives and reduce healthcare costs?

A 2,304 channels 3D-Flow for the 3D-CBS device for early cancer detection is very competitive with other instrumentation such as oscilloscopes and Logic State Analyzers. As you can see from Table 1, Table 2 and Table 3, the cost per channel supported by the quotes from reputable companies reported in the budget justification is much lower than oscilloscopes and Logic State Analyzers.
C. Justification of the Proposed Project

The proposed project is justified from the need to create a powerful tool with the capability to extract all valuable information from radiation more specifically to execute experimenters' programmable complex Object Pattern Real-Time Recognition Algorithm at the Level-1 trigger with neighboring data exchange while sustaining an input data rate of 80 MHz from 8,000 channels, each receiving a 16-bit word (or 40 MHz with a 32-bit word, or 20 MHz with 64-bit word) with zero dead-time. This proposed project is also justified by the need to develop a similar powerful tool capable to extract all valuable information from radiation in the application of medical imaging to reduce the radiation dose to the patient, to enable an effective early cancer detection and reduce healthcare cost.

An aggressive schedule following a prompt funding could target the installation of the first prototype in one of the large experiments at CERN before Jan. 31, 2018. In the event CMS and Atlas management will not agree to adopt the 3D-Flow trigger, arrangements should be made to derive the 8,192 trigger signals from the two experiments and send them in parallel to the ATCA-PRAI crate (Patch Panel Regrouping Associates Ideas, see FIG. 4) that will transfer the trigger data at 1.3 TB/sec to the 3D-Flow system consisting of 43,008×3D-Flow processors to work in parallel to the official trigger system.

In the event it is not possible to split the signals of trigger raw data and send one to the official Level-1 Trigger and the other to the 3D-Flow system it will still be fine to receive a copy of the data buffered from the official Level-1 trigger. Even if these data will arrive to the 3D-Flow with a latency, I am confident that the 3D-Flow system can process the raw data and generate a Global Level-1 trigger decision within the time of a few microseconds allocated for Level-1 Trigger decision.

No matter how many superlative adjective one can use to describe success, experimental results tell the truth that only 40 Higgs boson-like events found on Jul. 4, 2012 out of 100,000 generated by LHC is a failure of the Level-1 Trigger. Moreover, these 40 Higgs boson-like events were not found by the Level-1 trigger but from analysis of trillions of data recorded casually.

These facts & data are confirmed by CERN slides and admitted in CERN-Atlas and CERN-CMS experiments upgrade official documents and it is also stated by the presenters of the trigger of the large experiment at CERN at the IEEE conference. See references: [²⁵], [²⁶], [²⁷], [²⁸], [²⁹]
10. Excerpts from my attempt to address scientifically and analytically the discrepancy between the Higgs boson-like measurements of 125.3 GeV (CMS) and 126.5 GeV (Atlas) with the General Chairman of the 2014 IEEE-MIC conference; instead of addressing the discrepancy analytically, he tried to justify it with an inconsistent statement and then walked away

My attempts to follow scientific procedures discussing issues based on the analysis of equations, calculations and scientific evidence was denied when I proposed two workshops at the 2014 IEEE-NSS-MIC conference. I was stopped by the General Chairman, Anthony Lavietes of the 2014 IEEE-NSS-MIC-RTSD conference from asking a legitimate question to the NSS keynote speaker about the discrepancy between CMS 125.3 GeV and Atlas 126.5 GeV measurement of the energy of the announced Higgs boson-like particle.

When I asked Lavietes after the event why he stopped me from addressing this issue with the keynote speaker he told me because I was wrong. He said that CMS and Atlas measurements are very accurate and that they have made extensive measurements.

He stated that even the same instrumentation never gives the same measurement when performed at different times because differences in temperature, humidity, etc., can make a difference at 15 figures after the zero.

I agreed with him, but said CMS at 125.3 GeV and Atlas at 126.5 Gev have a 0.9% difference in their measurements and not 0.000000000000009%.

At that point, instead of resolving the issue based on the evidence of these calculations, Lavietes ran away with the excuse that he had other important things to do. . . . and in 2015 my papers submitted to IEEE-NSS-MIC were again rejected with claims of “propaganda” but refusing to discuss my equations, calculations and scientific evidence that would demonstrate the solid scientific evidence which is not propaganda.
F. Excerpts of my answer to the Director of DOE-HEP's request dated Aug. 4, 2015

The following is an excerpt from my 20 page answer to Dr. Siegrest's request to provide “the most important ingredient” supporting a “compelling case for you [me] to visit” that I sent on Sep. 20, 2015. (See Dr. Siegrist's original request on page Error! Bookmark not defined.)

Thank you Jim for answering my email and inviting me to submit a “compelling case for you [me] to visit.” My highly compelling case is summarized in the following paragraph, presented at many conferences, published in articles, and sent in emails, as in the material related to my last abstract, slides and presentation that I sent to you in my email on Jul. 16, 2015:

“The innovative 3D-Flow parallel-processing architecture, and fault-tolerant system, recognized valuable by major, public, scientific reviews, proven feasible and functional in hardware, breaks the speed harrier in real-time applications such as in High Energy Physics (HEP) and Medical Imaging. It is flexible, scalable, programmable, modular, technology-independent meaning it can migrate to the most advanced and cost-effective technology, improving since 2003 its speed by over 13 times, lowering power consumption to 1/10 and lowering the cost to less than 1/200. In addition the 3D-Flow innovative architecture offers system performance at a speed higher than the technology performance. It offers the most cost-effective and powerful tool to researchers in High Energy Physics to discover new particles and can save many lives with a cost-effective early cancer detection tool. In HEP applications, the 3D-Flow architecture, is capable ofexecuting experimenters' desired programmable complex Object Pattern Recognition Level-1 Trigger (OPRT) algorithms, while sustaining an input data rate of over 80 million events per second from over a billion collisions per second, with zero dead-time, at a lower cost (compared to current approaches) per each good event captured and fully meeting 1994 LHC experiment requirements, 2012, today and future requirements for a higher LHC luminosity. In Medical Imaging applications the 3D-Flow as one of the basic inventions of the 3D-CBS (3-D Complete Body Screening), offers a powerful, cost-effective diagnostic tool capable of extracting all valuable information from radiation (radioisotope) associated with biological processes and provides an unprecedented means to effectively detect anomalies in biological processes such as cancer and many others diseases at an early curable stage, administering a very low radiation dose and a very low examination cost. This has the potential to save millions of lives and significantly reduce Healthcare costs. This is not propaganda or advertisement. Each statement above is supported by the 3D-Flow feasibility and functionality proven in hardware, by calculations, by correct equations, by scientific evidence. The speed, silicon die size, cost, power consumption from nW/MHz/gate to the power consumption of the 3D-Flow chip, to the power consumption of the VME 9U or 6U electronic boards, to the kW consumption in a crate, for a specific technology are confirmed by the parameters specified by world's major companies, achievable with their technology and manufacturing process to make Integrated Circuits, electronic boards and crates. If the current DOE HEP budget does not allow the funding of a generic R&D project like the 64×3D-Flow chip, boards and crate, because our duty is to serve our leaders and together serve humanity, it will be important for everyone to inform them to provide separate funding and the media inform the public about innovations beneficial to humanity that are supported by calculation and scientific evidence in order not to continue to miss this opportunity to save money, lives, and to create a powerful tool to advance HEP research”
11. The entire Level-1 3D-Flow Readout-Processing Boards and Global Trigger for 8,192 Trigger Towers can be implemented in one of the following platforms:
1. Eight 1024 channels 3D-Flow boards 9U VME, 366 mm×400 mm with the capability to execute up to 30 Object Pattern Real-Time Recognition Algorithm 3D-Flow steps @40 MHz 32-bit input data rate or 15 steps at @80 MHz 16-bit input data rate and one 3D-Flow Global Trigger board. The boards consist of eight 9U VME Readout-Processing boards with partial pyramid for channel reduction and one 9U VME Global Trigger board with the final section of the pyramid, the calculation of the global quantities that are provided to the Global Trigger for the final Level-1 trigger decision. The global 3D-Flow system can sustain 80 million events per second from over a billion collisions per second, with zero dead-time. Each of the 8000 input channels receive 16-bit data every 12.5 ns. Each 3D-Flow program step has the capability to perform up to 26 operations of addition, subtraction, compare with multiple values, etc. This provides a complete, thorough Object Pattern Recognition capability using information from multiple detectors (calorimeter, tracking, etc.).
   OR
2. Sixteen 512-channels 3D-Flow boards 9U VME, 366 mm×400 mm with the capability to execute up to 70 Object Pattern Real-Time Recognition Algorithm 3D-Flow steps @40 MHz 32-bit input data rate or 35 steps at @80 MHz 16-bit input data rate and one 3D-Flow Global Trigger board. The boards consist of sixteen 9U VME Readout-Processing boards with partial pyramid for channel reduction and one 9U VME Global Trigger board with the final section of the pyramid, the calculation of the global quantities that are provided to the Global Trigger for the final Level-1 trigger decision. The global 3D-Flow system can sustain 80 million events per second from over a billion collisions per second, with zero dead-time. Each of the 8000 input channels receive 16-bit data every 12.5 ns. Each 3D-Flow program step has the capability to perform up to 26 operations of addition, subtraction, compare with multiple values, etc. This provides a complete, thorough Object Pattern Recognition capability using information from multiple detectors (calorimeter, tracking, etc.).
   OR
3. Thirty-two 256 channels 3D-Flow boards 6U VME, 233 mm×160 mm with the capability to execute up to 50 Object Pattern Real-Time Recognition Algorithm 3D-Flow steps @40 MHz 32-bit input data rate or 25 steps at @80 MHz 16-bit input data rate and one 3D-Flow Global Trigger board. The boards consist of thirty-two 6U VME Readout-Processing boards with partial pyramid for channel reduction and one 6U VME Global Trigger board with the final section of the pyramid, the calculation of the global quantities that are provided to the Global Trigger for the final Level-1 trigger decision. The global 3D-Flow system can sustain 80 million events per second from over a billion collisions per second, with zero dead-time. Each of the 8000 input channels receive 16-bit data every 12.5 ns. Each 3D-Flow program step has the capability to perform up to 26 operations of addition, subtraction, compare with multiple values, etc. This provides a complete, thorough Object Pattern Recognition capability using information from multiple detectors (calorimeter, tracking, etc.).
   Many 9U Crates, Current Level-1 Trigger OR Two 6U Crates with 32×3D-Flow 256 Ch. Boards.

The 3D-Flow is able to extract all valuable information from radiation to discover new particles and save billions of dollars in physics research with a box of electronics containing 50,000×3D-Flow processors costing less than $1 each.

The study, verification of feasibility and functionality with FPGA and with the silicon foundries that can produce the 3D-Flow processor at less than $1 per processor and the 3D-Flow system in one box of electronics has been done.

Results are available to be PUBLICLY discussed and verified to technological detailed implementation of a silicon foundry that requires funding agencies or philanthropists interested to fund the NRE to sign an NDA to disclose the technology that can build the 3D-Flow processor at less than $1 per processor and a 3D-Flow system of 8000 channels in one large crate and 2,304 channels in a smaller box with the unprecedented capability to extract valuable information from radiation.

Both systems will have the capability to extract all valuable information from radiation by to acquiring and processing data in real-time with Object Pattern Real-Time Recognition Algorithms, sustaining an input data rate of over 80 million events/second from thousands of channels each providing a 16-bit word every 12.5 nanoseconds, or at 20 million events/seconds with 64-bit information every 50 nanoseconds.

In a nutshell, this opportunity has been available since 1992; however, funding for NRE was never provided. After my invention in 1992 it was recognized by many scientists who wrote letters of appreciation. In 1993, it underwent a formal scientific review at FERMILab gaining further recognition. In 1994, my invention that could be implemented in a cylinder of electronics that was 2.2 meters tall and 2 meters in diameter was published in the DOE Technology Transfer book as a breakthrough invention benefitting Science supported by DOE. In 1995, DOE funded the study of the implementation in 350 nanometer technology but not the NRE to build the chip in silicon. In 2001, I proved the 3D-Flow functionality in FPGA and presented it at the IEEE-NSS-MIC conference in San Diego, Calif. In 2003, I built the industrialized version of 68×3D-Flow processors on a board. With today's technology, the cylinder of electronics published by DOE in 1994 can be implemented in a single crate with 50,000×3D-Flow processors with a power exceeding the requirements of LHC or even a higher luminosity that will be reached 15 years from now.

After the opportunity was missed, cabled logic electronics or electronics with very limited programmability or having limited capability to sustain an input data rate of 40 million events/second, without zero dead-time, and without the capability to execute complex Object Patter Recognition Algorithms, were built instead.

The result was that the CMS and Atlas experiments captured only about 40 of the 100,000 Higgs boson-like particles produced by the LHC. In the 2013 official documents for the upgrade of the Level-1 Trigger, they have announced they plan to replace the inadequate electronics.

Specifically

This proposal addresses the important ingredients of the abstract and of my past presentations and documents published that can provide great advances in science, reduce the cost of High Energy Physics experiments and benefit humanity in several areas. I have proof of concept in hardware presented for the first time at the 2001 IEEE-NSS-MIC conference in San Diego, Calif., where attendees were able to verify the functionality of the 3D-Flow by selecting a cluster pattern on switches and verify the expected result displayed on LED, and the timing to execute the real-time algorithm was displayed on the oscilloscope. I have extracted a few slides and provided references to examples of 3D-Flow Object Pattern Real-Time Recognition Algorithms for Level-1 Trigger which can help reviewers understand the benefits of my invention.

I will now address the content of the long paragraph above; my calculations, equations and scientific evidence supporting them will make the compelling case a discussion with reviewers where I can answer their questions, clarify any doubts and show how my 3D-Flow invention can greatly advance science and benefit society.

The words “. . . capable of executing experimenters' desired programmable complex Object Pattern Recognition Level-1 Trigger (OPRT) algorithms” were recognized in several letters by top experts in the field when praising my innovative 3D-Flow architecture. This is reported in the attachment, which includes the letter dated Nov. 5, 1993, from Andy Lankford, former Deputy Spokesman of CERN-LHC-Atlas experiment, the letter dated Feb. 11, 1993, from Livio Mapelli, leader of the same experiment at CERN, the letter dated Feb. 27, 1995, from Joel Butler, head of the computing division at FERMILab, and in many other letters. Lankford wrote: “. . . a technique to perform fast, programmable triggers”, while Mapelli wrote: “. . . higher flexibility, coming from the programmability of algorithms and not only of parameters . . .” and Butler, one of the organizers of the major FERMILab review of my 3D-Flow invention wrote: “the 3D-Flow project is the only detailed study demonstrating the feasibility of executing several level-1 trigger algorithms of different experiments.” These are clear statements recognizing the capability (and later confirmed feasible and functional in hardware) of the 3D-Flow system to execute Object Pattens Recognition Trigger (OPRT) algorithms not possible at level-1 trigger before my invention. I invite anyone to provide a reference to any system developed during the past 23 years which has some level of programmability and compare the system's flexibility, performance and cost-effectiveness to the 3D-Flow system. On page 13 of the 2013 CMS and Atlas Level-1 Trigger upgrade official document, they admit their current Level-1 Trigger does not have OPRT capability and will be trashed. They plan to implement OPRT over the next 15 years making use of FPGA which is hundreds of times less efficient, less performant and more costly than my 3D-Flow solution.

The words: “while sustaining an input data rate of over 80 million events per second from over a billion collisions per second . . .” As you will see from the evidence presented at the end of this document, current technology allows me to build a 3D-Flow system with the capability to surpass, at zero dead-time, the requirements of 80 MHz that is planned for the LHC to run a few years from now. The 3D-Flow architecture would have also surpassed the 20 MHz requirements of the LHC for most of the runs in 2011-2012 with the 3D-Flow FPGA boards running at 31 MHz, and the 3D-Flow version in 350 nm standard cell technology running at 61 MHz compiled with Synopsys tools in 1996 (but without funding it was never built in silicon) would have fully satisfied with zero-dead-time the requirements of the few runs of the LHC in late 2012 at 40 MHz bunch crossings. All the 3D-Flow versions in FPGA or Standard cell 350 nm technology had the capability of analyzing ALL billion collisions per second.

I invite anyone to provide a reference to any system developed during the past 23 years which can cope with even 40 million events per second or one which does not have limitations in handling a billion collisions per second. This is no problem for the 3D-Flow architecture which can satisfy these requirements even with technology of 25 years ago when it first was announced.

The words: “with zero dead-time . . .” The 3D-Flow architecture clearly described in one page and simulated in an analogy by high school students show that data from all bunch crossings, with zero dead-time are thoroughly analyzed, even if the technology is slower than the input data rate. Even nowadays there is not a Level-1 trigger system having zero dead-time, and even those designed and built for the upgrades of the Atlas and CMS experiments at LHC for the next 15 years do not provide zero dead-time. The presenters of Atlas and CMS papers at the Trigger Session of the 2013 IEEE-NSS conference in Seoul, South Korea, even admitted that when Level-1 Trigger fires, the system is dead for 3 to 4 bunch crossings. What if during those bunch crossings the rare Higgs boson-like event which occurs only once every 10 billion events is missed? It was instead recognized in 1992 that my 3D-Flow invention provides the zero dead-time system capability. At the NSS-N5-4 presentation at the 2013 IEEE-NSS-MIC-RTSD conference, it was stated that the Atlas L1 Trigger be improved to cope with higher rates (slide 17). Slide 16 states that “no change to the detector is needed. Full replacement of front-end and back-end electronics” was needed instead. This was not because of aging radiation limits. The limitation of the current Atlas trigger was admitted.

The words: “. . . at a lower cost . . . ,” the 64×3D-Flow processors on a chip cost less than 1/200 the price of the 64×3D-Flow processors on the board I built in 2003 and displayed later in this document.

Each group of words in the long paragraph needs to be addressed in depth. However, when it comes to the words referring to Medical Imaging applications the urgency to address all words are even more important because it will prevent more years of lives from being lost which could have been saved with an effective early cancer detection that I made available more than a decade ago.

The words: “. . . offers a powerful, cost-effective diagnostic tool capable of extracting all valuable information from radiation associated to biological processes . . .” is not up to the competence of doctors but the competence of scientists expert in particle detection, because for a doctor in medicine it would be difficult to grasp the magnitude of the benefit of the invention providing the capability to extract more efficiently information from radiation. It is competence of scientists expert in particle detection understand the advantages of the right column “3D-CBS with innovations” with respect to the column to its left “Current PET & PET/CT” of FIG. 1 on page 2 of the article “The 3-D Complete Body Screening (3D-CBS) Features and Implementation” compares the advantages of the right column “3D-CBS with innovations” to the left column “Current PET & PET/CT”. It would take a scientist expert in particle detection to fully understand these advantages and the benefits of executing a 3×3 matrix algorithm with the 3D-CBS device instead of a 2×2 anger logic algorithm calculated on current PET devices. The 3×3 can provide more accurate information on the photon's total energy, rejecting more efficiently scatter events and capturing more good 511 keV pairs of photons. It would also allow an increase in spatial resolution interpolating the location of the incident photon in the crystal with information from detector elements to the left and to the right (up and down for “y” coordinate calculation) of the position where the photon hit the detector.

The capability to execute complex algorithms as shown in section “r” (bottom right of FIG. 1) allows to extract more information from economical crystals, to improve photon arrival time from economical crystals with long decay time, thus lowering the cost of the 3D-CBS device with a detector having a FOV ten times longer than current PET but costing only twice as much and capable of capturing hundreds of times more the number of signals from the tumor markers (photons emitted from the radioisotope). This makes the examination shorter, increases throughput which further lowers the examination cost, reduces the radiation to the patient and enables an effective early detection.

The entire layout of the 3D-Flow system to extract more valuable information from the radiation (radioisotope) associated to the tumor markers to provide accurate information about minimum anomalous biological processes in the body is explained in a figure later in this document. It would be important to discuss with medical doctors afterwards the implications of having higher capabilities to follow biological processes. However, until they understand the advancements in tools to detect particles, it is difficult to explain to them the advantages of breaking the speed barrier in real-time applications, or executing Object Pattern Real-Time Recognition Algorithms on signals arriving from radiation administered to the patient that if not captured is lost to the disadvantage of the patient and the doctor. If improving particle detection is key to improving early detection of cancer and other diseases that can save many lives, then it is essential to address in depth what is improving particle detection to provide benefits to humanity in saving lives and reducing Healthcare costs.

Jim, I see below that on Aug. 3, 2015 you copied Glenn on my email with links to the abstract, slides and video of my last presentation at the international conference “Energy Challenges & Mechanics” on Jul. 7-9, 2015 in Aberdeen, Scotland. Please, Glenn and Jim, let me know if there is anything in my explanation of my invention that is not understandable. I will be glad to answer any questions you may have.

Here are a few references among thousands of pages of documentation that could help your colleagues understand the development of my invention, its verification, advantages and benefits:
Jim Siegrist (and many others who wrote letters of appreciation of my invention and approved its publication and dissemination) included my article in the book “Calorimetry in High Energy Physics”, edited by you and published by World Scientific in 1993, after I presented it in Corpus Christi, Tex., in 1992, the scientific community was informed about my invention. As you may recall, on page 555 of your book “Calorimetry in High Energy Physics” reporting my article, I describe the Trigger Tower, on page 557 I give algorithm examples that could be executed by the 3D-Flow at the Level-1 Trigger, and on page 562 I invite Glenn to follow the steps of the assembly code of the OPRT 3D-Flow Level-1 Trigger algorithm “to find Eₜ, electrons, isolations and jets”. This created awareness to the point that the same year the Director of the Superconducting Super Collider requested a major formal scientific review of my 3D-Flow invention by world experts, which included a representative from CERN, and experts from universities and industries in the field to be held at FERMILab. I passed the review and my invention was officially recognized in a written report. I received $150,000 to document my work so it could be continued under another funding project. The FERMILab report of the formal review on page 2 officially recognized my invention1 as being a “unique concept” that went beyond the imagination of future science as stated on page 6 “. . . experimenters would probably think of clever uses not now possible”. A few years later, I confirmed this vision by demonstrating the feasibility aad functionality of the inventive concept in hardware. In presenting a poster and an article at the 2013 IEEE-NSS-MIC-RTSD Conference, where I documented the history of this innovation.
In 1995, I presented improvements I had made in the 3D-Flow trigger algorithm for detecting electrons in the LHCb experiment that were published on pages 44-48 in the proceedings of the 1995 IEEE Conference on real-Time Computer Applications in Nuclear Particle Physics held in East Lansing, Mich. I would like to invite Glenn to look at page 48 of these proceedings to see how the 3D-Flow OPRT trigger algorithm for detecting electrons at the Level-1 Trigger was improved from my previous publications. The steps are described in small diagrams showing the data flow and operation performed in the 3D-Flow mnemonic assembler and in hexadecimal code.
In 1998 Eric Eisenhandler at the Fourth Workshop on Electronics for LHC Experiments in Rome, Italy, presented an overview of the “Hardware Triggers at the LHC” and on page 53 of the proceedings reviewed the 3D-Flow system application for finding high-pT electrons, photons, hadrons and muons at Level-0 trigger. A more detailed description of the 3D-Flow Level-0 tracking trigger application can be found in an article I wrote at pp. 625-627 in 1993 and presented at the June 21-July 2, Snowmass Workshop on B Physics at the Hadron Accelerators and another article I wrote with Sergio Conetti, G. Corti and B. Cox from the University of Virginia. (See “An implementation of the L0 muon trigger using the 3D-Flow, note LHCb 98-001).
I received DOE SBIR Phase I and Phase II grants totaling $906,307 from Sep. 1, 1995 to Mar. 23, 1998, and as a result I was able to further demonstrate the feasibility of my invention by developing simulators, assemblers, and software tools, and by improving the architecture of the processor, as well as generating test vectors which targeted three different FPGA technologies (ORCA from Lucent Technologies, Xilinx and Altera) and a standard cell/gate 350 nm technology. However, building it in silicon would have cost a lot more than I had. The grants were used to pay my employees for over three years, to pay for the most experienced consultants in IC design like Synopsys who did the porting of four 3D-Flow processors to a chip into the 350 nm Standard cell/gate technology, and allowed the purchase of expensive tools from Cadence such as Concept HDL to enter schematics, Allegro to design printed circuits boards (PCB), and Spectra for auto-routing.
In 1999, to document the result of my work as requested by DOE grant, I wrote a comprehensive 45-page article that was published in the peer-reviewed scientific journal Nuclear Instruments and Methods in Physics Research A 436 (1999) pp 341-385. It describes a complete application of the 3D-Flow system for the Level-0 trigger of the LHCb calorimeter. Unfortunately, that same year, DOE decided to support only projects for CMS and Atlas LHC experiments and cut the funding to projects like mine targeted to the LHCb experiment. I invite Glenn to read my NIM article found on page 353, Section “6.1 3D-Flow mixed-signal processing board” where I describe the 3D-Flow steps of the LHCb Level-0 trigger algorithm. Also that same year, I presented the 3D-Flow design advantages when targeted to current and future technologies used in other applications, at the 11^{th} IEEE NPSS Real Time Conference in Santa Fe, N. Mex., Jun. 14-18, 1999, comparing cost and performance with other approaches adopted by other experiments such as CMS, LAL and HERA-B. I presented the software tools I had developed using the DOE grants. I invite Glenn to read the proceedings of this conference beginning on page 338, “Figure 3, The evolution of IC design” describing how the technology-independent 3D-Flow design can migrate to future technologies; page 331, FIG. 4 showing the interrelation between 3D-Flow software tools in the Real-Time Design Process of 3D-Flow systems; page 335, “Table 1 Trigger cost implementation comparison between hardwired systems and the 3D-Flow programmable system”; and page 336, “Table 2, Fast data acquisition and processing implementation: Features and Performance” comparison between the 3D-Flow system and CMS, LAL and HERA-B.
Because I was seeing undeniable proof from calculations, simulations and recognition from experts in the field that my invention can advance science and benefit humanity, I used all the DOE grant money including the amount that had been designated as my salary to further advance the research. I continued to use my own money and contributions from friends to purchase components and to demonstrate the feasibility of my invention in hardware. The FPGA company Altera provided free of charge two prototype boards with two large FPGA where I tested the first eight 3D-Flow processors that I presented at the 2001 IEEE-NSS-MIC conference in San Diego, Calif.

In 2000, I wrote a technical-scientific book “400+ times improved PET efficiency for lower-dose radiation, lower-cost cancer screening” describing in detail the implementation of a 3D-Flow system in VME and IBM PC for High Energy Physics and Medical Imaging applications and I distributed 200 free copies of the book to the leaders and senior scientists who attended the 2000 IEEE-NSS-MIC conference in Lyon, France, where I also presented two articles “A modular VME or IBM PC based data acquisition system for multi-modality PET/CT scanners of different sizes and detector types” and “Real-time, programmable, digital signal-processing electronics for extracting the information from a detector module for multi-modality PET/SPECT/CT scanners.” Based on the positive test results of the 3D-Flow small system of eight 3D-Flow processors on two FPGA Altera prototype boards, I moved forward, again with my own money and contributions from friends, to design and build two modular IBM PC boards as described in the book, each with 68 processors. The successful testing of the communication between these two modular boards proved that a 3D-Flow system for any detector size in HEP or in Medical Imaging applications can be built, with the advantages of extracting all relevant information from radiation at the lowest cost per valid signal captured, to discover new particles, and to implement an effective, low-dose radiation, low-cost early cancer detection. I invite Glenn to look on page 114 of my technical scientific book to see the 3D-Flow photon detection algorithm for finding the local maxima, and calculating the total energy on a 3×3 matrix, and page 116 where the same algorithm calculates the cluster on a 5×5 matrix in 9 steps.
In March 2008, after being invited to a meeting at CERN with former CERN General Director, Horst Wenninger, and other experts in particle physics appointed by Antonio Zichichi to verify she solidity of my invention and technology, I was further invited to give an all-expense paid seminar in August 2008 before world leaders, including Nobel Laureates at the World Laboratory's 40^{th} Session of “Planetary Emergencies”. (My presentation with Q&A was video recorded and my article “Logical Reasoning and Reasonable Answers Consistent with Declared Objectives for the Benefit of Mankind” was published by World Scientific, 2009, pp. 531-560). Glenn please let me know if this video could be useful material for the determination of a compelling case and I will provide it
In 2008, I gave a seminar at CERN from its CMS video conference room which was broadcasted to the world via EVO system. The following day, I had a one-on-one meeting with CERN Director General and leaders of the Trigger of the main experiments: CMS, Atlas and Alice (I was familiar with LHCb because I was part of it until 1999). No one could refute the superiority of my 3D-Flow programmable parallel-processing architecture compared to current trigger developments or it being the only cost-effective solution that could fully satisfy the trigger needs of all experiments. Glenn please let me know if this video could be useful material for the determination of a compelling case and I will provide it. Please also look at pages 15 to 17 of the article published by World Scientific reporting this event and the communications with top leaders in the field of Triggers for large experiments.
In September 2009, I was invited to give a Seminar at Brookhaven National Laboratory before experts in Physics, Biology and Medical Imaging. Among the audience was Dr. Joana Fowler who had just received the National Medal of Science award from U.S. President Barak Obama. This event was possible because of the interest of Ralph James, Associate Director of BNL, who contacted the people at BNL who organized this seminar. No one could find any flaws or refute the advantages of my invention. The entire event lasted about two hours and was video recorded, including the Q&A session. Glenn please let me know if this video could be useful material for the determination of a compelling case and I will provide it.

After receiving Jim's email inviting me to submit a compelling case for me to visit, I met with Ruben Sonnino, former Vice-President of STMicroelectronics, the fifth leading company in semiconductors in the world, and we contacted five among the best IC design houses in the world and the largest silicon foundries for semiconductors to find the most cost-effective approach to build the 3D-Flow chip with 16×, 64×, or 256×3D-Flow processors in a chip targeted to different technologies from 180 nm down to 40 nm. After several conference calls with their offices in different locations in Europe and in the U.S., we selected two quotes.

After assessing the available technology, we realize we can offer the High Energy Physics community a programmable 3D-Flow system for level-1 Trigger that will be a very powerful and cost-effective tool for discovering new particles. It will be invaluable to experimenters in executing their desired complex real-time algorithms, surpassing the requirements of any present or future experiments even at bunch crossings greater than 80 MHz, and a luminosity even higher that what is foreseen for LHC experiments in the next 15 years.

The 3D-Flow system is basically a “decision box” or “Trigger” having the task to process accurately and at great speed each high resolution image and decide whether to keep it or trash it without the possibility to store all of them because in one day the data would fill all the hard drives on the planet.
Glen, earlier I highlighted for you in purple several examples of Object Pattern Recognition Trigger (OPRT) algorithms coded in “steps” that can be executed in a single cycle by the 3D-Flow processors. This was because these powerful “steps” (or 3D-Flow instructions) are key tools in giving experimenters the power to nail down their new particle and detect it among an increasing background noise that occurs with an increased luminosity of LHC. These instructions/steps can also resolve pileup by detecting a slope change in the signal. Many experts in the field including Nick Ellis, Andy Lankford, Livio Mapelli and Joel Butler understood and recognized over 20 years ago the power of the 3D-Flow architecture that can execute several of these programmable “steps”. Several put it on writing like Joel Butler who wrote: “the 3D-Flow project is the only detailed study demonstrating the feasibility of executing several level-1 trigger algorithms of different experiments.” For this reason I also highlighted in the specifications of the “3D-Flow decision box, or Trigger” the number of “steps” different configurations of 8 boards 9U, 16 boards 9U or 32 boards 6U can perform.

The specifications and requirements as listed in the basic documents (page 124, last paragraph) and in the 2013 upgrade (page 79, Section 7.2.1) for the Level-1 Trigger of the largest, billion dollars experiments at LHC consist of 8,000 trigger towers (or channels) which were each receiving up to 10-bit information every 50 ns (20 MHz) until 2012, then 25 ns (40 MHz) in the current run with the prospect of up to 12.5 ns bunch crossings (80 MHz) in future upgrades.

Current requirements of the Level-1 Trigger for LHC experiments:
8000 channels or Trigger Towers with a detector granularity of Δ_{η}×Δ_{φ}=0.1×0.1
8 to 10-bit information of the E_{T} from the 8000 Trigger Towers in the Hadronic, Electromagnetic, and HF calorimeters every 25 ns
40 MHz bunch crossing
Capable of executing simple algorithms with limited programmability and performing simple operations that do not support a thorough object pattern recognition

The 3D-Flow system exceeds all current requirements of the Level-1 Trigger for LHC experiments but because it is technology-independent, it can increase its performance as needed to satisfy future upgrade requirements, increasing the Triggers' power to discover new particles.
8000 channels or Trigger Towers with a detector granularity of Δ_{η}×Δ_{φ}=0.1×0.1
16-bit information of the E_{T} from the 8000 Trigger Towers in the Hadronic, Electromagnetic, HF calorimeters, and from tracking detectors and other detectors with a detector granularity of Δ_{η}×Δ_{φ}=0.1×0.1, that can provide useful information for Level-1 Trigger every 12.5 ns
80 MHz bunch crossing
Capable of executing complex algorithms with full programmability from 30 to 70 steps @40 MHz input data rate or 15 to 35 steps @80 MHz input data rate. Each step with the capability to perform up to 26 operations such as addition, subtraction, comparison with multiple values, etc. This provides a thorough object pattern recognition capability using information from multiple detectors (calorimeter, tracking, etc.).

If necessary, it can handle a higher input data rate or more information at each of the 8000 input channels exceeding the 16-bit every 12.5 ns. For example, if additional information from tracking or other sub-detectors could be useful at the Level-1 Trigger decision, the 3D-Flow system can be designed to handle 24-bit or 32-bit words received from each of the 8000 channels every 12.5 ns.
The staggering cost-performance advantages of the 3D-Flow system compared to other trigger approaches is because:

The tested functionality of the 64×3D-Flow processors housed in 16 FPGA chips, each with 4×3D-Flow processors in this 3D-Flow IBM PC board can now be implemented in a 35 mm×35 mm chip with 64×3D-Flow processors with staggering performance increase and lower cost.

3D-Flow BAQ-BSP IBM PC modular board with 68×3D-Flow processors, 2,211 components, over 20,000 contact pins connected through only 8 layers printed circuit board for signals and 6 layers for power and ground. The 68×3D-Flow processors are housed in 17 large FPGA from Altera, each with 4×3D-Flow processors. The board and the system worked at the first prototype. The signals transmitted over LVDS connections provide stability and noise immunity to the system even when several signals switch at the same time. The board designed and built by Crosetto in 2003 had unprecedented performance in guaranteeing the clock distribution to the pin of every component in a system made of several of these modular boards in different crates with a maximum difference between any two pins of 40 picoseconds. In comparison, it was stated on Sep. 23, 2011 by the spokesman of the OPERA experiment [³⁰], [³¹], [³²] at CERN that their system had an error in clock distribution among components in different boards and crates of 4,000 picoseconds, similar to the error of other systems such as CMS and Atlas. The article describing the board is available at this link and the circuit achieving max difference of 40 picoseconds is available at this link.

This board can now be replaced by a chip with 64×3D-Flow processors
Over 13 times faster
Comsuming less than 1/10 the power
Costing less than 1/200

### 64×3D-Flow Processors IC, 35 mm×35 mm

To migrate the technology-independent 3D-Flow design of 4× processors per chip to either 16×, 64×, or 256×, optimized for the best cost-performance, a study was conducted for the 180 nm, 130 nm, 90 nm, 65 nm and 40 ns for the generic process, the LP (low power) and for the ULP (Ultra Low Power) technology processes. The 28 nm leading edge or 14 nm bleeding edge technologies were not considered because of the high cost and low volume that would not justify the NRE cost. Another reason is that the 3D-Flow architecture offers performance breaking the speed barrier of the technology used, therefore optimization is focused on price-performance and not on absolute maximum performance. The major IC design houses and silicon foundry in the world were contacted to ensure all options were considered. Considering the speed, power consumption, cost per 3D-Flow processors, cost and performance of IP such as LVDS, PLL, USB, etc. for different technologies, the optimization was achieved in selecting 64× processors per chip with 112 pins carrying signals to/from each group of processors facing the North, East, West and South (NEWS) sides, 256 pins for signals sent out from the Bottom port, 256 pins receiving signals from the Top port and 26 control signals. The risk involved in this migration from 4×3D-Flow processors per chip to 64× is minimal or close to zero because its functionality has been already tested with simulators and in hardware on the 4×3D-Flow processors per chip and 68×3D-Flow processors on two boards. Accurate calculation of power consumption from nW/MHz/gate to the power consumption of the chip, of the power consumption of the VME 9U or 6U electronic boards, to the kWatt consumption in a crate were performed as well as the number of signals and speed that needs to be transmitted from chip-to-chip, from board-to-board and from crate-to-crate.
The entire 3D-Flow system for the 3D-CBS extracting all valuable information from radiation (radioisotope) to provide an effective Early Cancer Detection at a very low radiation dose and examination cost requires 2304 channels that can fit in one 6 U VME crate

Nine 256 channels 3D-Flow boards 6U VME, 233 mm×160 mm with the capability to execute up to 100 Object Pattern Real-Time Recognition Algorithm 3D-Flow steps @20 MHz 64-bit input data rate and one 3D-Flow Coincidence Detection board. The system consist of nine 6U VME Readout-Processing boards with partial pyramid for channel reduction and one 6U VME Coincidence Detection board with the final section of the pyramid to further reduce the number of channels and the detection of 511 keV pairs of photons in time coincidence. The global 3D-Flow system can sustain the highest possible data rate which is limited by the speed of the crystal detector capable of capturing millions of pairs of photons in time coincidence per second, with zero dead time. Each of the 2304 input channels receive up to 64-bit data every 50 ns. Each 3D-Flow program step has the capability to perform up to 26 operations, such as addition, subtraction, comparisons of multiple values, etc. This provides a complete, thorough Object Pattern Recognition capability using information from multiple sensors (SiPM front and back, or PMT).

One 6U VME Crate with 9×3D-Flow 256 Ch. Boards and 1×3D-Flow Coincidence board 3D-Flow photon detection algorithm max 100 steps @20 MHz 64-bit input data rate
The 3D-CBS is a safe revolutionary device for Early Detection and Prognosis of cancer, and to screen for cancer on asymptomatic people when it is most curable
It can also detect other diseases presenting abnormal biological processes
Experimental data demonstrates that Early Cancer Detection saves over 50% of lives

I also understand that no one wants to put anyone through the experience of interrupting their research; this happened to me twice: once when the SSC was shut down in 1993 and again in 1999 when DOE could no longer afford to support four experiments at LHC, and decided to stop funding two of them: LHCb and Alice. My 3D-Flow project had been adopted by LHCb, and for a second time its development was interrupted.

I understand too that because of all the ongoing HEP projects DOE has to fund in the coming years, there may not be funds available in the future for the 3D-Flow project if it is not adopted by an experiment; and since no one can force leaders of experiments to adopt it, experiments might continue to make small improvements to their electronics for years to come without adopting my revolutionary approach. And, of course, as long as the 64×3D-Flow chip and the 9U VME board are unavailable, they have an excuse to ignore it and even reject my papers at conferences.

If the current DOE HEP budget does not allow the funding of a generic R&D project like the 64×3D-Flow chip boards and crate, then it will be important to meet, go through my calculations, and if found correct, inform the leaders at a higher level to provide separate funding in order not to miss this opportunity to save money, lives, and to create a powerful tool to advance HEP research.

Please let's meet and verify my calculations and technical feasibility related to the existing technology. I spent much of August verifying these with Engineers in the design and manufacturing of integrated circuits from top industries in the world. We reviewed equations and calculations representing the reality of what current technology can offer. Let's collaborate also with the scientists assigning funds for advancing the field of particle detection and verify together my equations and calculations as I did with the Engineers from industry, and then announce to the leaders at a higher level if my calculations were correct.

I believe that my duty and our duty is to serve our leaders and together serve our country and humanity by informing them about innovations beneficial to humanity that are supported by calculations and scientific evidence.

In summary, my breakthrough invention is a 64×3D-Flow processors chip that is over 13 times faster, consumes less than 1/10 and costs less than 1/200 the previous 64×3D-Flow processors version which was already proven feasible and functional in a board.

These staggering improvements mean that several 9U VME crates of the Level-1 Trigger at the largest HEP experiments can be replaced with a single 9U VME crate with either 9 boards or 17 boards, increasing the power of the tools to discover new particles and provide benefits in many other field such as in Medical Imaging to save lives and reduce healthcare costs.

I would welcome another open international review like the one at FERMILab held on Dec. 14, 1993, when for the entire day I answered all questions satisfactorily regarding my 3D-Flow and supported my calculations that ultimately were proven correct by implementing the two 68×3D-Flow processors boards in FPGA. This would save the development of many trigger crates of electronics planned for the upgrade of CMS and Atlas experiments costing millions of dollars.

To migrate the technology-independent 3D-Flow design of 4× processors per chip to either 16×, 64×, or 256×, optimized for the best cost-performance, we conducted a study for the 180 nm, 130 nm, 90 nm, 65 nm and 40 ns for the generic process, the LP (low power) and for the ULP (Ultra Low Power) technology processes. The major IC design houses and silicon foundry in the world were contacted to ensure all options were considered. The risk involved in this migration from 4×3D-Flow processors per chip to 64× is minimal or close to zero because its functionality has been already tested with simulators and in hardware on the 4×3D-Flow processors per chip and on two modular boards, each with 68×3D-Flow processors. Accurate calculation of power consumption from nW/MHz/gate to the power consumption of the chip, to the power consumption of the VME 9U or 6U electronic boards, to the kWatt consumption in a crate were performed as well as the number of signals and speed that needs to be transmitted from chip-to-chip, from board-to-board and from crate-to-crate.
G. The Specifications, Drawings and Requirements I provided when requesting quotes from different companies for the LHC TER/DSU board

I first asked the companies interested in bidding on this project to sign a Non-Disclosure Agreement (NDA), and then provided them the three items listed below. I followed up with several phone calls, emails, and visits to their sites (for companies in Dallas) for a period of two to three months.

I provided:
1. a text description of the specifications.
2. drawings of the logical block diagram showing functionality of the board/chip for which I needed a quote.
3. an Excel spreadsheet with guidelines of the items I suggested in order to have a clear separation between NRE (Non-Recurring Engineering) and a small production of 32 boards, and a larger production that could be purchased in the future from other groups or universities.

As you will notice in the Budget Justification, some companies have filled in the three-page spreadsheet I provided where on the first page there is the summary of the NRE cost which has been separated into the cost of a small production of 17 to 33 electronic boards and a larger production from 100 to 500 boards. Other used their own template to provide their quote.

Following is an example of specifications, a drawing of the logical block diagram, the layout of the components on the board, and a template to prepare the quote for the LHC TER/DSU board.

The template that I prepared for the different companies that were interested in bidding on this project were the following as reported in the Excel spreadsheet of Table 4
D. Functional Description of the 320 MHz and 640 MHz ER/DSU Board

320 MHz ER/DSU Board

The primary feature of the 320 MHz ER/DSU board is to transfer data from onboard DDR memory at a maximum rate of 40.96 GB/sec to 512 LVDS DDR channels, for an effective throughput of 640 Mbps for each LVD channel. All 512 bits on the 512 LVDS channels will be presented at the same time, with new being clocked out at a rate of 1.56 ns per bit on the front panel connector. In addition to transmitting data at this speed, the board will be capable of receiving data over the 512 LVDS channels and writing the data to the onboard DDR memory at a rate of 640 Mbps per channel without missing any data.

The 512 LVDS data lines on the front panel are grouped in four connectors, each with 128 LVDS data lines. A separate connector provides control lines such as: External clock, Start transfer and Stop transfer.

The clock determines at which speed the data is transferred. A continuous increment from 1 MHz to 320 MHz (640 Mbps at each channel, presenting at the same time 512-bits on the 512 LVDS channels at a max speed of a new data every 1.56 ns) will not be required, but some frequencies will not be possible if they will be in conflict with the synchronization between read/write with RAS/CAS and other control signals at the DDR3 memory. The “Start Transfer” signal will initiate the transfer to/from the memory of its entire content. At any time the device receiving data can issue a “Stop Transfer”.

While the ER/DSU board is required to transfer data continuously from/to the front panel to/from the memory with no interruption and without missing any data, its interface to the VME bus or to a PCIe host will allow a host computer connected to the same interface to access the memory randomly and/or read and write via a DMA. The amount of data that can be sent or received is limited by the amount of onboard DDR memory. When transmitting, the data in the onboard DDR memory can be transmitted once as a single-shot output, or it can be sent repeatedly in a continuous loop until stopped by the host. In the case that a different data set needs to be transmitted, the host computer will stop transmission and reload the memory with new data. It is envisioned that this reloading would take place at a much slower rate than the 320 MHz/640 MHz rates of the LVDS outputs. Likewise, the receive mode will write incoming data to memory until the memory is full, at which point, the receive logic will stop receiving data and will alert the host that the memory buffer is full. The host computer can then read all of the data from the onboard DDR memory at a reduced data rate before starting to receive new data.

There is no need for arbitration to access the memory from VME (or PCIe) and the front panel connector. The ER/DSU board is either communicating with VME (or PCIe, depending which host is connected to the crate) or it is transferring/receiving data to/from the front panel connector. During these operations VME (or PCIe) cannot access the memory. In order to access the memory, the VME bus (or PCIe) must take control of the memory and disable the communication with the front panel connector.

The ER/DSU board should be accessed from a driver developed for VxWorks and/or from Windows 7 (8 or 10) and should have JTAG. The driver is not a part of this project estimate.

640 MHz ER/DSU Board

The 640 MHz ER/DSU board is similar to the 320 MHz board, with the following differences:
1. The maximum data speed is increased from 320 MHz DDR (640 Mbps/channel) to approximately 640 MHz DDR (1200-1280 Mbps/channel).
2. Each 640 MHz board will only have 2 front panel data connectors instead of 4, each outputting 128 LVDS data channels, for a total output of 256 LVDS channels per 640 MHz board.
3. Different FPGA's and other components will be utilized in order to provide for the faster data transmission and receive rates.
4. All other functionality will match that of the ER/DSU 320 MHz board.

I have also provided specifications, drawings, the layout of the components on the board and the template for the items to quote for the VME board housing 25 chips, each with 64×3D-Flow processors, the VXI board with 66 of these chips and one with 84 chips.

I have also specified which other Integrated circuits are necessary for the ancillary logic, the connectors etc. I verified the feasibility of building each part and made sure that the company could answer questions addressing all aspects relative to the job that needed to be performed.

Notice in the following Excel spreadsheet that I matched the number ID in the PCB board layout with the number ID in the first page of the Excel spreadsheet in the Build of Material (BOM).

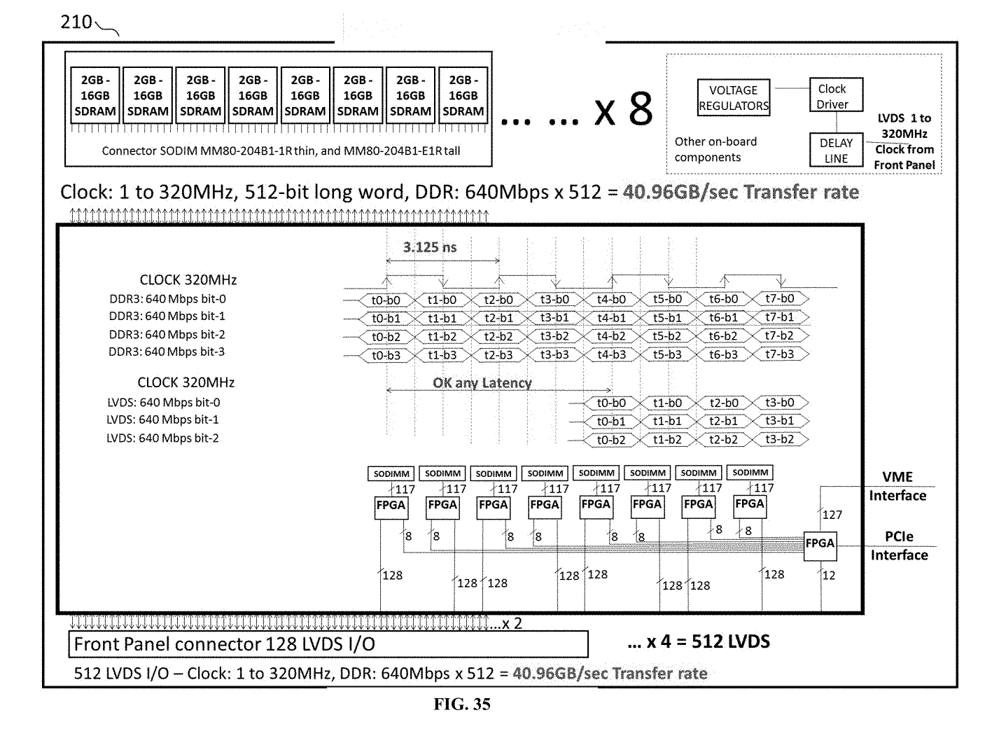

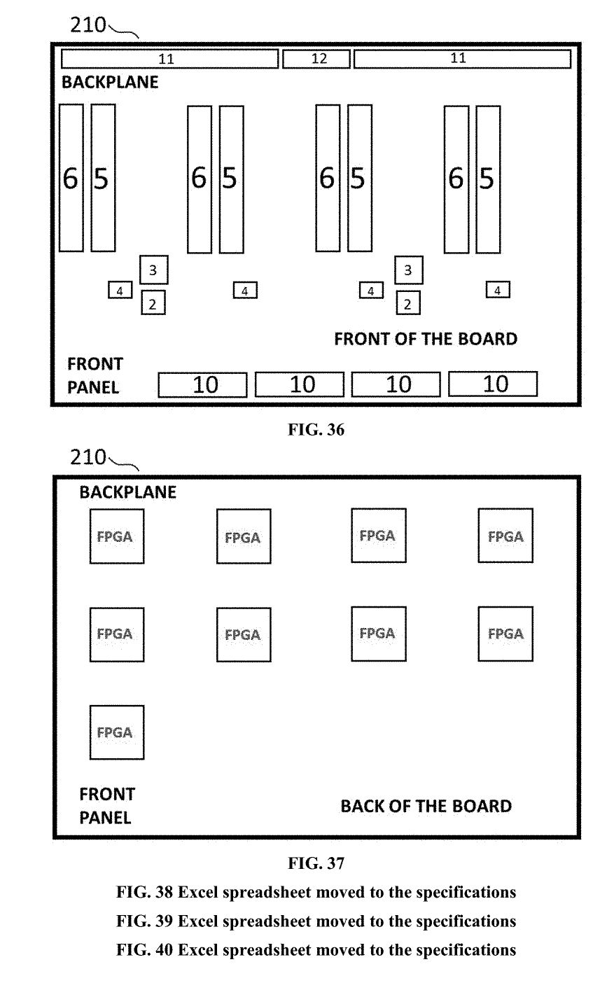

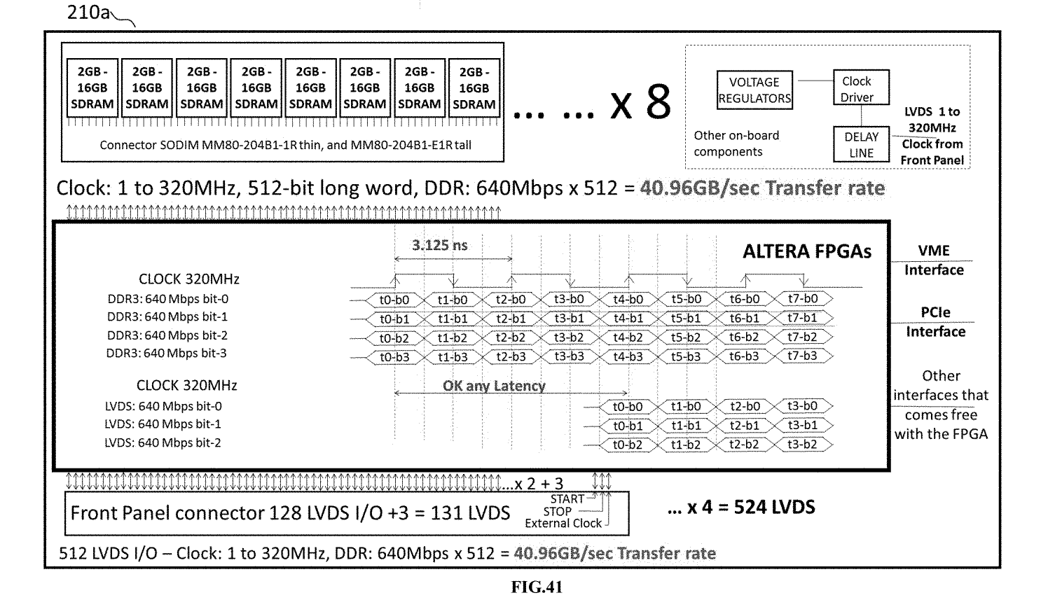

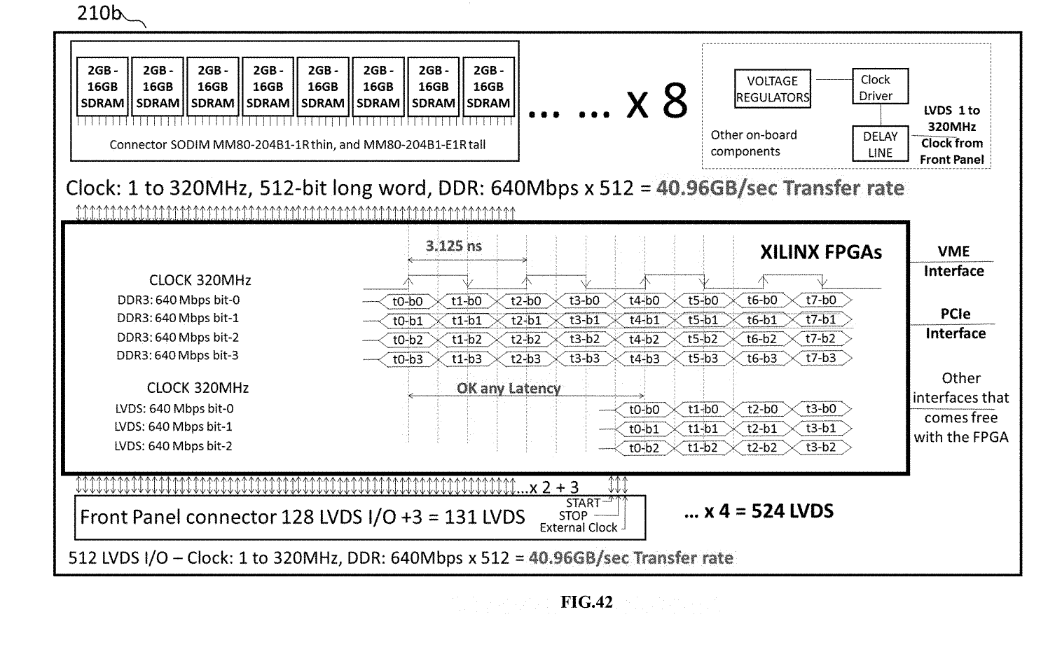

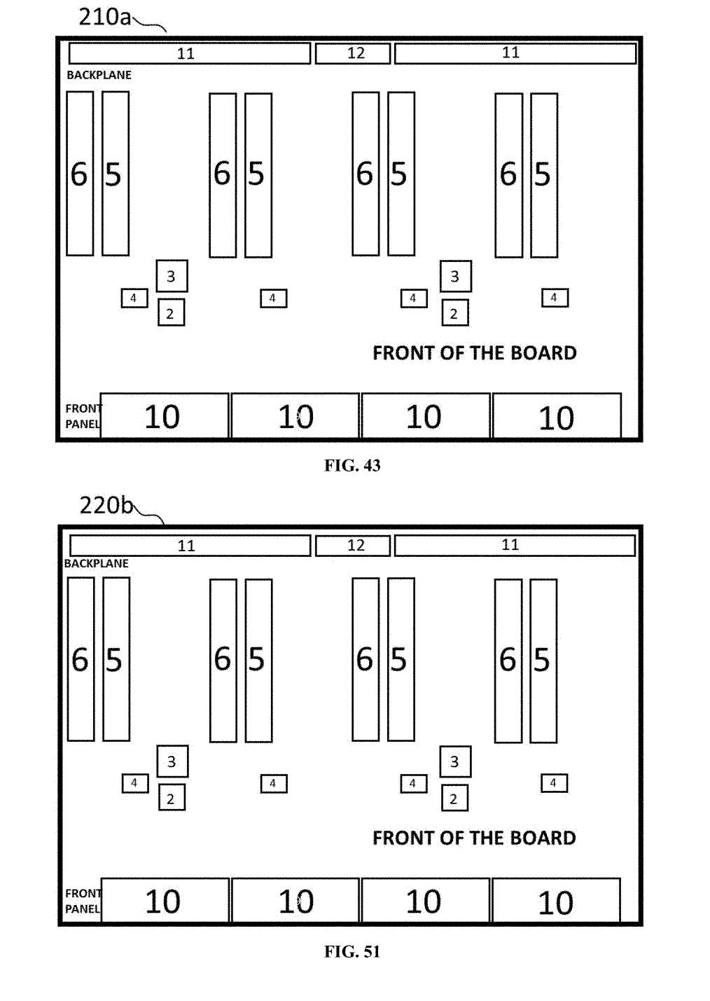

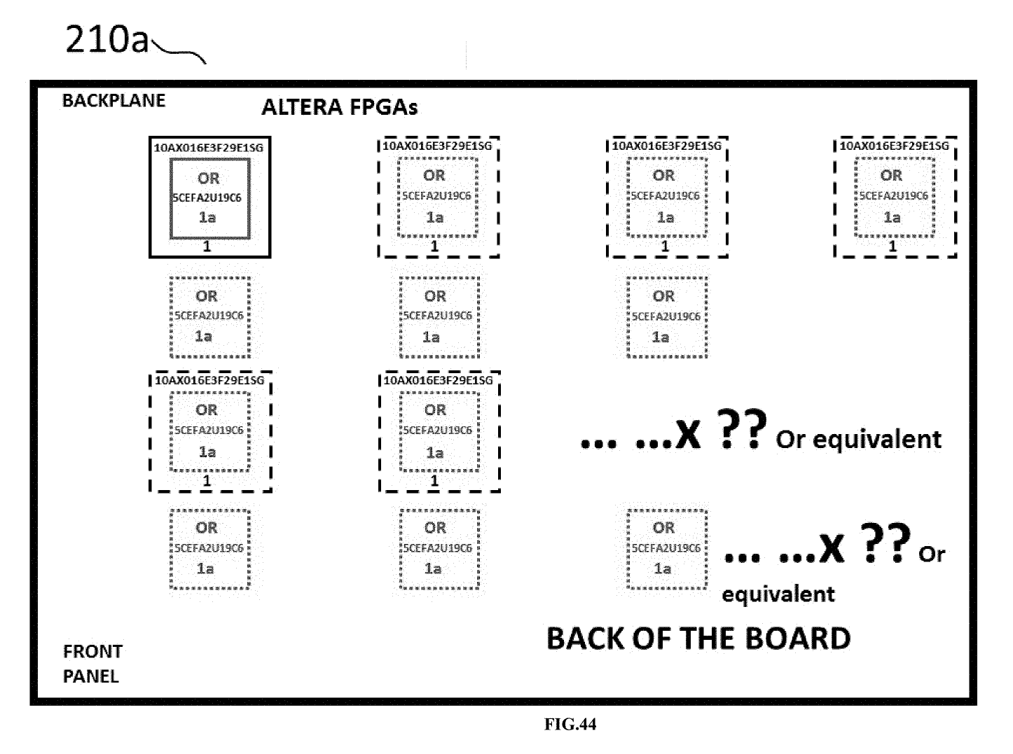

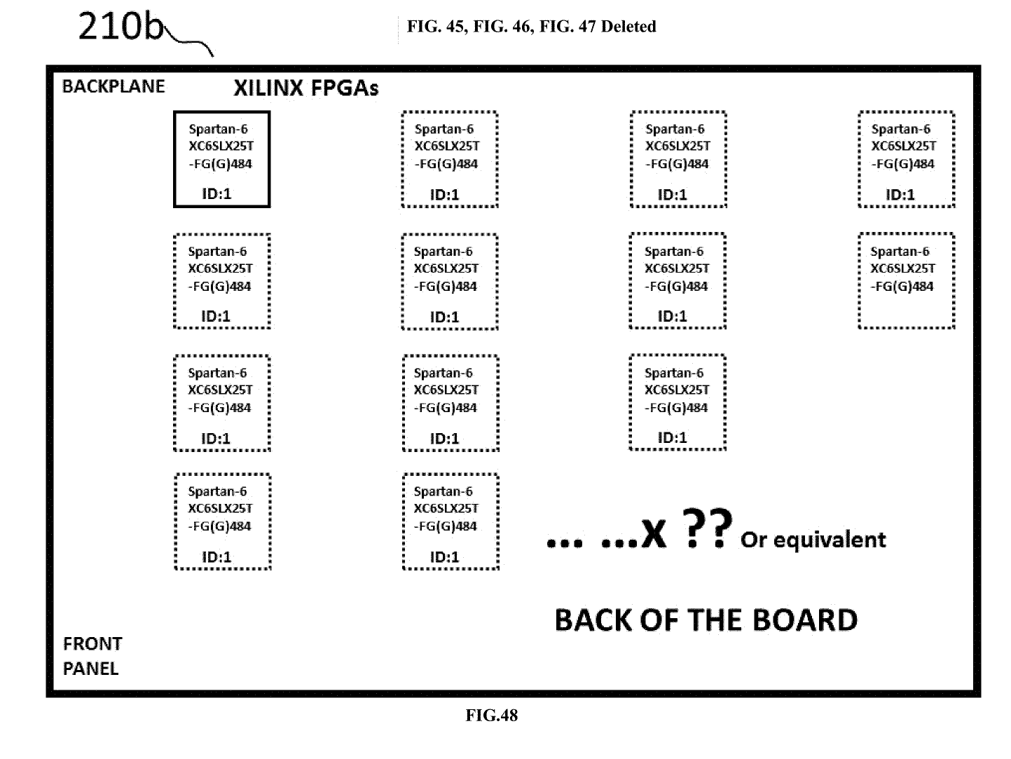

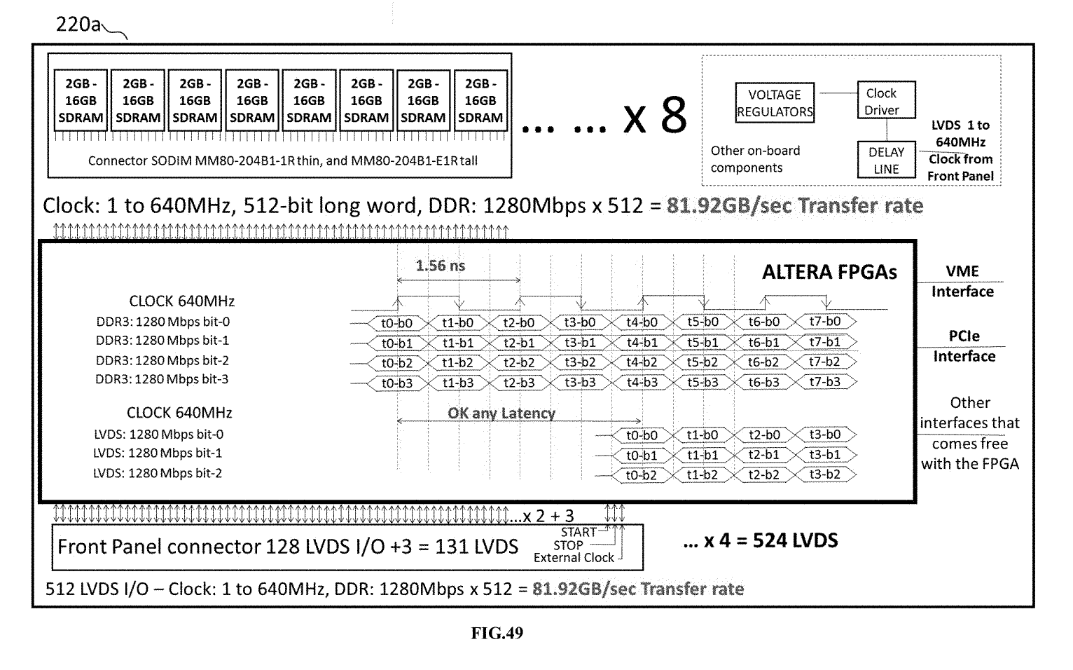

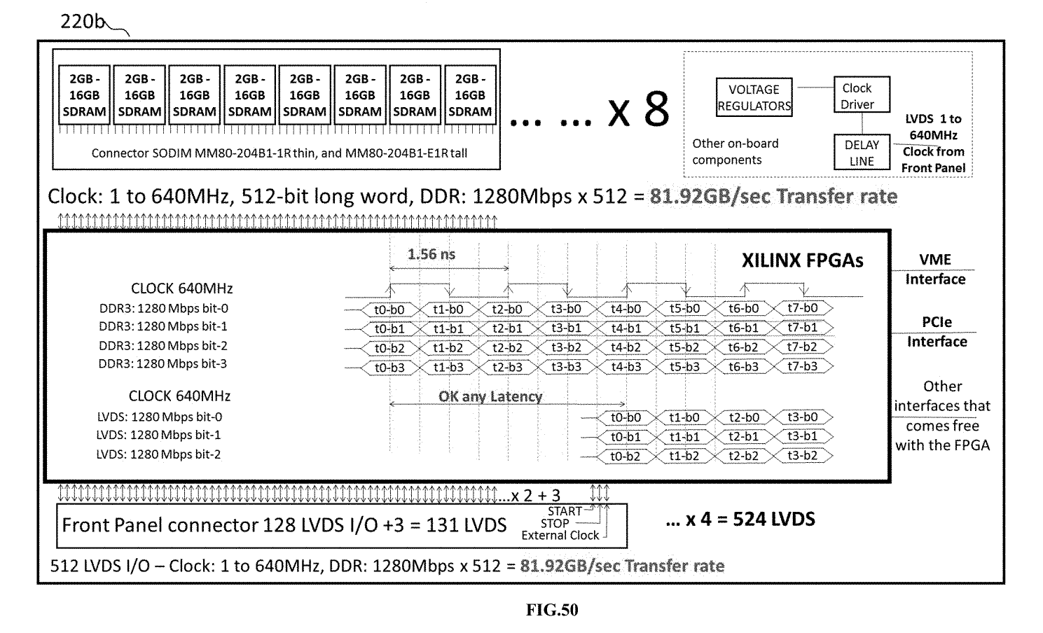

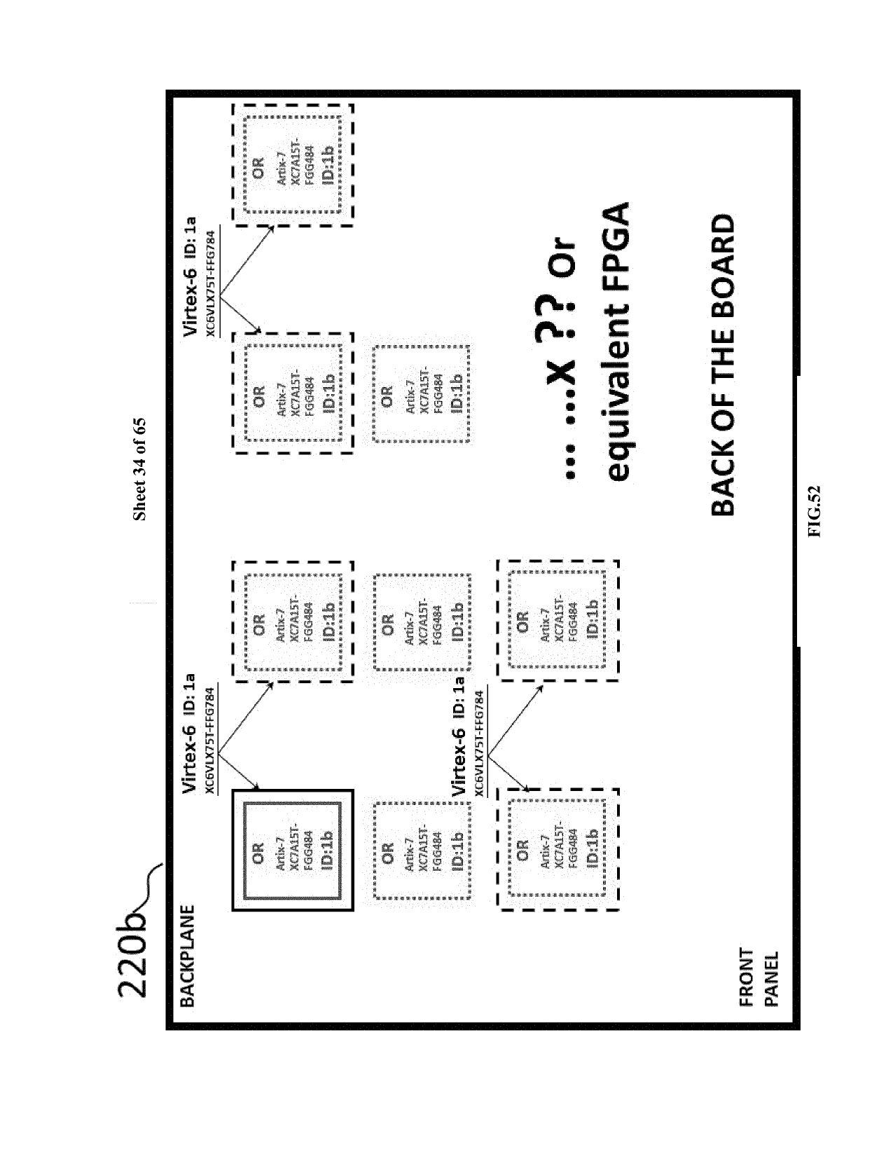

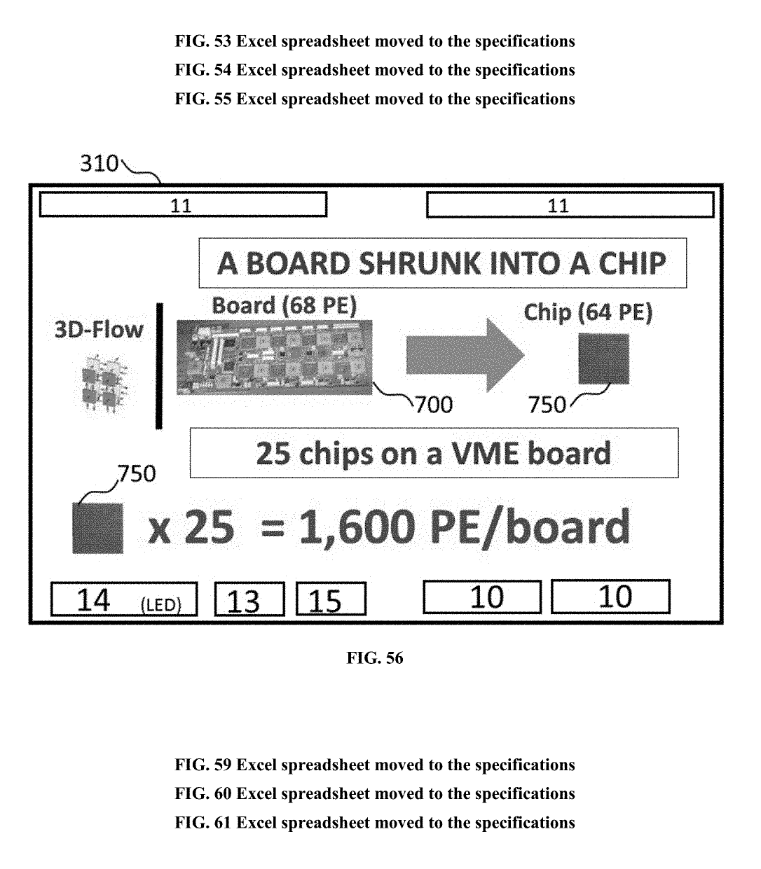

See FIG. 35, FIG. 36, FIG. 37, FIG. 41, FIG. 42, FIG. 43, FIG. 44, FIG. 48, FIG. 49, FIG. 50, FIG. 51, FIG. 52.
H. Examples of Implementations of the 3D-Flow Multiprocessor System on a VME Form Factor

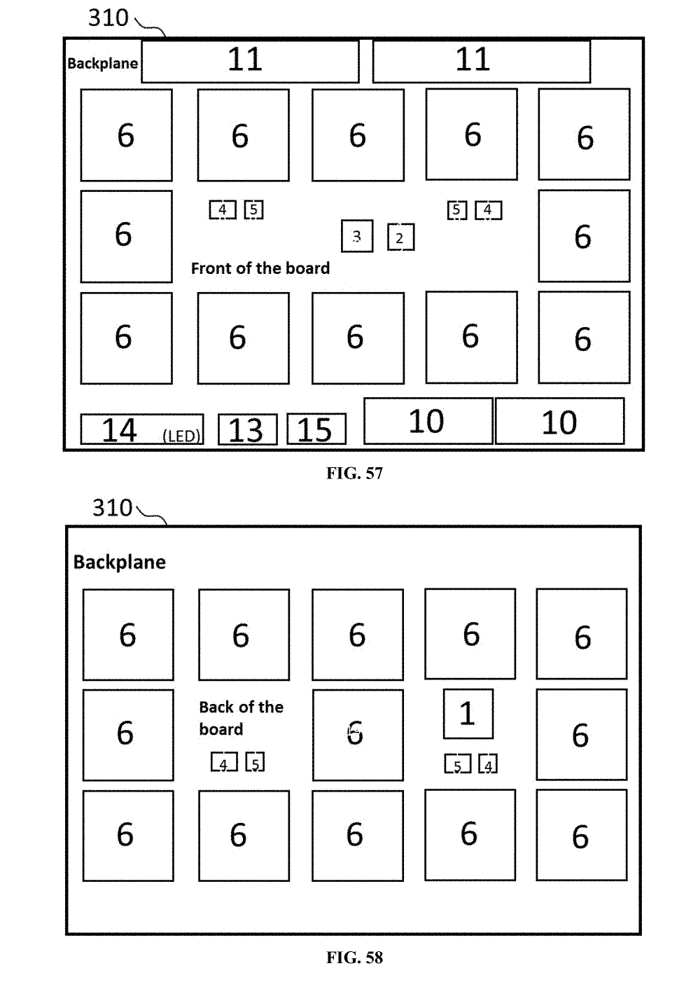

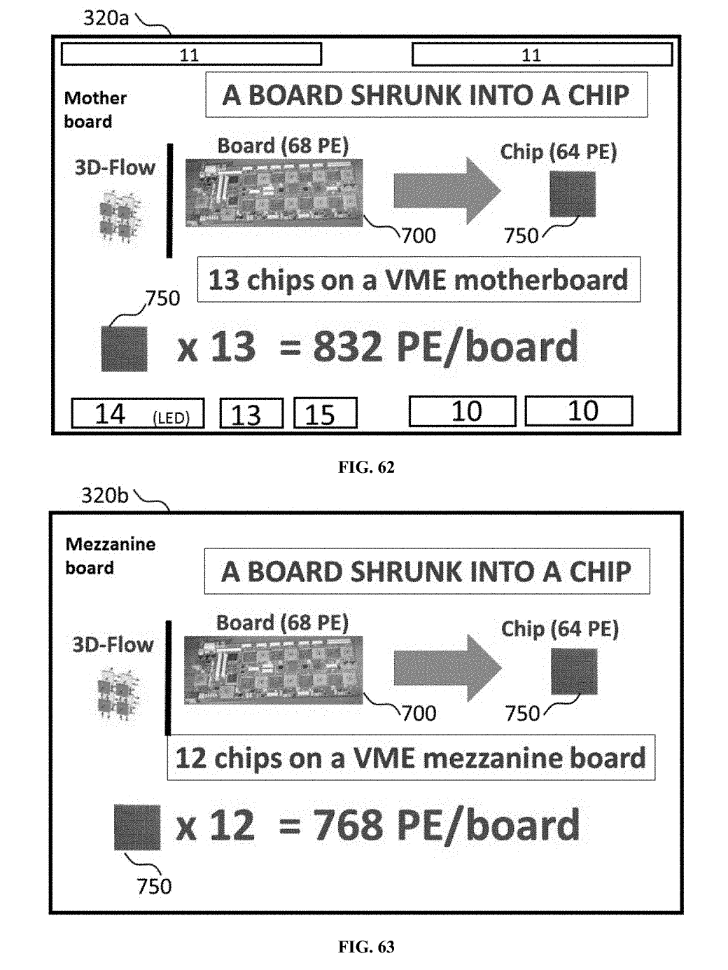

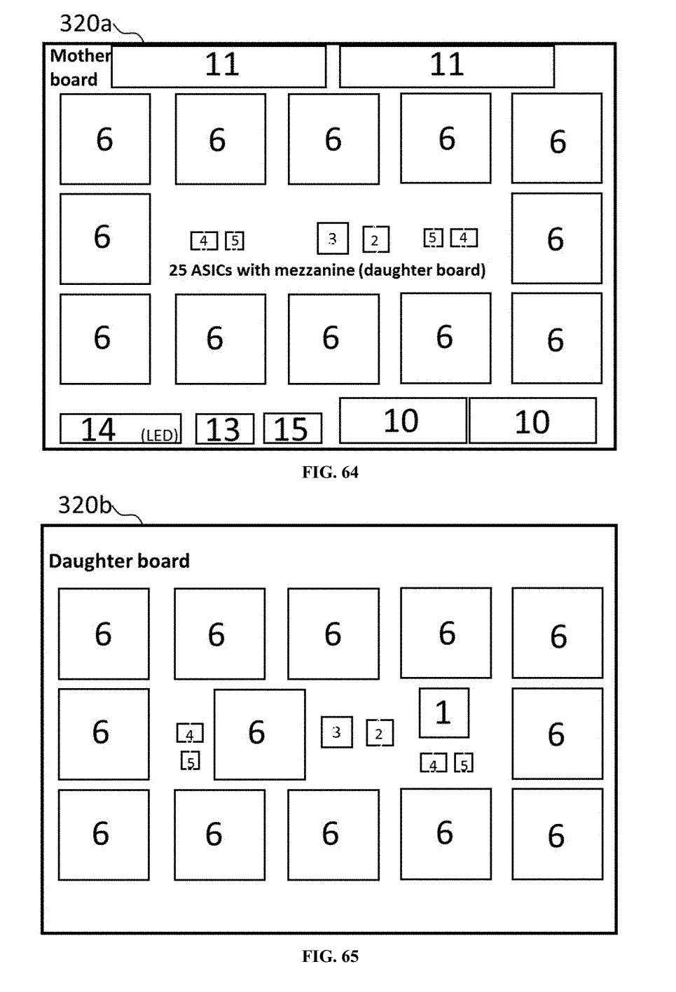

See FIG. 56, FIG. 57, FIG. 58, FIG. 62, FIG. 63, FIG. 64, FIG. 65.

Following is an example of specifications, a drawing of the logical block diagram, the layout of the components on the board, and a template to prepare the quote for the 3D-Flow VME form factor electronic board.

The template that I prepared for the different companies that were interested in bidding on this project were the following as reported in the Excel spreadsheet of Table 5
J. Step-by-Step Development of the Project Showing Feasibility and Functionality

My professional experience in the field lead me to approach the problem by looking at the objectives of creating a system with the capability to identify objects (through pattern recognition) satisfying experimenter's desired characteristics (modifiable through a programmable complex real-time algorithm) from data arriving at a speed up to 1.3 TB/sec, propose a solution that offers deliverables of measurable results on a test bench of a lab and ease integration into the target system.

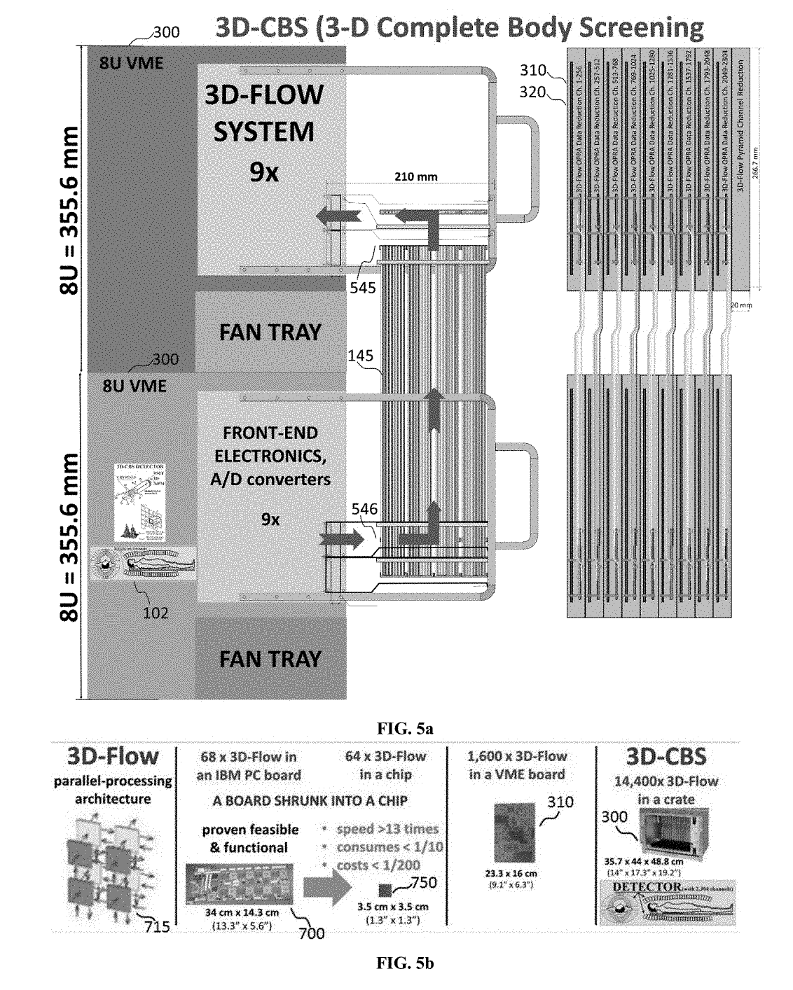

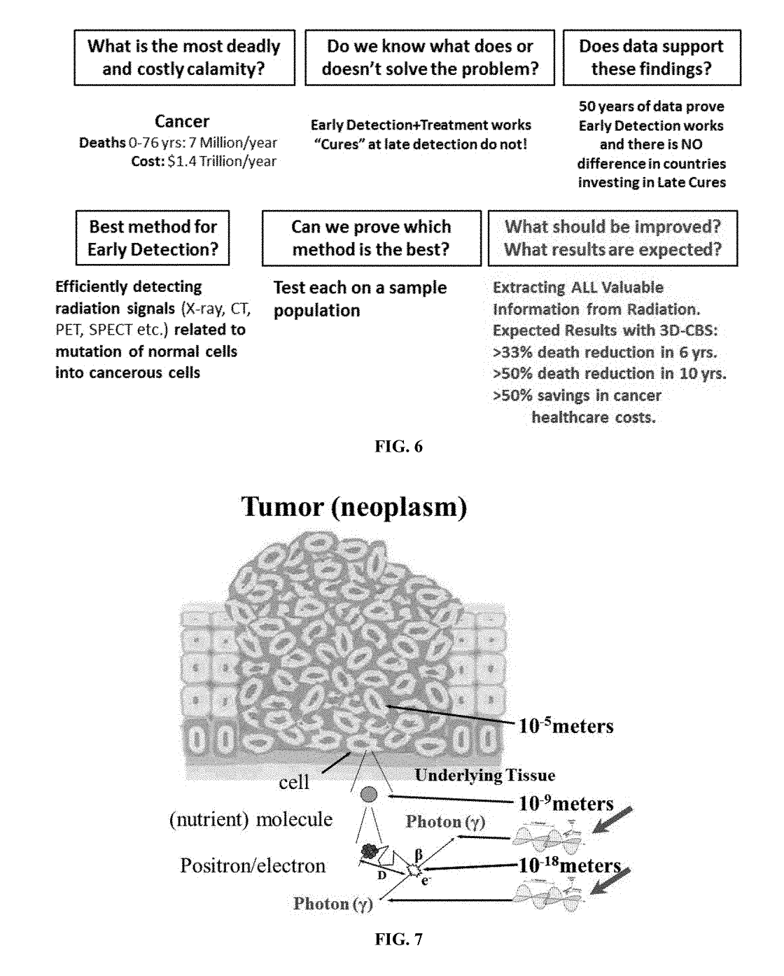

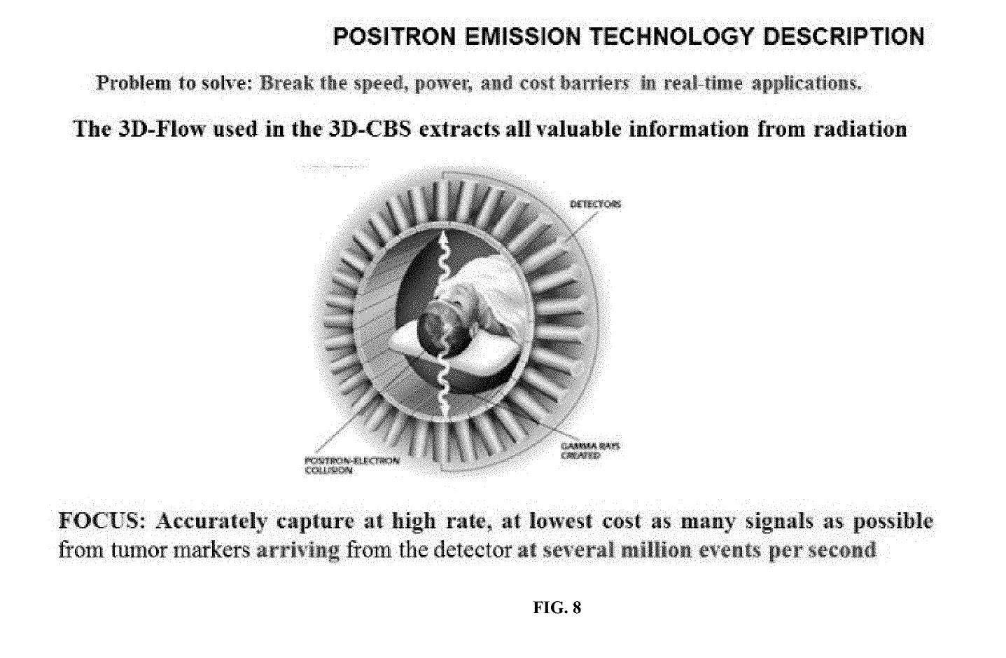

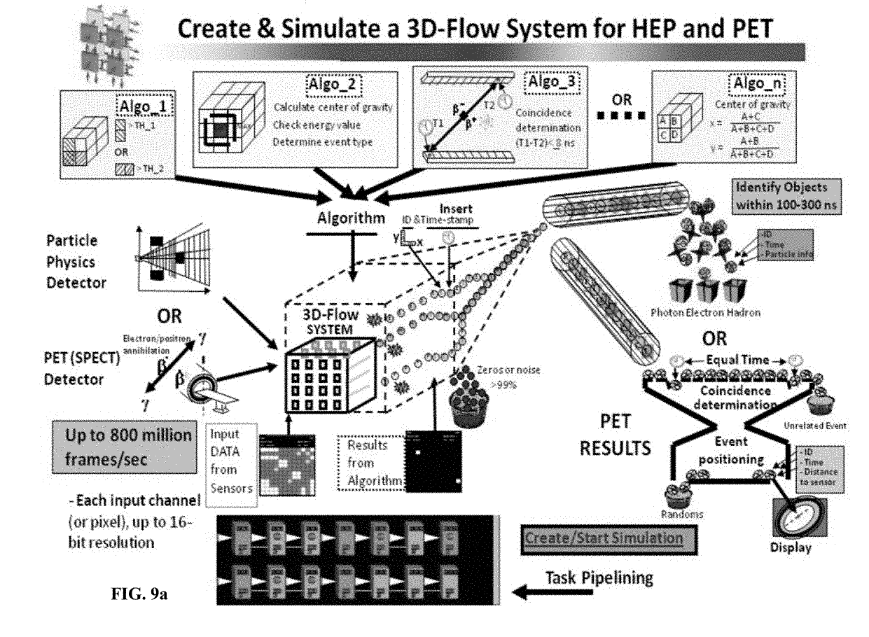

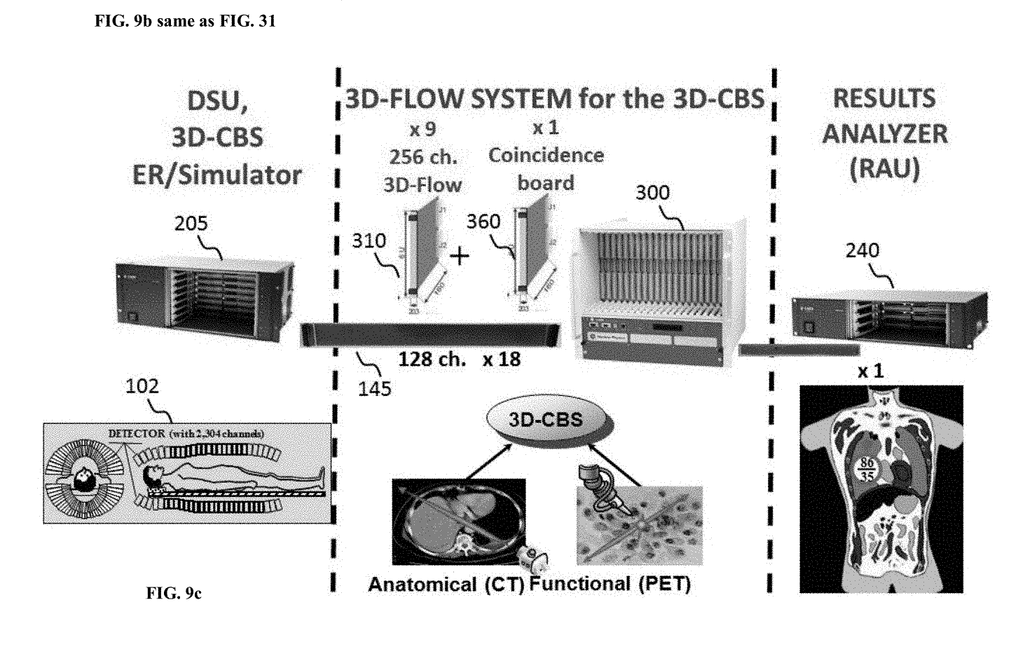

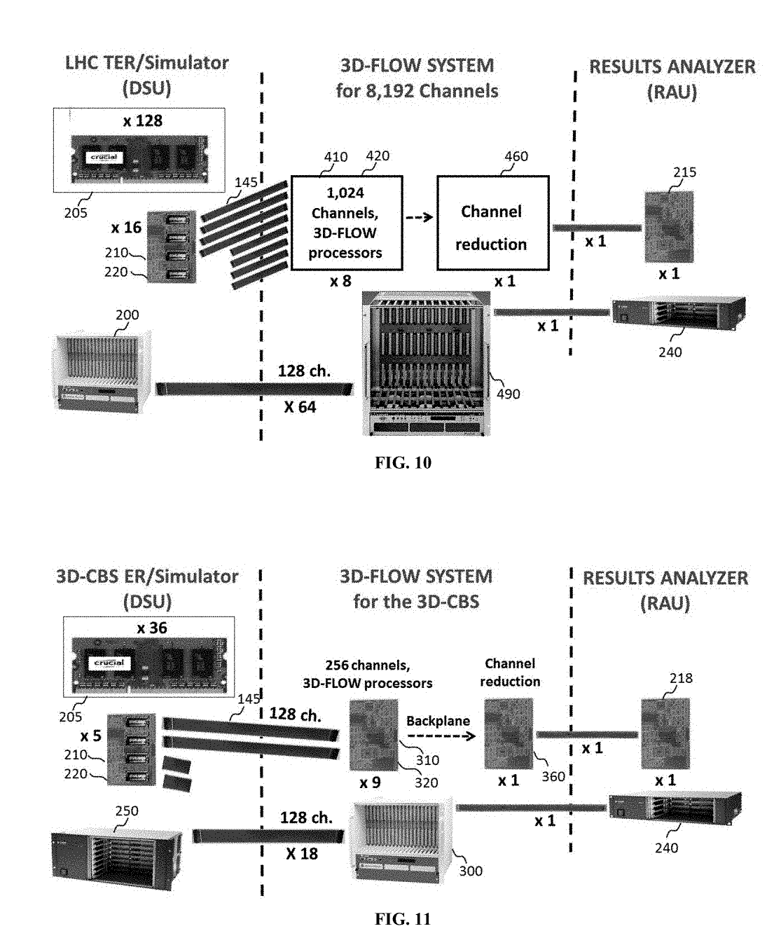

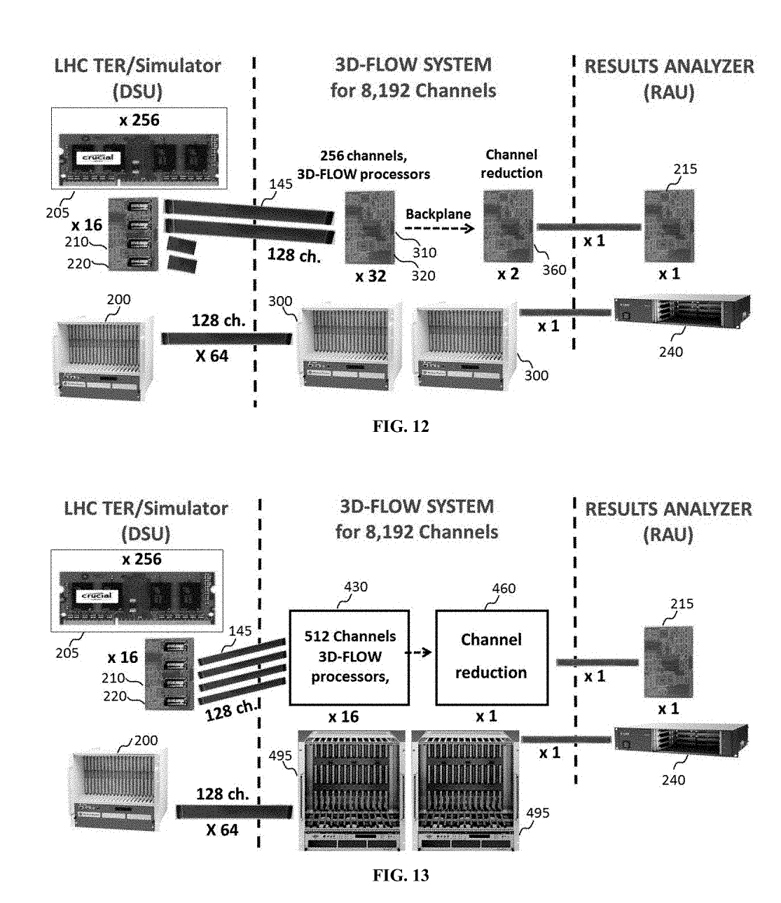

I therefore observed the entire HEP application system for 8,192 channels at 1.3 TB/sec transfer rate in FIG. 4 and the medical imaging application for 2,304 channels at 368 GB/sec transfer rate in FIG. 5 and identified the key parameter on which I needed to focus the data rate for a single channel at source and destination which is 0.16 GB/sec, which is equivalent to 1280 Mbps (160 MB/sec×8-bit=1280 Mbps).

Whether CERN decides to bundle several of these 8,192 signals together and transport 10 of these on a cable or optical fiber at 10 times the speed, or 100 at 100 times the speed will be determined by distance, environment and cost/performance of 10 GbE, vs. 100 GbE, vs. SATA, etc.; however, data integrity at destination should reflect the same content and integrity of the signals at the source generated at 1280 Mbps per channel, equivalent to data commuting every 781 picoseconds (ps), represented by the “eye” shown in FIG. [symbol] .

This speed of data arriving at each channel every 781 ps satisfies the requirements for extracting all valuable information from radiation with a system like the 3D-Flow having the capability to execute Object Pattern Real-Time Recognition Algorithms on multiple data arriving at this speed. It satisfies the need for future upgrades of the LHC either running at frequency higher than the actual 40 MHz or at a higher luminosity. According to articles published by CERN, the LHC could not run at 80 MHz due to beam heating, however, it is planned to run at a higher luminosity.

See FIG. [symbol] for an overview of the test bench system implemented using the VXI form factor and FIG. [symbol] implemented using the VME form factor that will allow experimenters' to test their Level-1 Trigger system (3D-Flow system or any other Level-1 Trigger approach).

Creating a 3D-Flow Trigger system with 781 ps data rate capability at each channel will satisfy either acquiring 16-bit data from the LHC at 80 Mhz if the beam heat problem is solved (16×781 ps=12.5 ns) or 32-bit data from LHC at 40 MHz generating more data with a higher luminosity that is planned for future upgrades at CERN as stated in their documents (32×781 ps=25 ns LHC bunch-crossings).

The planned workflow of this proposal that will optimize the use of resources and taxpayer money to build a system that can provide measurable results on a test bench of a laboratory and easily integrate into applications such as LHC, Medical Imaging, etc., is to advance from purchasing instrumentation available on the market to generate and record/display signals similar to those generated by radiation at LHC, medical imaging, video cameras, etc. for two signals and create instrumentations to generate and record/display 8,192 signals.

The instrument from a vendor catalogue at $55,000 generating 2 signals similar to those generated from radiation at LHC, medical imaging, etc., will allow 512 and 256 channel VME boards generating similar signals to be built.

At this point the 512 and 256 channel VME boards costing about $2,250 each to duplicate, become the instruments to test equivalent VME boards recording/displaying 512 and 256 channels at that speed.

Sixteen 512 channel VME boards for a total cost of approximately $40,000 for 8,192 channels in a VME crate become the DSU (Detector Simulator Unit) instrument providing the signals similar to (with a little additional performance in providing 16-bit information for each data instead of the current 10-bits provided by LHC experiments) to those provided by the LHC experiments until 2018.

This DSU 8,192 channel instrument replaces the Tektronix waveform generator and becomes the signal generator to test an equivalent 8,192 channel TER unit (Trigger Event Recorder).

Once we have tested the 8,192 channel TER unit, we can take this to CERN to record raw trigger data at LHC and send the data to Scientific Associates of the universities collaborating to the experiment who can use real data on a DSU unit reproducing all the functionality as data generated at the LHC to test the performance of the 3D-Flow system (or any other trigger system) in finding the rare Higgs boson-like particle, or any particle that theoretical physicists can provide characteristics for, that will then be translated into real-time algorithms to be downloaded into the 3D-Flow system (or any other trigger system). The Scientific Associates can even edit the raw data and create very difficult conditions such as adding pileup events, adding noise generated by spurious particles, etc., to verify the efficacy of the 3D-Flow system (or any other trigger system) to filter the particles matching the characteristics set by the theoretical physicists or experimenters for the new particle.

What we will have accomplished at this stage is to have created a 512 channel board and an 8,192 channel DSU and TER from the basic 2 to 4 channel instrumentation purchased on the market for approximately the same cost, when duplicated, as the 2 channel instrument available on the market. They can be used to test each other (first one crate is set to work as a DSU and the other as TER, next the functions of both are swapped, so the functionalities on both are verified).

At the same time we have created two new instruments to be used at CERN to record raw data trigger events and as an 8,192 channel parallel data generator, generating signals similar to those generated by LHC to be used on test benches at laboratories of Scientific Associates located remotely from CERN to test the 3D-Flow system (or any trigger system).

The same occurs when building the 256 channel VME board at 1280 Mbps per channel. Building two such boards with the help of the Tektronix waveform generator and oscilloscope will allow to both VME boards to be verified when set to work as DSU or as TER or vice-versa. Similarly, building two crates, each with 16×256 boards will allow the system to be tested for 4,096 channels, one case working as a DSU and the other as TER and vice-versa. The test will be considered complete only with 4,096 channels because what is important to test is the signal integrity, crosstalk free, power dissipation and the capability to work at full speed for several hours. Additional crates coupled one-to-one to build systems of 8,192 channels, 12,288 channels or 16,384 channels will present additional challenges in power dissipation, signal integrity, etc. because all these characteristics and challenges will be confined within the crate.

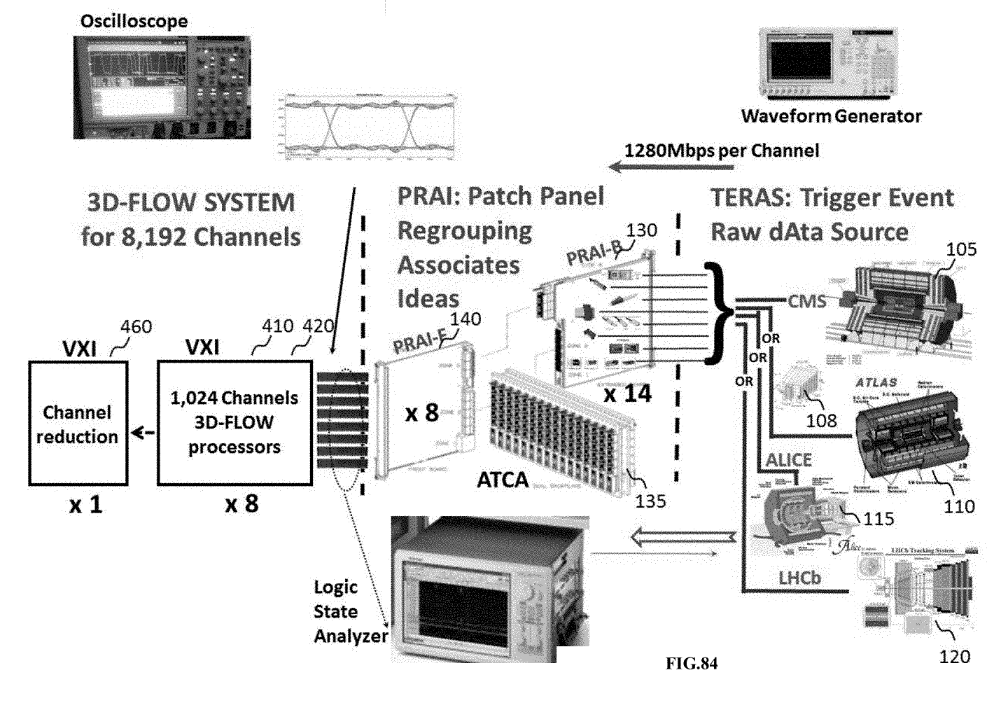

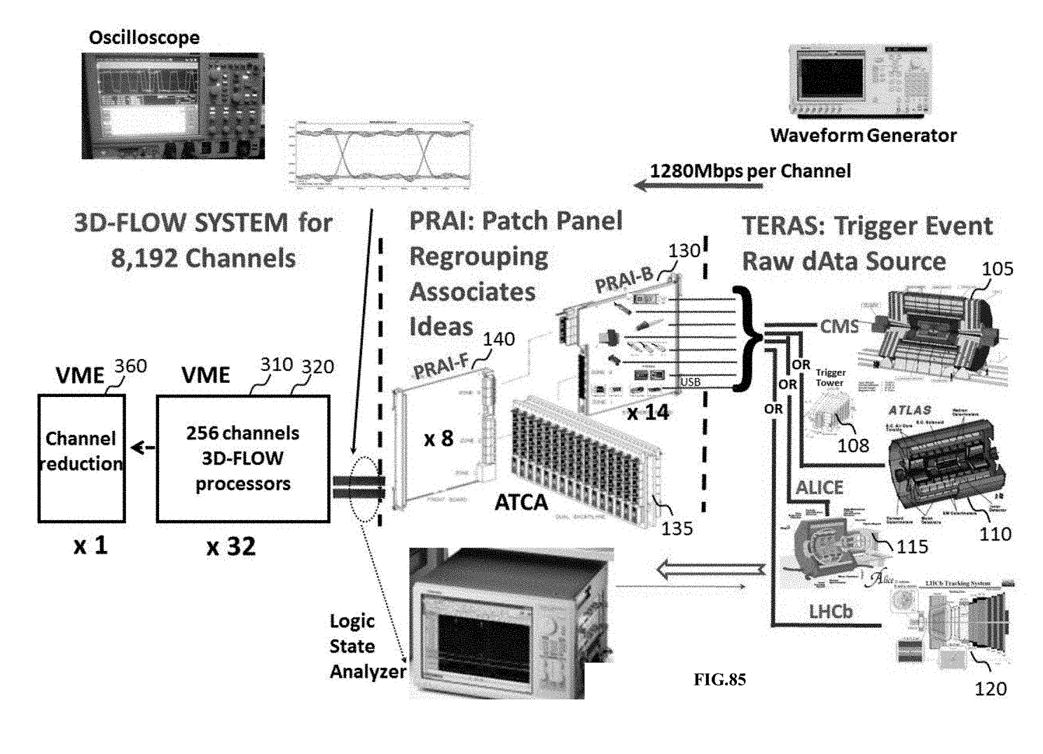

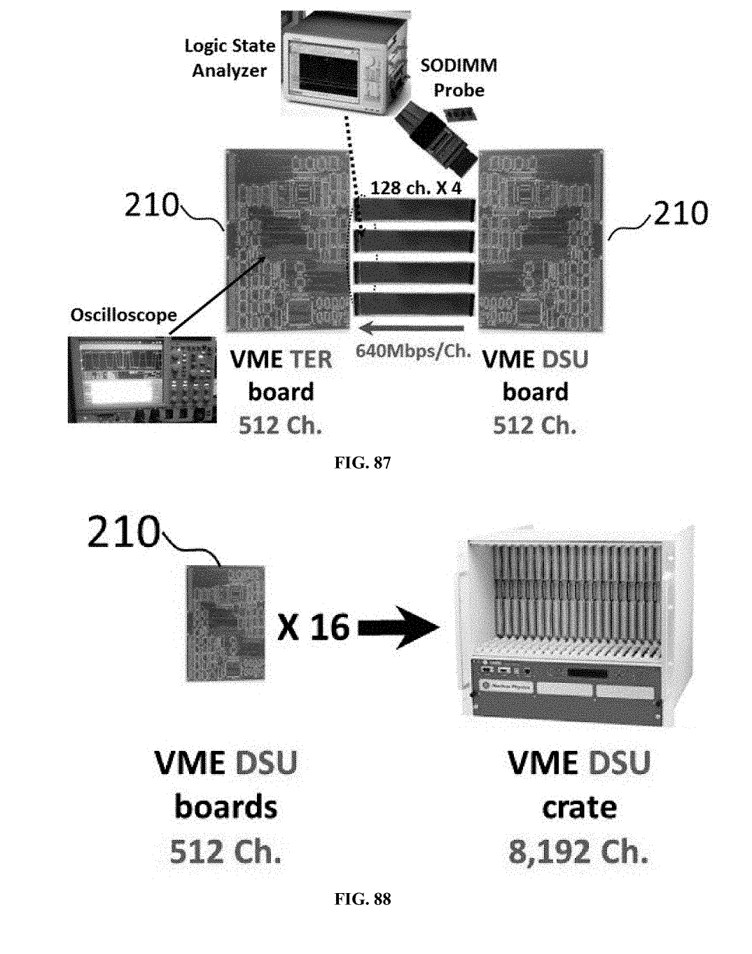

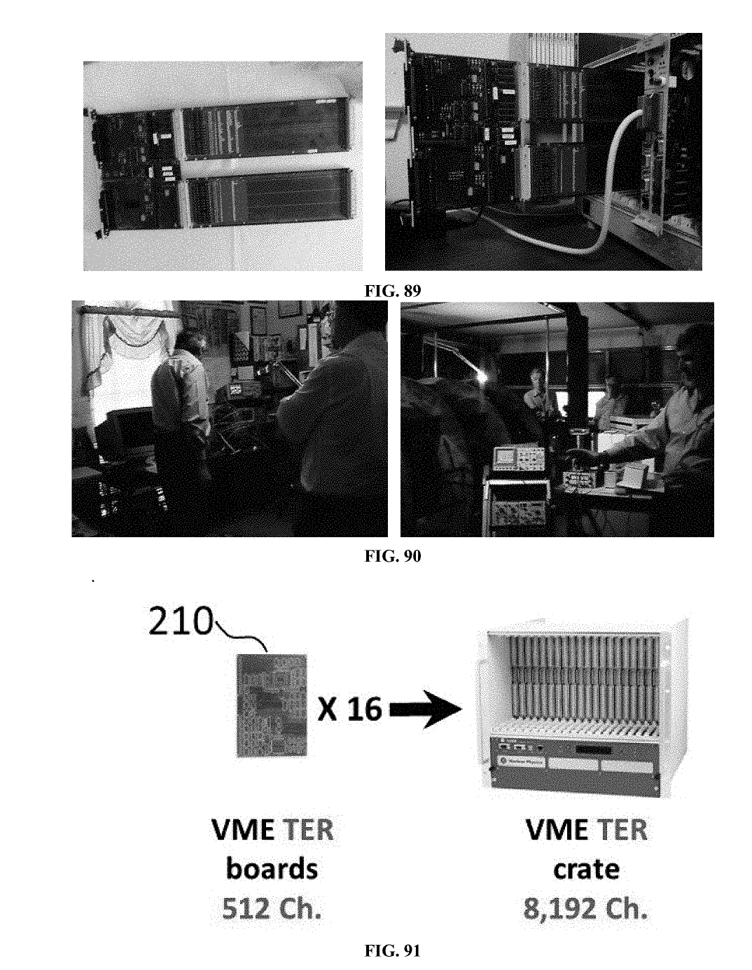

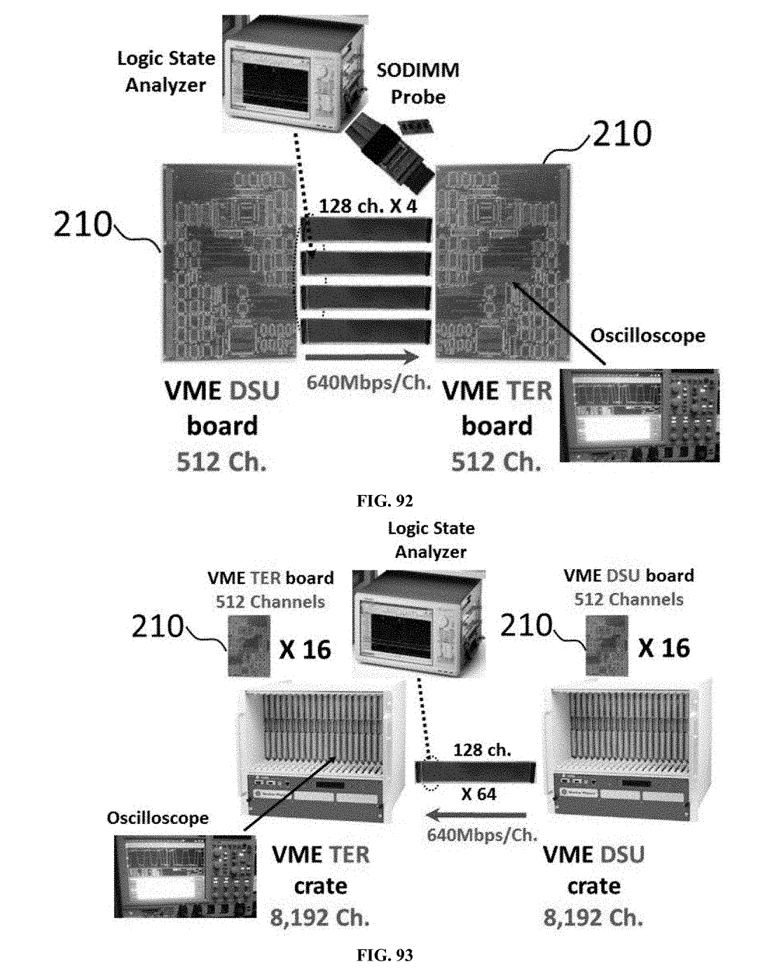

A more detailed flow of the work for this proposal follows:
1. Build a DSU test bench for 2 channels, at 1280 Mbps each. Generate 2 signals (cost of instrument from vendor catalogue is $55,000. This arbitrary waveform generator is all I strictly need for this project, although the catalogue does offer instruments with higher performance costing up to $120,000).
   DONE I have identified a commercially available instrument made by Tektronix, Model AWG5012C, 1.2 Gsamples per second, 14-bit that can generate signals on 2 channels similar to those generated by the radiation at LHC, a PET device, a CT scan, multi-lens movie cameras, etc.
   NEEDED to advance science and for saving lives: funds to pay for the instrument costing approximately $53,000.
2. Build a TER test bench for 2 channels, at 1.280 Mbps each. Record and display 4 signals (cost of instrument from vendor catalogue is $65,000. This oscilloscope at 4 GHz is all I strictly need for this project, although the catalogue does offers oscilloscopes at 33 GHz with higher performance, larger memory, etc., costing >$450,000).
   DONE I have identified an oscilloscope made by Tektronix, model MS070404C, 4 GHz with 4 analogue channels and 16 digital channels that could record and display all parameters relative to signals with an “eye” of 781 picoseconds.
   NEEDED to advance science and for saving lives: funds to pay for the instrument and probes costing approximately $65,000.
3. Design a TER/DSU test bench board for 512 channels, at 640 Mbps each for LHC 2017-18, Medical Imaging, and other applications. Generate and record signals to/from a sequence of data stored into memory (cost for each duplicate instrument built with this proposal is $2,250). Design a TER/DSU VME board for 512 channels @640 Mbps per channel. Contact reputable companies to provide a quote for the cost of an NRE to manufacture electronics boards offering a turnkey solution.
   DONE.
   NEEDED to advance science and for saving lives: Funds to pay the company for the implementation of the NRE at their average rate of $110/hour.
4. Build a TER test bench for 102 channels, at up to 1280 Mbps each. Record and display 102 signals (cost of instrument from vendor catalogue is $160,000. This Logic State Analyzer at 50 GHz is all I strictly need for this project, although the catalogue offers Logic State Analyzers with higher performance, memory, etc., costing >$200,000).
   DONE I have identified a Logic State Analyzer and relative probes made by Tektronix, model TLA7012, 50 GHz, 102 channels, that can test for signal integrity, crosstalk between signals, etc. on busses with several data lines and signals in parallel at a DDR3 memory interface; it can also record and display characteristics of all parameters relative to 102 signals in relation to one another with probes for a SODIMM slot interposer.
   NEEDED to advance science and for saving lives: funds to pay for the instrument and probes costing approximately $160,000.
5. Build a DSU test bench board for 512 channels, at 640 Mbps each for LHC 2017-18, Medical Imaging, and other applications. Generate signals using the arbitrary waveform generator AWG5012C and using data stored in memory and send them to the front panel connectors @640 Mbps of the DSU unit (cost for each duplicate instrument built with this proposal is $2,250). Using the arbitrary waveform generator AWG5012C, the oscilloscope MS070404C, and the Logic State Analyzer TLA7012, work with the company that has manufactured the VME 512 channel board @640 Mbps to test it as a DSU unit. Hire personnel selected in a competitive hiring process at an average cost of $38/hour, which includes undergraduate students, graduate students, and postdoctoral researchers, to be trained in this field to work with the company to test the 512 channel board @640 Mbps as a DSU unit. Students of physics, electronic engineering, and computer science will interact with the company testing the VME board, possibly spending some time at their facility. The company will benefit from the students performing some routine electronic tests and from the development of software which tests the board, and the students benefit by learning from experienced engineers in the field (see Budget Section for details, FIG. 86 and quote #35a on page 137 of BJ, quote #35b on page 143 of BJ).
   NEEDED to advance science and for saving lives: Funds to pay our team of 12 people an average rate of $38/hour and to pay the company for the implementation of the NRE at their average rate of $110/hour.
   See FIG. 86.
6. Build a TER test bench board for 512 channels, at 640 Mbps each for LHC 2017-18, Medical Imaging, and other applications. Record signals from a sequence of data arriving from 512 channels at the front panel connectors at 640 Mbps into memory (cost for each duplicate instrument built with this proposal is $2,250). Using the DSU board tested in the previous phase of this proposal as the instrumentation in place of the arbitrary waveform generator AWG5012C, work with the company that has manufactured the VME 512 channel board @640 Mbps to test this second board as a TER unit. Hire personnel selected in a competitive hiring process at an average cost of $38/hour, which includes undergraduate students, graduate students, and postdoctoral researchers, to be trained in this field to work with the company to test the 512 channel board @640 Mbps as a TER unit. Students of physics, electronic engineering, and computer science will interact with the company testing the VME board, possibly spending some time at their facility. The company will benefit from the students performing some routine electronic tests and from the development of software to test the boards, and the students benefit by learning from experienced engineers in the field (see Budget Section for details, FIG. [symbol] and quotes 35a on page 137 of BJ and 135b on page 143 of BJ).
   NEEDED to advance science and for saving lives: Funds to pay our team of 12 people an average rate of $38/hour and to pay the company for the implementation of the NRE at their average rate of $110/hour.
   See FIG. 87
7. Build a DSU test bench crate for 8,192 channels, at 640 Mbps each for LHC 2017-18, Medical Imaging, and other applications. Generate a sequence of signals from the data stored in memory and send them in parallel every 1.56 nanosecond (ns) to the 8,192 channels at the front panel connectors of the 16× DSU boards (cost for each duplicate instrument built with this proposal is $40,000). Design a DSU VME system for 8,192 channels @640 Mbps per channel. Hire personnel selected in a competitive hiring process at an average cost of $38/hour, which includes undergraduate students, graduate students, and postdoctoral researchers, to be trained in this field to integrate 16× DSU VME boards in a crate, purchase a VME CPU, a VME crate and other instrumentation, test the overall hardware, develop software, etc. (see Budget Section for details, Error! Reference source not found.88 and quotes 36a on page 140 of BJ, 36b on page 142 of BJ and 36c on page 153 of BJ).
   Each university and research center involved in the 2017 Level-1 trigger upgrade of any LHC experiment should have on their laboratory's test bench this DSU instrument costing only $40,000. This would be like having the trigger data generated by the LHC experiment (collider plus detector) costing billions of dollars at their fingertips but at a fraction of the cost. They could test the efficacy of their trigger electronics in executing Object Pattern Real-Time Recognition Algorithms (OPRA) with zero dead-time on data arriving from the DSU unit at 640 Mbps per channel from 8,192 channels as if they were generated by the actual LHC experiment.
   Having a DSU instrument on their laboratory's test bench to test their trigger electronics would avoid wasting millions of dollars building Level-1 Trigger electronics without sufficient capability to execute complex OPRA able to extract all valuable information from new particles, filtering the background noise caused by a higher luminosity at LHC until 2018 and able to resolve pileup events.
   This would also avoid the additional wastage of billions of dollars and millions of hours of work by the over 10,000 people who built other electronics and analyzed useless data because the Level-1 Trigger did not have the capability to select the good events by executing complex OPRA at zero dead-time. This would further avoid discovering that after more than 20 years of work by over 10,000 people and spending billions of dollars, the CMS and Atlas detectors at LHC did not work as expected as it could find only 40 Higgs boson-like particles casually recorded from among the 1,000 trillion events generated by the LHC Collider when 100,000 were estimated to be Higgs boson-like. Even those who built the trigger (CMS and Atlas) admit at conferences and in official documents describing the upgrade of CMS and Atlas that the electronics of the Level-1 trigger needs to be trashed because it does not have object pattern recognition capability.
   This DSU is a valuable instrument for experimenters to create a low-cost controlled environment in their lab capable of not only using raw trigger data recorded at the CERN experiments but also having the possibility to edit the data manually, adding the most difficult patterns they may think could occur to test the efficacy of the 3D-Flow Trigger system (or the Level-1 Trigger system they have developed).
   To avoid re-inventing the wheel . . . , before designing the DSU and TER boards, crates and system, I checked if there was a similar board commercially available. The closest that I could find was the VPX board of quote #36c (see quote #36c on page 153 of the Budget Justification), consisting of 32 channels @250 Mbps per channel and costing $12,595. To build 8,192 channels @250 Mbps per channel, requires 256 of these VPX 57610 boards and 22 VPX crates costing approximately $8,000 each to house them. The total cost using the commercially available boards would cost over $3.4 million.
   My TER/DSU boards and system of 512 channels per board @640 Mbps per channel for any system size has a patent pending. I am willing to offer 80% of my patent licenses to philanthropists and/or investors who will commit to using a percentage of the income generated to pay for free cancer screening examinations for low income people.
   NEEDED: Funds to purchase additional items listed in the budget section and to pay oar team of 12 people average rate of $38/hour.
   See FIG. 88, FIG. 89 and FIG. 90.
8. Build a TER test bench crate for 8,192 channels, at 640 Mbps each for LHC 2017-18, Medical Imaging, and other applications. Record signals from a sequence of data each arriving @640 Mbps in parallel from 8,192 channels at the front panel connectors of the 16× TER VME boards (cost for each duplicate instrument built with this proposal is $40,000). Using the DSU board tested in the previous phase of this proposal as the instrumentation, design, build and test a second crate with 16× TER VME system for 8,192 channels @640 Mbps per channel. Hire personnel selected in a competitive hiring process at an average cost of $38/hour, which includes undergraduate students, graduate students, and postdoctoral researchers, to be trained in this field to integrate 16× TER VME boards in a crate, purchase a VME CPU a VME crate and other instrumentation, test the overall hardware, develop software, etc. (see Budget Section for details, FIG. 91 and quotes #36a on page 140 of BJ, quote #36b on page 142 and quote #36c on page 153 of BJ).
   NEEDED to advance science and for saving lives: Funds to purchase additional items listed in the budget section and to pay our team of 12 people an average rate of $38/hour. See FIG. 91 and FIG. 92.
   Swap DSU and TER functionality on a test bench crate for 8,192 channels, at 640 Mbps each for LHC 2017-18, Medical Imaging, and other applications. Swap the functionality of the two DSU and TER units tested earlier. Make sure that each crate with 8,192 channels can work either as a DSU unit or as a TER unit (cost for each duplicate instrument built with this proposal is $40,000: TER and DSU). Change the functionality of the 8,192 channels, 16× DSU boards in the crate tested earlier to 8,192 channels, 16× TER boards by changing the FPGA code, e.g. inserting a 100 ohm termination resistor at each LVDS receiving line. Change the functionality of the 8,192 channels, 16× TER boards in the crate tested earlier to 8,192 channels, 16× DSU boards by changing the FPGA code, e.g. removing a 100 ohm termination resistor at each LVDS sending line. Hire personnel selected in a competitive hiring process at an average cost of $38/hour, which includes undergraduate students, graduate students, and postdoctoral researchers, to be trained in this field to fully test both functionalities: TER and DSU on the entire crate of 8,192 channels.
   These two new instruments will provide a valuable tool to over 12,000 people working on the LHC experiments at CERN to test the efficacy of their Level-1 Trigger system by placing one TER unit at CERN recording raw Trigger data and sending the recorded data to the Scientific Associates at several universities equipped with a DSU unit.
   These Scientific Associates will have the great advantage of being able to recreate in their lab with only $40,000 the real-time environment of the particle event generator from the detectors at CERN costing billions of dollars. They could use raw trigger data recorded from real experiments at CERN, edit manually the data to include even more difficult situations like high background noise and pileup events, test the functionality and efficiency of their hardware by implementing the Level-1 trigger, improve their electronics and algorithms to assure they have the capability to identify new particles having the characteristics sought by experimenters or provide conclusive test results disproving a theory.
   These two instruments will largely satisfy the needs of the most stringent requirements of experiments running at LHC until 2018. Thereafter, they will still be useful to develop and test the efficiency of any trigger system at a very low cost and help develop the new algorithm and Level-1 Trigger systems for experiments with higher luminosity at LHC after 2019. These should be tested for full performance with the two new DSU and TER units capable of sustaining the double speed of 1280 Mbps per channel described in the next items.
   Building these two instruments is very important because it would save billions of dollars and the time of thousands of engineers and scientists who would otherwise continue for years to build detectors and electronics churning out non-valuable information recorded by inefficient Level-1 Triggers and then spend years analyzing this non-valuable data that could not be tested on a bench of a laboratory. (see Budget Section for details, FIG. 93 and quote #36a on page 140 of BJ, quote #36b on page 142 of BJ and quote $36c on page 153 of BJ).
   NEEDED to advance science and for saving lives: Funds to pay our team of 12 people an average rate of $38/hour. See FIG. 93
9. Design a TER/DSU test bench for 256 channels, at 1280 Mbps each for LHC 2020, medical imaging and other applications. Generate and record signals from/to a sequence of data stored into memory (cost for each duplicate instrument built with this proposal is $2,500). Design a TER/DSU VME board for 256 channels @1280 Mbps per channel. Contact reputable companies that would provide a quote for an NRE cost to manufacture electronics boards offering a turnkey solution.
   DONE (see quote #17a on page 109 of BJ, and quote 17b on page 138 of BJ).
   NEEDED to advance science and for saving lives: Funds to pay the company implementing the NRE at their average rate of $110/hour.
10. Follow same steps from 5 to 9 to develop 16× DSU units, 256 channels boards at 1280 Mbps, 16× TER units, 256 channels at 1280 Mbps, one DSU crate with 4,096 channels at 1280 Mbps, one TER crate with 4,096 channels. Test full functionality of both crates at maximum speed for several hours.
11. Build a DSU test bench crate for 4,096 channels, at 1280 Mbps each for LHC 2020, medical imaging and other applications. Generate a sequence of signals from the data stored in memory and send them in parallel every 781 picosecond (ps) to the 4,096 channels at the front panel connectors of the 16× DSU boards (cost to duplicate each instrument of 8,192 channels built with this proposal is $130,000). Design a DSU/VME system for 4,096 channels @1280 Mbps per channel. Hire personnel selected in a competitive hiring process at an average cost of $38/hour, which includes undergraduate students, graduate students, and postdoctoral researchers, to be trained in this field to integrate 16× DSU VME boards in a crate, purchase a VME CPU, a VME crate and other instrumentation, test the overall hardware, develop software, etc. (see Budget Section for details, quotes 18a on page 109 of BJ and 18b on page 141 of BJ).
   Each university and research center involved in the Level-1 trigger upgrade of any LHC experiment for data taking after the year 2020 with much greater luminosity, with additional sub-detectors providing more information to the Level-1 Triggers and requiring the transfer of more bits per channels at 40 MHz LHC bunch-crossing, should have on their laboratory's test bench this DSU instrument costing only $130,000. This would be like having the trigger data generated by the LHC experiment (collider plus detector) costing billions of dollars at their fingertips but at a fraction of the cost. They could test the efficacy of their trigger electronics in executing Object Pattern Real-Time Recognition Algorithms (OPRA) with zero dead-time on data arriving from the DSU unit at 1280 Mbps per channel from 8,192 channels as if they were generated by the actual LHC experiment.
   Having a DSU instrument on their laboratory's test bench to test their trigger electronics would avoid wasting millions of dollars building Level-1 trigger electronics without sufficient capability to execute complex OPRA able to extract all valuable information from new particles, filter the background noise cause by the much greater luminosity at LHC beginning in 2020, able to resolve pileup events and to handle more data from additional sub-detectors.
   This would also avoid the additional wastage of billions of dollars and millions of hours of work by the over 12,000 people who built other electronics and analyzed useless data because the Level-1 trigger did not have the capability to select the good events by executing complex OPRA at zero dead-time. This would further avoid discovering that after more than 20 years of work by over 10,000 people and spending billions of dollars, the CMS and Atlas detectors at LHC did not work as expected as they could find only 40 Higgs boson-like particles casually recorded from among the 1,000 trillion events generated by the LHC Collider when 100,000 were estimated to be Higgs boson-like. Even those who built the trigger admit it needs to be trashed because it does not have object pattern recognition capability.
   This DSU is a valuable instrument for experimenters to create a low-cost controlled environment in their lab capable of not only using raw trigger data recorded at the CERN experiment, but also have the possibility to edit data manually, adding the most difficult pattern they may thing could occur to test the efficacy of the 3D-Flow trigger system (or the Level-1 Trigger system they have developed).
   To avoid re-inventing the wheel . . . , before designing the DSU and TER boards, crates and system, I checked if there was a similar board commercially available. The closest that I could find was the VPX board of quote #36c (see quote #36c on page 153 of the Budget Justification), consisting of 32 channels @250 Mbps per channel costing $12,595. To build 8,192 channels @250 Mbps per channel requires 256 of these VPX 57610 boards and 22 VPX crates costing approximately $8,000 each to house them. The total cost wsimg the commercially available boards would cost over $3.4 million
   My TER/DSU boards and system at 256 channels per board @1280 Mbps per channel for any system size has a patent pending. I am willing to offer 80% of my patents licenses to philanthropists and/or investors who will commit to using a percentage of the income generated to pay for free cancer screening examinations for low income people.
   NEEDED: Funds to purchase additional items listed in the budget section and to pay our team of 12 people an average rate of $38/hour.
12. Swap DSU and TER functionality on a test bench crate for 4,096 channels, at 1280 Mbps each for LHC 2020, medical imaging and other applications. Swap the functionality of the two DSU and TER units tested earlier. Make sure that each crate with 4,096 channels can work either as a DSU unit or as a TER unit (cost for each duplicate of an 8,192 channel instrument built with this proposal is $130,000: TER and DSU). Change the functionality of the 4,096 channels, 16× DSU boards in the crate tested earlier to 4,096 channels, 16× TER boards by changing the FPGA code, e.g. insert 100 ohm termination resistor at each LVDS receiving line. Change the functionality of the 4,096 channels, 16× TER boards in the crate tested before to 4,096 channels, 16× DSU boards by changing the FPGA code, e.g. remove 100 ohm termination resistor at each LVDS sending line. Hire personnel selected in a competitive hiring process at an average cost of $38/hour, which includes undergraduate students, graduate students, and postdoctoral researchers, to be trained in this field to fully test both functionalities: TER and DSU on the entire crate for 4,096 channels.
   In order to prove full feasibility and functionality of the Triggers' 8,192 channels for CERN's CMS and ATLAS experiments at 1280 Mbps per channel, twice the speed of previous versions of DSU and TER at 640 Mbps, it would be sufficient and would save money to build and test only two crates containing 4,096 channels because all tests are exhausted within the crate (power consumption, crosstalk, speed, signal integrity, etc.). Both crates when set to work as DSU units would provide 8,192 channels, sufficient to test the functionality of a complete 3D-Flow Level-1 trigger system (or any other trigger system) for the largest LHC experiments. These TER and DSU instruments are defined within a crate, but in the previous case of the lower speed there were 8,192 channels per crate and therefore it was necessary to build 16,384 channels to complete all tests.
   It is very important to build these two instruments because it would save billions of dollars and the time of thousands of engineers and scientists who would otherwise continue for years to build detectors and electronics churning out non-valuable information recorded by inefficient Level-1 triggers and then spend years analyzing this non-valuable data that could not be tested on a bench of a laboratory.
   These two new DSU and TER instruments at double speed would provide a valuable tool to the over 12,000 people preparing the construction of a more powerful Level-1 Trigger system for the experiments at LHC with a higher luminosity planned to be operational in 2020.
   The Associates, members of the large LHC experiments, will have the great advantage of being able to recreate in their lab with only $100,000 the real-time environment of particle events that will be generated from the detectors at CERN costing billions of dollars even before its construction. They could use raw trigger data from real experiments previously recorded at CERN, edit manually the data to include even more difficult situations like high background noise and pileup events, test the functionality and efficiency of their hardware by implementing the Level-1 trigger, improve their electronics and algorithms to assure they have the capability to identify new particles having the characteristics sought by experimenters or provide conclusive test results disproving a theory, (see quote #18a on page 109 of BJ and quote #18b on page 141 of BJ).
   NEEDED to advance science and for saving lives: Funds to purchase additional items listed in the budget section, to pay our team of 12 people an average rate of $38/hour, and to pay the company implementing the NRE at their average rate of $110/hour.
13. Invent a method and apparatus that breaks the speed barrier in many real time applications including having the capability to extract ALL valuable information from radiation providing a powerful tool to discover new particles and enabling an effective early detection, improved diagnosis, prognoses and efficient monitoring the treatment of cancer and many other diseases.
   DONE. The 3D-Flow breaking the speed barrier in real-time applications with the capability to extract ALL information from radiation providing a very powerful tool to discover new particles and reduce the cost of HEP experiments was invented in 1992 (see scientific publications). The 3D-CBS (3D-Complete Body Screening) application for medical imaging that makes use of the 3D-Flow architecture that enables early cancer detection that could have already saved many lives if funded announced its invention in the year 2000 (see scientific publications).
   NEEDED to advance science and for saving lives: a fair PUBLIC merit review by funding agencies to fairly compare and fund the complete implementation of the 3D -Flow and 3D-CBS inventions, capable of extracting ALL valuable information from radiation, found superior in terms of advancing science, saving money, and saving lives, so that humanity will no longer be deprived of the benefits from these inventions and taxpayers will no longer be burdened by funding less efficient and more costly approaches.
14. Recognize the 3D-Flow invention as feasible and valuable when compared to other approaches, have it published in scientific journals, present it at conferences, and defend its advantages and novel idea in public formal scientific reviews, in public debates, and in a thorough and exhaustive examination by patent examiners.
   DONE. The 3D-Flow invention was acclaimed since 1992 in many letters from scientists and experts in the field. In 1993, it was recognized as feasible and valuable by academia, industry and the world's most prestigious research centers in a formal international scientific review reported in detail at this link. The final report by the review panel stated “The committee finds this project an interesting and a unique concept. We believe the concept will work . . . We see no technical reasons why the proposed ASIC processor could not be built . . . We do not believe that there are any major flaws . . . We see little risk in this approach to the processor chip design itself. The documentation should be relatively easy since most of what is required currently exists.” They also recognized that it could be used in future applications that go beyond their imagination when referring to current scientific knowledge stating: “given this feature experimenters would probably think of clever uses not now possible”.
   It was recognized feasible and valuable by DOE who published the 3D-Flow architecture on page 216 of their 1994 Technology Transfer book DOELM-002 DE94005148.
   It was recognized feasible and valuable by thousands of scientists of the GEM (Gamma Electron and Muon) experiment at the Super Conducting Super Collider (SSC) who adopted the 3D-Flow architecture for their Level-1 Trigger as reported on pages 7-10 to 7-14 of the GEM-TDR-TN-93-262, SSCL-SR-1219, until the project was terminated by the U.S. Congress.
   It was recognized feasible and valuable by hundreds of scientists of the LHCb collaboration, one of four major experiments at CERN-LHC, who adopted the 3D-Flow for the Calorimeter and Muon Level-0 (or Level-1) trigger, as reported on pages 83-84 in their Letter of Intent CERN/LHCC 95-5, LHCC/I8 written Mar. 28, 1996 and on pages 103-108 of the LHCb Technical Proposal CERN/LHCC 98-4 LHCC/P4 written Feb. 20, 1998 and in the peer-review NIM article Vol. A 436, 1999, pp. 341-385, before funding for Alice and LHCb experiments was cut by DOE for budget reasons.
   It was officially recognized an invention when its patent was granted.
   However, instead of funding to complete the implementation of the 3D-Flow invention on its own that could have already provided a powerful tool to discover new particles and to save lives, it was always dependent upon the adoption of other experiments. Unfortunately, the experiments it depended on were either terminated as was the case for the SSC, or the funding agency stopped funding a particular experiment as was the case for the LHCb. Meanwhile, less efficient and more costly projects were being funded because funding agencies do not require researchers to compare directly in a public debate their proposals against each other to justify/defend the cost-effectiveness of their approach, and because each experiment gave the excuse that they did not have the funds to pay for the NRE of the 3D-Flow that was also serving other experiments. As a result, they ending up spending more money for their less efficient, mostly “cabled logic” approaches or having little programmability that could not execute complex real-time level-1 trigger algorithms at zero dead-time.
   NEEDED to advance science and for saving lives: a fair PUBLIC merit review by funding agencies to fairly compare and fund the complete implementation of the 3D-Flow and 3D-CBS inventions capable of extracting ALL valuable information from radiation, found superior in terms of advancing science, saving money, and saving lives, so that humanity will no longer be deprived of the benefits from these inventions and taxpayers will no longer be burdened by funding less efficient and more costly approaches.
15. Recognize the 3D-CBS invention feasible and valuable when compared to other approaches, published in scientific journal, presented at conferences, defending its advantages and novel idea in public formal scientific reviews and in public debates, in thorough and exhaustive examination by patents examiners who could not object to the novelty of the claims granting the patent and without finding any scientist or expert in the field who could point out another approach that could provide higher performance solutions at a lower cost invention valuable.
   DONE. The 3D-CBS invention based on the 3D-Flow architecture to extract ALL valuable information from radiation was recognized feasible and valuable in several PUBLIC international scientific reviews, when presented at conferences, research centers, and hospitals. No one could object its superiority in performance and cost-effectiveness in extracting ALL valuable information from radiation providing staggering advantages in 400 times improved efficiency compared to current PET, which enabled for the first time an effective early cancer detection of cancer and many other diseases at a highly curable stage and at an affordable examination cost.
   The 3D-CBS was recognized feasible and valuable when in 2011 it won the Leonardo da Vinci Prize for the most efficient solution using particle detection for early cancer diagnosis. This competition was open, announced in several press releases including Business Wire to over 6000 news outlet. [³³] published on May 12, 2011 [³⁴] and on May 26, 2011 [³⁵] by and newspapers, television, radio stations, and online news forums and held at the University of Pavia, Italy, during its 600^{th} anniversary.
   It was officially recognized an invention when it received its patent in both the U.S. and in Europe. The European patent was granted in a few minutes after I was given the opportunity for a face-to-face meeting with the committee of patent examiners where I could explain the new inventive steps and answer directly their doubts and questions. Until then my patent had been represented by patent attorneys from three major international law firms in the fourteen years since its application, but their letters and email exchanges had been insufficient to persuade the examiners. I am grateful to my last patent attorney who realized the need for the face-to-face meeting and made the necessary arrangements.
   Unfortunately, the reviewers of funding agencies and IEEE anonymous reviewers continue to be led by the misconception that the most important feature for all medical imaging devices is spatial resolution because they receive such requests from doctors, hospitals, pharmaceutical companies, cancer organizations and leaders in the field who want to measure the smallest differences in tumor size because they want to monitor the effect of a new drug for example; By focusing instead on extracting ALL valuable information from radiation, they will not be throwing away over 90% of useful data from radiation by using a poor geometry design, and they will receive signals that would provide them with even better information on spatial resolution.
   NEEDED to advance science and for saving lives: a fair PUBLIC merit review by funding agencies to fairly compare and fund the complete implementation of the 3D-Flow and 3D-CBS inventions capable of extracting ALL valuable information from radiation, found superior in terms of advancing science, saving money, and saving lives, so that humanity will no longer be deprived of the benefits from these inventions and taxpayers will no longer be burdened by funding less efficient and more costly approaches.
   I designed the cylinder structure shown in FIG. 17 so that it could be easily assembled and accessed as shown in FIG. 18 in the event there needed to be repairs made by providing an opening along a line wall accessible on both sides. Its cylindrical structure reflected the cylindrical structure of the calorimeter and tracking detectors and would have given maximum efficiency by allowing every detector element to communicate within a short distance of five inches to its neighbors in the adjacent mini-crates.
   I assembled the mini-crate, connected the DAQ board at 90 degrees to the mother board that was the base to stack as many daughter boards as needed (see FIG. [symbol] ) to satisfy the most demanding requirements of experimenters' desired real-time algorithms that would select the particles with their desired characteristics from the background or from other non-interesting particles. The daughter boards (see FIG. 23—Details of the Mini-crate showing the rear view of the motherboard of the 3D-Flow system to optimize short cable length in a cylindrical geometry.
   FIG. [symbol] ) have the footprint for the BGA 3D-Flow integrated circuit (whose NRE was never funded) with all I/O to processors in the previous and subsequent stack of daughter boards, as well as to their North, East, West, South 3D-Flow processors on the same board or on the neighboring mini-crate. I tested the data transmission speed from board to board across the flexible printed circuit for speed, signal integrity and for crosstalk between adjacent channels.
   It was disappointing to learn that all my careful painstaking work that you see in the photo had been trashed.
   I was able to salvage only one mini-crate, one mother-board, a few daughter-boards, and a few flexible printed circuits with connectors before they were trashed. One of the boards is still assembled with the fast inverter MC74F04, the octal buffer, and line driver 74ABT244C. My 1994 design of the 3D-Flow system was feasible and functional proving that I made available an invention having the capability to execute experimenters' programmable Level-1 trigger algorithms while sustaining 40 million events per second with billions of collisions per second and zero dead-time.
   More disappointing though is to know the positive results in the development of all components of my 3D-Flow system on a shoe-string budget (using $906,000 in grants from DOE from 1995 to 1997, nearly all my economical resources and over fifteen years of full-time days, evenings and most weekends of unpaid work), prove day after day that what was formally recognized by the review panel of my invention in 1993 at FERMILab and expecting it would have been used in some future applications that went beyond their imagination at that time: “given this feature experimenters would probably think of clever uses not now possible”, was a dream that could have already been a reality if NRE funding of the 3D-Flow processor and funds to complete one 3D-Flow system had been provided.

Instead, even after recognizing the feasibility and value of my invention, over $50 billion was spent on 20 years of work by over 10,000 people to build CERN experiments (CMS and Atlas) having Level-1 triggers that do not have the capability to execute Object Pattern Recognition Algorithms, do not have zero dead-time, and could only find 40 of the estimated 100,000 Higgs boson-like particles. Furthermore, these 40 events were recorded casually and found after analysis of the recorded data. Now, Atlas and CMS are once more making the same error of choosing FPGA which is more costly and less performant than the 3D-Flow and refusing to compare its performance and cost in an open public review where the advantages of the 3D-Flow invention could be recognized and its claims defended in a public debate with CMS, Atlas, and other trigger experts who support the use of FPGA or other trigger systems.
The cylinder implementation of the 3D-Flow would have provided two decades ago the capabilities to execute programmable Level-1 Trigger algorithms at 40 MHz bunch-crossing with zero dead-time, but now it is more cost-effective to build the technology-independent 3D-Flow system in a 19 inch×19 inch×25 inch box, which is even smaller, faster, and has a lower power consumption and lower cost satisfying LHC requirements beyond 2020. See in the description of the steps that follow the implementation of the 3D-Flow system with current technology.
NEEDED to advance science and for saving lives: a fair PUBLIC merit review by funding agencies to fairly compare and fund the complete implementation of the 3D-Flow and 3D-CBS inventions capable of extracting ALL valuable information from radiation, found superior in terms of advancing science, saving money, and saving lives, so that humanity will no longer be deprived of the benefits from these inventions and taxpayers will no longer be burdened by folding less efficient and more costly approaches.
16. Demonstrate the 3D-Flow invention is technology-independent, easily migrating to future technologies having the advantage of lowering the price and increasing performance as technology improves.
   DONE. During the implementation of the DOE $906,000 grant from 1995 to 1998, I proved the 3D-Flow design to be technology-independent by compiling the 3D-Flow processor/chip in three different FPGA (Field Programmable Gate Array) technologies (ORCA, from Lucent, Xilinx and Altera) and to a standard cell 350 nanometer (nm) CMOS technology using Synopsys tools. I also paid them as a consultant to provide the tape-out file to be sent to the silicon foundry that would manufacture the chip (Synopsys is recognized as having one of the best software tools and being one of the best ASIC Design companies in the world). Upon request, I can provide the gate count, speed performance, and power dissipation for the four designs.
   Unfortunately, the funds to pay the NRE to the silicon foundry to make the 3D-Flow chip with 350 nm technology was never provided, and although Synopsys tools, because of the success they have had with many designs in the past, guarantees that the silicon chip would have the same characteristics and performance as the design they provided in the tape-out file to the foundry, we could never test the chip as it was never funded. Later, in 2003, I used my own savings and money I received from friends, to implement four 3D-Flow processors in an Altera FPGA that was tested in Altera prototypes boards and later included in a modular industrialized IBM PC board with 68×3D-Flow processors. See in the following steps.
   NEEDED to advance science and for saving lives: a fair PUBLIC merit review by funding agencies to fairly compare arid fund the complete implementation of the 3D-Flow and 3D-CBS inventions capable of extracting ALL valuable information from radiation, found superior in terms of advancing science, saving money, and saving lives, so that humanity will no longer be deprived of the benefits from these inventions and taxpayers will no longer be burdened by funding less efficient and more costly approaches.
17. Demonstrate feasibility to implement a 3D-Flow chip in 350 nanometer technology with 4×3D-Flow processors per chip
   DONE. During the implementation of the DOE $906,000 grant from 1995 to 1998, I proved feasibility to implement four 3D-Flow processors in a chip with 350 nanometer technology. Synopsys, the consultant who generated the tape out files to be sent to the foundry for the fabrication of the chip with 3D-Flow processors successfully simulated each processor at 61 MHz (Synopsys is recognized as having one of the best software tools and being one of the best ASIC Design companies in the world). This allowed to execute more than one cycle of up to 26 operations such as add, subtract, compare one value with 24 values, etc. and the bypass switch of the 3D-Flow architecture, together with the stacking assembly of several daughter boards of the 3D-Flow system already built in 1994 at the Superconducting Super Collider allowed to increase the number of cycles to implement any experimenters' complex real-time algorithm at the Level-1 Trigger.
   Synopsys provided a report of silicon characteristics of each 3D-Flow processor synthesized in 350 nanometer technology as follows: Logic 2,565,989 units*0.0378=96,940; Memory 2,247,513 equivalent to 90K gates.
   Unfortunately, the funds to pay the NRE to the silicon foundry to make the 3D-Flow chip with 350 nm technology was never provided, and although Synopsys tools, because of the success they have had with many designs in the past, guarantees that the silicon chip would have the same characteristics and performance as the design they provided in the tape-out file to the foundry, we could never test the chip as it was never funded.
18. Develop the software tools to simulate thousands of processors in C++, four processors in a chip at the gate level in VHDL, and the real-time design software tool to allow users to create a project of different size and complexity, simulate it, verify the performance and feasibility from user system algorithm to gate-level circuits, interface the 3D-Flow software tools to third-party Electronic Design Automation (EDA) tools, allow implementation of user's conceptual idea into the fastest programmable system at the gate circuit level, etc.
   DONE. The 3D-Flow Design Real-Time software tools were developed with a DOE grant from 1995 to 1998 and are summarized in Error! Reference source not found.
   NEEDED to advance science and for saving lives: funds to pay our ream of 12 people at an avenge cost of $38/hour to do the porting of all this software from NT operating system and the software environment when it was developed in 1996 to Windows 10, Linux, OS-2, MsWorks, to write user manuals of the software to facilitate users.
19. Demonstrate the 3D-Flow system with the capability to execute Level-1 Trigger algorithms in real-time that can replace an entire Level-1 (or Level-0) trigger system in any of the large experiments at CERN-LHC
   DONE. A complete description of the 3D-Flow system for the Level-0 trigger for LHCb experiment was approved in less than one month from submission for publication by peer-review scientific journal.
   NEEDED to advance science and for saving lives: a fair PUBLIC merit review by funding agencies to fairly compare and fund the complete implementation of the 3D-Flow and 3D-CBS inventions capable of extracting ALL valuable information from radiation, found superior in terms of advancing science, saving money, and saving lives, so that humanity will no longer be deprived of the benefits from these inventions and taxpayers will no longer be burdened by funding less efficient and more costly approaches
20. Demonstrate feasibility in hardware of the concept of the 3D-Flow invention.
   DONE. The first demonstration of the 3D-Flow invention to be feasible and functional in hardware was presented at the 2001 IEEE-NSS-MIC industrial exhibition in San Diego (Calif.). See more detail at page Error! Bookmark not defined.
   NEEDED to advance science and for saving lives: a fair PUBLIC merit review by funding agencies to fairly compare and fund the complete implementation of the 3D-Flow and 3D-CBS inventions capable of extracting ALL valuable information from radiation, found superior in terms of advancing science saving money, and saving lives, so that humanity will no longer be deprived of the benefits from these inventions and taxpayers will no longer be burdened by funding less efficient and more costly approaches
21. Demonstrate feasibility in hardware to build & 3D-Flow system for detectors of any dimension for HEP and Medical imaging applications by developing modular electronic industrialized boards.
   DONE. In 2003, I proved the feasibility and functionality of 3D-Flow systems for HEP applications and Medical Imaging by building industrialized IBM PC modular electronic boards, at my own expense, with 68 processors each. The successful testing of the communication between these two modular boards proved that a 3D-Flow system for any detector size in HEP or in Medical Imaging applications can be built, with the advantages of extracting all relevant information from radiation at the lowest cost per valid signal captured, to discover new particles, and to implement an effective, low-dose radiation, low-cost early cancer detection.
   NEEDED to advance science and for saving lives: a fair PUBLIC merit review by funding agencies to fairly compare and fund the complete implementation of the 3D-Flow and 3D-CBS inventions capable of extracting ALL valuable information from radiation, found superior in terms of advancing science, saving money, and saving lives, so that humanity will no longer be deprived of the benefits from these inventions and taxpayers will no longer be burdened by funding less efficient and more costly approaches
K. Scientific Innovations and Technical Merits of the Project
12. Example of the sequence of ideas, followed by analytical thinking, calculations, and verification of feasibility with existing technology which led to one of the many inventions in this project

The invention I have chosen as an example provides the most cost-effective approach to transfer 1.3 TB/sec data to the most compact, lowest power consumption, and most performant 3D-Flow system.
Step-by-Step analytical thinking which led to my INVENTION of Transferring Data
28 TB/sec @<$40K
Testable up to 60 TB/sec (480 Tbps) to check when it fails
Between 2 ATCA or VXI Crates

This is just one example of one invention among several that I created for this project in order to optimize all aspects of the design that would provide the most powerful tool in discovering new particles and in the most effective device for early cancer detection, both benefitting from the advantages of extracting ALL valuable information from radiation by executing complex Object Pattern Real-Time Recognition Algorithms on thousands of data arriving in parallel from the detector at a million frames (or events) per second.

I followed a similar sequence of ideas, analytical thinking, calculations, etc., for the selection of the most cost-effective implementation of the 3D-Flow processor chip. In that case I had to evaluate which technology was most cost-effective (from 350 nanometer to 14 nanometer) for the volume of units (chip or ASICs, Application Specific Integrated Circuits) we anticipate needing, whether it was better to house 16, 64, or 256×3D-Flow processors in a chip, which I/O multiplexing rate was more advantageous for communicating with neighboring processors, and which was more cost-effective for Bottom to Top port of the processor, power consumption vs. speed and cost, etc. I am confident that all possible approaches have been considered and that the analytical thinking led to the choice of the most cost-effective approach of $1 per 3D-Flow processor for 55,500 chips with this price lowering to $0.42 per 3D-Flow processor for much larger quantities.

I also followed a similar sequence of ideas, analytical thinking, calculations, etc. in the selection of the most cost-effective FPGA to be used in my design of the LHC TER/Simulator (DSU) VME board. As you can see from the quotes, this process was able to identify the components for a board costing $3,000 using the most cost-effective FPGA having the same functionality as a board costing $18,000 with expensive FPGAs.

The variables I had to consider to find the best approach to transfer 10 Tbps from the PRAI crate to the 3D-Flow crate in Error! Reference source not found. were many. Here is a list of a few:
1. Is it globally at the system level more cost effective to use 8,192 cables at 1.28 Gbps per cable or 1048 Ethernet cables at 10 Gbps per cable?
2. What is the component cost of each approach?
3. Can the dimensions of the connectors carrying the information from the 1024 cables in the 1.28 Gbps approach fit on the front panel of a 322 mm ATCA blade module?
4. Can the dimensions of the Ethernet connectors carrying the information from the 131 cables in the 10 Gbps approach fit on the front panel of a 322 mm ATCA blade module?
5. Considering the 1.28 Gbps approach, is it more cost-effective to use connectors with 0.8 mm pitch or 1.27 mm pitch?

I started answering these questions augmented by several others that arose in the process by first looking for a connector in the catalogue of Amphenol, Molex, Tyco and SamTec.

I found the SamTec series SEAM and SEAF and first selected the right angle 300-pins SEAF-30-01-S-10-2-RA-TR, with a total length of 49.28 mm. Four of these connectors just fit on the front panel of a 6U VME module 266.7 mm×20 mm and could be soldered on the PCB board 233 mm (4×49.28 mm=197.12 mm).

Next, I verified that this 300-pin connector carrying 131 LVDS signals would meet the speed requirements. Although the catalogue stated 36 Gbps, I asked the local SamTec representative and my question was copied to the regional representative, to the headquarter office in the U.S. and to the Signal Integrity (SIG) group at the manufacturing plant in Taiwan. The answer came back no, it would not support 1.28 Gbps because there were too few ground pins.

With a more in-depth study, I learned that to achieve 36 Gbps it was necessary to have only 75 differential lines and 150 ground pins with a pinout as reported in FIG. [symbol] .

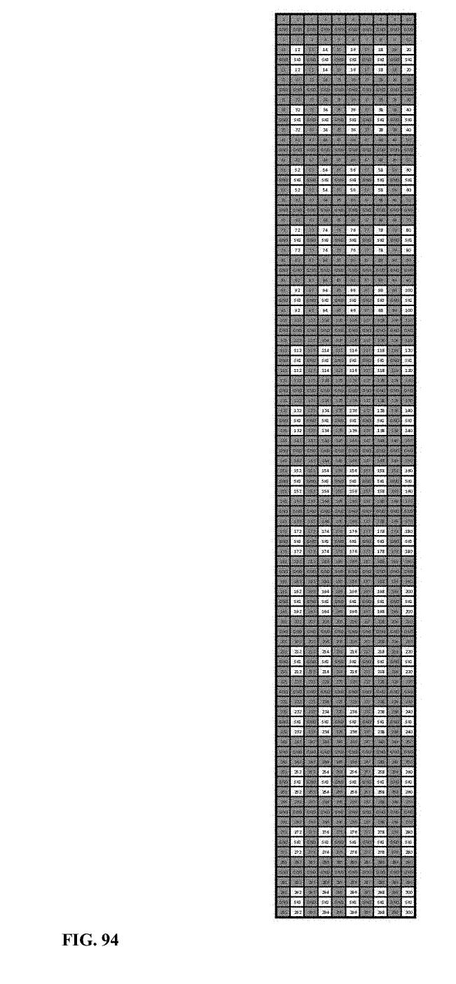

See FIG. 94.

I studied in more in detail several other options from the catalogue and felt intuitively that stacking connectors with only 7 mm pin length might reduce the crosstalk that was coupling in the 15+ mm pin length in the right angle connectors.

The engineer at the manufacturing company did not believe it would make much of a difference. I asked if he would do a simulation, and the next day I received an email from the same engineer in Taiwan stating “As you expected, 7 mm SEAM/SEAF crosstalk looks okay with only a few ground pins”, and provided graphs of the insertion loss and crosstalk at different frequencies showing the 7 mm stacking connectors working even when using fewer ground pins.

After learning the characteristics of these connectors that could achieve 36 Gbps and have the advantage of using 7 mm stacking connectors requiring fewer ground pins, I decided to build a system with the capability of 10 Gbps per Micro Twinax cable with a 50% safe margin of reliability in all components (I selected a connector with more pins and a cable that would support 10 Gbps). Therefore, I selected 400-pin stacking connectors SEAM-40-03.5-S-10-2-A for 131 LVDS signals (see FIG. [symbol] ) and I gladly accepted the compromise of good signal integrity in exchange for the necessity to extract the entire board to remove each connector instead of the ability to remove the connectors one at a time from the front panel of the board. I have used stacking connectors in the design of previous boards where one connector is on the PCB and the other on a FlexPCB where it was necessary to extract the board in order to remove the FlexPCB, and found it to be both reliable and easy to insert/extract the connector.

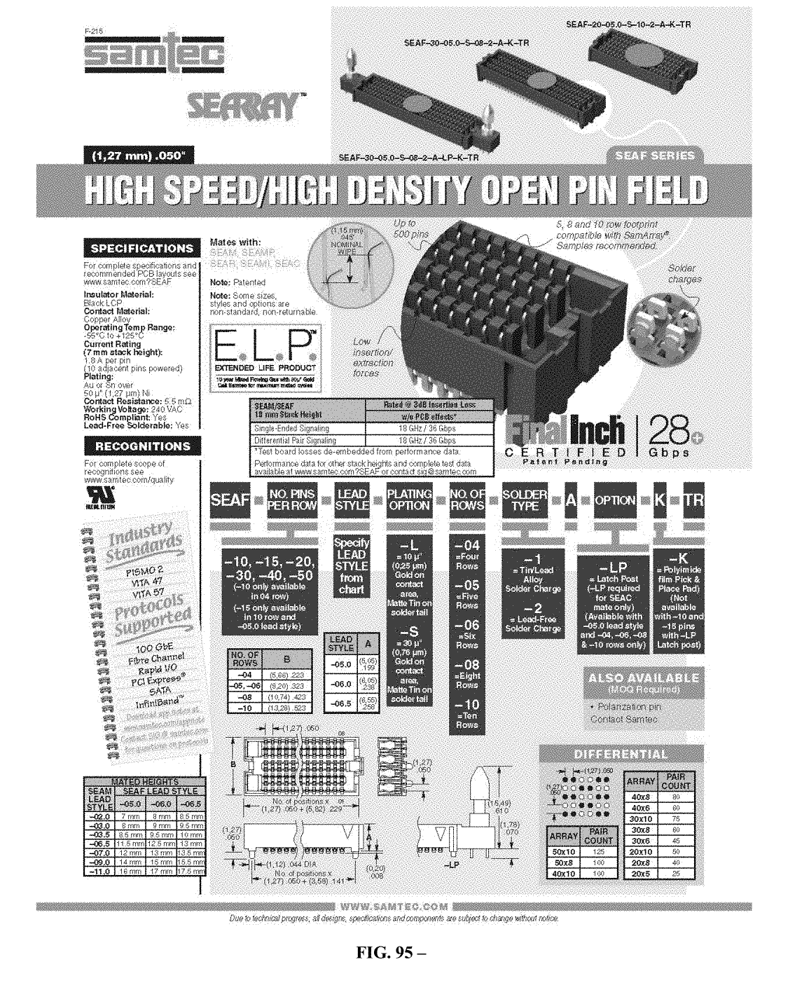

See FIG. 95

Next I needed to select the cable and made enquiries with a few companies on whether they could manufacture a FlexPCB connecting two SEAM-40-03.5-S-10-2-A connectors carrying 131 LVDS signals. Although none said it was impossible, there were some technical difficulties and I never received a quote. One of the companies that bid for the construction of the LHC TER/Simulator (DSU) board received a quote for a FlexPCB connecting the two 400-pins connectors, however it cost more than the solution I had come up with using Micro Twinax cables with the characteristics reported in FIG. [symbol] (see http://suddendocs.samtec.com/notesandwhitepapers/ttf-32100-xx-xx_datasheet.pdf)

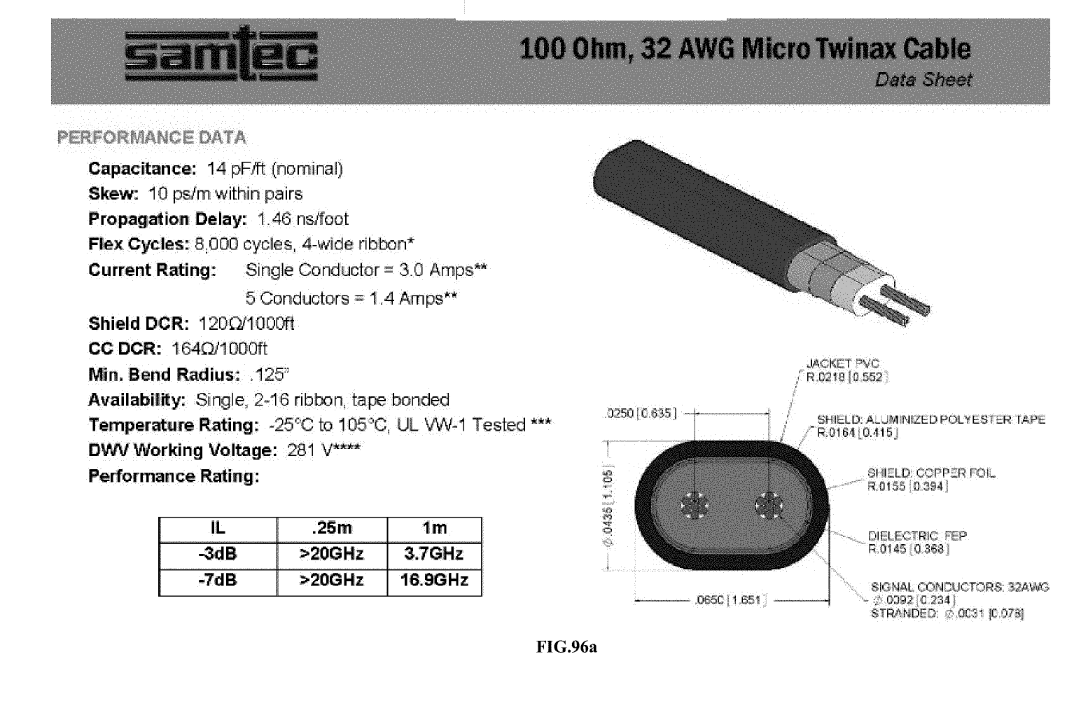

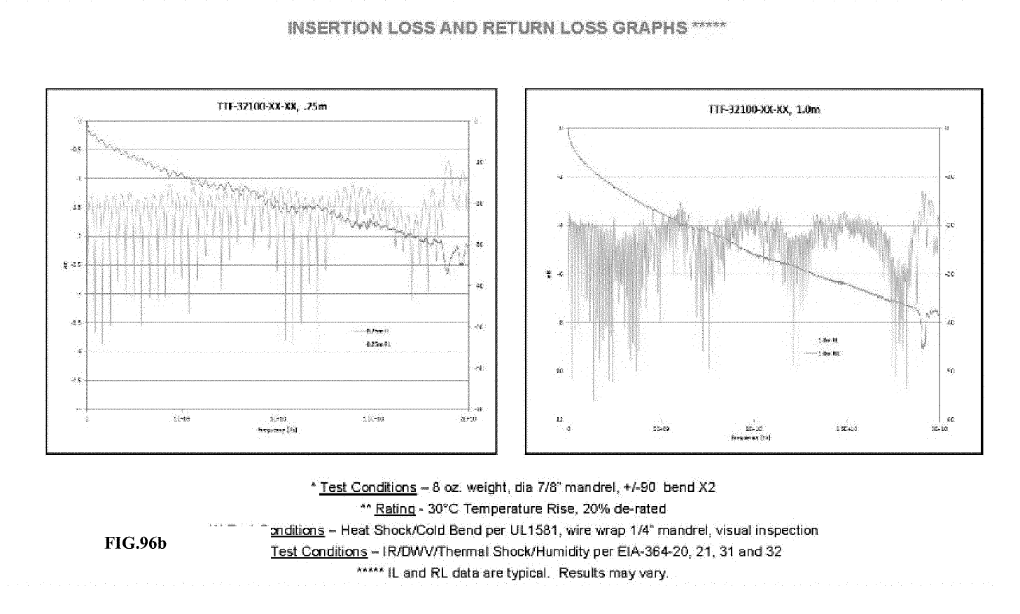

See FIG. 96

The Micro Twinax cable exceeds by over 10 times the speed needed in my application; it also has much lower insertion loss and crosstalk than needed. FIG. [symbol] shows the graph of the insertion loss at −3 dB for a cable length of 0.25 m working at a speed greater than 20 GHz and for 1 m working at a speed greater than 3.7 GHz. It follows that it could comfortably reach 10 GHz at 0.5 m length used in this application as shown in Error! Reference source not found. (11U+2 inch=494.03 mm). Therefore, I am selecting the components (connectors and cables) targeting a system having the capability to transfer 10 Gbps per Micro Twinax with a margin that could reach a 50% higher frequency although I am using it only for 1.28 Gbps. This will provide a very reliable system.

I started communicating via phone and email with SamTec high speed cable application group in Costa Rica and sent the signal pin assignments of my design to SamTec connector SEAM-40-03.5-S-10-2-A as reported in FIG. [symbol] .

Considering the Twinax ribbon cable was made of 16×2 wires, I gave up the three control lines START, STOP and CLOCK from this connector and I planned to carry those signals on another connector.

On Nov. 19, 2015, I created a series of drawings, improving from one drawing to the next by thinking analytically, verifying the new drawings were satisfying all system requirements in the three applications described in Error! Reference source not found, Error! Reference source not found, and Error! Reference source not found. Sometimes during verification I would notice some incompatibility or obstacle in some area, go back to the drawing board, modify my PowerPoint drawings, recheck, remodify, until all calculations, part dimensions, etc., harmoniously fit together.

This analytical process lead me in one day to invent a cable/connector assembly capable of transferring (with a margin of reliability of 50% higher speed), up to 28 TB/sec (or 224 Tbps) between two ATCA or VXI crates at a cost less than $40,000, which is exceeding all transfer rates used at CERN between two crates and it satisfies the requirements of CERN experiments for a few decades. For this specific application requiring the transfer of only 1.3 TB/sec between 8 electronics boards in one ATCA crate and 8 electronics boards in a VXI crate, the cost of the cables with connectors is approximately $10,000. It will also support future upgrades transferring up to 10 times this speed, being able to transfer up to 13 TB/sec without the need to purchase other cables. At the end of this section I will provide the calculations showing how this staggering transfer rate between two crates can be achieved.

I therefore created the first drawing for the assembly of the 128 Twinax cables connecting the two SamTec connectors at both ends of SEAM-40-03.5-S-10-2-A as reported in FIG. [symbol] . This assembly consists of two PCBs each soldered at one end to the connector; the other two ends of the PCB carries on each side two columns of two rows of 16×2 pads staggered 13 mm to avoid crosstalk between signals, and to leave room for UV epoxy covering the soldering of the wires of the Twinax ribbon cables.

To provide high reliability during vibrations or if connectors become loose during insertion/extraction of the boards, I provided four screws at each corner of the connector. Unlike the larger holes for two screws at the center on both sides of the connector as commonly used in commercial products, I provided holes on the PCB for four smaller screws ( 3/32 inch) at the four corners of the connector drilled in the position that will keep the width of the PCB the same as the length of the connector. This will allow more connectors to be placed at the edge of the front panel of the application PCB.

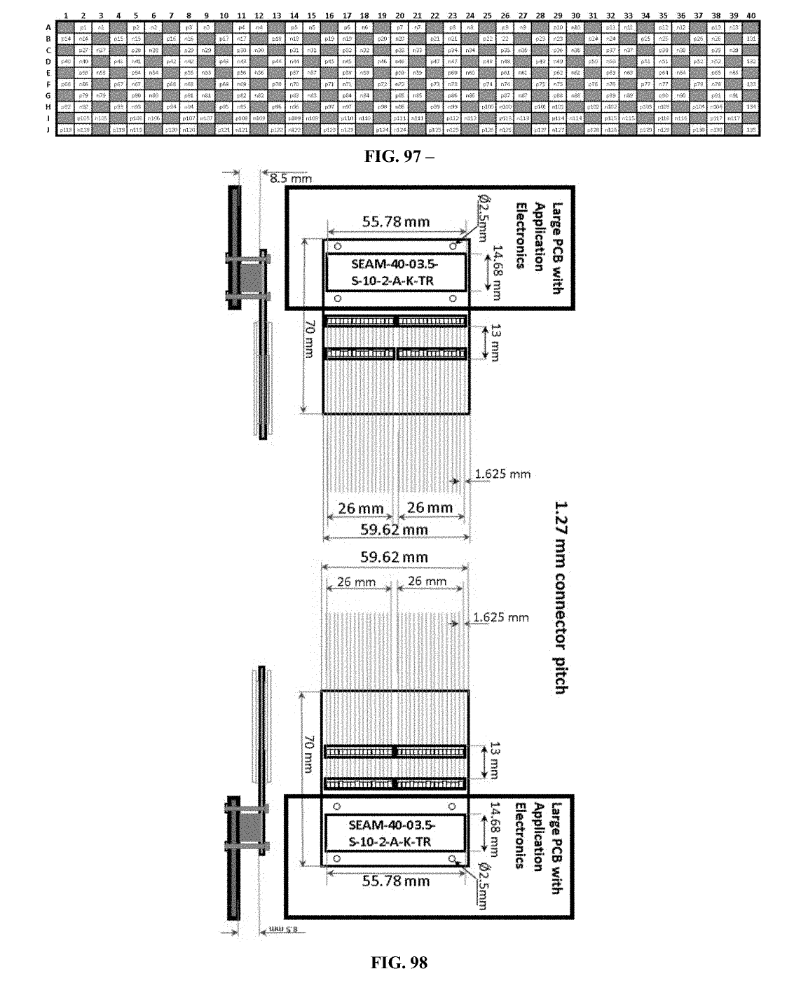

See FIG. 98.

I realized that I forgot to secure the ribbon cables to the small PCB to remove strain solicitations to the cable that could break the soldering of the wire to the PCB. However, I also realized that 56 mm width PCB was required because the length of the connector is 55.78 mm which will only allow 3 connectors to be placed at the edge of the VME board facing the front panel VME module (210 mm usable space), 5 connectors at the ATCA board (310 mm usable space), and 6 connectors at the VXI board (352 mm usable space). This requires more boards and more crates to house the same number of 3D-Flow processors, increasing the length of the cables which will increase the delay of the signals, which will increase its power consumption and cost.

An optimized modularity of LVDS channels per connector, connectors per board, boards per system is: 128 LVDS channels per connector, 2 to 4 connectors per VME board, 8 connectors per VXI or ATCA board, 8 or 16 VME boards per VME crate, and 8 boards per ATCA and VXI crate.

Upon further study of other options from the SamTec catalogue, I identified the series SEAF8, 0.8 mm pitch connectors with the specifications listed in FIG. [symbol] that would provide 400-pin with a length 37.84 mm which would satisfy the requirements of placing the desired number of connectors per VME, VXI and ATCA boards listed before.

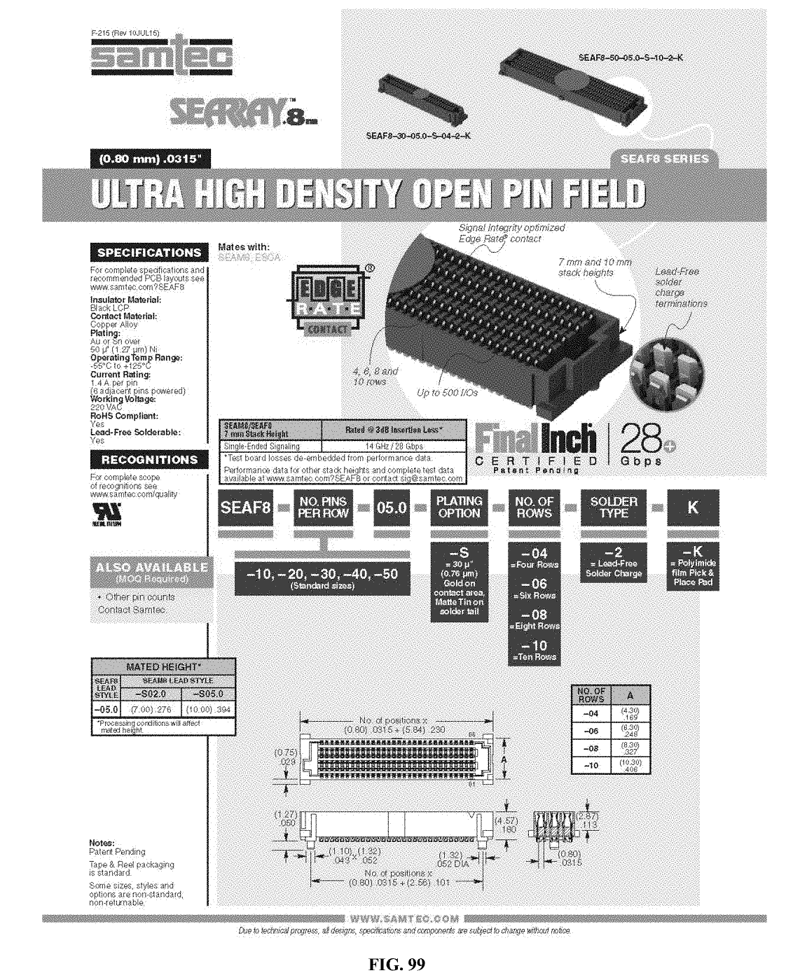

See FIG. 99.

I went back to the drawing board of my Power Point and designed the layout of the small board 75 mm×37.84 mm housing the connectors SEAF8-SEAM8-40-S02.0-S-10-S-K, 0.80 mm pitch and the pads for 128 Micro Twinax Cables. The connector is assembled at one end of the board, while at the other end, on both sides of the board are four columns of pads. Each column has 16×2 pads for one ribbon cable. There are four ribbon cables on each side of the board. I then provided a strain reliever that keeps the eight ribbon cables tight together to the small 75 mm×37.84 mm boards and a second strain reliever tightening this bundle to the larger application PCB board.

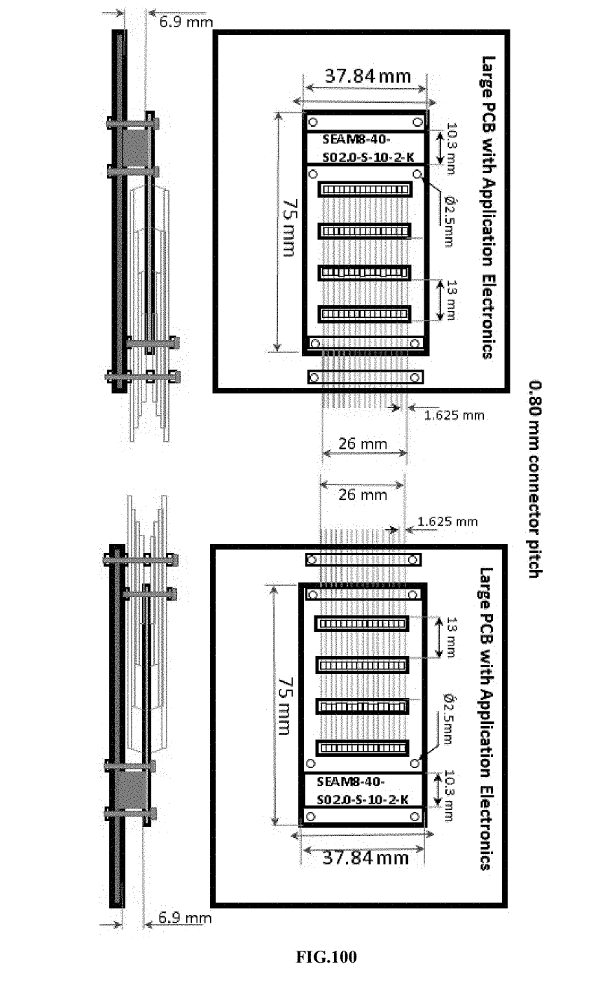

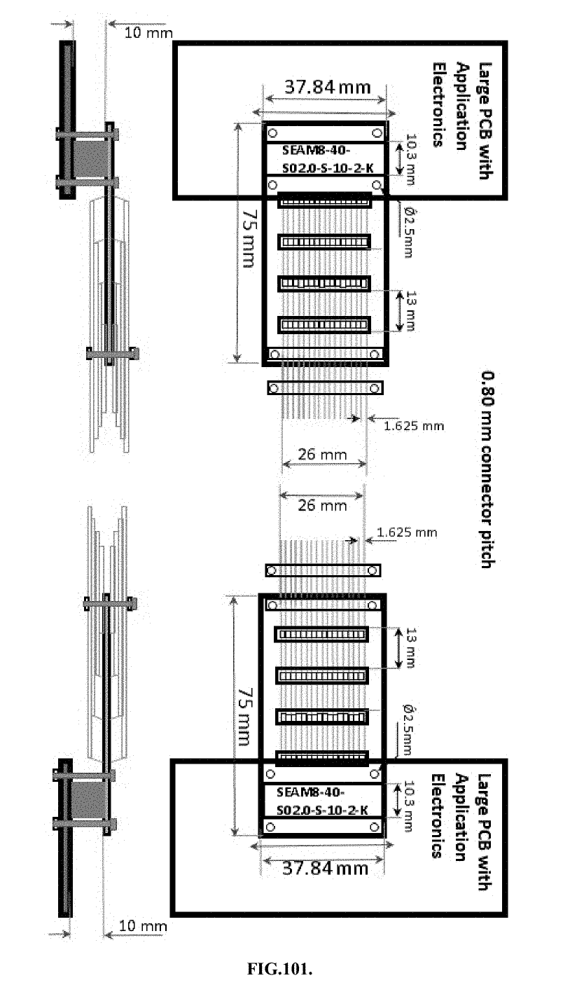

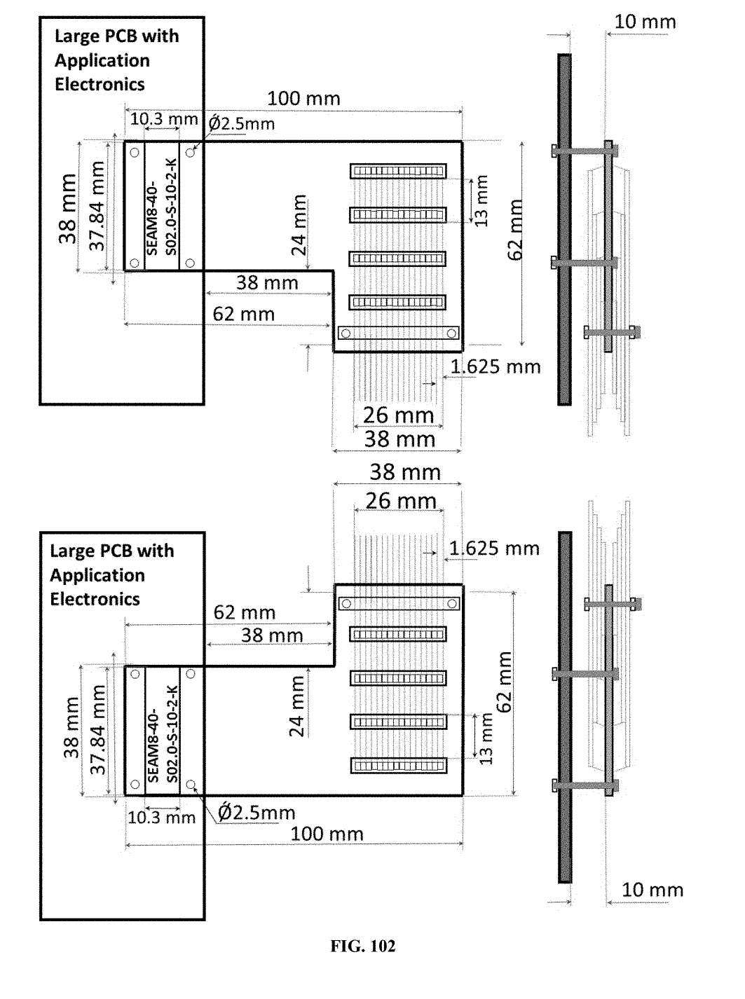

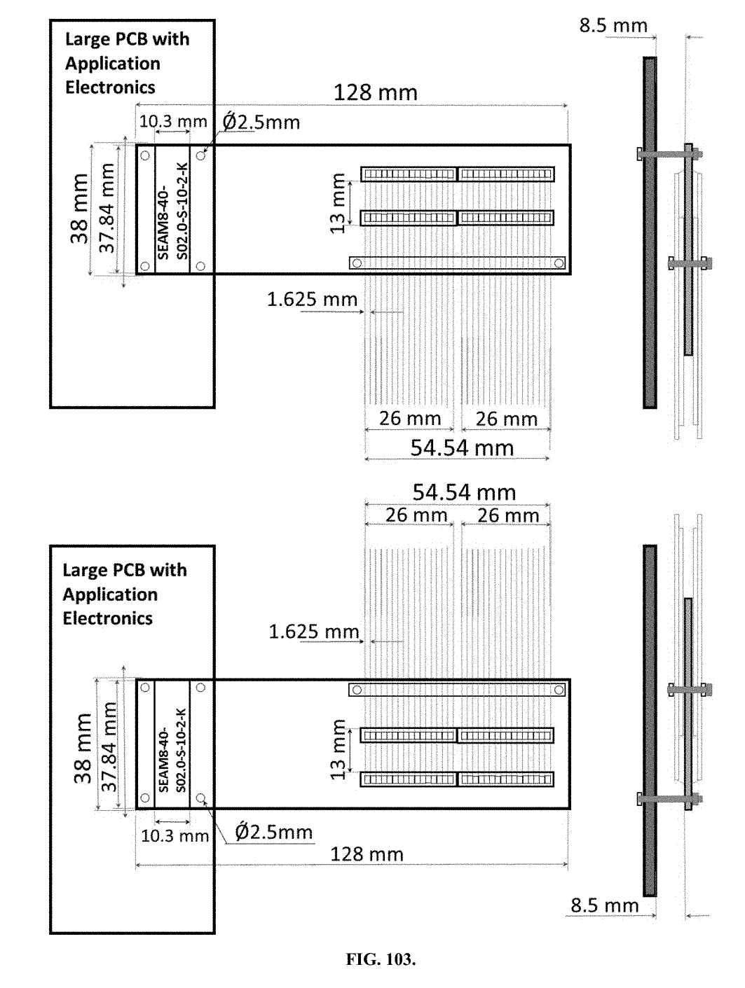

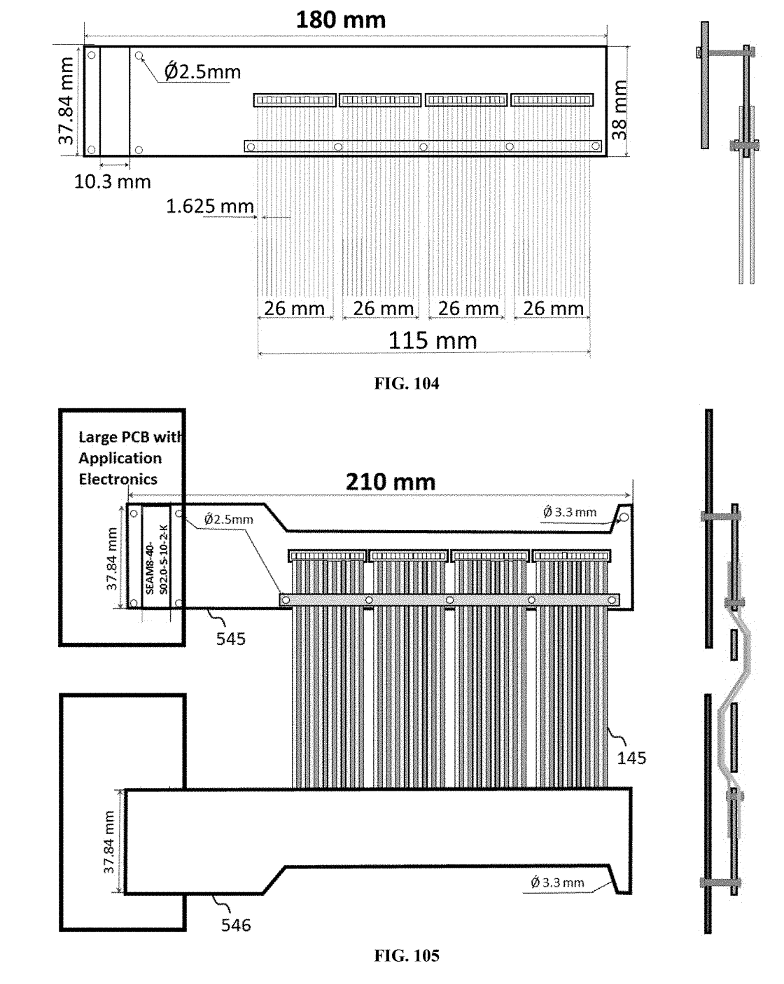

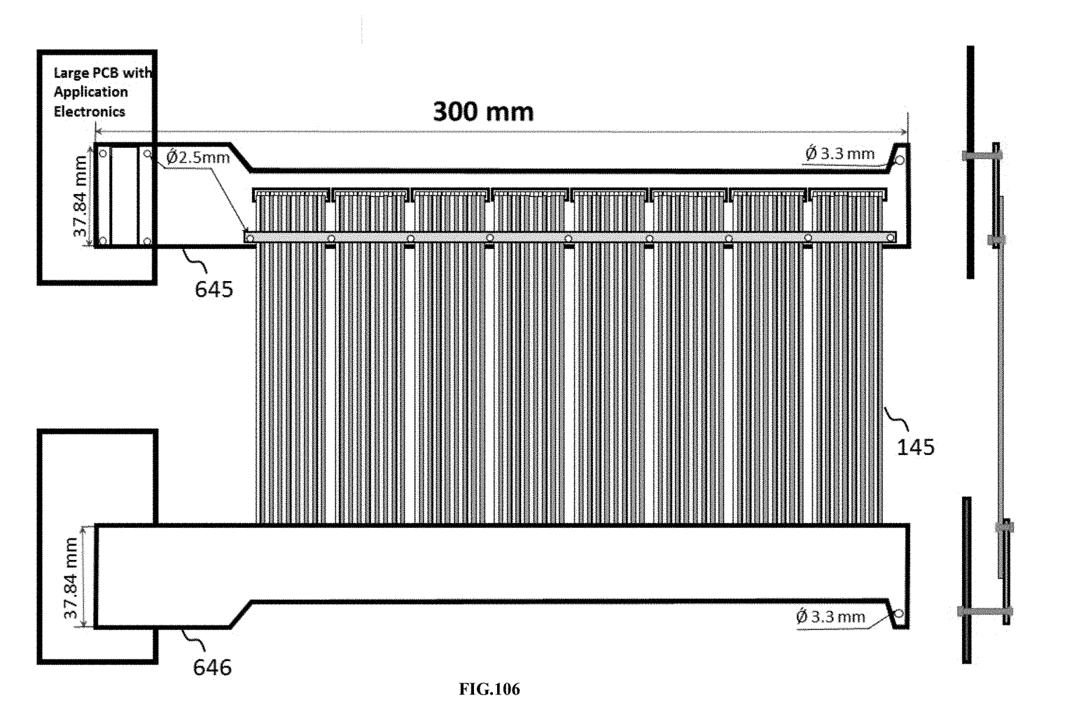

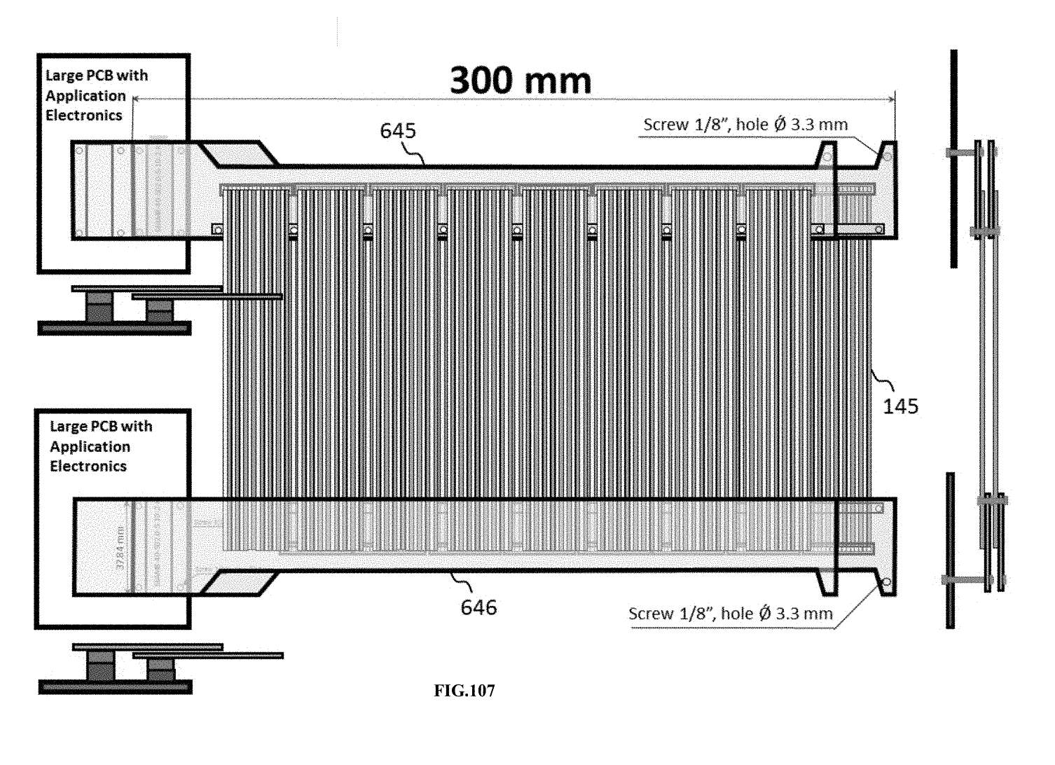

See FIG. 100.

I realized that 6.9 mm stacking connectors may not provide sufficient space to accommodate four ribbon cables which are each only 1.1 mm thick, but they have also the UV epoxy resin protecting the soldering that increases the overall thickness. To remedy this, in FIG. [symbol] , I selected the connector mating pairs SEAM8-40-S05.0-S-10-2-K mating with SEAF8-40-05.0-S-10-2-K, which provides 10 mm stacking height and removed the second strain reliever tightening the bundle of 8 ribbon cables with the larger application PCB because it was taking a lot of the application PCB area.

See FIG. 101.

Although the cable assembly of FIG. [symbol] provides flexibility to connect boards on two different crates, when several boards of both crates in Error! Reference source not found, Error! Reference source not found, and Error! Reference source not found. needs to be connected with 1024 Micro Twinax, the space to place these thick bundles of eight ribbon cables becomes a challenge. To improve utilization of the available space in the situation where several modules in one crate connect to modules in another crate, I gained space by orderly placing bundles of ribbon cables one next to the other and making the 90° cables turn on the small PCB as I drew in the next FIG. [symbol] . I provided a distance between the connector and the first ribbon cable of 38 mm to stagger two 128 Micro Twinax assembly to the larger PCB application board. The connector SEAF8-40-05.0-S-10-2-K of one assembly mates with connector SEAM8-40-S02.0-S-10-2-K at the larger PCB soldered at the edge, giving a stacking height of 7 mm, while another mates with connector SEAM8-40-S05.0-S-10-2-K, placed at 25 mm from the edge giving a stacking height of 10 mm. This will allow to increase the I/O to the board as also detailed in the caption of FIG. [symbol] .

The assembly of FIG. [symbol] helps to better utilize the available space; it can be useful in some applications with a small number of boards, but the thickness of the bundle of ribbon cables of approximately 12 mm is still too high when more than one connector is needed to connect VME boards with an interconnector distance of 20 mm.

Therefore, I went back to the drawing board of my Power Point and created assembly of FIG. [symbol] placing side-by-side two ribbon cables on both sides of the small PCB board.

The cable assembly of FIG. [symbol] helped to solve the problem of the limited space to place ribbon cables between crates for some applications; however, placing four ribbon cables side by side and further reducing the thickness of the ribbon cables going from crate to crate as shown in FIG. [symbol] , helps to solve more challenging problems requiring a high data transfer rate.

Further analytical verification of FIG. [symbol] made me realize that if a customer needs to place one of these little PCB boards next to the other because there is limited space on the front panel of the application board, then I should cut a piece of PCB along the width of the 4 ribbon cables as I did in FIG. [symbol] to create a window where the four ribbon cables can cross from one side of the PCB to the other side. I should also provide a hole at the corner of the small PCB board opposite to where the connector is assembled that will be used as shown in Error! Reference source not found.

See also on the right of FIG. [symbol] how the ribbon cables are crossing over from one side of the boards to the other in order to keep an identical cable length.

After a careful verification of the design of the assembly of FIG. [symbol] , I concluded that it would be reliable for signal integrity, speed requirements and mechanical robustness. It satisfies the need to achieve a very reliable high data transfer rate between different boards located in different crates using up to four of these 400-pin connector-cable assemblies per VME board. It satisfies the need for the specific application of the LHC TER/Simulator (DSU) board with 512 channels requiring four of these cable assemblies per VME board. For more stringent requirements, where 8 to 16 of these 400-pin connector-cable assemblies are needed, or board-to-board space is narrow, I designed FIG. [symbol] assembly having only one layer of ribbon cables.

Lastly, I verified the difference in cost between this version of connector-cable assemblies using a 1.27 mm connector pitch as shown in and 0.8 mm connector pitch used in all subsequent drawings.

First I was told that the small PCB would cost much more because 1.27 mm pitch allows two signal traces in between, while 0.8 allows only one trace requiring an increase in the number of PCB layers to route all signals to the pads of the cable. Additionally, if the user's application PCB board having very simple circuits and components with pin pitch higher than 1.27 mm allowing the construction of a very economical PCB, the presence of a connector with 0.8 mm pitch will force the customer to add layers to the larger application PCB, increasing its cost.

This is not the case for this project because the ASIC with 64×3D-Flow processors used in the boards described in FIG. 4, Error! Reference source not found, Error! Reference source not found. has a pitch of 0.8 mm, therefore the PCB board should have many layers.

However, it turns out the price of FIG. [symbol] cable assembly using 1.27 mm connector pitch (see quote HRD-190421-01 on page 133 of BJ) compared to the cable assembly using 0.8 mm pitch, the difference is a nominal 8%.

At this point one cannot dismiss all previous drawings and state that only the last two of FIG. [symbol] and FIG. [symbol] are valuable because for specific applications a previous drawing and approach might be more cost-effective.

Likewise, I found it useful to report all analytical thinking in optimizing the design for a cost-effective connector-cable assembly application to achieve the highest transfer rate at the minimum cost, capable of speeds ten times the current transfer rate and satisfying requirements of future upgrade of the electronics. The considerations made in this analytical thinking might help optimize the connector-cable assembly to achieve cost-effectiveness in other applications, although I must say that it took less time to invent this cost-effective connector-cable assembly than writing this report. I did most of the Power Point drawings related to this connector-cable assembly on Nov. 19, 2015, and requested the quote from SamTec for the difference in price between the version using 1.27 mm pitch connectors and 0.80 mm pitch connectors that was received the next day (see quotes on page 133).

One must be ready to modify the specifications as the work is progressing and feedback is received from those implementing it. For example, the need to cut the PCB on one side to create a window to let ribbon cables cross from one side of the PCB board to the other when there is limited space on the front panel of the application larger PCB as shown on the right side of FIG. [symbol] . However, if the engineer who lays out the small PCB board for the version with eight ribbon cables placed side-by-side in FIG. [symbol] tells me he has difficulties routing equal length traces from the pads of the ribbon cable closest to the connector to the one further away because he does not have real estate PCB and is running out of number of layers he can add, I would agree to eliminate the cut which is shown to be not strictly necessary for the application of Error! Reference source not found.

This assembly satisfies all needs for very large boards, crates and systems to achieve maximum transfer rate. It not only satisfies all system requirements in the three applications described in Error! Reference source not found, Error! Reference source not found, and Error! Reference source not found. but exceeds them when staggered as show in FIG. [symbol] accommodate up to 16 of these 400-pin connector-cable assemblies in an ATCA board and up to 18 in a VXI board.

Using 64 of the 400-pin connector-cable assemblies of FIG. [symbol] to transfer data from 8,192 channels from the ATCA/PRAI crate of 4to the VXI/3D-Flow crate will satisfy today's requirements of transferring 1.3 TB/sec, and without the need to change the cable assembly it can sustain future upgrades of the electronics at both ends up to 13 TB/sec.

For one entire application of a large experiment at CERN such as Atlas or CMS, the total cost of these connector-cable assemblies for 8,000 trigger channels is approximately $10,000 which can be verified from quote #34a on page 133 of BJ. It would be a good investment as it will still satisfy upgrades which are 10 times faster at a transfer rate of up to 13 TB/sec.

At this point we have much information that can help (with the help of catalogues from companies available on the web) answer the five questions asked at the beginning of this chapter. I report the answers in the same sequence they were listed at the beginning of this section.
1. At this time it is more cost-effective (and more reliable) to use 8,192 cables at 1.28 Gbps per cable instead of 1048 Ethernet cables at 10 Gbps, not just because of the lower price but more importantly because it provides a more powerful tool to execute complex Object Pattern Real-Time Recognition Algorithms with zero dead-time on 16-bit data arriving at 80 million events per second from over a billion collisions per second.
   Adding FPGA receivers to de-serialize the 10 Gbps to 1.28 Gbps that can be received by the 3D-Flow chip in the 3D-Flow board (either in the VME or VXI version) will increase the power consumption of the board.
   This will require distributing the same number of 3D-Flow chips across more boards because there is a limit the power each board can dissipate.
   Consequently, the size of the 3D-Flow system will increase, requiring longer cables for each chip in one board to communicate to its logical neighboring chip (in the global map of the detector array which is transferring data to the 3D-Flow processor array) on another board.
   A longer cable connection increases the time each 3D-Flow processor needs to exchange data with its neighbors to execute the Object Pattern Real-Time Recognition Algorithm, thus the number of layers of 3D-Flow processors needs to be increased to make a bigger system, requiring longer cables, further increasing the size of the electronics, and increasing the overall cost to achieve the same performance the 3D-Flow system would have obtained had it been contained in a smaller volume using shorter cables.
   To optimize performance and cost I need to keep the smallest possible number of extra components in the 3D-Flow board that are not the 3D-Flow chips.
   Using 10 Gbps data rate to the 3D-Flow boards if the 3D-Flow chip could receive directly 10 Gbps input was considered, but an informal enquiry at the ASIC design house, revealed the NRE would be over four times as expensive. This issue could be discussed with the funding agency. There is no preclusion from my side to go against CERN's standardization of 10 Gbps, I am just providing the cost-performance data and I would be glad to comply for the higher speed. In any event, the cables proposed from this study would satisfy the requirements of both the 1.28 Gbps and the 10 Gbps approach.
2. The component cost of the 8,192 cables at 1.28 Gbps is $10,000 (see quote #34a on page 133 of BJ).
   The component cost of 1048 Ethernet cables at 10 Gbps is over $120,000 calculated as follows:
   The cost to assemble one cable with two Ethernet connectors is $41 (see Amphenol website for price), ×1024=$41,984, plus the cost of approximately $40,000 for the transmitter FPGAs serializing the bits from 1.28 Gbps to 10 Gbps and $40,000 for the receiver FPGA de-serializing from 10 Gbps to 1.28 Gbps (See DigiKey website for FPGA prices).
   Using the FPGA approach of serializing and de-serializing will waste a lot of money because these FPGA chips combine transmitters and receivers in the same chip, therefore at the transmitter board the receivers' functionality will be wasted and vice versa. I found the answer I received from the clerk at a computer store in Dallas interesting when I asked whether they had any unidirectional Ethernet boards. His answer was no as they are targeted only to people who communicate in both directions.
   In our application, instead we have the need to transfer data at 1.3 TB/sec in one direction and a few 1 KB/sec in the other direction (see FIG. 1—Breakthrough invention. 3D-Flow OPRA—a revolutionary electronic instrument for multiple applications: advancing science, saving lives, fighting terrorism, . . . The figure illustrates 3D-Flow OPRA electronic instrument that can be implemented in a 36 cm cube of electronics, which is capable of executing pattern recognition algorithms in real-time of multidimensional objects (different ideas, or algorithms are represented as a light bulb) by analyzing all data arriving at ultra-high speed from a matrix of thousands of tranducers at over 20 TB/seconds with zero dead time. It provides three examples of possible applications: a) discovering new particles (Level-1 Trigger); b) saving millions of lives and reducing healthcare costs with the 3D-CBS (3-D Complete Body Screening); and c) fighting terrorism (identifying potential threats, find a needle in a haystack)
   FIG. [symbol] ), therefore we cannot copy the world of electronics form (ATCA, VPX, etc.) developed for telecommunication. Although in both fields, HEP and telecommunication, we have a need for a high data transfer rate, HEP is different because it is mainly unidirectional.
3. FIG. [symbol] , FIG. [symbol] , detail how eight SamTec SEAM8-40-S02.0-S-10-2-K connectors 0.8 mm pitch, 37.84 mm in length carrying 1024 LVSD differential signals fit on the front panel of a 322 mm ATCA blade (with a usable space of 310 mm), while FIG. [symbol] details how 2048 LVDS differential signals can be received by an ATCA board using a column of connectors at the edge of the board SEAM8-40-S02.0-S-10-2-K providing a stacking height of 7.0 mm, and at 25 mm from the edge of the ATCA board a column of connector SEAM8-40-S05.0-S-10-2-K, which provide a stacking height of 10 mm which would allow two connector-cable assemblies to be staggered.
4. Because the dimensions of the Ethernet connector cannot fit 128 connectors on one ATCA blade or VXI board, the electronics will increase the number of boards and number of crates needed, increasing the overall cost. (See dimension of the 10 Gbps Ethernet connector at http://www.cablesondemand.com/category/SFP+%20CBL/product/SFSFPP2EPASS/URvars/Items/Library/InfoManage/.htm?gclid=CNLe8JPZuMkCFQIHaQodo_8DqA)
5. There is only an 8% increase in cost to have the 0.8 mm pitch connector instead of the 1.27 mm pitch connector solution as reported in quote HDR-190421-01 on page 133 of BJ. However, the 0.8 mm pitch connector has many technical advantages including increased system performance because it allows four 0.8 mm pitch connectors to be placed on the front panel of a VME board and 8 connectors in the VXI and ATCA boards. This will reduce the size of the system and the power consumption and increase the performance while greatly reducing the overall cost.

In summary, to have the best price of the connector-cable assembly it is necessary to maximize the use of one type of connector-cable assembly to reach the rock-bottom price for 500 units in the quote #34a on page 133 of BJ.

What will drive the universities and laboratories to order one type or the other is which form factor has been chosen. This will depend on whether the funding agencies will fund more electronics using large boards such as VXI and ATCA or smaller boards such as VME.
a. Implementing FIG. [symbol] with eight Micro Twinax 16×2 ribbon cables assembled all on one side of the small PCB is best for the larger VXI and ATCA boards. This assembly requires a deeper mounting frame occupying more space in front of the crate to support the weight of the cables as shown in Error! Reference source not found.4. The maximum performance of this connector-cable assembly used at 1.28 Gbps per channel for the full use of the 12 slots of a VXI crate with 8 connectors per board would be 15.7 Tbps (calculated as 1024×1.28 Gbps×12=15.7 Tbps), or when used at 10 Gbps, the overall performance would be 122.8 Tbps (calculated as 1024×10 Gbps×12=122.8 Tbps). The price of all connector-cable assemblies will be the same—approximately $16,320.
6. Implementing FIG. [symbol] with four Micro Twinax 16×2 ribbon cables assembled on both sides of the small PCB is be the best for the smaller VME, VPX boards. This assembly requires a smaller mounting frame occupying less space in front of the crate to support the weight of the cables as shown in FIG. 5. The maximum performance of this connector-cable assembly used at 1.28 Gbps per channel for the full use of the 20 slots of a VME crate with 4 connectors per board will be 13.1 Tbps (calculated as 512×1.28 Gbps×20=13.1 Tbps), or when used at 10 Gbps, the overall performance will be 102.4 Tbps (calculated as 512×10 Gbps×20=102.4 Tbps). The price of all connector-cable assemblies will be the same—approximately $13,600.

The estimated performance and cost when using FIG. [symbol] , connector cable assembly, taking advantage of its full transfer rate capability between ATCA, VXI and VME crates, staggering two connectors, one at the edge and another 25 mm from the edge of the board, with the pinout recommended by SamTec (FIG. [symbol] ) is as follows:
a. The maximum data transfer rate achievable between 2 ATCA crates with 14 slots assuming 16 connector-cable assemblies per board, and each channel having a transfer rate of 10 Gbps will be 28 TB/sec (or 224 Tbps). This is a very conservative estimate because SamTec catalogue provides the performance of these connectors at 22 Gbps and 0.5 m Twinax can provide >11 MHz at −3dB insertion loss. In fact the transfer rate can be tested from 28 TB/sec up to 49 TB/sec to see when it fails. The price of all connector-cable assemblies will be approximately $38,000.
b. The maximum data transfer rate achievable between 2 VXI crates with 13 slots assuming 18 connector-cable assemblies per board, and each channel having a transfer rate of 10 Gbps will be 29 TB/sec (or 234 Tbps). This is a very conservative estimate because SamTec catalogue provides the performance of these connectors at 22 Gbps and 0.5 m Twinax can provide >11 MHz at −3dB insertion loss. In fact the transfer rate can be tested from 29 TB/sec up to 51 TB/sec to see when it fails. The price of all connector-cable assemblies will be approximately $39,000.
c. The maximum data transfer rate achievable between 2 VME crates with 21 slots assuming 8 connector-cable assemblies per board, and each channel having a transfer rate of 10 Gbps will be 21 TB/sec (or 168 Tbps). This is a very conservative estimate because SamTec catalogue provides the performance of these connectors at 22 Gbps and 0.5 m Twinax can provide >11 MHz at −3dB insertion loss. In fact the transfer rate can be tested from 21 TB/sec up 37 TB/sec to see when it fails. The price of all connector-cable assemblies will be approximately $28,000.
13. Likelihood of achieving valuable results

Because the 3D-Flow invention has been proven to be feasible and functional in hardware FPGA it can be stated that results can be achieved.

Simulation of large 3D-Flow system in C++ down to the RTL level with VHDL and synthesized in standard cell of a 350 nanometer technology is an additional proof that valuable results can be achieved.

Errors in developing the electronic boards and ASIC can only delay the results in requiring two to three spins (or versions) of the prototypes, however, my past experience in developing very complex boards with a high density of components in a small PCB area such as the FDPP board [] and the 3D-Flow board with 68×3D-Flow processors and successfully working at the first version produced makes me feel confident that the 3D-Flow system (boards and chip) with the new ASIC with 64×3D-Flop processors per chip would work at the first version built or in worst case at the second version.
14. How results of the proposed work might impact the direction, progress, and thinking in relevant scientific fields of research

The two graphs on the ieft show data from the World Health Organization with cancer as the most deadly premature (less than 70 years of age) calamity. The central picture is an announcement in a National Geographic issue showing that we were aware since 1946 that Early Detection can save over 50% of lives. The table on the right reports data from U.S. NIH-NCI-SEER (Surveillance Epidemiology End Results) showing in the second column of data from the left that when cancer is detected early, 49.5% of lung cancer patients . . . 100% of prostate cancer patients survive, but when detected late only 2.8% of lung cancer and 31% of prostate cancer patients survive (right column). The ultra-sensitive 3D-CBS can provide an effective Early Cancer Detection, saving lives.

Doctors and journalists who claim that screening can cause unnecessary risky procedures should work to identify the incompetent doctors who diagnose false positives and help to improve training of medical personnel. When an astronaut or engineer at NASA makes a mistake and a Shuttle crashes we do not abolish NASA. When a pilot makes a mistake, journalists not write articles to ban air transportation. When drivers make an error and cause an accident, journalists do not write articles to ban motor vehicles traveling faster than 20 mph but we all help to improve car safety and better train drivers. We cannot stop advancement in science that, will provide more accurate medical instrumentation to doctors because they do not know how to use their information; we should all help to better train doctors, so they will avoid to make mistakes of false positives.
15. How the proposed project compares to other efforts in the field, in terms of scientific and technical merits and originality
   a. In HEP applications it replaces many crates of electronics with a single crate of electronics containing a much more powerful system

I have summarized in one picture (See FIG. 3) the important ingredients that should justify my visit.
b. In Medical Imaging applications the 3D-CBS is hundreds of times more efficient than current PET and PET/CT enabling early cancer detection, using a low radiation dose at an affordable examination cost. It has shown to be superior at the public review during the Leonardo da Vinci competition
L. Appropriateness of the proposed approach
16. How the research approaches are logical and feasible I started my investigation in this field of applications by typing some key words into the Google search Engine such as “Trigger HEP”, “Trigger LHC experiments” “Trigger CMS”, “Trigger Atlas”, etc. and among the first on the list was an article by Wesley Smith (2013), one by Philipp Grabs (2014) and several others.

I read both and several others. Wesley Smith's article http://arxiv.org/ftp/arxiv/papers/1307/1307.0706.pdf after the first chapter “Findings” reporting the requirements (some requirements are also found in the Chapter “Executive Summary”), which are also found in several CERN documents, I read some very confusing statements in the Chapter “Recommendations” and “Executive Summary”.

By listing all components, technologies, form factors, CPUs, protocols, links at different speeds, making a big salad without describing what is important for this application in processing power, in link speed vs. price, etc., it clearly reveals that the author does not have a clue which analytical path to follow to find the most cost effective solution to capture and accurately measure the characteristics of new particles (objects).

An analytical thinker would start by looking at the number of channels from different subdetectors participating to Level-1 Trigger (calorimeter, tracker, muon . . . , etc.), the timing relation between them, and whether those with a larger number of channels can be tunneled to 8,000 channels of the calorimeter Trigger Tower. They should then ask themselves how many bits are necessary for each subdetector Trigger Tower or generally speaking, for all detectors within a certain view angle, and what is the total size of the words per channel needing to be transferred to the Trigger Processor every 25 ns; is 16-bit sufficient, or is 32-bit or 64-bit necessary? What kind of operation is necessary to perform on those bits or group of bits? Do we need to exchange data with neighboring processors to execute object pattern recognition? How efficient is FPGA to perform these operations?

Instead, in Chapter 2 and Chapter 3, Smith lists FPGA, ATCA, Micro-TCA, Advanced Mezzanine Cards standard, GPUs, ARM, Xeon Phi, TCA for backplanes GbE, SATA/SAS, PCIe, SRIO, High Performance Computing (HPC) interconnect, Network Interface Cards (NICs), 40 Gbps, 100 Gbps, 100 Tbps which should be achievable with today's switches at 32 Tbps . . . Why? If I can transfer 234 Tbps across 23,400 Twinax cables between two VXI crates with $39,000, as detailed on page 102, why do we need to use expensive telecommunication equipment designed to communicate in both directions when we need only to transfer data in one direction? Perhaps to complete the panorama of the long list mentioned before we can also add Hypercube. However, which one of all those components listed by Smith finds the desired particle more cost-effectively?

I am not surprised to read these statements by Smith. I have been trying to help him follow analytical thinking since 1992 when he was in charge of the Trigger of the SDC experiment at the Superconducting Super Collider, but when I invented the 3D-Flow architecture offering programmable object pattern recognition at Level-1 Trigger, he was one of a handful of people who stated that “we do not need programmability at Level-1 trigger”. I have been trying to help him understand for many years. In 1998, I attended a workshop on the electronics for LHC at Snowmass (Colo.), and experiencing a closed door from him as Co-Chairman and Co-chairman Peter Sharp, who would not let me present my invention and my analytical thinking. The last time I paid him a visit was in his office at CERN in 2008 when he recanted many statements and positions he had taken before; however, he continues to be closed to analytical thinking, following instead his original ideas, which at the time of the Super Collider was his cluster finding ASIC, protecting his own idea instead of openly comparing its effectiveness with other approaches, and supporting and funding the author with the better idea.

His approach precludes the birth and raising of innovative ideas; he does not see how his approach which crushes analytical thinking is detrimental to the scientific community and to taxpayers; he does not see the evidence of the failure of the Level-1 Trigger that could not and cannot identify particles with the characteristics defined by experimenters. Yet it is clear from the results of experiments that could not find even a small percentage of the 100,000 Higgs boson-like particles predicted to be present, and from the admission by CMS collaboration that Level-1 Trigger must be trashed and a new one built, that the current Level-1 Trigger is a failure.

Luckily the LHC collider worked, producing particles at a higher energy than the Tevatron at FERMILab, allowing us to go one step further in the understanding of our universe with a more powerful tool, and despite the trigger being ineffective, the data acquisition of the LHC experiments was able to casually record some events different than others. Now, however, it is necessary to build a very effective trigger that can nail down the nature of these Higgs boson-like particles. As I mentioned in one of my previous documents, the Nobel Prize should have been assigned to those who built the LHC collider working at an energy level never before achieved.

Ignoring, refusing, boycotting or blocking open public forums which would exercise analytical thinking should not be allowed because it is damaging the scientific community and the public who trust scientific procedures are being followed. The decisions of one person with funding power but who is not supporting the best approach, as in the case of Wesley Smith, is driving into the ditch thousands of scientists who worked for 20 years to develop software and hardware to analyze noise data because Level-1 Trigger did not work, as it happened for CMS and the collaboration decided to trash it. The same thing happened for Atlas who also decided to trash their Level-1 Trigger.

Wesley Smith is not the only scientist unwilling to have an analytical discussion where everyone would gain. For example, over the years I have tried having an analytical discussion on the Level-1 Trigger with Patrick LeDu, a senior organizer of the IEEE-NSS conferences. Our relation has always been cordial; however, the typical course of events at the IEEE conferences is on day one I ask him for a meeting; he schedules our meeting during a coffee break but then never shows up; we set another meeting, and again he does not show up. He never answers email on technical-scientific issues. When he has spoken a few words to me, it is to lament that the government does give him enough funds to keep the bright students at the university after graduation, or that he had some bright idea like mine for the trigger; but he never wants to discuss and compare his idea with my invention, providing the excuse that he is very busy attending several meetings to plan the future.

At the 2013 conference in Seoul, I noticed that he was the convener at the Trigger Session. Hoping to be able to address analytically trigger issues, I went to the session and asked the presenters questions. They clearly stated that Level-1 Trigger was not fully programmable nor did it operate with zero dead-time. LeDu did not chair his session because he was attending meetings to plan the future which he apparently believed is more important than discussing analytically the essence of the future in HEP with his colleagues, presenting different trigger ideas and hearing comments from other participants.

Another leader, Ugo Amaldi, told me before the keynote speaker's lecture at the 2012 International Conference on Translational Research in Radiation Oncology in Geneva, not to ask the speaker any questions and not to contact newspapers. This surprised me as I believed a renowned physicist like Amaldi would be eager to hear an exchange of questions and answers and to share new inventions with the public, just as the Chairman of the ICATPP conference (Pier Giorgio Rancoita) had in 2005 when he called for a Press Conference to announce my 3D-Flow and 3D-CBS inventions that create a revolution in the field.

Or Amaldi could have organized a public debate where physicists that he knows could challenge my analytical thinking by asking questions; the outcome would only help to advance science. Others who denied analytical, scientific discussion and transparency in science are: Ingrid-Maria Gregor, Chair, and Adam Bernstein, Deputy Chair, of the 2014 IEEE-NSS (Nuclear Science Symposium Conference), who denied analytical discussion and transparency in science when they rejected my proposed workshop on transparency (see details at: http://blog.u2ec.org/wordpress/?p=150); Anthony Lavietes, General Chairman of the 2014 IEEE-NSS-MIC-RTSD conference, interrupted my legitimate question to the 2014 IEEE-NSS Keynote speaker, provided a statement inconsistent with science, and then walked away as reported on page 41.

In the field of Medical Imaging, Alberto del Guerra is considered one of the leaders. However, I have never been able to discuss analytically his review of my book, or why his reasoning that we do not need a PET device with a FOV longer than the size of the largest organ (16 cm) is flawed. He has been invited several times by organizers of workshops on my 3D-CBS technology from the University and San Matteo hospital in Pavia to be a member of the panel of experts or reviewers to publicly express his evaluation regarding my invention which would allow me the opportunity to answer his questions, doubts and/or concerns, but has never accepted. I would also like to discuss with him the rejection of my papers, but he repeats they were rejected for their low score in scientific merit without wanting to discuss what they consider as “scientific merits”. (See his latest rejection based on a score that he cannot support http://blog.u2ec.org/wordpress/?p=1363). Others who denied analytical, scientific discussion and transparency in science regarding medical imaging are: George Alfakhri & Katia Parodi, Chair and Deputy Chair of the 2014 IEEE-MIC (Medical Imaging Conference), who denied analytical discussion and transparency in science by rejecting a workshop (see details at: http://blog.u2ec.org/wordpress/?p=956); Craig Levin, Deputy Chair of the 2013 IEEE-MIC conference, who built and published PET with very low sensitivity and high spatial resolution but rejected my papers for a very high sensitivity and high spatial resolution.

When I had to choose the form factor to implement the ER/DSU and the 3D-Flow ASIC boards, I was attracted at first by VPX in place of VME because I read articles that CERN was moving toward VPX standard and I did not want to stay behind. I had similar considerations toward ATCA compared to VXI; however, a deeper investigation made me realize that there are no advantages, and in fact it costs more.

The Telecommunication Industry is doing an excellent job defining new standards such as ATCA, VPX, Micro-ATCA, etc. However, HEP's needs are different. It needs unidirectional communication, and because SERDES Ethernet, cables, components, connectors, are bi-directional, either a transmitter is going to waste on one side or a receiver on the other side. In addition, the price of VME connectors compared to VPX connectors is considerably lower.

I asked colleagues and professionals in the industry to point out the differences or advantages of VPX compared to VME. I asked professionals to provide names of people at CERN who were advocates of VPX, and was directed to read this article http://iopscience.iop.org/article/10.1088/1748-0221/10/01/C01008/pdf. The article did not provide a comparison in price or features that showed great advantages for CERN to use VPX instead of VME; in fact, for my application I found advantageous in using VME.

We should focus on the objective of creating very powerful tools that allow experimenters to discover new particles at the lowest cost per experiment. We should be free to choose the more cost-effective form factor and transfer speed: ATCA, VXI, VME, VPX, 1.28 Gbps, 10 Gbps, 40 Gbps, 100 Gbps, etc. in each specific application as I did in this proposal. I used ATCA form factor for the PRAI crate (Patch-Panel Regrouping Associates Ideas), VME for small 3D-Flow systems, and VXI for large systems. Today it is more cost-effective to use 8,192 Twinax cables at 1.28 Gbps (up to 10 Gbps) to reduce power consumption in the 3D-Flow boards that do not need a de-serializer because their input port speed is 1.28 Gbps. In the future, when it becomes cost-effective to build a 3D-Flow chip with Top and Bottom port I/O speed at 10 Gbps, then using 10 Gbps all the way from the detector to the 3D-Flow electronics will be most cost-effective, and eliminate the cost of serializers and de-serializers.

I hope that you find my analytical thinking satisfactory and useful to justify a paradigm change in the way the scientific community evaluates papers and projects by implementing bidirectional communication based on analytical thinking as supported by President Barack Obama in a letter he wrote to me on Sep. 25, 2015. Implementing bidirectional communication between authors and reviewers, applicants and funding agencies, is needed to provide maximum benefits to taxpayers, and unidirectional communication as shown in FIG. 1—Breakthrough invention. 3D-Flow OPRA—a revolutionary electronic instrument for multiple applications: advancing science, saving lives, fighting terrorism, . . . The figure illustrates 3D-Flow OPRA electronic instrument that can be implemented in a 36 cm cube of electronics, which is capable of executing pattern recognition algorithms in real-time of multidimensional objects (different ideas, or algorithms are represented as a light bulb) by analyzing all data arriving at ultra-high speed from a matrix of thousands of tranducers at over 20 TB/seconds with zero dead time. It provides three examples of possible applications: a) discovering new particles (Level-1 Trigger); b) saving millions of lives and reducing healthcare costs with the 3D-CBS (3-D Complete Body Screening); and c) fighting terrorism (identifying potential threats, find a needle in a haystack)

FIG. [symbol] should be saved for electronic equipment in HEP which analyzes data observing natural phenomena.
E. Significant potential problems and alternative strategies to resolve them

Potential problems are not underestimated and are anticipated as well is anticipated their solution.

As you can see in the Timeline Section on page 108 and Error! Reference source not found, I have planned the development of the VME 25 ASICs and VXI 68 ASICSs after the final calculation of the power consumption of the 3D-Flow ASIC with 64×3D-Flow processors. In the event it is greater than 4 Watt the design of the boards will be modified to accommodate fewer ASICs to be able to handle boards and crate power dissipation.

Instead for the possible failure of connections on connectors or cables or failures on some processors, I had already developed with the previous DOE $906,000 grant in 1997 a fault tolerant program that is identifying the faulty cable, connector, ASIC or component and is isolating the offending components permitting to continue data acquisition with one or a few dead-channels.

### The 3D-Flow System is Fault Tolerant

During operation the entire 3D-Flow system made of thousands of 3D-Flow processors is monitored in real-time through the USB port at each chip that is accessing the status bits of each of the 64×3D-Flow processors in the chip.

Fault tolerant programs have been already developed that can detect broken processors or cables.

When a problem is detected, such as a non-responsive processor or a broken cable, this fault-tolerant monitoring program loads a modified program into each neighboring processor where there is a broken cable or broken processor.

This will allow the system to continue operation with one dead channel out of 8000 channels until the next shutdown of the data acquisition system when the maintenance team can access and repair the faulty component or cable.

This fault tolerant monitoring program also saves time diagnosing the problem because it can point out the chip ID or location of the broken cable.
M. Timeline of major activities

The purpose of this proposal is to create a general purpose 3D-Flow OPRA (Object Pattern Real-Time Algorithm) instrumentation that can identify objects in real-time, display their characteristics, and provide flexible triggering features using fast neighboring data exchange and by analyzing data for a time longer than the time interval between two consecutive input data which arrive in parallel from a matrix of thousands of transducers at ultra-high speed.

The 3D-Flow OPRA instrument can also solve multiple applications in different fields where the requirements are to identify among millions of other non-relevant signals cluttering the good information, the signals related to specific objects such as pairs of 511 keV photons, the profile and details of a face among thousands of faces, the signals characterizing the Higgs boson particle, etc.

By allowing the user to define a complex real-time algorithm which analyzes signals arriving from a matrix of transducers that respond to physical stimuli it allows to measure all kinds of phenomena and identify all kinds of objects in 3-D.

The 3D-Flow OPRA instrument for example can recognize a shape of different colors, a shape of different levels of heat, a shape of different levels of sound volume and frequencies, a shape of different energies, a shape of different mechanical stress, a shape of different pressure, a shape of different light, the characteristics of a specific subatomic particle measured from signals generated by CCD, APD, PMT, SiPM, PADs, silicon strip detectors, wire-chambers, drift-chambers, etc.

The inventor considers relevant to provide the patent examiners, facts, data, and testimonials reported in the following pages proving that professionals responsible in the field who should have stood for the scientific truth have instead taken actions suppressing the scientific truth and suppressing innovations.

Rather than making generic statements summarizing these actions which could be interpreted as unsubstantiated allegations, the inventor is providing excerpts of the original documents proving that these actions of suppressing the inventor's innovations for more than two decades are real, are damaging the public and future generations. (In addition to the excerpts provided herein, the inventor can provide upon request documents on their entirety if they could be useful for a patent search of prior art).

As part of the patent search of prior art if there exist any idea or approach similar and/or more advantageous because of an “inventive step over the available prior art” (as required to be considered an invention) to what claimed by the inventor in this non-provisional patent, the following pages provide information that the inventor made and extensive search, by addressing his inventions and claims with the top experts in the field who receive billions of dollars of taxpayers money to develop similar instruments and become expert in the field.

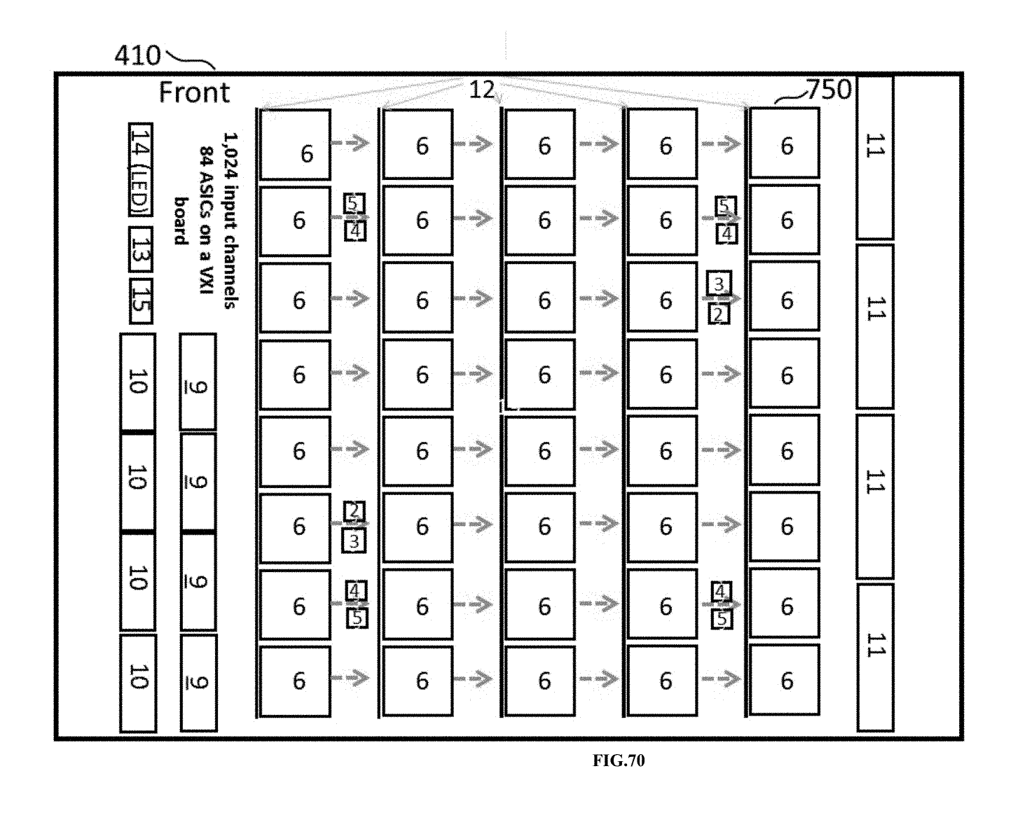

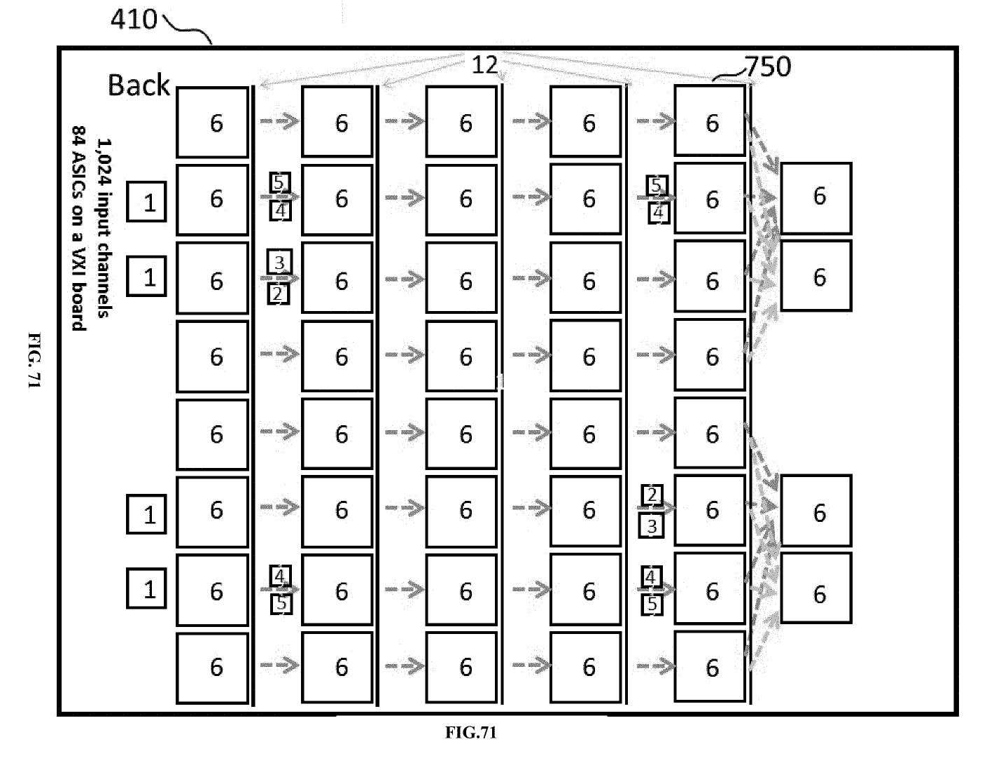

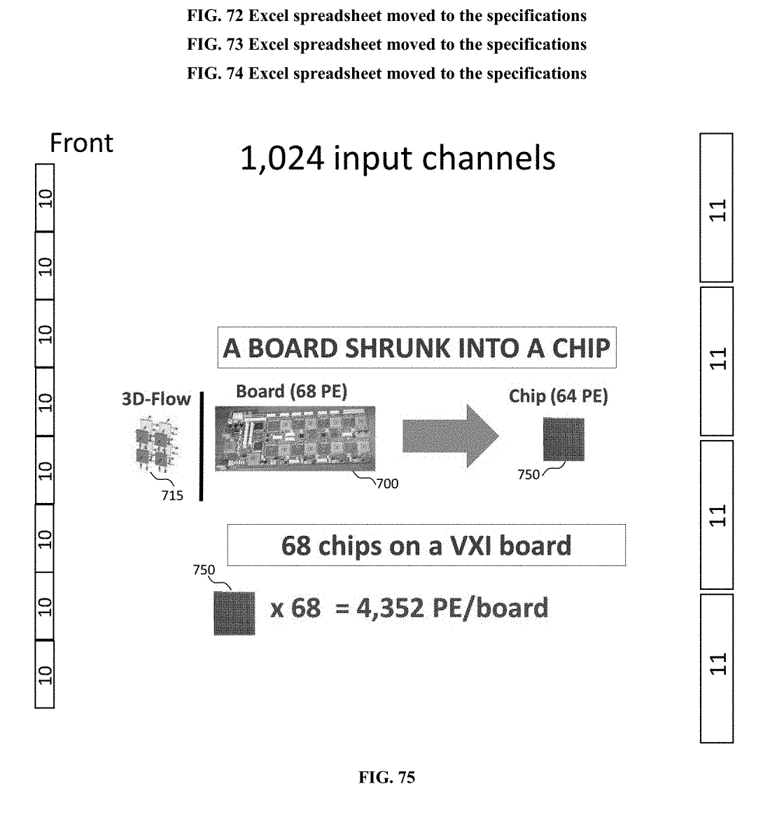

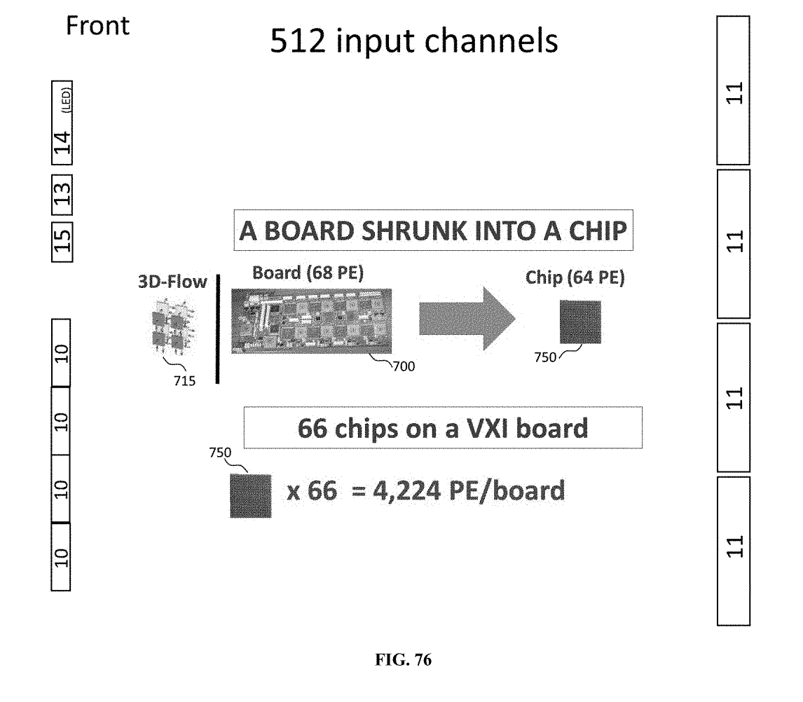

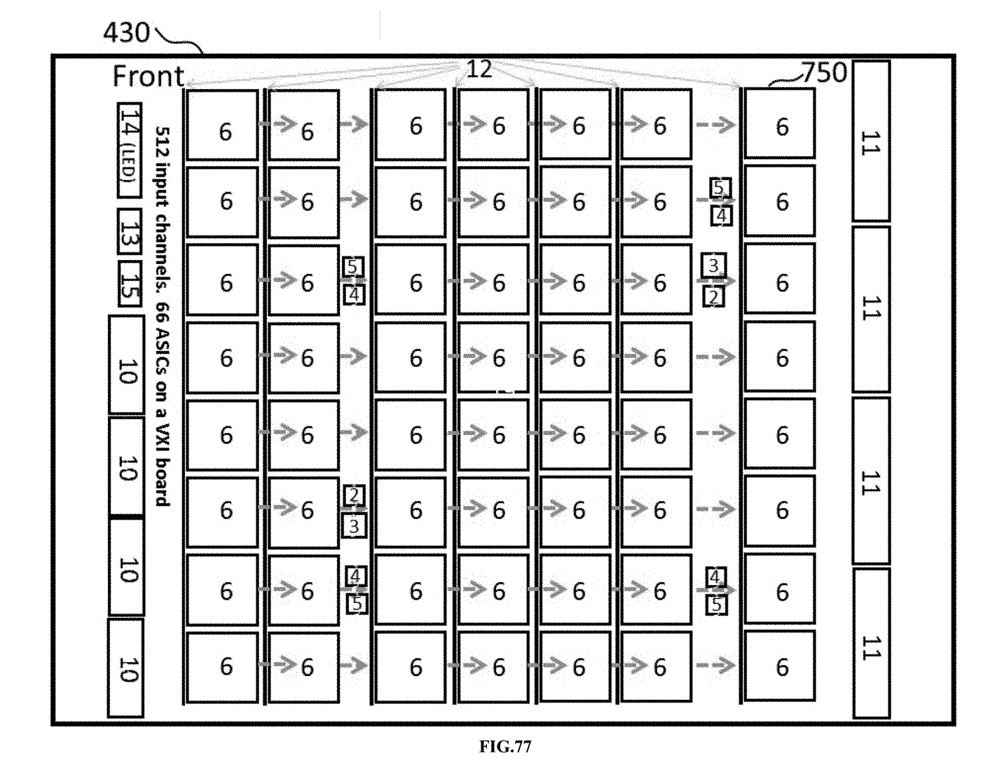

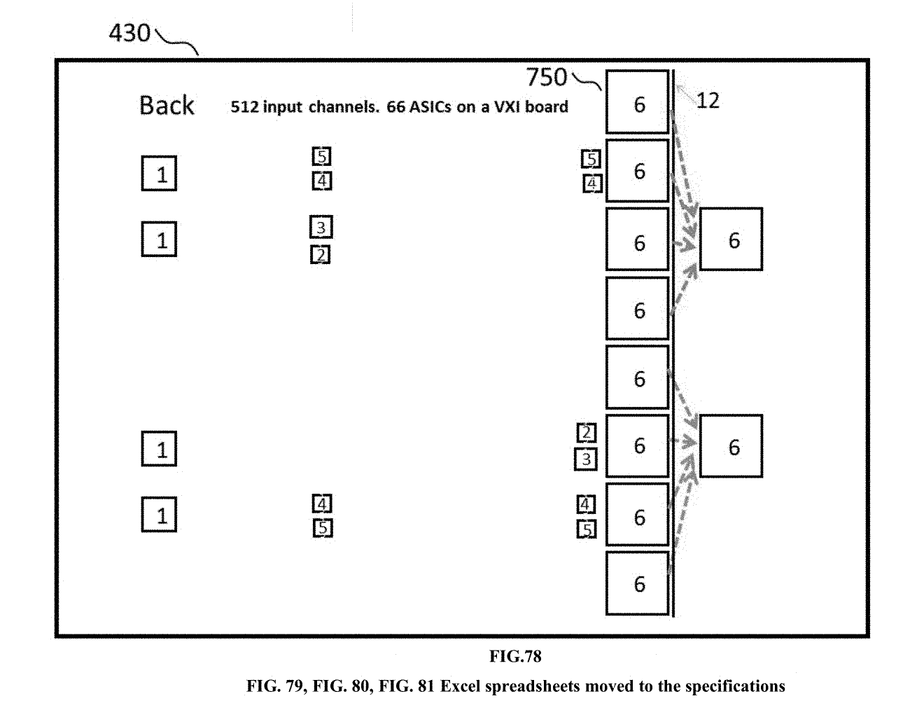

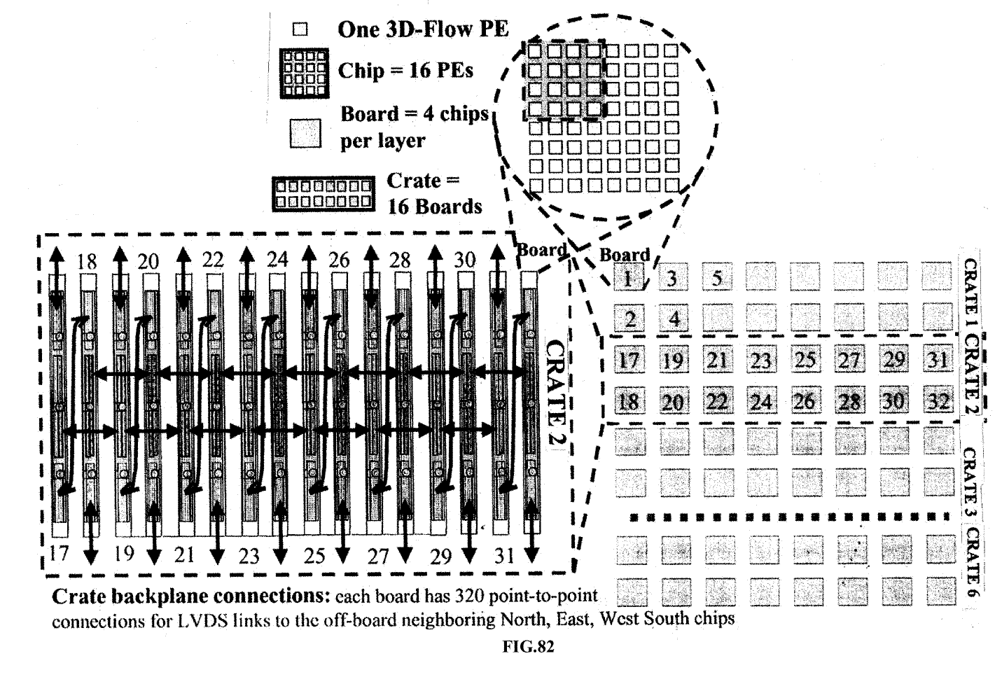

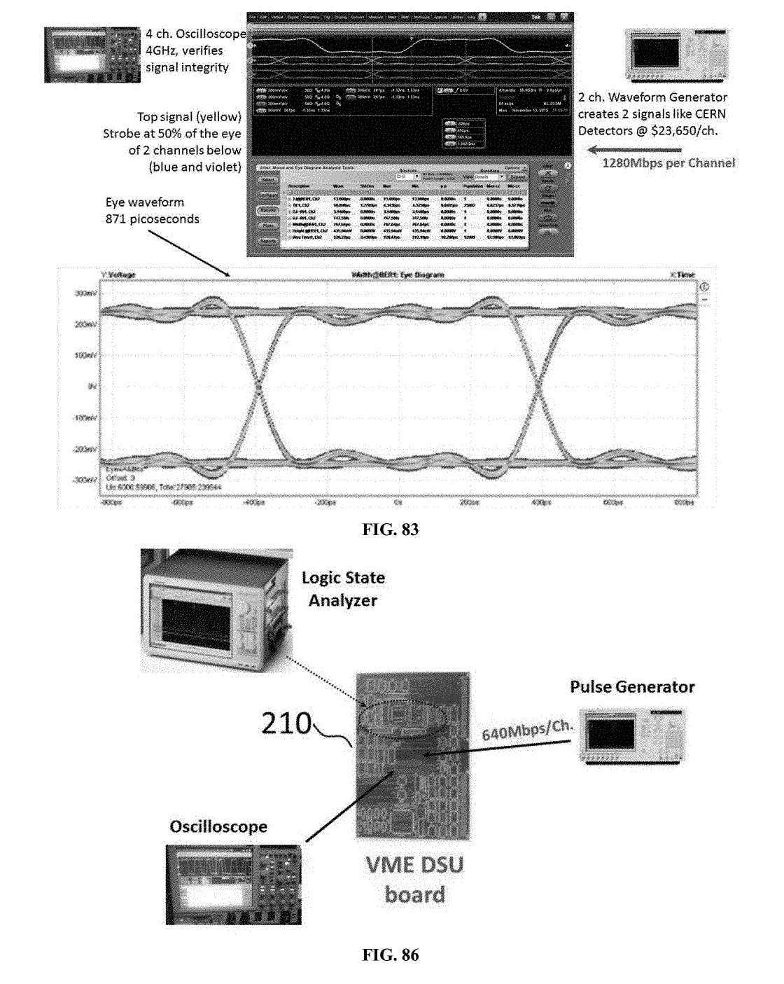

The result of this search for patents, ideas, and approaches similar and/or more advantageous to the one claimed by the inventor it shows that the inventions described in this non-provisional patent allows to replace 4,000 electronic boards CMS Level-1 Trigger system costing over $100 million at CERN at the biggest and most expensive experiment in the history of the planet costing globally to taxpayer over $50 billion, with 9 electronic data processing 3D-Flow OPRA boards of FIG. 69, FIG. 70, FIG. 71 and FIG. 75, assembled in a VXI crate of FIG. 4, which provide an enormous performance improvement at one thousandth the cost of the CMS system. This is a disruptive advantage and benefit provided by these inventions not only to the field of uncovering the unknow and confirming or excluding the existence of a particle predicted by theoretical physicists, but more important in enabling an effective early cancer detection when these inventions are applied to medical imaging with the potential to save millions of lives.

It will be logical to expect that any inventor in the world claiming more cost-effective advantages would have to discuss their claims as I did and compared their approach/idea with the major research center in the world at CERN that is receiving billions of dollars to develop these types of instrumentation.

Therefore, the relevance to the patent examiner of the following pages are precious information for the search and comparison of this invention with prior art and the specific answer the inventor received from the leaders in the field who should defend the cost-effectiveness and higher performance of their ideas/approaches.

In contrast, the answers from these leaders and people who develop instrumentation in the same field as this invention are unsubstantiated and non-scientific.

The patent examiner could ask questions on his own to these experts or to any person developing instrumentation in the field related to this non-provisional patent. Nevertheless, the following pages also provide the names of the people holding position of high responsibility in this field as well as the institutions and departments in the event their term will reach an end and they will be replaced by others.

If it could be useful for a patent search of prior art, the inventor could provide additional information about the answers he received from top experts in the field contacted by him with whom he discussed and compared his inventions and claims with other approaches (he did this after filing a provisional and non-provisional patent). Among them are the Chairpersons of the IEEE-NSS-MIC-RTSD conference in Strasbourg, France from October 31 to Nov. 5, 2016 and other leaders in the field of particle physics, including in the field of Medical Imaging. He also had a 100 minutes meeting on Nov. 5, 2016 with the current president of the IEEE-NPSS, Dr. John Verboncoeur and the new elected President who started on Jan. 1, 2017, Dr. Stefan Ritt. After going through the main innovative points in the inventor's 271-page document included herein they stated that did not hear anything like this before. (In addition to the excerpts provided herein, the inventor can provide upon request documents on their entirety if they could be useful for a patent search of prior art). IEEE is the world's largest technical professional organization with over 400,000 members dedicated to advancing technology for the benefit of humanity.
350 years after Galileo was threatened for stating scientific truths, Crosetto is threatened for stating scientific truths.
X. SUMMARY: Innovation is suppressed when scientists do not stand up for SCIENCE.

Galileo was threatened by the Church for asserting that the Earth is not at the center of the Universe.

Dario Crosetto has been threatened by the U.S. Department of Energy (DOE) and Citigroup to involve Security if he continues to insist on the feasibility of his inventions in medical imaging, high energy physics and other fields capable of replacing hundreds of crates of electronics with one crate at 1/1000 the cost, while providing staggering performance improvements: in discovering new subatomic particles, saving millions of lives with a cost-effective early cancer detection and reducing healthcare costs.

Why did it take 350 years for the Church to recognize Galileo was right, and why for 25 years have hundreds of millions of dollars of taxpayer money continued to fund less efficient, more costly systems than Crosetto's life-saving and money-saving inventions, which continue to be ignored? Why does Crosetto receive threats as Galileo did?

History repeats itself. If influential scientists of his time had stood strong in defense of science, Galileo might not have been forced to recant empirical evidence to avoid the consequences of the Church's threat to chop off his head, and humanity would have received the benefits of his discoveries earlier.

If the majority of scientists (and specifically those handling taxpayer and donated funds) stood in defense of science today, Crosetto would not receive threats from the U.S. Department of Energy (DOE) and Citigroup, and humanity would enjoy the benefits of his inventions.

The value of Crosetto's inventions have been recognized and endorsed by hundreds of scientists, the concepts have been proven feasible and functional in hardware, providing staggering performance improvements as confirmed in a public scientific review held at FERMILAB in December 1993, where Crosetto answered objections from other scientists. Recently it was proven feasible by 59 quotes from reputable industries to replace hundreds of crates of electronics with one crate at 1/1000 the cost . . . Yet Crosetto is boycotted, ignored and prevented from presenting his papers at conferences, even having his microphone removed when he asks legitimate and pertinent questions.

It is akin to an athlete with superior skill being blocked from Olympic competition by competitors who would lose the competition should the better athlete be allowed to participate.

Instead of implementing a world of reason/science, critical members of the scientific community are succumbing to a world of power/money and influence because of the rigged peer-review process which invites corruption.

To implement independence for the peer-review process expressed by the National Academy of Science, reform is needed. See Crosetto's answer to the President of IEEE, the world's 400,000 member largest technical professional organization dedicated to advancing technology for the benefit of humanity.

Decision makers in the field of medical imaging research who consider Crosetto a competitive adversary and prevent him from publicly discussing the utility of his 3D-CBS (3-D Complete Body Screening) invention, should realize there is only one true adversary and it is one we share—the plague of cancer. Everyone must, cooperate to advance science and technology against cancer at every opportunity, which will enable us to reduce the economic burden, suffering, and loss of lives which this calamity represents. We must allow the science of medical imaging to proceed freely through fair, open, public competition.

Likewise, colleagues in high energy physics research at DOE and at CERN should not consider Crosetto an adversary, but a collaborator who can provide experimental physicists the most powerful tool available for efficiently executing their preferred Level-1 Trigger algorithms, enabling them to quickly and economically confirm or rule out the existence of new particles, and do so with the highest certainty.

Governments allocate vast sums of money for eradicating cancer, while leaders of funding agencies do not want to hear solutions from inventors, giving the excuse that they want to be fair to all scientists, while their circle of scientist friends meet behind closed doors to split taxpayer money among themselves for their own interest and are not fair to the public. The Media and everyone should take responsibility by passing along this information and stand up for JUSTICE.

Be the change you want to see in a world of reason and respect for your children and future generations by demanding that the DOE Director or Research and Technology who wrote an 8-line email in which he incriminates himself and proves he cannot do his job, reflect and resign because he broke government rules and reveals how corruption works in assigning taxpayer money to research projects. Demand also NIH organize a PUBLIC scientific review to learn how Crosetto's invention which can slash cancer mortality by 50% and reduce healthcare costs compares to the Explorer project funded by NIH for $15.5 million that can do neither.
XI. Crosetto's inventions endorsed by top world experts in the field and other professionals

Research scientist Dano Crosetto worked on leading edge physics experiments at the world's largest laboratories (CERN in Geneva, Superconducting Super Collider—SSC—Texas, FERMILAB and Brookhaven National Laboratory—BNL), won the Leonardo da Vinci Prize for his invention for early cancer detection, was awarded one million dollars in government grants, and received $10,000 for the best Business Plan reviewed by Venture Capitalists in a start-up competition.

His inventions were endorsed in writing by over 50 top scientists and experts in the field as reported in excerpts from their letters. Here is a list of a few letters in their entirety which represent less than 50% of the entire set:
Andrew Lankford. University of California, Irvine, former Deputy Spokesman of the CERN-Atlas experiment, now Chairman of the HEPAP (High Energy Physics Advisory Panel), who was charged in 2013 by DOE and NSF to develop an updated strategic plan for the U.S. High Energy Physics (HEP) that can be executed over a 10-year timescale, in the context of a 20-year global vision for the field, and was recently charged by the DOE Director of the Office of Science, Dr. Cherry Murray, to assemble a COV committee to assess the operations of HEP during fiscal years 2013, 2014, 2015;
Joel Butler, head of the Computing Division at FERMI National Laboratory, now Spokesman of the CERN-CMS experiment;
Barry Barish, California Institute of Technology (see in session 3, pp. 15-23 an interview about the SSC, CERN-Courier GDE 2008, and Dr. Barish's lecture at CERN 2016), now Plenary Speaker at the 2016 IEEE-NSS Conference on Oct. 31, 2016;
Ralph James, Associate Director of Science and Technology at the Savannah River Nat. Lab., Co-Chair of the 2016 IEEE-RTSD workshop;
Livio Mapelli, CERN leader in Atlas experiment, member of the review panel of the 3D-Flow basic invention held at FERMILAB in 1993;
Maris Abolins, Michigan State University, who endorsed Crosetto's 3D-Flow invention to be used in the D0 experiment in 1994;
Michael Shaevitz, Columbia University, Co-leader, electronic group at the SSC-GEM experiment;
Mike Harris, Chief Engineer at the SSC-GEM experiment;
John People, Director of the SSC (also Director of FERMILAB and representing Universities Research Association, Inc. and the U.S. DOE)
Sergio Cittolin, CERN Group Leader, Readout Architectures Group;
Francoise Bourgeois, Deputy Division Leader of CERN Electronics and Computing for Physics;
Pier Giorgio Innocenti, Division Leader of CERN Electronics and Computing for Physics;
Walter Selove, Emeritus Professor of Physics at the University of Pennsylvania offers collaboration.
Laura Mantegazza, Medical Information Division, Radiology Department, University Hospital of Geneva, Switzerland
Paul Jerabek, University of Texas, PET Division Chief, San Antonio, Tex., who endorsed Crosetto's 3D-CBS to be used at his Research Imaging Center,
Joseph Dent, Assistant Professor, Department of Biology, McGill University, Montreal, Canada
Hon. Jane Nelson, Texas State Senator, who raised $3 billion for cancer research, however, Crosetto's 3D-CBS was triaged out and not even reviewed
Stephen Fluckiger, Partner at Jones Day, among the world's largest law firms covering 5 continents, Dallas, Tex.
Hon. Kay Bailey Hutchison, United State Senator,
Robert Turner, Partner at Jones Day, among the world's largest law firms covering 5 continents, Dallas, Tex.
Paul Bartholdi, Observatory of Geneva, Switzerland;
Catharinus Verkerk, Group Leader Data Handling Division at CERN and Director of the ICTP Colleges on Microprocessors;
Domenico Sannicchio, Medical Physics Director at University of Pavia, Italy, who endorsed Crosetto's 3D-CBS to be used at his hosp.

The superiority in efficiency of Crosetto's 3D-CBS (3-D Complete Body Screening) medical imaging invention made Siemens take a second look at their PET (Positron Emission Tomography) devices and had to agree that the electronics could be improved after insisting (in a meeting with Crosetto that lasted an entire day) the efficiency of their electronics had reached the limit.

Crosetto's basic 3D-Flow invention was recognized valuable by a major public scientific review held at FERMILAB in December 1993, endorsed for the D0 experiment at FERMILAB, adopted by thousands of scientists at the GEM experiment at the SSC and the LHCb experiment at CERN, but never funded to completion, while alternative projects were funded, ultimately failed and were trashed. Recently the 3D-Flow was proven feasible by 59 quotes from industries to replace 4,000 CERN electronic boards with 9 boards.

Crosetto's 3D-CBS invention that can slash cancer mortality by 50% was endorsed for use in hospitals in S. Antonio, Tex., the RHD hospital and Southwestern Medical Center in Dallas, Tex., and S. Matteo hospital in Pavia, Italy, but was never funded, while NIH funded the Explorer project for $15.4 million that cannot make such a claim. Crosetto's 3D-Flow and 3D-CBS inventions along with his subsequent inventions were recognized valuable in several scientific reviews including most notably:
Silvio Turrini, co-inventor in 1993 at DIGITAL Inc. of the first 300 MHz 115W 32b Bipolar ECL Microprocessor
Jerry Merryman, co-inventor with Nobel Prize winner Jack Kilby of the pocket calculator.

Crosetto has also proved the concepts of his inventions feasible and functional in hardware.
F. Facts proving Government's internal arm of the corruption in HEP

Explanation of how Dr. Glen Crawford, Director of the DOE Research of Technology Division, and his predecessors in his position managed to crash Crosetto's 3D-Flow breakthrough inventions and gave $50 million to Wesley Smith for the CMS Level-1 Trigger, which provided him power to convince and drag into the ditch other scientists who applied for funding in other universities in other countries, for an additional $50 million to build the CMS Level-1 Trigger, a system made of 4,000 electronic data processing boards and 40 computers running 200 processes that had to be trashed because incapable to select subatomic particles with specific characteristics.

On Nov. 16, 2015, Crosetto asked on the phone DOE Director of Detector R&D, Dr. Helmut Marsiske, who works in Dr. Crawford's Division, if the current Level-1 Trigger systems they were funding provided zero dead-time and he answered that they were close to designing and implementing a Level-1 Trigger with zero dead-time. Dr. Marskiske was not aware that Crosetto's invention solved this problem in 1992, providing also other powerful features of programmability, higher flexibility and capability to execute complex object pattern recognition algorithms as a lower cost, all characteristics that were formally and officially recognized valuable in a major scientific review paid for by DOE, whose scientific merits were praised and published in DOE 1994 publication DE94005148.

Zero dead-time means that the Level-1 Trigger system has the capability to analyze all frames arriving every 25 nanoseconds, instead of being blind for three to four 25 nanosecond cycles after the Level-1 Trigger fired. Because the probability for the LHC (Large Hadron Collider) to generate a Higgs-like boson particle is one out of 10 billion events, if the Higgs-like boson occurs during those three to four cycles when the Level-1 Trigger system is blind, those events are lost forever.

Despite Smith failure having wasted $50 million taxpayer's money, DOE having funded for 25 years Level-1 Trigger systems not providing zero dead-time, whereas Crosetto had solved this problem 25 years ago and designed a more efficient system at a lower cost, Dr. Crawford continued to give Wesley Smith from $3 to $4 million per year as Smith reports on pages 12 to 16 in his curriculum vitae ($4.2 million in 2016, $3.18 million in 2015, $2.8 million in 2014, $3.38 million in 2013, $4.1 million in 2012 . . . ), eluding a fair scientific competition.
2. Facts that triggered Crawford's 8-line self-incriminating email

In 2014, The Director of DOE-SBIR, Manny Oliver, sent an email to Crawford and Crosetto asking Crawford to answer a question related to particle detection but he never responded. Other emails followed but each time Crawford ignored the emails even though Crosetto was told by many that Crawford should answer Crosetto's questions and address his arguments as this is one of the duties of a DOE Program Manager like Crawford which are defined in several DOE documents (e.g. 2016 DOE solicitation DE-FOA-0001414).

Since May 5, 2015, Crosetto was solicited both verbally and in writing to formally submit a proposal of his inventions to the Department of Energy by his former supervisor at the Superconducting Super Collider, Dr. Jim Siegrist, who is now Director for the Office of High Energy Physics—HEP—at the Office of Science of the Department of Energy. During this time, Dr. Siegrist and several DOE employees told Crosetto he would need to communicate with Glenn Crawford as he is responsible for Research and Technology. However, he never once answered Crosetto's technical emails, phone calls, or addressed/discussed any scientific issues with Crosetto.

To satisfy Siegrist's request, Crosetto worked hard from August to November 2015 with several reputable companies who prepared 59 quotes showing feasibility of a 8,192-channels Level-1 Trigger for LHC in 9 electronics data processing 3D-Flow OPRA boards replacing at 1/1000 the cost the CMS Level-1 Trigger system made of 4,000 boards or the most recent SWATCH made of 100 boards (two slides).

He then submitted a formal proposal of his 3D-Flow OPRA invention (see pp. 1-36, and pp. 125-271 of the proposal to DOE #0000222704, responding to the 2016 solicitation DE-FOA-0001414) to DOE on Dec. 22, 2015, whose review was assigned to Dr. Glen Crawford

Then on May 16, 2016, Crosetto was shocked to be told by the executive officer of the office of Director of Science, Dr. Cherry Murray, that he was not being cooperative in providing information to HEP Office of Science. This is false; Crosetto had never received any requests from Dr. Crawford or any other DOE department—therefore, he could not have been uncooperative.

Crosetto realized that he was trapped in a rigged review that would reject his proposal based on false accusations and then banned it forever, based on the following flaws:
a) During the past years Crawford has never discussed scientific issues with Crosetto and has never answered Crosetto's emails but continued to fund projects significantly less efficient than Crosetto's 3D-Flow invention,
b) Helmut Marsiske, who works on his team told Crosetto on Nov. 16, 2016, that he was funding Level-1 Trigger projects that were not yet dead-time free, hoping to solve that problem and build a dead-time free Level-1 Trigger system (unaware that Crosetto's 3D-Flow invention solved that problem in 1992),
c) When a project is rejected some Program Managers write the statement: “Any further submissions of substantially the same work will be declined without review” (See details at http://blog.u2ec.org/wordpress/?p=1745)

As there was no possibility to talk to Crawford, the only choice Crosetto had to uncover the scientific truth through a DIALOGUE was to withdraw his proposal so he could be in the position of being able to talk to DOE agents to clarify these false accusations and to answer any questions or concerns regarding its feasibility and to explain the quotes, if necessary, involving the professionals from the reputable companies who made the quotes and then resubmit his proposal.

This unexpected withdrawal caught Crawford off guard and triggered a series of events which reveal how corruption is alive and well.

Crawford's use of the words “Any further submissions of substantially the same work will be declined without review” were not valid as Crosetto withdrew his proposal on May 17, 2016, his project was no longer up for review and Crawford made this statement on May 19, 2016 before Crosetto's review was completed as Crawford admitted in the same email that he wanted to complete the “technical merit review” in the following days—in fact after May 17, 2016 Crosetto was now legally in a position to discuss scientific issues with the DOE Office of Science.
3. Crawford's 8-line self-incriminating email and line-by-line comments

Here in its entirety is Dr. Crawford's 8-line email sent to Crosetto on May 19, 2016, where he reveals how the corruption works in eliminating from a fair competition other projects such as the most recent 3D-Flow OPRA invention which can replace hundreds of crates of Smith's electronics with one crate at 1/1000 the cost providing staggering performance improvements of a programmable Level-1 Trigger with higher flexibility capable of executing more complex object pattern recognition algorithms at a lower cost per valid object recognized.

Crawford's 8-line email states one thing and the opposite in the next of following lines. It refers to several false or inexistent rules that neither he or his colleagues can refer the source. He might have invented these rules in serving his plan of corruption, demonstrating that if an innovation conflicts with his own plans or those of the circle of friends of scientists he is serving, it can never be funded by DOE, which clearly fall into an investigation of abuse of power.

On May 19, 2016 Crosetto received the following 8-line email from Dr. Crawford.

################# Dr. Crawford's 8-line email ####################
1. We will honor your request to withdraw application #0000222704 as a matter of professional courtesy.
2. The withdrawal will be made effective upon the completion of the technical merit review you consented to by submitting the application.
3. We plan to take no action on this application: it will neither be declined nor recommended for award.
4. All current and future funding opportunities will be published on our website and the Government-wide portal at www.Grants.gov.
5. In the interest of fair competition, we do not meet with potential applicants to discuss the details of potential applications:
6. our processes are described in each Funding Opportunity Announcement.
7. The work described in your application and in your correspondence is not technically sound and feasible, as required by 10 CFR 605.10.
8. Any further submissions of substantially the same work will be declined without review.

Sincerely, Glen Crawford glen.crawford@science.doe.gov. Director, Research and Technology Division. Office of High Energy Physics. Phone 301 903 4829

################### end Dr. Crawford email ##########################

This is Dr. Crawford's technique to bypass any accountability and any DOE rule, to fund the projects he likes, crush innovations and waste millions of dollars of taxpayer money. In his email, Dr. Crawford reveals the path of abuse of power, stating one thing and the opposite in the next or following lines, inventing and/or referring to non-existent DOE rules serving his plan of corruption, and demonstrating that if an innovation conflicts with his own plans or those of his circle of friends, it can never be funded at DOE.
Line 7: Crawford's statement “The work described in your application and in your correspondence is not technically sound and feasible” without detailing what in his and his reviewer's opinion is not sound and feasible would mean that all scientists who endorsed Crosetto's inventions, the FERMILAB international review panel and engineers and reputable industries who wrote the 59 quotes showing feasibility, are all foolish and incompetent. If Crawford would specify what in his opinion is not sound and feasible, either Crosetto or engineers who wrote the quotes could provide calculations and explain the technology used that he or his reviewers might not be aware of, that prove to be sound and feasible;
Line 7 contradicts what he stated in line 3: “We plan to take no action . . .” because line 7 is making a judgement/evaluation of the project while in line 3 says it will take no action;
Line 7 is inconsistent with the fact that he writes this evaluation on May 19 when the review is not completed as he stated in line 2: “. . . upon the completion of the technical merit review . . .” and its completion was confirmed on May 31 when Crosetto received a message that his proposal was withdrawn. Crawford evaluation/rejection before completion of the review can only be a prejudice or part of a plan to reject it to serve a priori a circle of friends of scientists.
Rule 10 CFR 605.10. lists requirements that Crosetto's proposal proves to satisfy. Following are the 10 CFR 605.10 requirements:
   “DOE shall evaluate new and renewal applications based on the following criteria which are listed in descending order of importance:
      (1) Scientific and/or technical merit or the educational benefits of the project; (The scientific merits to advance science and benefits to humanity of Crosetto's inventions have been recognised in several PUBLIC scientific reviews)
      (2) Appropriateness of the proposed method or approach; (Crosetto's 3D-Flow OPRA invention can replace hundreds of crates of electronics funded by DOE for the Level-1 Trigger at High Energy Physics experiments with one crate of electronics at 1/1000 the cost while providing a staggering performance improvement)
      (3) Competency of applicant's personnel and adequacy of proposed resources; (Crosetto's competence has been proven in scientific articles, in public scientific reviews and endorsed by many letters of top experts in the field (51-53 & 75-83 of the proposal to DOE #0000222704, responding to the 2016 solicitation DE-FOA-0001414). Outsourcing the construction of the ASIC, electronic boards, cables and other components which will be assembled by a team of 12 professionals, 10 of whom will be hired on a competitive basis, and purchasing new instrumentation integrating the one already in house will assure adequacy of the proposed resources)
      (4) Reasonableness and appropriateness of the proposed budget; and (The cost of the components outsourced to companies at an average cost of the labor of $110/hours on a competitive basis with at least two quotes for each component, the cost of the new instrumentation, travel and all items specified in DE-FOA-0001414 solicitation (page 227 of the proposal to DOE #000222704, responding to the 2016 solicitation DE-FOA-0001414) including the five-year salary for a team of 12 people at an average cost of $38/hour, totally to $13.4 million is reasonable and appropriate for developing, building and testing an ASIC, a hardware simulator with the functionality in generating signals similar to a $50 billion LHC apparatus and two Level-1 Trigger systems one in VXI and the other in VME crate which could also be used for three 3D-CBS units, each with 8,192 electronic channels with 32-bits per channel at 40 MHz, capable of executing with zero dead-time experimenters' complex programmable Level-1 Trigger algorithms in real time). Additional Level-1 Trigger systems for other HEP experiments would cost approximately $100,000 per unit.
      (5) Other appropriate factors established and set forth by ER in a notice of availability or in a specific solicitation.” (No other requests were made by DOE to Crosetto).
Line 1 states: “We will honor your request to withdraw application #0000222704” but in the next line Crawford states that he will continue the technical merit review and did not execute the withdrawal immediately on May 17 as Crosetto was told by several DOE officers, including Procurement Director, Dr. Patricia Schuneman, Grant Analyst, Dr. Kimberlie Laing, and Contracting Officer, Dr. Jason Dozier would happen. No one, not even Crawford can cite the DOE rule that the technical merit review should continue so as to prevent the applicant from submitting the same or a similar proposal in the future.
Line 5: Crawford states “. . . we do not meet with potential applicants to discuss the details of potential applications”. One should notice Crawford's words “potential applicants” and “potential applications” which means outside the review process. The statement that DOE Program Managers cannot exchange emails on scientific issues, talk and/or meet with potential applicants outside the review process is false.
This would be contrary to DOE's mission to maximize results with taxpayer money by learning from groups and individuals through meetings and presentations the details of the best project/technology that would solve their problem.
It would not be in the interest of taxpayers, for DOE records shows several minutes of meetings between DOE staff and applicants discussing details of projects of interest by DOE before submission.
It would make no sense for DOE distributing public money to influential scientists without discussing the technical details, comparing it with other projects and asking authors questions and quotes from industries proving feasibility of their claims.
During review of the project DOE rules allow DOE staff and applicants to discuss details as also stated in the rules.
This statement from Crawford is false because Crosetto gave a seminar to his predecessors in his office on Sep. 2, 1999, and no one cited a change of rule in this regard.
Crawford's supervisor, Jim Siegrist, stated the opposite during several phone conversations and in emails with Crosetto from May through July 2015 when he was attempting to set up a presentation at DOE with Crosetto in July or August 2015 (most likely he was prevented by Crawford).
On Jul. 23, 2015, Jim Siegrist wrote to Crosetto: “Too many people are out of the office so no date has been set. We are working on arrangements and will let you know.”
On Aug. 3, 2015, Jim Siegrist wrote to Crosetto: “Dario, I began the process of arranging your visit last week . . .”
On Aug. 4, 2015, Jim Siegrist wrote in an email to Crosetto: “. . . it may be possible to make a future arrangement if we have a compelling case for you to visit. The most important ingredient is your presentation containing a description of the work plan and scope you plan you propose to DOE. If you can send that to Glen and I we will lake a look and get back to you about the need for a visit.”

On May 6, 2016, Jim Siegrist told Crosetto over the phone that they will resume their conversation when his proposal is not under review.

Crawford's statement “. . . we do not meet with potential applicants to discuss the details of potential applications” is false because it is stated in several documents (page 32 of DOE 2016 solicitation DE-FOA-0001414) and he also advertises at his seminars around the Nation (his slide 20) regarding the duty of all Program Managers like him who must communicate by phone, email, or in one-on-one meetings with potential applicants. This statement by Crawford prohibits any future analytical, scientific discussion of the project based on calculations and scientific evidence with the applicant. No one, not even Crawford can cite the rule that DOE cannot discuss science, calculations or scientific evidence with applicants.
Line 8: “Any further submissions of substantially the same work will be declined without review” contradicts what is stated in line 3 “We plan to take no action . . .” This action is devastating to the advancement of science and to serving taxpayers. Crawford prohibits any resubmission of Crosetto's application that was never evaluated because it was withdrawn. This is really crushing an invention forever without analytical discussion, without working through calculations, scientific evidence and comparison with other projects.

Recently, Crosetto updated the advantages/benefits of his inventions in three abstracts/summaries papers submitted to the 2016 IEEE-NSS-MIC-RTSD conference which were rejected for no scientific reason.
4. Questions regarding fundamental inconsistencies within DOE and the scientific community

Here are listed a few questions regarding fundamental inconsistencies.
Why has the DOE, which has knowledge of the official, formal review paid for by DOE of Crosetto's 3D-Flow invention held at FERMILAB in 1993, knowing it was recognized a breakthrough invention superior to any other approaches for Level-1 Triger, capable of solving the Level-1 Trigger of several experiments and also published in the DOE Technology Transfer book DE94005148 in 1994, stopped funding the NRE to build the 3D-Flow ASIC which was proven feasible and in 2001 proven functional in FPGA and $50 million in taxpayer money was given to Wesley Smith to build a Level-1 Trigger made of 4,000 electronic data processing boards, 40 computers that had to be trashed?
Why didn't the DOE provide any calculations or scientific evidence to show how they came to their conclusion that Crosetto's invention to replace hundreds of crates of electronics with one crate at 1/1000 the cost and provide staggering performance improvement was not sound and feasible (which would mean that all scientists who endorsed Crosetto's inventions, the FERMILAB international review panel and engineers and industries who wrote the 59 quotes showing feasibility, are all foolish and incompetent), but instead appointed Security to threaten Crosetto into never contacting any DOE employee regarding this issue?
Why, instead of addressing publicly very important scientific issues that can provide staggering advancements in science and benefit taxpayers in lower costs for physics experiment and save millions of lives with early cancer detection, have scientists and those with personal agendas and/or interests for over 20 years opposed transparency in science for the benefit of humanity and attempted to crush inventions by trying to destroy Crosetto's inventions and eliminate evidence? Is it just a casual coincidence that out of the entire website only the slides of the seminar given by Crosetto on Sep. 2, 1999 at the DOE Office of Science received a cyber attack? Is it just a coincidence that when Crosetto Foundation for the Reduction of Cancer Deaths pursued the crowd-funding initiative, checks from the Crosetto foundation were counterfeited causing the Foundation to close the account? Or is the evidence concrete and clear from the rejections of his presentation of papers at conferences, rejections of articles in scientific journals, rejections of funding, having the microphone taken away from him when he asks legitimate and pertinent questions and the threats he received if he continues to state that his invention proven feasible by 59 quotes from reputable industries can replace hundreds of crates of electronics with one crate at 1/1000 the cost while providing staggering performance improvements?
5. Crawford documents and actions proving he is not doing his job

Analyzing the facts during the past year, it becomes clear how Crawford's corruption works in eliminating from a fair competition other projects. He does not answer to any email addressing scientific issues for the past two years when Crosetto was referred to him by his colleagues as the person responsible to do so. He creates all possible obstruction not to receive scientific information from Crosetto, blocking his emails, denying a presentation of his technology at the DOE office as Crosetto did to Crawford's predecessors in 1999, promising phone appointment and never setting a date and time, forcing Crosetto to send material via certified U.S. mail. Asking Crosetto to submit a formal application, giving the application to his reviewers to be evaluated and formulating a judgement that “it is not sound and feasible” before the evaluation is completed, continuing the evaluation after Crosetto withdrew it. Crawford did not stop the technical merit evaluation because he wanted to formulate the key sentence: “Any further submission of substantially the same work will be declined without review” (which he did even before the technical merit review was completed) and also stating that “we do not meet with potential applicants to discuss the details of potential applications”. All this strategically planned to be in the position to request DOE Security to make a threat to Crosetto if he contacts by email, phone or any form any DOE employee, contractor or associate.

These facts, documents and data should make Crawford reflect that he cannot do the job that he signed for and for respect to the taxpayers who have been paying his salary and provided their money he distributed with no accountability, without doing his job properly, he should reflect, take responsibility and resign. Crawford's job was to maximize the use of taxpayer money to advance science for the benefits of taxpayers, he should communicate with inventors and researchers who can contribute to DOE programs as stated at page 32 of the DOE solicitation DE-FOA-0001414 and also advertised by him at seminars he gives around the Nation (his slide 20) and not to ignore or crush innovations. He didn't have the courage or knowledge to address scientific issues and answering Crosetto's emails, talking to him over the phone, organizing a meeting with experts in different fields who could address scientifically Crosetto's contributions to make the scientific truth for the benefit of taxpayer and humanity prevail. Crawford's methodology/actions show that he is not suitable for his job.
6. Tricks used by Crawford to reject Crosetto's proposals are common to other funding agencies anywhere in the world and have roots in the international scientific community

This trick of eliminating other projects from a fair competition is not only used by Crawford but is the result of the rigged peer-review process in approving/rejecting articles and in assigning taxpayer money to research projects which exposes to corruption several other government agencies and/or cancer organizations because at the end, the circles of friend experts in Trigger or PET who meet behind closed doors splitting taxpayer and donation money among themselves are always the same.

Whether the review or evaluation of a project/idea/innovation is requested by Agency A, B, or C, or is requested by a philanthropist or cancer organization A, B or C in any country in the world, the circles of friends of scientists controlling a specific field are always the same, they are the ones who meet at international conferences such as IEEE-NSS-MIC.

This explains why Crosetto after his $906,000 grant received from DOE could not receive any other grants from any scientific organization, cancer organization, philanthropist or Bank Foundations, not even $2,150 as he applied on July 2016 to the Rockefeller Foundation. Those who control the field have decided to ignore it, to label and spread the words: “not sound, not feasible” without looking at the details and crush his innovations because it might subtract money to their projects, take away their control in the field and overall losing the trust from the public for the scientific community.

In fact, journalists told Crosetto that they were not informing their readers about these facts because they were told by experts in the field that Crosetto's invention was flawed, but did not want to provide their names.
XII. Responsibility of scientists to stand up for SCIENCE to stop taxpayer and donation money from being wasted and innovations that would benefit humanity from being crushed

After Crosetto was solicited verbally and in writing beginning on May 5, 2015, to formally submit a proposal of his inventions to the Department of Energy from his former supervisor, Dr. Jim Siegrist (now Director for the Office of High Energy Physics—HEP—at the Office of Science of the Department of Energy, but a former supervisor when they both worked at the Superconducting Super Collider), Crosetto worked hard for four months to explain under NDA (Non-Disclosure Agreement) his inventions to several reputable companies who prepared 59 quotes based on their component/technology data sheets and results from their simulations.

After the several months Crosetto worked with these companies, he provided their reliable information in the 59 quotes to Dr. Siegrist. On a regular basis, Crosetto received inquiries from the companies asking if he had received any comments/questions from Dr. Siegrist regarding their work. These professionals who spent a great deal of time to perform simulations and provide information about components and technology data sheets, had a legitimate right to know whether there were any technical concerns/objections/questions from Dr. Siegrist or the DOE about the work described in their quotes.

Dr. Siegrist or anyone from DOE ever expressed any concern regarding any component or technology used in the proposal.

These 59 quotes proved it is feasible to build a 3D-Flow OPRA system for Level-1 Trigger at LHC:
a) in one VXI (or ATCA) crate with a 36 cm cube of electronics and 8,192×16-bit channels (or pixels in a frame) with data arriving at 80 million frames per second (or 40 million 8,192×32-bit frame per second, equivalent to a data rate of 1.3 TB/sec), with the capability of executing up to 20 OPRAS at an approximate $100,000 production cost per crate, (OPRAS: Object Patter Recognition Algorithm Step executing up to 26 operations such as add, subtract, compare with 24 values, etc. in a single cycle of 3 nanosecond) or
b) in two VME crates each with a 16 cm cube of electronics and 4,096×16-bit channels (or pixels in a frame) with data arriving at 80 million frames per second (or 40 million 8,192×32-bit frame per second, equivalent to a data rate of 1.3 TB/sec), with the capability of executing up to 30 OPRAS at an approximate $160,000 production cost in total.

The value of Crosetto's inventions have been recognized and endorsed by hundreds of scientists, the concepts have been proven feasible and functional in hardware, providing staggering performance improvements as confirmed in a public scientific review conducted in December 1993 (see pp. 56-74) where Crosetto answered objections from other scientists.

The recent 59 quotes from top engineers of several reputable industries based on components and technology data sheets prove with calculations and simulations that Crosetto's innovations can use in synergy the components and technology to break the speed barrier in real-time applications in object pattern recognition data processing systems with staggering advantages in higher performance and lower costs. Two examples are provided here:
1. Replacing hundreds of crates of electronics of the Level-1 Trigger systems at LHC such as the CMS costing over $100 million made of 4,000 data processing boards, 40 computers running 200 processes (or the most recent SWATCH made of 100 data processing boards) with one 3D-Flow OPRA VXI crate described above at 1/000 the cost with staggering performance improvements. The 3D-Flow OPRA can provide experimental physicists the most powerful tool available for efficiently executing their preferred Level-1 Trigger algorithms, enabling them to quickly and economically confirm or rule out the existence of new particles, and do so with the highest certainty.
2. Changing the molecular imaging paradigm with the 3D-CBS device, the first truly “total body” PET. Replacing the Explorer device recently funded by the U.S. National Institutes of Health (NIH) for $15.5 million made of 491,520 expensive LYSO crystals, 12 crates with 120 data processing boards and 6 racks of computer processing 40 TB data acquired and stored on hard drives every day for a total power consumption of 60 kW, which cannot save many lives because it cannot acquire and process 40,000 TB data per day, with the 3D-CBS device which makes use of the recent 3D-Flow OPRA invention capable of acquiring and processing over 40,000 TB data each day, by 9 data processing boards housed in one 3D-Flow OPRA VME crate capable of extracting all valuable information from tumor markers, from fewer than 3,000 economical BGO crystals, requiring only 1 GB data storage per day, zero racks with computers, for a total power consumption of the entire system of only 4 kW, at less than 1/10 the cost of the Explorer. The 40,000 TB data acquisition and processing per day of the 3D-CBS enables an effective early detection of cancer and several other diseases at a curable stage which can save many lives and reduce healthcare costs.

Why waste millions of dollars of taxpayer money on less efficient, more costly technologies knowing that Crosetto's inventions described in a 271-page proposal proven feasible by 59 quotes form reputable industries can save millions of dollars to taxpayers, advance science and save millions of lives?

Scientists should stand up and request a PUBLIC SCIENTIFIC review of Crosetto's new 3D-Flow OPRA and 3D-CBS inventions similar to the one requested by the Director of the Superconducting Super Collider of Crosetto's basic 3D-Flow invention that was recognized valuable by the panel of experts from academia, industry and research centers at the 1993 FERMILAB review.

Instead of following scientific procedures and addressing analytically calculations, scientific evidence where scientists can question each other and compare in a public forum Crosetto's 3D-CBS and 3D-Flow inventions, Smith's and SWATCH CMS Level-1 Trigger, Atlas Level-1 Trigger and Moses et al., the Explorer's project description, data processing boards, components and technology data sheets, scientists do not take responsibility, do not answer emails when they have to recognize the advantages of inventions, ignore improved scientific approaches, do not do their job, but take action to suppress inventions for the power, personal interest and/or agenda, even copying ideas, breaching basic professional ethics rules.

Because scientists do not stand up for science, Crosetto Foundation received several cyber-attacks and when he tried to raise money through crowdfunding—his Foundation's checks were counterfeited causing the Foundation to close the account; his papers with valuable innovations are rejected for no scientific reasons, funding of his innovations are rejected for less efficient and more costly projects, and when he has attempted to point out these facts to prove all of the above and legitimately ask for scientific reasons why less efficient and more costly approaches are being funded, he is threatened with security being called.

In other words, the louder Crosetto speaks up in defense of science and humanity, spelling out the scientific benefits that can be received from his inventions, the more he receives threats and actions to silence him.

The scientific truth for the benefit of humanity could prevail if enough scientists STOOD UP FOR SCIENCE, but many more would be needed than the number of scientists who wrote letters of endorsement to Crosetto and people who supported his inventions such as Antony Montgomery, head of the Office of Research and Technology Transfer at the Superconducting Super Collider and his wife Ann Montgomery, who in an interview with the Dallas Morning News on Jul. 12, 2013, stated: “You had to be someone special to get Tony's attention . . . He wouldn't reach out to just anyone.” See the article at: http://www.dallasnews.com/news/community-news/best-southwest/headlines/20130712-desoto-inventor-crosetto-eyes-early-cancer-detection-technology.ece. Or the scientific truth could prevail by placing people in key positions who consider it more important to serve the public than to be complicit in corruption.

For this specific case, Crosetto respectfully requests from the bottom of his heart, in defense of science, taxpayer and cancer patients that Crawford, Marsiske and Laurer refer to the ethics of scientists, and be fair to taxpayers and cancer patients by allowing the judgement of analytical discussions, calculations, scientific evidence and ultimately the judgement of experimental results. Because the actions or non-actions reported below show they are not able to do their job or believe their job is to be fair to scientists who are unfair to the public, Crosetto is respectfully asking them to reflect and resign their positions of responsibility to others who can maximize benefits to taxpayers through public scientific procedures that make the scientific truth emerge for the benefit of humanity.

Their leaders, U.S. President Barack Obama, Vice-President Joe Biden and many others are promising in their speeches to the public to “provides the biggest bang for their buck”, to “defeat once and for all cancer”, etc. and Crawford, Marsiske, Laurer's actions or non-actions reported below do not use their position of responsibility to achieve what was promised by their leaders and what is expected from taxpayers. Following is the list of the actions or non-actions from Crawford, Marsiske and Laurer:
1. Glen Crawford with his 8-line email revealed how he is the internal arm in the government agency of corrupt scientists.
2. Helmut Marsiske who stated on Nov. 16, 2015, that they were getting close to designing a Level-1 Trigger with zero dead-time without being aware that Crosetto solved this problem 24 years ago and Marsiske continues to waste taxpayer money on less efficient projects and
3. Michael Laurer, Director of extramural Research of the National Institute of Health who does not want to listen to Crosetto's inventions that go a long way to defeating cancer, not even when it was explained to high school students because Laurer says he wants to be fair to the scientists, without realizing that he is being unfair to the public because the scientists are unfair among themselves and toward humanity. The last information that Crosetto received from the office of the Director of NIH, Dr. Collins, was that they had a meeting on Jul. 19, 2016, to find a person at NIH responsible to address scientific issues justifying the giving of $15.5 million for the Explorer which is less efficient, ten times more expensive than the 3D-CBS device. He promised an answer late afternoon on Jul. 19, 2016, or on the following day; however, to date Crosetto has not receive an answer.
XIII. Responsibility of the media and everyone to pass on this information and stand up for JUSTICE

Everyone should STAND UP FOR JUSTICE to stop wasting taxpayer money and to stop the damage to humanity by those who suppress innovations preventing benefits which would advance science, reduce cancer deaths and healthcare costs.

The damages listed below is just the tip of the iceberg of the much greater damage caused by a rigged peer-review system of articles and the funding of research projects with taxpayer and donation money because scientists control both through a peer-review system that has no accountability.

It is not necessary for the media or laymen to be expert in particle detection, photons, electronics, etc. to understand the inconsistencies and the injustices to taxpayers and humanity. The inconsistencies are easy to understand so if everyone takes responsibility and demands they be addressed and fixed, corruption will be eliminated and everyone will benefit.
A. Stand up for justice to stop the waste of $4 million per year of your money to Wesley Smith who has already wasted over $50 million because he makes power/money triumph over science/reason depriving humanity of the benefits from the 3D-Flow OPRA invention which offers a more powerful tool to discover new particles at a fraction of the cost

Why did DOE give $4.2 million of taxpayer money in 2016 to Wesley Smith (as he reported on page 16 of his curriculum vitae, as well as $3.18 million in 2015, $2.8 million in 2014, $3.38 million in 2013, $4.1 million in 2012 . . . ) after he wasted over $50 million building hundreds of crates with 4,000 electronic data processing board of the Level-1 Trigger systems at LHC such as the CMS costing over $100 million which did not work and had to be trashed (as reported on the first page of the May 2016 article by his colleagues) knowing that it can be replaced with one 3D-Flow OPRA VXI crate (described in 2 pages summary, detailed in 271 pages) at 1/1000 the cost with staggering performance improvements.

If reviewers who gave Wesley Smith $4.2 million this year and over $50 million the previous years for the CMS Level-1 Trigger were not corrupt, they should demonstrate that all scientists who endorsed Crosetto, the FERMILAB international review panel of his 3D-Flow invention, engineers and industries who wrote the 59 quotes showing feasibility, are all foolish and incompetent and point out errors in the simulations, components and technology data sheets of the industries who wrote the quotes.

For your interest, read the 8-line email by DOE Research and Technology Division Director, Glen Crawford which reveals abuse of power with contradictory statements, inventing and/or referring to non-existent DOE rules, demonstrating that if an innovation conflicts with his own plans or those of his circle of friends, it can never be funded at DOE.
B. Stand up for justice to stop the waste of $15.5 million of your money for the Explorer project funded by NIH which is flawed whether used for research or clinical examinations because its authors make power/money triumph over science/reason depriving humanity of the benefits from the 3D-CBS invention offering a more powerful tool, safe to the patient, to detect minimum abnormal biological processes in the body at an early curable stage and at a fraction of the cost.

Why did NIH give $3,314,184 of taxpayer money in 2016 to the team of the Explorer project (as well as $3,054,873 in 2015, out of the $15.5 million) made of 491,520 expensive LYSO crystals, 12 crates with 120 data processing boards and 6 racks of computer processing 40 TB data acquired and stored on hard drives every day for a total power consumption of 60 kW, which cassssot save many lives because it cannot acquire and process 40,000 TB data per day, knowing that the 3D-CBS device can make a paradigm change in molecular imaging, because it has the capability to acquire and process over 40,000 TB data each day, using one 3D-Flow OPRA VME crate for a total power consumption of the entire system of only 4 Kw (described in 2 pages summary, detailed in 271 pages), at less than 1/10 the cost of the Explorer.

The 3D-CBS also offers the 3D-CBS ER/DSU unit at a production cost of approximately $23,000 per unit as described in Table 3 on page 12 and from page 149 to 170 of the proposal capable of acquiring raw data from tumor markers for research studies at a higher detector granularity and time resolution than the Explorer.

The 40,000 TB data acquisition and processing per day of the 3D-CBS enables an effective early detection of cancer and several other diseases at a curable stage which can save many lives and reduce healthcare costs.

Many of the 160 million people who have died from cancer (12 million were Americans) since Crosetto first claimed to improve PET medical imaging efficiency, increase the FOV, improve the electronics, etc., could have been saved if NIH would have requested a public review of Crosetto's invention similar to the major scientific review held at FERMILAB in 1993 with an open analytical discussion based on calculations and scientific evidences.

It took 15 years from when Crosetto wrote his technical-scientific book and 19 years from the first rejection for funding by NIH, for those in power in the field such as Bill Moses, leader of the Explorer project, to reverse their position toward Crosetto's approach and direction of research that they had obstructed for decades. Moses' 2015 press release states: <<We're developing the electronic interface between the detectors and the computer algorithm—and the electronics for this scanner is an order of magnitude more complicated than what's been done before.” says Moses>>.

However, the influential leaders in the field such as Moses, DeRenzo, etc. who set the main stream approach of research in medical imaging pursued the opposite direction during the past decades as it was also stated by DeRenzo in his review of Crosetto's book: “We do not view the electronics as a problem, either in terms of performance or cost”. NIH reviewers were reflecting this opposite direction in rejecting all Crosetto's proposals as well as the reviews of papers at IEEE conferences, for more than a decade, supported by Joel Karp, the General Chairman of the IEEE-NSS-MIC conference in 2002 (who is also among the authors of the Explorer project) who rejected all Crosetto's papers.

The Explorer, headed by Moses, has an extended FOV, improved electronics, and focuses on sensitivity. The authors now praise the advantages of these features that they objected to with Crosetto, telling him to focus instead in improving only spatial resolution and to reduce the cost of the PET device.

These were all ideas copied from Crosetto which the authors of the Explorer and their supporter of the main stream research in medical imaging had rejected and obstructed for decades and for which Crosetto received no recognition in references in their articles. However, the conceptual design of the Explorer is flawed because the influential people who will build it refused an analytical discussion and do not understand what it takes to save lives and reduce healthcare costs. Yes, it has improved electronics, extended FOV, increased sensitivity, and intensified computation, but at the back-end, without understanding that in order to be able to use economical crystals and extract all valuable information (signals) from radiation (tumor markers) at the lowest cost per valid signal captured, it is necessary to increase computation at the front-end with the 3D-Flow invention.

The Explorer is a monstrous device costing over $15.5 million with a farm of computers in six racks, processing an enormous amount of data at the back-end. The conceptual design of the Explorer is flawed whether it is intended for research study or for clinical use. If intended to be used in list mode, recording raw data to be analyzed by a farm of computers in six racks for research study, then the 12 crates with 120 Explorer detector boards can be replaced with one crate with 5 VME 3D-CBS ER/DSU boards described in detail from page 149 to 170 of Crosetto's proposal capable of acquiring raw data from tumor markers at higher detector granularity and time resolution than the Explorer. If intended for clinical study, then the cost of the Explorer is prohibitive and cannot save many lives because it does not have the capability to acquire and process over 40,000 TB data per day. For clinical use, the six racks of computers and 120 detector boards of the Explorer can be replaced by 9 VME 3D-Flow data processing boards described on pages 154-155 and 142-147 of the proposal.

If it took 19 years from Crosetto's first submission to understand some of his ideas of the 3D-CBS, how many more years and how many more needless cancer deaths will be lost before leaders of NIH allow the science of medical imaging to proceed freely through fair, open, public competition?
XIV. Crosetto's Response to the President of IEEE, the world's 400,000 member largest technical professional organization dedicated to advancing technology for the benefit of humanity
   A. Statements made by Crosetto on Sep. 19, 2016 in an email to Dr. Verboncoeur which support his abstracts/summaries with quantitative details Technical Merit, Originality & Innovation and Significance

I respectfully would like to point out that my three abstracts/summaries submitted to the 2016 IEEE-NSS-MIC provide quantitative data, Technical Merit, Originality & Innovation and Significance within the text of the abstracts and figures in the summary that are worth thousands of words, as well as in the email I sent you on Sep. 19, 2016 at 2:30 am:
1. Quantitative data & technical merit provided in the text of my email dated Sep. 19, 2016.

“In my abstract/summar I am describing a 3D-Flow OPRA programmable system for Level-1 Trigger for 8,192×16-bit channels with the capability to extract ALL valuable information from radiation using Object Pattern Real-Time Recognition Algorithms (OPRA) from 80 million events/second (radiation) @1.3 TB/second transfer rate from over a billion collisions/second, using 43,008×3D-FLOW processors @$1 each in a single VXI crate with zero dead-time costing approximately $100,000.”
2. Innovation and significance of the project/research provided in the text of my email dated Sep. 19, 2016.

“This 3D-Flow OPRA system in one crate has the capability to replace hundreds of crates of electronics wth 4,000 data processing boards, 40 computers running 200 tasks of the Smith's CMS Level-1 Trigger.”
3. Figures available in the Abstracts/Summaries of papers 2484, 2493 and 2505 are worth thousands of words.

“For example, the quantitative data stated in the four lines above are detailed in half page figure at page 2 of abstract/summary 2493 showing an overview of the main components of the Level-1 Trigger of large experiments. Page 3 of 2493 show the mechanical dimensions represented in scale of the electronic board, cables, crates, etc. for the Level-1 Trigger. Each component, electronic board, cable, their functionality, performance, speed, connector insertion loss, etc., their cost provided in written formal quotes from at least two different companies for each component, the overall performance of the system handling 1.3 TB/sec data with the capability to execute programmable Object Pattern Recognition Algorithms with zero dead-time is described in detail in 271-page 3D-Flow OPRA proven to be feasible by 59 quotes from reputable industries.”

Following I am addressing point-by-point reviewers' opinions for rejecting my three abstracts and I am requesting action in line with what we discussed and with the IEEE mission stated at the IEEE website: “IEEE is the world's 400,000 member largest technical professional organization dedicated to advancing technology for the benefit of humanity”
B. Review of paper2493 by Eckhard Elsen and Susanne Kuehn responsible with their reviewers to accept/reject papers submitted to the 2016 IEEE-NSS Conference:

“I do not understand how 3D flow differs to various standard network topologies currently used in HEP (this is after reading a poster or two on his website).

Technical Merit poor, Originality & Innovation poor, Significance poor”
1. Response to Eckhard Elsen, Susanne Kuehn and their reviewers' opinion/question/concern:

“I do not understand how 3D flow differs to various standard network topologies currently used in HEP (this is after reading a poster or two on his website).”

The 3D-Flow OPRA Level-1 Trigger system (www.UnitedToEndCancer.org/doc/900.pdf) “differs from various standard network topologies” such as the CMS Level-1 Trigger reported on the first page of the article presented at the Real-Time conference on Jun. 6, 2016, consisting of 4,000 electronic data processing boards confined in a room 2,200 cm×1,600 cm with approximately 136 racks, each rack with 3×9U crates, each crate with 10 data processing boards. In stark contrast, the digital Level-1 Trigger system with the 3D-Flow OPRA is confined in a 36 cm cube of electronics in a single crate with 8×3D-Flow OPRA data processing boards and one 3D-Flow OPRA pyramid board for channels and data reduction (See last page of the summary of paper 2493 )

Because the Level-1 Trigger system needs to provide high performance in executing complex Object Pattern Recognition Algorithms on data arriving at an ultra-high rate as a typical object pattern recognition algorithm needs to exchange data with neighboring processors (or circuits) and cannot move the data of an event (frame or picture) out of the processor (or circuit) until the algorithm is complete, a topology minimizing the distance between electronic components is required to minimize the time to exchange the data, which in turn minimizes the volume of the electronics, the power dissipation, etc.

The CMS topology in 4,000 boards housed in a room with 136 racks of electronics covering a floor of 2.200 cm×1,600 cm as reported in Figure 18.1 on page 579 of CERN/LHC 2000-38, requires some signals to travel 4,000 cm that will take longer than 133 nanoseconds (limited by the speed of light 300,000 km/sec), while signals travelling the longest distance in the 36 cm cube 3D-Flow OPRA system will require approximately 1.2 nanoseconds (100 times faster in moving data). No matter how fast the circuit is executing the algorithm, choosing the wrong topology will limit and provide a less performant system.

Even future 16 nanometer FPGA technology cannot compete in performance with a system having better topology. For example, a 16 nanometer technology might perform 2 to 4 times faster than less expensive technology, however, the CMS topology with electronics in a 700 million cubic cm, limits the data rate and complexity of the algorithms that can be executed to 100 times lower than the 3D-Flow OPRA in 46,656 cubic cm. The use of the FPGA rather than the 3D-Flow ASIC further limits the overall performance and the component cost is another big disadvantage assuming it accommodates 64×3D-Flow processors in a 16 nanometer FPGA at $4,000 per FPGA component, the cost would be 60 times the 3D-Flow ASIC costing $67 with 64 processors and the power dissipation would also increase considerably.

The 3D-Flow OPRA offers by far the best and most balanced topology when one considers cost-effectiveness, power consumption, speed, nanometer technology, component costs, the coupling between detector and electronics, the system architecture, the processor architecture, etc. providing the highest performance in its capability to sustain an ultra-high speed input data rate and the execution of complex real-time algorithms at the lowest possible cost.

Wesley Smith's CMS Level-1 Trigger made of 4,000 data processing boards or the new SWATCH Level-1 Trigger system made of 100 data processing boards are limited in the performance, input data rate and complexity of the algorithms they can execute that is hundreds of time lower compared to the 3D-Flow OPRA due to all factors listed above. However, the first wrong step was made when it was chosen as the topology.

In 1994, I choose the topology of the Level-1 Trigger that was a cylinder of electronics 100 cm in diameter×180 cm tall shown on page 106 of my proposal, reflecting the topology of the trigger towers in the detector.

My last 3D-Flow OPRA design is in the VME and VXI form factor as shown in detail on the second page of the Summary (and it can be easily ported to the ATCA form factor which has a higher cost with no advantages in performance).

I chose VME and VXI form factor for the lower cost and standardization with the other electronics used in the same experiment; however, the topology that will allow to achieve the highest performance for a Level-1 Trigger continues to be my original 1994 design of the cylinder which now it would be approximately 0.4 m in diameter and 1 m tall and would achieve a higher performance in the input data rate and execution of complex pattern recognition algorithms.
2. Quantitative data & technical merits of the project/research provided in the text of the 2493 abstract.

This abstract contains extremely valuable quantitative data & technical merit of the project/research:
a. The 3D-Flow processor has the capability to execute billons algorithms vs. 128 algorithms of CMS experiment.
b. The 3D-Flowprocessor has the capability to execute at each “step” up to 26 operations in less than 3 ns.
c. The 3D-Flow OPRA system has the capability to extract ALL valuable information from radiation . . .
d. The 3D-Flow processor has the capability to execute specialized instructions (or “OPRA steps” for an optimized Object Pattern Real-Time Recognition Algorithm) to identify particles.
e. My 3D-Flow invention was proven feasible and functional in hardware using two FPGA chips in 2001 and two modular industrialized boards in 2003, suitable to build 3D-Flow systems for detectors of any size.

The most valuable quantitative data & technical merit are:
f. The 59 quotes from reputable industries prove it is technically sound and feasible to replace hundreds of crates of electronics with one 3D-Flow OPRA crate at 1/1000 the cost while providing staggering performance improvements.

In light of the results that the Level-1 Trigger of large experiments did not work, the 4,000 CMS data processing boards had to be trashed because very few Higgs-like boson particles were found after analyzing trillion of events recorded casually and not for merit of the Trigger, it is clear that most of the papers on Level-1 Trigger during the past two decades should have been rejected and rated “poor” in quantitative data & technical merit, while papers on the 3D-Flow that was recognized valuable by top experts in the field since 1993 as having the capability to execute programmable complex object pattern recognition algorithms at the Level-1 Trigger with zero dead time should have been approved.

The Sep. 19, 2016 rejection statements by Eckhard Elsen and Susanne Kuehn responsible with their reviewers to accept/reject papers at the 2016 IEEE-NSS conference imply that all scientists who endorsed my inventions, the FERMILAB international review panel and engineers of the industries who wrote the 59 quotes showing feasibility, are all foolish and incompetent.

Although it may not be their intention, by rejecting my papers and making their statements the circumstance they have created must draw to this conclusion.

Eckhard and Susanne should therefore reflect and allow the scientific truth to emerge, by referring to their professional ethics and scientific integrity that requires them to point out the papers that they believe provide quantitative data and have technical merits that they approved for the conference to be superior to the 3D-Flow system. To make the scientific truth emerge, they should give a chance to all authors, including myself, to present their project/research and organize a public workshop to discuss the details and let each author support their claims and question each other.

Furthermore, after my answer and technical explanation regarding how “the 3D-Flow OPRA differs from various standard network topologies” decision makers should reflect and realize that the current peer-review system is flawed and that for twenty years reviewers of my papers have shown they are unqualified to do their job. Ethical scientists should demonstrate their competence and qualification to do the job with pertinent and scientific answers supported by technology, calculations, scientific evidence and not by silencing the questions from the applicant and/or rejecting his papers. The devastating consequence of incompetent reviewers is that decision makers at higher level in the hierarchy, funding agencies, etc. repeat and pass on these non-scientific opinions from the reviewers including the question on the topology which denotes the level of incompetence of the reviewer to do his job that is crushing innovations and detrimental to humanity.

Would Intel, Apple or Samsung place in charge for the vision and planning the future direction of their company a person who claim to be expert in the field who does not know the difference between a relay and an integrated circuit?

Would the U.S. President place in charge a secret service agent who should protect his life and be alert from anything moving in the back yard of the White House to a person who does not know the different it might pose the presence of a small lizard, or a small squirrel compared to an intruder, hundreds of times bigger like a man armed with several weapons breaking the fence of the perimeter of the White House?

If reviewers of IEEE papers who are supposed to be knowledgeable and expert in the field cannot see the difference in advantages between a topology hundreds of times bigger than the other, how many other advantages that requires a deeper understanding such as a different architecture, flexibility, scalability, technology-independent, advantages in using one type of connector and cable rather than another etc., is this reviewer going to miss and deprive humanity from advancement in science and the subsequent benefits they could receive?

The only way to fix this is to allow science to proceed freely through a reform of the peer-review procedure allowing the presentation of my inventions and those by others at conferences and at funding agencies, comparing their advantages publicly, requesting other authors and reviewers' responsible to make the best use of taxpayer and donation money to express their concerns, ask their questions, such as in this case, let authors respond and support their claims.
3. Innovation and significance of the project/research provided in the text of the 2493 abstract

This abstract contains extremely valuable innovations and significance of the project/research:

The 3D-Flow performance is further increased by its bypass switch and North East West South communication channels with neighbors.

The basic 3D-Flow invention was first acclaimed in 1992 in many letters from scientists.

The 3D-Flow invention was recognized valuable by academia, industry, and research centers in a formal scientific review held at FERMILAB on Dec. 14, 1993.

The 3D-Flow invention has the capability of extracting ALL valuable information from radiation, which can save taxpayers millions of dollars in HEP experiments . . .

It can replace many crates of electronics of the current experiments at CERN with a single crate, at a staggering increase in performance, and at a fraction of the cost.

The innovations provide a staggering significance in advantages as:

The 59 quotes from reputable industries prove it is technically sound and feasible to replace hundreds of crates of electronics with one 3D-Flow OPRA crate at 1/1000 the cost while providing staggering performance improvements.

It is inconceivable that 24 years after my basic 3D-Flow processor architecture invention, system architecture, bypass switch, etc. which enabled a dead-time free Level-1 Trigger in 1994 to be built in a cylinder 1 m×1.8 m and its evolution to today's 3D-Flow OPRA system with higher performance housed in a 36 cm cube for 8,192 electronic channels at 80 million events/sec per each 16-bit channel, reviewers still claim they cannot understand my invention and for 20 years have approved articles and funding of a 700 million cubic cm CMS Level-1 Trigger without zero dead-time, having much lower performance and a much greater cost.

Because the reviewers of articles are the same as the reviewers for funding projects, this would explain the statement of DOE Director of the Detector R&D, Helmut Marsiske, who funds HEP projects, when he stated on Nov. 16, 2015, during our phone conversation that they were close to designing and implementing a Level-1 Trigger with zero dead-time and did not know that I solved this problem in 1992.

It is inconceivable that reviewers of this abstract are not aware of the many endorsements of my inventions, several in writing and in letters by many scientists who are experts in the field, and by the panel of experts of the major scientific review held at FERMILAB on Dec. 14, 1993.

It is inconceivable that reviewers of this abstract/summary still cannot understand the value of my invention and the advantages compared to the other abstracts/summaries they are approving.

The advantages of my 3D-Flow invention have been understood to be valuable by many scientists, accepted for publication in several scientific journals, including being published after a peer-review in a 45-page article in the journal Nuclear Instruments and Methods in Physics Research in 1999. It has been published in several books; it has been understood by middle and high school students as shown in the YouTube video (see at minute 7:58) when a group of student were solving the problem of analyzing for 30 seconds every envelope arriving at a rate of one every 6 seconds. which explains how to build a Level-1 trigger system dead-time free with the capability to process each event for a time longer than the interval between two consecutive input data.

How can reviewers of this abstract/summary and IEEE-NSS reviewers of the past 16 years continue to reject my papers with a “poor” score in innovation and significance and approve hundreds of other papers that cannot claim advantages from innovations as mine and build a 700 million cubic cm Level-1 Trigger?

The statement in my abstract: “It can replace many crates of electronics of the current experiments at CERN with a single crate, at a staggering increase in performance, and at a fraction of the cost” is very clearly addressing the significance of my work. If reviewers disagree with this statement, it is their ethical duty to refute my claim with calculations, references to technology, scientific evidence and references to other articles/projects/approaches with greater significance (e.g. reducing Smith's 700 million cubic cm Level-1 Trigger to a system smaller than my 36 cm 3D-Flow OPRA) proving their competence and not covering up corruption by silencing my invention.
4. Supporting material to the quantitative data, technical merits, innovation and significance of the project/research provided in the text and the figures worth a thousand words in the 2493 summary

I respectfully request that Eckhard Elsen and Susanne Kuehn carefully study the three figures of the summary that are worth more than a thousand words. For better understanding, FIGS. 1 and 3 of the 2493 summary should be viewed electronically in order to zoom-in and see the details which are drawn to scale relative to the dimensions reported for the crates.

I respectfully request that Eckhard Elsen and Susanne Kuehn provide a Summary or article in any scientific journal which provides an equivalent amount of information as the two-page 2493 summary from the top down design of the entire system to the detail of each component with reference to the 271 -page document explaining in detail each component supported by 59 quotes from several reputable industries with at least two quotes from different industries for each component.

The first figure in the first page of the 2493 summary provides an overview of the entire Level-1 Trigger for four large HEP experiments. On the right of the figure are represented the four experiments at CERN: CMS, Atlas, Alice and LHCb and the “Trigger Tower” which provides the raw data information from the detectors at each of the 8,192 electronic channel.

This data that in current detectors are less than 16-bit (typical 11-bit) per channel every 25 nanosecond (however, the 3D-Flow OPRA provide additional performance for future detector upgrade accepting up to 32-bit every 25 nanoseconds per channel), are sent with different cables, connectors and protocols to the backplane of the dual backplane PRAI crate (Patch Panel Regrouping Associates Ideas).

Signals from different subdetectors are aligned to the same event with the same time-stamp, formatted in 11- to 32-bit words, and sent simultaneously to the 8,192 electronic channels of the 3D-Flow OPRA VXI through the connectors located on the front panel of the 8-blade ATCA boards plugged into the front-plane of the dual backplane PRAI crate. The 3D-Flow OPRA VXI crate on the right of FIG. 1 of the 2493 summary contains the entire Level-1 digital Trigger system consisting of 8×1,024 channel data processing boards and one 3D-Flow Pyramid board for data reduction and channel reduction.

FIG. 3 on the second page of the summary 2493 details the system providing the mechanical dimensions to scale of each component, whose functionality is described in detail in the 271 page document available at the hyperlink provided in the abstract and in the summary and whose cost and feasibility is supported by 59 quotes from several reputable industries.

On the bottom left of FIG. 3 of the summary 2493 is shown the ATCA-PRAI crate with the information arriving from the detector on different connectors to a board plugged into the backplane of the dual backplane ATCA.

Each of the eight ATCA blades plugged into the front of the dual backplane house 8×400-pin connectors which each carry 128 signals at 640 Mbps (DDR at 320 MHz) speed per signal, transported to the 3D-Flow OPRA boards through 128 Micro Twinax cables. The Micro Twinax data sheet is reported on page 184, the 400-pin connector data sheet on page 187 and pin assignments using many pins for ground to improve signal integrity and eliminate crosstalk is reported on page 185 of the proposal. To assure feasibility and reliability, I have used components that can offer performance much higher than what is used in this application—in some case ten times the requirement which will be useful for future speed upgrades.

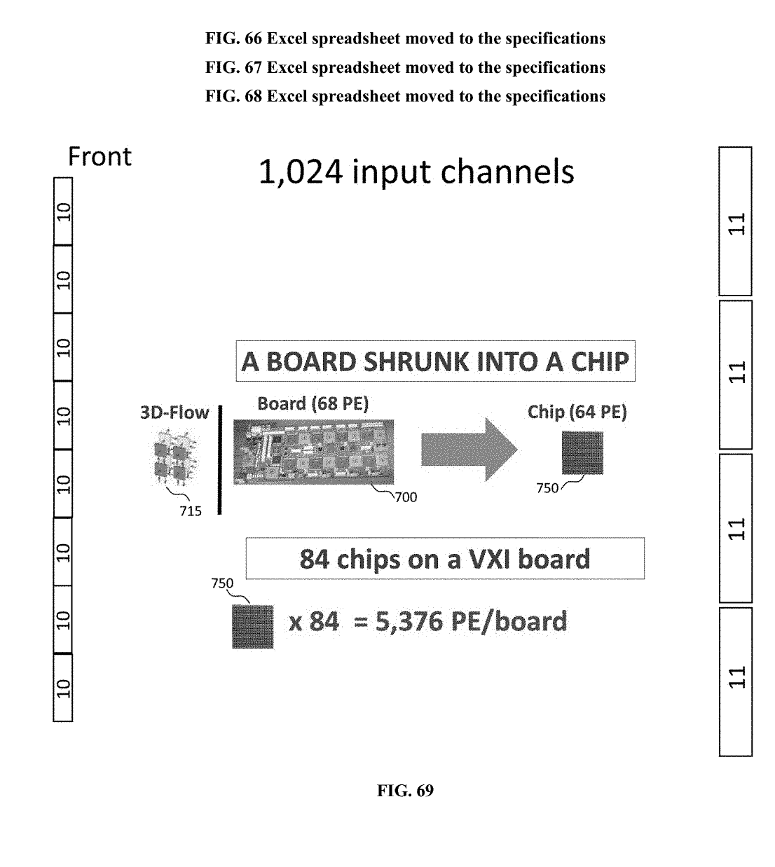

The design of the two boards and 128 Micro Twinax assemblies carrying the signals from the ATCA crate to the 3D-Flow OPRA crate is detailed on FIGS. 66-67 on pages 195-198 of the proposal. Each receives 3D-Flow OPRA boards for 1,024 channels and is described in FIGS. 35 and 36 on pages 155-156 of the proposal.

The text on page 1 and 2 of the summary lists the main features of this project, and the last page explains how valuable information is extracted from radiation. One very important provision of the project is the verifiable capability mentioned on the first page of the summary described in detail from pages 149 to 170 of the proposal to DOE #0000222704, responding to the 2016 solicitation DE-FOA-0001414. It is the TER/DSU unit costing approximately $50,000 that can generate the functionality of the LHC apparatus allowing any Level-1 Trigger system to be tested on a test bench even far from the $50 billion LHC collider-detector at CERN in Geneva.

FIG. 2 at the bottom of page 1 of the 2493 summary, summarizes the significance of this project which would replace hundreds of crates of electronics for the level-1 Trigger of current experiments with one crate.
5. Crosetto's comments to Eckhard Elsen and Susanne Kuehn responsible with their reviewers to accept/reject the 2493 paper

Dear Eckhard and Susanne,

I believe that scientists should not fight over scientific issues, but cooperate in understanding the laws of nature that will provide well-being to humanity.

A scientist has an ethical duty to explain the reason(s) for rejecting the claims made by applicants by providing calculations and logical reasoning, not just rejecting it for no scientific reason.

On behalf of taxpayers and cancer patients who are deprived of the benefits of my inventions, and referring to our ethics and duty as scientists to pursue the scientific truth, I am asking Eckhard Elsen and Susanne Kuehn and the reviewers appointed by them to reflect about their actions which demonstrate that they and the reviewers appointed by them are not competent to do the job required in their position as Chairman and Deputy Chairman of the IEEE-NSS conference and resign, leaving this responsibility to other people more competent in the field.

In the event Elsen and Kuehn disagree with the analysis of the facts showing that they did not provide sound scientific reasons to reject my papers, I respectfully request them to provide references to other abstracts/summaries that they and their reviewers accepted in the field of Level-1 Trigger that they believe have provided an abstract and summary with more quantitative details, higher technical merit, innovation and significance and we could continue the discussion referring to some concrete cases.

I am providing some background information that should make you realize why the rejection of all my IEEE-NSS papers for the past 16 years does not comply with the ethics of scientists/reviewers and is not in agreement with the IEEE mission as stated on their website. This should open an investigation into the unfairness of the current peer-review system that is highly damaging to taxpayers and cancer patients.

Here are some facts:

a) In 1993, I received many letters of endorsement and appreciation of my 3D-Flow invention from scientists and experts in the field and passed a major scientific review with experts from academia, industry and research centers. In 1995, I received $906,000 in grants from DOE to develop the 3D-Flow software tools and the RTL files to be sent to the silicon foundry to produce the 3D-Flow ASIC with four processors in a chip. The money to complete the project was never provided. Wesley Smith, responsible for the trigger at SDC experiment at the Superconducting Super Collider, contrary to all other scientists and experts in the field, wrote me an email stating that programmability at the Level-1 Trigger was not necessary. After the closure of the SSC, Smith became responsible for the Trigger at CMS experiment at CERN. Beginning in 1999, I receive strong opposition to present my work at the IEEE conferences and articles to the IEEE Transaction on Nuclear Science. In 1998, my 3D-Flow system was listed by Eric Eisenhandler on page 53 of the CERN/LHCC/98-36 (Fourth Workshop on Electronics for LHC Experiments) in his presentation among all possible solutions to the Level-1 Trigger for LHC experiments and my article was reported on pp. 517-522. The following year, Wesley Smith and Peter Sharp were co-Chairmen of the same conference in Snowmass, Colo., and they prevented me from attending the conference as they knew my solution was superior to theirs and they did not want to “lose”, just like an athlete with higher skill is prevented to the Olympics from others because they know will lose the competition. I can provide a long list of rejections, boycotts, and difficulties encountered which are not scientific and not fair to taxpayer, cancer patients and humanity who have been deceived, have sustained a high economic burden of wasted money and deprived from the benefits of my inventions.
b) Over the years I have tried having an analytical scientific discussion on the Level-1 Trigger with Patrick Le Du, a senior organizer of the IEEE-NSS conferences. Our relation has always been cordial; however, the typical course of events at the IEEE conferences is the following: on day one I ask him for a meeting; he schedules our meeting during a coffee break but then never shows up; we set another meeting, and again he does not show up. He never answers emails on technical-scientific issues.

I hope that this information will make you realize that there are some individuals within the scientific community who are not only are unethical and unfair to taxpayers, to science and to their colleagues but discredit the entire category with their behavior (if these facts do not convince you that ethical conduct has been broken within the IEEE community, I can provide additional facts).

To restore the prestige of the scientific community, give those with innovations the opportunity to present their papers with their inventions, it is necessary to reform the peer-review system and organize public workshops where the skills of the participants are compared publicly and the best ideas are revealed, just like an athlete skill is revealed at the Olympics.

Your position as Chairman and Deputy Chairman of the IEEE-NSS conference gives you the possibility to restore the prestige of the scientific community referring to the ethics of a scientist serving science and the interest of taxpayers rather than the personal agenda of the greed for power of a few scientists. I am just serving the community and have done my part by answering as I did in this document your and your reviewers' questions about the different topology of the 3D-Flow and the other trigger system. If you disagree with my answer, please provide your scientific consideration and I will give you my consideration.

In the event you realize that the best action would be to allow science to proceed freely through a fair, open, public scientific discussion but you are under pressure as I imagine Chairman Uwe Bratzler was in the 2008 IEEE-NSS-MIC conference when he was unsuccessful at receiving the reasons for the rejection of all my abstracts/summaries, then you should consider resigning in order to send the right message to those who do not respect your position and authority as Chairmen who are responsible for making the scientific truth emerge.
C. Review of paper 2484 by Eckhard Elsen and Susanne Kuehn responsible with their reviewers to accept/reject papers submitted to the 2016 IEEE-NSS Conference:

“this is pure nonsense. duplicate with #2493”

And from a second reviewer: The last several sentences does a lot of damage to the credibility of this abstract.

The architecture seems like a standard parallel algorithm, with pipes that feed multiple CPU's—so I'm missing the innovation here

Technical Merit poor, Originality & Innovation poor, Significance poor”
1. Responses to Eckhard Elsen, Susanne Kuehn and their reviewers' opinions/questions/concerns:

“this is pure nonsense, duplicate with #2493”

This project makes a lot of sense and its feasibility is proven in fine details of the architecture, the electronics, mechanics, algorithms, software, speed of components, power dissipation, etc.

It is surprising the reviewers do not understand the merits of the 3D-Flow OPRA which now can execute programmable pattern recognition algorithms in one VME crate containing 4,096 electronic 16-bit channels (or pixels in a frame) at 80 million frames per second (650 GB/sec) at approximately $80,000 production cost per crate, or in one VXI crate with 8,192 electronic 16-bit channels (or pixels in a frame) at 80 million frames per second (1.3 TB/sec) at approximately $100,000 production cost per crate, with the potential to build 3D-Flow OPRA systems in one VXI crate capable of sustaining an input data rate of 20 TB/sec.

This breakthrough invention has evolved from the 3D-Flow invention recognized valuable and endorsed by hundreds of scientists, by a panel of experts from academia, industry and research centers in a major review held at FERMILAB in 1993, accepted for publication in prestigious peer-reviewed scientific journals, understood by middle and high school students and their teachers with whom I wrote a book and now, this new 3D-Flow OPRA topology, architecture, system with staggering performance in breaking the speed barrier in real-time application at the lowest possible cost is proven feasible from 59 quotes from reputable industries.

It is surprising the reviewers do not understand the advantages of the 3D-Flow OPRA that can replace hundreds of crates housing 4,000 electronic data processing boards, 40 computers, running 200 processes of the Wesley Smith's CMS Level-1 Trigger with one crate housing 8×3D-Flow OPRA data processing boards and one pyramid board for data and channel reduction at 1/1000 the cost with a staggering performance improvement.

Either the reviewers do not understand or they have a different agenda like Wesley Smith and Peter Sharp who prevented me from attending the Workshop on Electronics for LHC at Snowmass in 1999 and many others who rejected all my papers submitted to IEEE-NSS and to TNS during the past 16 years and who refuse a scientific forum proposed in 2001 by the General Chairman of the IEEE-NSS-MIC conference, Uwe Bratzler. Why was it that after DOE granted me $906,000 from 1995 to 1998 to develop the 3D-Flow software tools and the tape out to send to the Silicon Foundry to build the 3D-Flow ASIC, suddenly all funding stopped and all papers within the scientific community of the most influential people who shape the future of High Energy Physics and Medical Imaging were rejected? All funding stopped too, even from funding agencies who were not participants at these conferences; is it because they and cancer organizations, philanthropists and laymen receive information from the media about the best technological breakthroughs from reviewers in a specific field of particle detection and medical imaging who are always the same and are led by influential people who have their own interest on how to spend taxpayer and donation money and provide evaluations like this reviewer: “this is pure nonsense.”

Historical data shows that 1998 was the turning point when in the same year my scientific work and inventions were crushed by influential people in the field and plans were made to spend millions of dollars on projects that were much less efficient and more expensive and ultimately failed.

This failure could have been proved analytically before construction it had underwent an open, fair, public scientific review as my 3D-Flow and 3D-CBS projects were proven analytically to work.

My projects proved analytically the capability to sustain high input data rate, execute programmable complex object pattern recognition algorithms with zero dead-time, to find new subatomic particles and to find all possible signals from the tumor markers enabling an effective early cancer detection, but it was not funded.

Instead the CMS and other Level-1 Trigger proved analytically that could not sustain LHC input data rate with zero dead-time, could not execute programmable complex object pattern recognition algorithms, costing 1000 times my 3D-Flow level-1 Trigger and it was funded.

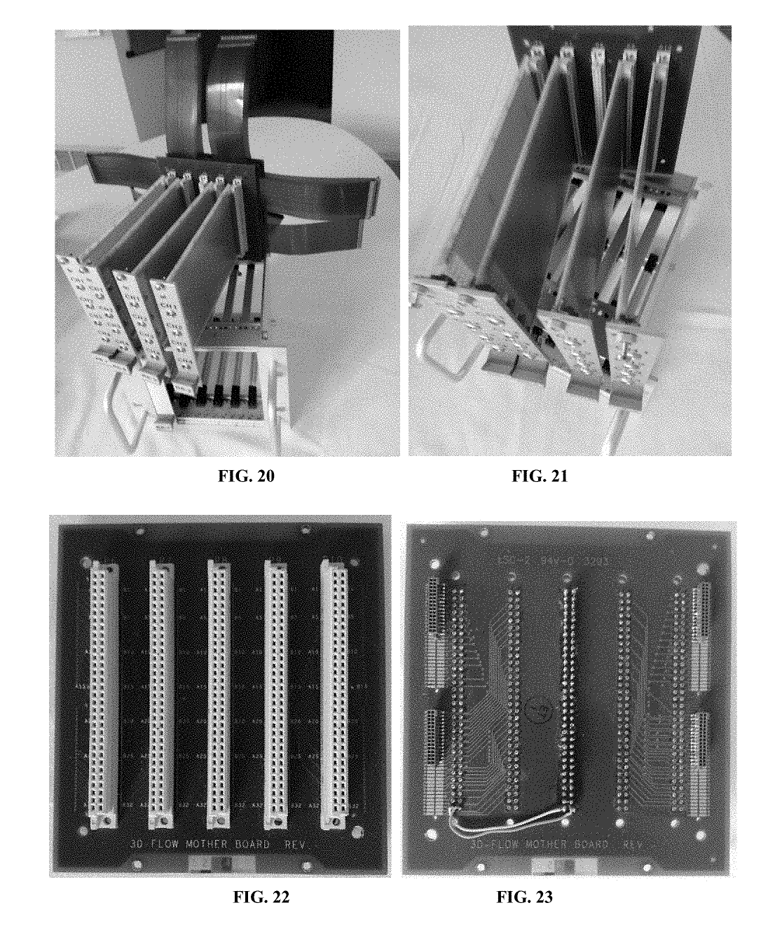

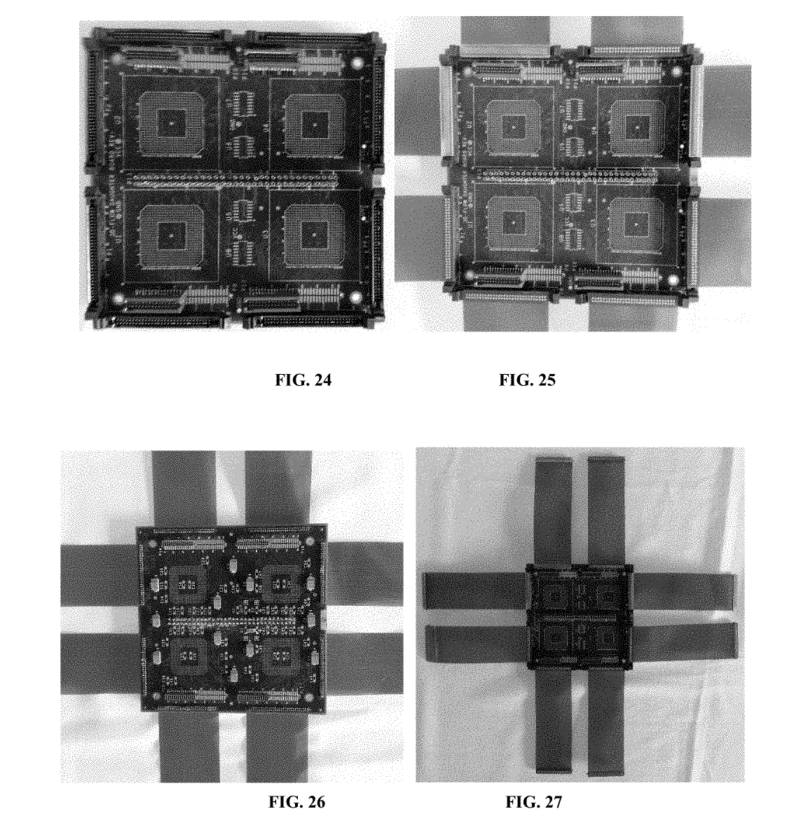

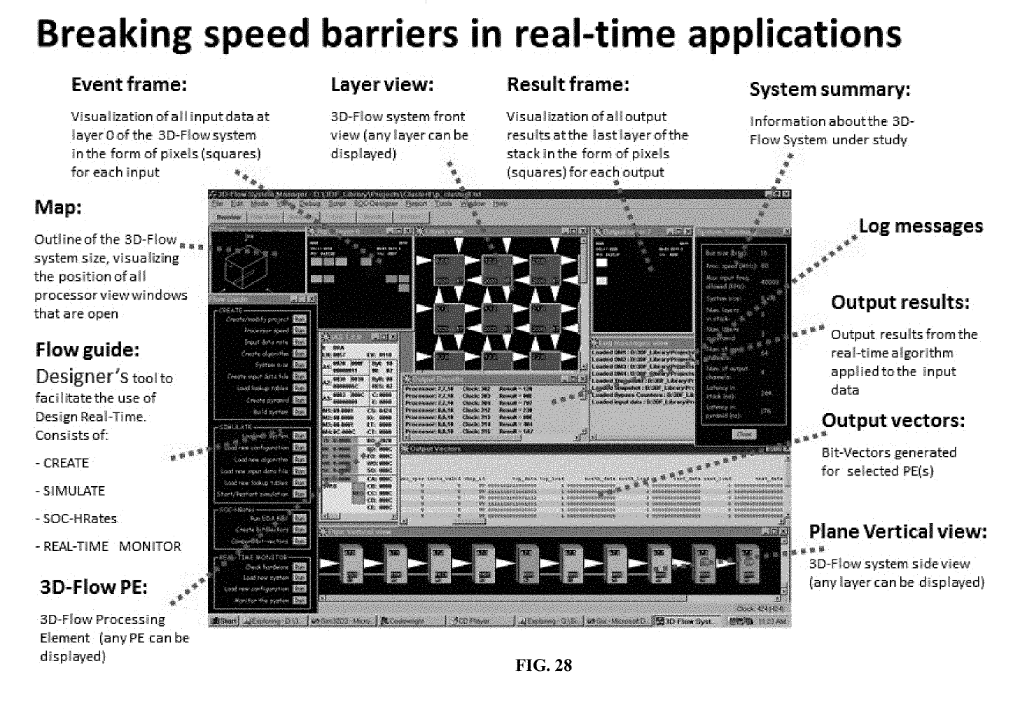

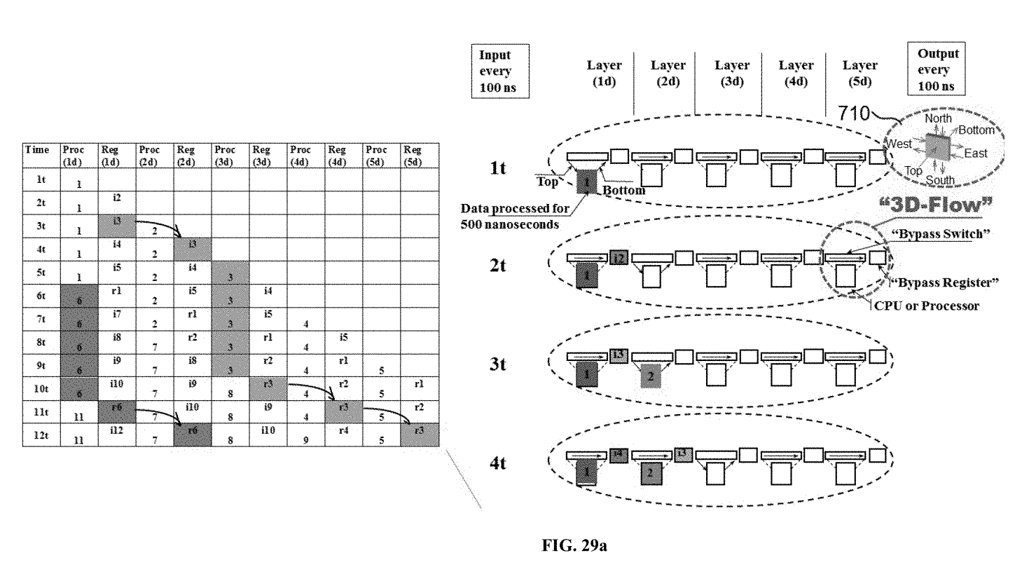

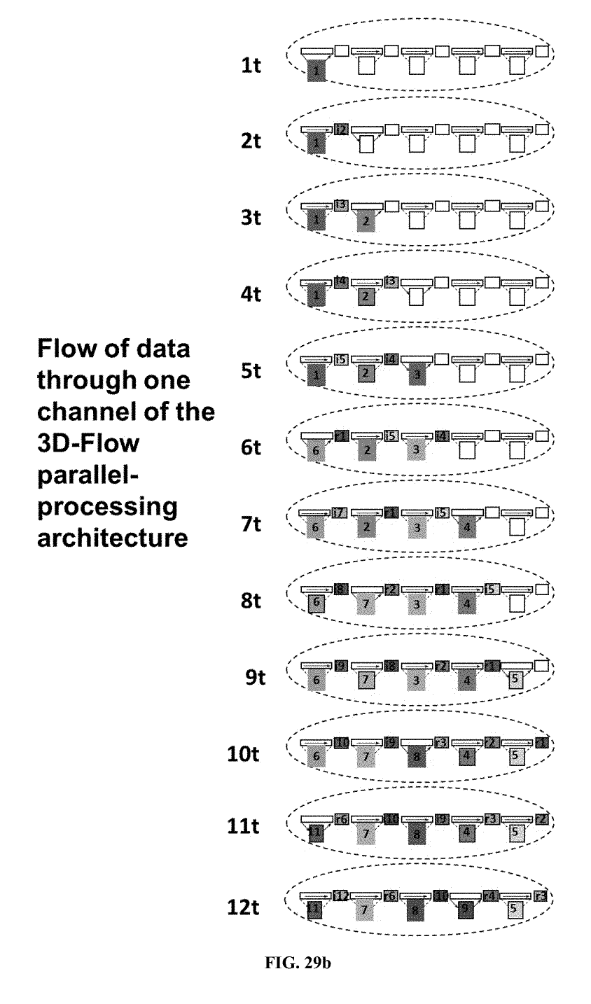

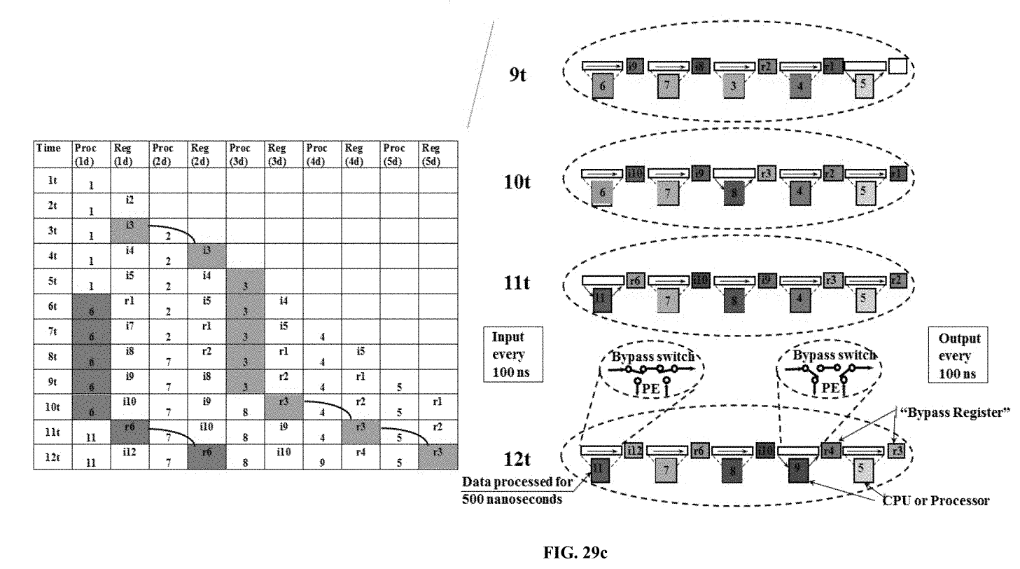

Then in 1999, I was prevented by Wesley Smith and Peter Sharp from attending the Workshop on Electronics for LHC Experiments in Snowmass, Colo., and following rejections by many reviewers of my papers, articles and funding. At this turning point, Wesley Smith and his friends laid out the CMS Trigger Schedule from 1998 to 2005, reported in FIG. 20.1 on page 589, in the 2000 CMS Level-1 Trigger Technical Design Report CERN/LHCC 2000-038. On page 579, FIG. 18.1 of the same document, was also reported the layout of hundreds of racks, each containing either 5 VME crates or 3 VXI crates. The $50 million DOE and NSF funding had to go in the direction of Smith and his colleagues which meant ignoring any fair transparent scientific procedure; this explains the actions on all fronts to crush publications and funding of my project that had been recognized superior.

This impediment to advancing science and the scientific truth for the benefit of humanity only increased during the course of these past 16 years. On Sep. 2, 1999, I was able to give a presentation to DOE at their office. However, influential scientists were able to use their power to stop the funding and the completion of my 3D-Flow project, block any review similar to the one held on my invention at FERMILAB in 1993. Now, after several attempts by the Director of the Office of High Energy Physics, at the Department of Energy, Dr. Jim Siegrist, to organize a presentation of my work at their office since June 2015, this is no longer a possibility as it was in 1999.

It is surprising that the reviewers do not understand the advantages in Medical Imaging that the 3D-CBS device can bring, allowing an effective early cancer detection with the potential to reduce over 50% of cancer deaths.

Responses to Eckhard Elsen, Susanne Kuehn and their reviewers' opinions/questions/concerns:

“The last several sentences does a lot of damage to the credibility of this abstract.”

Because my abstracts/summaries that justify a high score have been rejected for the past 16 years without providing scientific reasons and because reviewers have approved other abstracts that would justify a lower score in technical merit, innovation and significance, it is legitimate and appropriate in order to accelerate the transfer of my inventions to benefit humanity to add to my abstract:

“I have faced many adversities and obstacles these past years that have delayed implementation of my invention, and the resulting monetary burden to taxpayers as well as curbing the advancement of science call for . . . a PUBLIC scientific review of my 3D-Flow OPRA invention with funding agencies—similar to the one I had in 1993 of the 3D-Flow which recognized it valuable but where there were not enough funds available for its completion”

Response to Eckhard Elsen, Susanne Kuehn and their reviewers' opinions/questions/concerns:

“The architecture seems like a standard parallel algorithm, with pipes that feed multiple CPU's—so I'm missing the innovation here.”

No, it is not. I explained the difference orally beginning with my 90-minute presentation to hundreds of scientists at the major public scientific review of my 3D-Flow invention requested by the Director of the SSC held at the FERMILAB auditorium on Dec. 14, 1993, which lasted an entire day.

I explained the difference between my 3D-Flow pipeline architecture and the classical pipeline at several conferences and in several published articles; however, I faced strong resistance to the paper presented (see FIG. 4 explaining the different parallel and pipeline systems) at the 1999 IEEE-NSS conference that was never published in Transaction of Nuclear Science (TNS). Despite IEEE senior scientist (Aaron Brill) intervening in defense of a fair review involving renowned scientist (Les Roger) to make scientific merit prevail, and despite having the majority of the reviewers approve my paper for publication in TNS, the paper was never published because two anonymous reviewers claimed (without providing scientific reasons) that the 3D-Flow was flawed (although it was analytically explained and simulated in 1999 that it was not flawed and in 2001 proven feasible and functional in hardware).

It was presented again the following year at the 2000 IEEE-MIC conference and the difference with respect to the classical pipeline architecture is explained on pages 12-87, 12-88.

In 2000, after explaining the different parallel processing and pipeline architectures in several lessons to the middle school students of St. Alcuin Montessori school in Dallas and learning the differences hands-on with practical exercises and analogies, together with the students and the teachers I wrote a book which was available on Amazon.com for several years and which has been brought to the public again in 2016, explaining the different parallel processing and pipeline architectures from page 36 to page 66 on the blog http://blog.u2ec.org/wordpress/?p=1541#more-1541.

My 3D-Flow invention is not “The architecture . . . like a standard parallel algorithm, with pipes that feed multiple CPU's”, is not an algorithm whose execution is split into several CPUs, instead the entire algorithm is executed in a single CPU. It is not a task split into several subtasks pipelined like the assembly line of a car, television, refrigerator, manufacturing company, but the entire task (algorithm) is executed in a single pipeline station (CPU).

It breaks the speed barrier in real-time applications providing the capability to execute programmable uninterruptable algorithms for a time longer than the time interval between two consecutive input data sets (events or frames) with zero dead-time at the lowest possible cost per each valid event captured. Zero dead-time means that ALL frames are analyzed.

This is different from the alternative Level-1 Trigger systems developed during the past 25 years for large HEP experiments which missed events when the trigger fired and failed to trigger on valid events. Had these alternative Level-1 Trigger systems worked, the cost would have been exorbitant for each valid event captured.

Had the expensive trigger apparatus for CMS and Atlas undergone an open/transparent analytical study/review before construction in 1998, and been compared to my 3D-Flow Level-1 Trigger System, then millions of taxpayer dollars and the work of thousands of scientists would not have been wasted when it was trashed because they would have known in advance that it was not dead-time free and could not execute complex object pattern recognition algorithms. When the 3D-Flow Level-1 Trigger System underwent such a review in 1993, it was formally and officially recognized as not only capable of achieving zero-dead time and executing programmable complex object pattern recognition algorithms but at a fraction of the cost while sustaining a very high input data rate.

Instead of building the 4,000 CMS data processing board using hundreds of crates in a 700 million cubic cm volume room that did not work, they could have built the 3D-Flow system at one thousandth ( 1/1000) the cost.

In 1994, the 3D-Flow system would satisfy the requirements of the Level-1 Trigger for different experiments at LHC such as CMS, Atlas LHCb, etc. in a cylinder 1 m×1.8 m as shown on page 106 of the proposal.

In 1999, the 3D-Flow system for the Level-1 (or Level-0) Trigger of LHC experiment could be built in 6×9U crates as described on page 376 in the 45-page peer-review article published in Nuclear Instruments and Methods in Physics Research A 436 (1999) pp. 341-385.

In 2003, I built two modular IBM PC boards, each with 68×3D-Flow processors in FPGA (that needed NRE funding to be converted into an ASIC) shown in FIG. 15 that is providing a topology in 3-D assembly through the backplane found on page 157 of the proposal to DOE #0000222704, responding to the 2016 solicitation DE-FOA-0001414 and suitable to build 3D-Flow systems for detectors of any size.

Recently, my added inventions were proven feasible by 59 quotes from reputable industries to build 4,096 electronic 16-bit channels (or pixels in a frame) on a VME crate (see FIG. 14) with a 16 cm cube of electronics at 80 million frames per second (0.65 TB/sec) executing up to 30 OPRAS at approximately $80,000 production cost per crate; or in one VXI crate (see FIG. 4) with a 36 cm cube of electronics and 8,192 electronic 16-bit channels (or pixels in a frame) at 80 million frames per second (1.3 TB/sec), executing up to 20 OPRAS at approximately $100,000 production cost per crate; and the potential to build 3D-Flow OPRA systems in one VXI crate capable of sustaining an input data rate of 20 TB/sec (see FIG. 1). In FIG. 56, FIG. 57, FIG. 58, FIG. 62, FIG. 63, FIG. 64 and FIG. 65 are shown VME boards to build a 3D-Flow OPRA system in a 3-D topology in a crate with a 16 cm cube data processing board. (OPRAS: Object Patter Recognition Step executing up to 26 operations such as add, subtract, compare with 24 values, etc. in a single cycle of 3 nanosecond).

The bottom line is that Eckhard Elsen, Susanne Kuehn and their reviewers still do not understand the differences and advantages of the 3D-Flow architecture and topology of the higher performing 3D-Flow Level-1 Trigger system using ONE 36 cm cubic crate, and the CMS Level-One Trigger system using HUNDREDS of crates contained in a 700 million cubic cm volume and costing 1000 times the 3D-Flow OPRA. And Helmut Marsiske, DOE Director of Detector R&D, who assigns taxpayer money to research projects with Glen Crawford have been giving $3 to $4 million every year to Wesley Smith to build the flawed CMS Trigger system at a cost of over $100 million. On Nov. 16, 2015, Marsiske stated in a phone conversation that I had with him that they had almost succeeded to design a Level-1 Trigger system with zero dead-time, obviously unaware that I had already accomplished this 24 years ago and was published this success in the 1994 DOE/LM-0002, DE94005148.

To avoid wasting any further taxpayer money, it will be necessary to allow inventors including myself to present their inventions at several conferences and public workshops soliciting comparison of my work with others, to reform the peer-review system and to organize a public scientific review of my invention similar to the one organized at Fermilab in 1993 for my basic 3D-Flow invention but conducted at CERN that has now became the center of activity in this field.

In summary, the differences between the 3D-Flow OPRA architecture and the “standard architecture for parallel algorithm, with pipes that feed multiple CPU's” is that the 3D-Flow OPRA architecture can a) sustain an ultra-high input data rate with zero dead-time, b) execute programmable complex object pattern recognition algorithms, c) be built in a volume of 65,000 cubic cm instead of 700 million cubic cm of alternative Level-1 Trigger systems that are limited in performance because of the distance between components (see more detailed explanations in the first answer to the difference in topologies), and d) be built at a fraction of the cost of other trigger systems.
2. Quantitative data & technical merits of the project/research provided in the text of the 2484 Abstract

This abstract contains extremely valuable quantitative data and shows high technical merit of the project/research:
a) The 3D-Flow OPRA system has the capability to execute Object Pattern Recognition Algorithms (OPRA) in real time on data arriving in parallel from a matrix of thousands of transducers at ultra-high speed that are sent to an equivalent matrix of thousands of 3D-Flow processors.
b) 3D-Flow processors can execute several different programmable “OPRA steps” (OPRAS) each consisting of up to 26 operations in less than 3 ns.
c) It compares the desired object (shape and detailed characteristics) with billions of objects per second while sustaining an input data rate of several million frames per second with zero dead-time.
d) It can measure all kinds of phenomena and identify all kinds of objects in 3-D that create an electrical signal in response to physical stimuli (e.g.from CCD, APD, PMT, SiPM, PADs, etc.). It provides advanced triggering capabilities.
e) It was proven feasible and functional in hardware using two FPGA chips in 2001 and two modular industrialized boards in 2003.

The most valuable quantitative data & technical merit are:
f) The 59 quotes from reputable industries prove it is technically sound and feasible to replace hundreds of crates of electronics with one 3D-Flow OPRA crate at 1/1000 the cost while providing staggering performance improvements.

This abstract informs the scientific community of the quantitative advantages and technical merit of the 3D-Flow OPRA with unprecedented performance that not only can replace hundreds of crates of electronics with one crate at a staggering increase in performance at the Large Hadron Collider, the biggest and most expensive experiment in the history of the planet, at CERN, Geneva, Switzerland but can also provide advantages in other applications in other fields.

It can also execute programmable object pattern recognition algorithms on data arriving in parallel from a matrix of thousands of transducers at ultra-high speed from different types of sensors (e.g. CCD, APD, PMT, SiPM, PADs, etc.) generating electrical signals in response to a physical stimuli.

The optimized balance between:
a) the topology of the 3D-Flow OPRA system whose feasibility is proven by 59 quotes from reputable industries and by the details at the link provided in the abstract, both prove it is feasible to build instrumentation confined in 36 cm cube electronics for 4,096 electronic 16-bit channels at 80 million frames per second (0.65 TB/sec) executing up to 30 OPRAS (Object Patter Recognition Algorithm Step at 3 nanoseconds execution time per step) at approximately $80,000 production cost per crate, or in a 36 cm cube of electronics for 8,192 electronic 16-bit channels (or pixels in a frame) at 80 million frames per second (1.3 TB/sec), executing up to 20 OPRAS at approximately $100,000 production cost per crate,
b) the power consumption,
c) the speed,
d) the chosen nanometer technology,
e) the cost of each component
   provides a very powerful cost-effective instrument that improves several fields such as medical imaging, biological research in studying biological processes, fighting terrorism, quality control in manufacturing, etc.
3. Innovation and significance of the project/research provided in the text of the 2484 Abstract

This abstract contains extremely valuable innovations and shows the high significance of the project/research:
a) The 3D-Flow OPRA is a new electronic instrument and device targeted to solving application problems of fast, multi-dimension Object Pattern Recognition (OPRA) in real time
b) The 3D-Flow performance is increased by its bypass switch allows the execution of uninterruptable algorithms for a time longer than the interval between two consecutive input data sets.
c) It can measure all kinds of phenomena and identify all kinds of objects in 3-D that create an electrical signal in response to physical stimuli (e.g. from CCD, APD, PMT, SiPM, PADs, etc.)
d) It provides advanced triggering capabilities
e) The basic 3D-Flow invention was first acclaimed in 1992 in many letters from scientists. In 1993 it was recognized valuable by academia, industry and research centers in a formal scientific review

The innovations provide significant advantages as:
f) The 59 quotes from reputable industries prove it is technically sound and feasible to replace hundreds of crates of electronics with one 3D-Flow OPRA crate at 1/1000 the cost while providing staggering performance improvements.

This abstract informs the scientific community of the 3D-Flow OPRA breakthrough invention that not only can replace hundreds of crates of electronics with one crate at a staggering increase in performance and a fraction of the cost the electronics at the Large Hadron Collider, the biggest and most expensive experiment in the history of the planet, at CERN, Geneva but also has advantages in other applications and can revolutionize other fields by opening doors to applications unthinkable before the 3D-Flow OPRA invention.

A similar statement was already made by the review panel during the major public scientific review of my basic 3D-Flow invention held at Fermilab in 1993 when they stated: “. . . given this feature experimenters would probably think of clever uses not now possible.” However, in this case the new features provided by the 3D-Flow OPRA invention will open the door for researchers to think of clever uses not now possible in many fields.

An in-depth analysis of the 271-page document at the link provided in the 2016 IEEE-NSS 2484 abstract proves it is feasible to build a 3D-Flow OPRA system of about 10,000 electronic channels in one VXI crate, 36 cm cube volume of electronics capable of sustaining an input data rate of 20 TB/sec (also shown in FIG. 1 of this non-provisional patent application). The mechanical structure, cable, connectors, assembly selected already support this feature. This overall system performance is achievable with the proposed 3D-Flow OPRA topology and system. It is now a matter of money to pay for a more expensive NRE (Non-Recurring Engineering) to port the 3D-Flow processor ASIC to the more advanced nanometer technology; however, all other components can support 20 TB/sec on 10,000 electronic channels per VXI crate.

The 3D-Flow OPRA invention is high significance because it can provide advantages in technology to advance science and benefit humanity in several fields such as: medical imaging, biological research in studying biological processes, fighting terrorism, quality control in manufacturing, etc., breaking the speed barrier in real-time applications in the most cost-effective manner.
4. Supporting material to the quantitative data, technical merit, innovation and significance of the project/research provided in the text and figures worth a thousand words in the 2484 summary

The figure on the first page of the 2484 summary is worth a thousand words (see also FIG. 1 of this non-provisional patent application). It has been understood by laymen and would not take long for a busy professional to understand. My 3D-Flow OPRA invention should be of enough interest to any professional with scientific integrity to investigate it in more depth, and if they cannot find a reason to substantially invalidate my main claims, it would be their professional responsibility toward taxpayers and humanity who trust their ethics and scientific integrity to support advancements in science and to work in their interest, to make a statement such as those made in the written report by the review panel of the major official formal scientific review held at Fermilab in 1993.

Likewise, it would be their responsibility if they make a statement that does not reflect the scientific truth but is crushing inventions with unsupported claims without calculations or scientific reasons, preventing advancements in science and preventing taxpayers and humanity from receiving the benefits from the invention.

The Fermilab report included in its entirety from page 64 to 71 of the proposal to DOE #0000222704, responding to the 2016 solicitation DE-FOA-0001414 states at page 2 of the report: “The committee finds this project an interesting and a unique concept . . . We believe the concept will work . . . We see no technical reason why the proposed ASIC processor could not be built in . . . We do not believe that there are any major flaws in the proposed system. . . . We see little risk in this approach to the processor chip design itself” in fact my 3D-Flow “unique concept” was proven feasible and functional in hardware and on page 3 of the same report: “. . . we see no technical reason why the proposed ASIC will be a problem to develop. . . . The committee was impressed with the work already completed by essentially one person operation . . . We see nothing fundamentally wrong . . .” On System Design on page 6 of the report is stated: “The committee believes there are no major flaws in the conceptual design”.

The figure on the first page of the 2484 summary shows a photo of several light bulbs each representing a different idea or algorithm (see also FIG. 1 of this non-provisional patent application). One light bulb can be lit at a time, which symbolizes the real-time algorithm that has the task to filter the object with desired characteristics from data arriving at 20 TB/sec shown on the left of the figure and the target of the object found shown on the right of the figure.

Below this figure there are three examples of possible applications of the 3D-Flow OPRA.

The bottom-left of the figure shows the application for discovering new subatomic particles. The light bulb represents the algorithm that would capture the data matching the characteristics of the Higgs-like boson. Input data is arriving at 1.3 TB/sec from the CMS (or Atlas) experiment at CERN. The instrument with the capability to filter this data and find the Higgs-like boson could have been implemented in 1994 with the 3D-Flow Level-1 Trigger system in 1 m×1.8 m reported on page 106 of the proposal to DOE #0000222704, responding to the 2016 solicitation DE-FOA-0001414, or it could be implemented now at a lower cost and higher performance in the new 2016, 3D-Flow OPRA in one VXI crate with 36 cm cube of electronics described in detail in the 2016 IEEE-NSS 2493 abstract/summary.

The bottom-center of the figure (see also FIG. 1 of this non-provisional patent application) shows the application for medical imaging which can detect anomalies in biological processes at an early curable stage and save many lives from cancer and other diseases. The light bulb represents the algorithm that should accurately capture all signals from the tumor markers (or tracers tagging anomalies in other diseases) that will provide the most accurate information to physicians on the minimum abnormality in the biological process under investigation at a very early curable stage, a very low radiation dose and a low examination cost. Input data is arriving at approximately 368 GB/sec from the 3D-CBS detector. The instrument with the capability of filtering this data and finding the signals (generated by a pair of 511 keV photons hitting the detector almost simultaneously) related to biological processes, such as an anomaly in metabolism typical for cancer cells, can be implemented in one VME crate with a 16 cm cube of electronics described in detail in the 2016 IEEE-NSS 2493 abstract/summary.

At the bottom/right of the figure (see also FIG. 1 of this non-provisional patent application) is shown the application for fighting terrorism. The light bulb represents the algorithm that should capture the data matching the characteristics of the face of an outlaw. Data in input are arriving at approximately 327 GB/sec from the 3D-CBS detector. The instrument with the capability to filter those data and find either a face among a large crowd in a stadium, theater, mall, etc. acquired in real-time from several cameras, matching an identikit or a few faces acquired from a camera matching a database of millions of faces of people at different events which might help to trace in seconds the movements of an outlaw, can be implemented in one VME crate with 16 cm cube of electronics described in more details in the 2016 IEEE-MIC 2505 abstract/summary.

The text at the bottom of the first page of the 2016 IEEE-NSS 2484 summary summarizes other possible applications of the 3D-Flow OPRA, while the second page provides more details on the invention, the performance of the 3D-Flow processor, the relation/similarity to other instruments such as the oscilloscope and the Logic State Analyzer, both designed to visualize the voltage variation in time of four signals or many signals. The 3D-Flow OPRA could be considered the third generation of electronic instruments with the capability of executing the desired programmable complex Object Pattern Recognition Algorithms (OPRA) comparing the sought after object to billions of objects per second arriving at an ultra-high input data rate.

It lists some of the features that the 3D-Flow OPRA can provide after it has triggered on the desired object and it provides two example of practical implementation of the 3D-Flow OPRA system for 8,192 electronic channels and for 2,304 electronic channels.
5. Crosetto's comments to Eckhard Elsen and Susanne Kuehn responsible with their reviewers to accept/reject the 2016 IEEE-NSS paper 2484

Dear Eckhard and Susanne,

Please see the comments that I wrote you at the end of the response to your and your reviewers' rejection of abstract/summary 2493. In addition, I would like to ask why you did not respond directly to my several emails starting from Jul. 22, 2016, and why you or someone working with you moved my 2484 paper submission from the 2016 IEEE-RTSD to the 2016 IEEE-NSS. I have asked for the name of this person so I can explain why my 2484 paper is for more general applications suitable for the IEEE-RTSD Workshop, unlike my 2493 paper specific for the Level-1 Trigger for High Energy Physics.
D. Review of the 2016 IEEE-MIC paper 2505 by Dimitris Visvikis and Suleman Surti responsible with their reviewers to accept/reject papers submitted to the 2016 IEEE-MIC Conference:

“scored all categories as poor, because: The text of this “so-called” abstract might be appropriate for a newspaper. But even for such a purpose there is insufficient information for the reader. For me it is totally unclear—even after having looked at the summary, which is of ultimately bad quality—what kind of system is the “ultrasensitive 3D-CBS”. I think it is not expected that the reviewer starts a literature research to find the lacking information.

Once again, the abstract and supporting data emphasize the difficulties in getting the project funded rather than submitting scientific material (as he's been told in the past). VERY strong reject!”
1. Response to Dimitris Visvikis and Suleman Surti and their reviewers' opinions/questions/concerns:
Response to: “what kind of system is the “Ultra-sensitive 3D-CBS”

it is the kind of system explained in the 2016 IEEE-MIC abstract 2505 with the words: “The ultra-sensitive 3D-CBS . . . capable of extracting ALL valuable information from radiation . . . (on spatial and time resolution, energy and sensitivity) it reduces the radiation dose to the patient, reduces costs, and provides valuable information to doctors on anomalies in morphological changes and biological processes.”

Suleman Surti, IEEE-MIC Deputy Chairman must know what is an ultra-sensitive PET because he wrote an article entitled: “An Ultra-Sensitive Total Body PET Scanner for Biomedical Research” and explained in slides 3 and 4 why it is necessary and what makes a PET “Ultra-Sensitive”, and in slide 3 stated that what they presented is “Not a New Idea!” citing my previous article, thus admitting that he and his co-authors copied several of my ideas.
Response to: “it is not expected that the reviewer starts a literature research to find the lacking information”.

All relevant and necessary information to compare the advantages of the 3D-CBS to current PET and the Explorer is contained in the 300 word abstract and the two-page 2016 IEEE-MIC summary 2505 where there is more information than the 10 slides and articles of the Explorer authored by Surti and others. Surti and his co-authors of the Explorer did not cite my previous work in their articles (except as mentioned in the previous paragraph when in slide 5 when my name and article are cited to explain that their Explorer was not a new idea) and/or have obstructed the presentation, publication and funding of my 3D-CBS (3-D Complete Body Screening) invention which is more efficient and more than ten times less expensive than their Explorer.

The figure in the last page of the summary 2505 shows the entire 3D-CBS system; details of each component are described at the link provided on the first line of the abstract. This figure shows:
The 3D-CBS system consisting of two crates, one for converting from analog-to-digital the signals received from the detector, the other 3D-Flow OPRA crate for data processing. The Explorer system is less efficient and more expensive and consists of the Analog-to-Digital electronics shown on the bottom of the block detector on the right of slide 7, plus twelve Detector Crates of slide 8, and four to six racks of computers cited on the UC-Davis website.
The 3D-CBS power consumption of the two crates of the figure at the last page of the summary is 3 to 4 kW. The power consumption of the Explorer is 40 to 60 kW. (40 kW listed on slide 9 to 60 kW listed on the UC-Davis website).
The 3D-Flow Data Processing Boards of the 3D-CBS system handle 256 channels per board (see figure in the summary 2305 and details on pages 154-156 of the link showing it can make available 512 channels and 1024 channels per VXI board). These 256-channel data processing boards also perform the functionality of the six racks of computers in the Explorer system which are therefore not needed. Nine Data Processing Boards handling 2,304 channels are housed in one crate. The Explorer Detector Boards can handle only 16-channels per board and do not have data processing capability. The 120 Detector Boards of the Explorer handling 1,920 channels are housed in 12 Detector Crates as shown on slide 8. All data are saved on hard drives and processed later by a farm of computer housed in 6 racks.
My breakthrough 3D-Flow OPRA invention used in the 3D-CBS captures all valuable data from tumor markers at the lowest possible cost per valid data captured and has the capability of processing over 40,000 TB data in one day compared to the 40 TB of data in one day acquired and stored for later processing by the farm of six racks of computers by the Explorer (see the UC-Davis website). The 3D-CBS requires a storage capacity of less than 1 GB to store results and no storage during acquisition because data is processed in real-time by the 3D-Flow system. The high processing capability of my 3D-Flow invention breaking the speed barrier in real-time applications allows all valuable information related to the characteristics of the crystals to be extracted, thus allowing the use of more ecouosnlcal crystals which reduces the cost of the 3D-CBS device.

TABLE 8
Features of the abstract/summary 2505 are compared to
the Explorer by Surti and his co-authors

| Description | EXPLORER | | 3D-CBS | |
|---|---|---|---|---|
| Crystal Type | Expensive LYSO | | Economical BGO | |
| | 491,520 | | <3,000 | |
| | crystals | | crystals | |
| Number of electronic channels | 1,920 | | 2,304 | |
| Number of Channels per Board | 16 | | 256 | |
| Number of Detector Boards | 120 | | 9 | |
| Number of Crates housing the Detector Boards | 12 | | 1 | |
| (the 3D-CBS crate is also housing the computer | | | | |
| to process valid data and provide | | | | |
| results in analytical and graphical form) | | | | |
| Capability to acquire and process data each day | 40 | TB | >40,000 | TB |
| Hard Drive's Drive's size needed each day | 40 | TB | 1 | GB |
| Number of racks containing | 4 to 6 | | 0 | |
| computers to process acquired data | | | | |
| Power Consumption | 40 to 60 | kW | 3 to 4 | kW |

| | | |
|---|---|---|
| Sensitivity | Less than the 3D- | Ultra-Sensitive |
| | CBS | |
| Cost of the Device | 30 to 50 times | 2 to 3 times current |
| | current PET | PET |
| Examination Cost | Higher than | Lower than current |
| (what matters to the patient) | current cost | cost because the |
| | because the | throughput can be |
| | throughput cannot | higher than 2 to 3 |
| | be 30 to 50 times | times current PET |
| | current PET | |
| Potential to save millions of lives | Cannot prove to | Can prove on a |
| (what should matter to humanity) | save lives on a | sample population |
| to solve the world's most deadly calamity) | sample population | to save many lives |
| | because each day | because each day |
| | it cannot process | it can process cost- |
| | 40,000 TB data | effectively over |
| | from tumor | 40,000 TB data |
| | markers | from tumor |
| | | markers |
| Potential to reduce healthcare costs | Increases | The lower |
| (what should matter to politicians to solve | healthcare cost | examination cost |
| the world's most costly calamity) | because of its | saves many lives; |
| | exorbitant cost; | those who live |
| | losing many lives | instead of dying |
| | lowers | return to be |
| | productivity | productive and are |
| | | removed from |
| | | healthcare bill |

Data for the Explorer reported in the above table are derived from publications, slide (goo.gl/BpqjAj) presentations and several (goo.gl/RG8COf) press (goo.gl/ovMZ5j) releases (goo.gl/TI95NN) made (goo.gl/NpNNNr) by the authors (goo.gl/xcBe0Q) of the Explorer (goo.gl/W6cZ9Y) and from calculations based on the data reported in the articles.

Data and feasability (goo.gl/6DS5oy) of the 3D-CBS (goo.gl/YGg04E) (3D-Complete Body Screening) is proven by the 3D-Flow (goo.gl/5EUkYe) innovative basic concept proven feasible and functional in hardware in two modular boards (goo.gl/ymgnXz) each with 68×3D-Flow processors and recently the 3D-Flow OPRA (goo.gl/goYPv9) proven feasible and cost-effective by 59 quotes from reputable industries.
a. How the peer-review system suppressed my innovations and enabled reviewers to copy them

The reason why the Explorer was funded with $15.5 millon of taxpayer money even though an analytical study could prove before construction that it would not be able to save many lives and would increase healthcare costs is because the peer-review system for approving/rejecting articles and funding research projects is rigged and the Director of Extramural Research of the National Institute of Health, Michael Laurer, refuses to have a dialogue or even read my work as he believes the peer-review system is fair. If he would have a dialogue or read my work he would know that many of my ideas have been copied by the authors of the Explorer, yet my 3D-CBS continues to go unfunded after 19 years from the first request for a grant to NIH even though, unlike the Explorer, it would be able to save millions of lives and reduce healthcare costs.

My inventions have received extensive recognition and endorsements in many letters from top experts in the field; my articles have been published in prestigious peer-review scientific journals; I was often invited to give seminars at prestigious events and conferences, yet influential scientists and agents at funding agencies in the specific field within IEEE, government and private organizations who approve/reject articles, presentations at conference and funding have obstructed, rejected, crushed my inventions for more than two decades.

These influential scientists and agents at funding agencies refuse to follow scientific procedures, refuse transparency in a public dialogue, refuse an analytical discussion based on calculations and scientific evidence and have rejected for twenty years my articles, presentations and requests for funding my 3D-CBS to improve PET sensitivity, intensify computation at the front-end of PET device and improve the electronics, claiming it was necessary instead to improve spatial resolution (to the detriment of sensitivity), intensify computation at the back end rather than at the front-end and improve crystals rather than electronics.

After receiving $906,000 in grants from DOE in 1995, and proving my basic 3D-Flow invention was feasible (generating the RTL files for the Silicon Foundry to manufacture the ASIC 3D-Flow processors and developing the software tools: simulator, assembler, test vectors, etc.) and would benefit many fields, including Medical Imaging, my requests for funding have been rejected for the past two decades.

Here are some examples of the recurring reasons given by reviewers for their rejections:
1996-NIH-1R43RRLM11544-010A reviewer claims intensive computation is needed at the back-end of the PET and not at the front-end.
2001-NIH-1R43CA93103-01, reviewer claims that electronics is not a problem, focus should be placed on spatial resolution and reviewer does not see the reason to increase sensitivity and reduce radiation dosage.
2002-NIH-1R01EB000900-01, reviewer claims “that there is no reason to expect that electronics will lead to any significant improvement in efficiency”.
2002-NIH-1R43EB00959-01, reviewer states: “The whole body imager envisaged by the proponent is optimized for efficiency but not for optimal spatial resolution”. (This is not true because the 3D-CBS maximizes all measurements: energy, spatial and time resolution and provides high signal-to-noise ratio).

I have used all means available such as appeals, talking to and exchanging emails with leaders at NIH and NCI, meeting NIH and NIBIB Directors at a press conference on Medical Imaging, requesting the help of U.S. Senator Kay Bailey Hutchinson, Texas State Senator Jane Nelson, Senator John Cornyn, U.S. Congressman Michael Burgess, U.S. Congresswoman Eddie Bernice Johnson, and many others to request transparency in science by having a fair public scientific procedure similar to the review of my basic 3D-Flow invention held on Dec. 14, 1993, at FERMILAB.

Despite all these efforts to have a public analytical discussion between the reviewer and the inventor based on calculations and scientific evidence and resolve any disagreements by setting up an experiment where the results would be the judge, funding agencies asked me to follow the reviewers' advice, give up on my inventions, modify my research, approach, objectives toward improving spatial resolution to the detriment of sensitivity, intensify computation at the back end rather than at the front-end and improve crystals rather than electronics.

In fact, the letter from Dr. Norka Ruiz Bravo, Deputy Director for Extramural Research at NIH to U.S. Senator John Cornyn who enquired on my behalf about the rejection of my proposals, made exactly these requests: “NIH staff have provided advice on being responsive to the peer reviewers' comments (focusing on both the strengths and weakness), and they have provided specific instructions for submitting a revised application. They have consistently recommended that he [Crosetto] spend time analyzing the results and gathering as much feedback as possible from the summary statements, senior investigators, and peers at his organization and/or in his field”.

Furthermore, Dr. Bravo, instead of identifying an expert in particle physics and the specific technology that is key to improving PET efficiency, suggested I submit my proposal to another review panel (SSS-8), Bioengineering and Physiology Review Panel, who are further away in expertise in particle detection and in accurately capturing as many 511 keV valid pair of photons (tumor markers) as possible at the lowest cost per valid pair captured.
b. It took fifteen years to recognize that the electronics in PET was a problem

Because it was my duty to inform the scientific community and those in power in the field about my invention that would benefit humanity, I wrote a technical-scientific book [40] entitled: “400⁺ times improved PET efficiency for lower-dose radiation, lower-cost cancer screening” 200 copies of which I distributed free of charge to the leaders in the field at the 2000IEEE-NSS-MIC Conference, and I asked Terry Jones (who is now one of the authors of the Explorer), a prominent leader in the field if he could review the book that I gave him (in 2000, Terry Jones was with Hammersmith Hospital in London and later moved to UC-Davis in California). Jones appointed Steve DeRenzo as the technical expert in the field to review my book. I knew DeRenzo's back in 1992 when I was working at the Superconducting Super Collider and I had difficulty addressing scientific arguments about one of my articles with him and his colleague, Bill Moses (who is now the leader of the Explorer project), I received the same rejections from DeRenzo and from Moses. They published many articles on measurements of crystal detectors which I found useful and I recognize their expertise in this field; however, contrary to the very favorable reviews of my book on Amazon.com, they obstructed my inventions beneficial to medical imaging and other fields as you can read from DeRenzo who stated in his review of my book:

“We do not view the electronics as a problem, either in terms of performance or cost”.

It took 15 years from when I wrote my book and 19 years from my first rejection for funding by NIH, for those in power in the field such as Bill Moses to reverse their position toward my direction of research that they have obstructed for decades. Read Moses' 2015 press release:

<<We're developing the electronic interface between the detectors and the computer algorithm—and the electronics for this scanner is an order of magnitude more complicated than what's been done before,” says Moses>>.

However, 160 million people have died from cancer (12 million were Americans) since I first claimed that to improve PET medical imaging efficiency it is necessary to improve the electronics and not the crystals. Many of these people could have been saved if the funding agencies would have requested a public review of my invention similar to the major scientific review of my basic 3D-Flow invention held at FERMILAB in 1993.

If funding agencies had requested an open, public analytical discussion of my 3D-CBS invention based on calculations and scientific evidence before assigning taxpayers and donation money, it would not have taken 19 years for influential people like Bill Moses, Terry Jones, DeRenzo and others to reverse their belief that there was no need to improve the electronics of PET, and the life-saving advantages and benefits would have been evident and taxpayer money would have been put to good use.

Steve DeRenzo had written a negative review of my technical-scientific in the year 2000, but his claim that in order to improve PET efficiency, crystal detectors should be improved and there was no need to improve the electronics, was proven wrong by many articles and projects built by third parties after DeRenzo's review. Even Siemens, a leader in this industry, had to recant their statements after I had a day-long meeting with the President of Siemens Nuclear Medicine and Director of PET, followed by conference calls with Siemens Head of Research and Chief of electronics. At first, they stated that it was impossible to improve PET efficiency by improving the electronics, and that there was no need anyway, but five years after our meeting Siemens recanted all their statements when they published on their website that they had increased PET efficiency by 70% by improving the electronics.
c. How many years will it take to recognize that the 3D-CBS can save millions of lives, is more efficient and over ten times less expensive than the Explorer that cannot save many lives?

We are now at the same junction as 19 years ago. Funding agencies are responsible to taxpayers and donors to use their money to fund research projects that gives them best return in saving lives and money. Here is a summary of the two projects they had to consider:
a) The 3D-CBS first described in the year 2000 in my technical-scientific book, updated in several subsequent articles and documents, and hand-delivered to the Director of NIH on Jan. 31, 2006, has now been proven feasible by 59 quotes from reputable industries to build the 3D-Flow OPRA data processing electronics with the capability to process over 40,000 TB per day in one crate of electronics, requiring less than 1 GB data storage per day, for a total power consumption of the entire 3D-CBS system of 4 kW. The 3D-CBS can prove on a sample population that it can save many lives because each day it can cost-effectively process over 40,000 TB data from tumor markers.
b) The Explorer project that has copied several ideas from the 3D-CBS. Although the authors of the Explorer after 19 years now understood the importance of improving the electronics in PET, it can be proven in an analytical discussion based on calculations and scientific evidence that 6 racks of computers with the capability of processing 40 TB data per day stored on hard drives, acquired by 12 crates of electronics for a total power consumption of 60 kW would provide a device that could not prove to save lives on a sample population because it cannot process 40,000 TB data every day from tumor markers. Furthermore, the Explorer is less efficient and more than ten times as expensive as the 3D-CBS.

Clearly, the advice by Dr. Ruiz Bravo, NIH Director of Extramural Research, in her letter dated May 10, 2004, to U.S. Senator John Cornyn that I should follow the reviewers' guidance and modify my research, approach, objectives toward improving spatial resolution (to the detriment of sensitivity), and improve crystals rather than electronics, essentially giving up on my invention turned out to be wrong because it has been recanted by influential leaders in the field who are now receiving $15.5 million from NIH to build the Explorer, which stated in its 2015 press release “the electronics for this scanner is an order of magnitude more complicated than what's been done before”.

Perhaps Dr. Ruiz Bravo believed in good faith in the fairness and professional integrity of her reviewers and in the NIH Center for Scientific Review (CSR); however, because the current peer-review system is subject to corruption and because it turned out that the advice from her reviewers was obviously wrong and more troubling facts prove that influential leaders copied several of my ideas, this issue should be addressed to avoid further needless loss of lives and wastage taxpayer money.

Now, I have informed my findings regarding misconduct in the peer-review system to NIH Director Francis Collins, NCI Director Doug Lowy, several agents at NIH, and NIH Director of Extramural Research Michael Laurer. Dr. Michael Laurer realized the seriousness of the allegations and suggested I report them to the Office of Scientific Integrity.

Out of the $15.5 million in taxpayer money for the Explorer, $3,054,873 was given to the Explorer team by NIH in 2015 and $3,314,184 in 2016. By the end of 2018 most of the $15.5 million will be delivered. An analytical discussion based on calculations and scientific evidence can prove that major conceptual flaws with the Explorer project will waste taxpayer money because it cannot save many lives, increases healthcare costs, is less efficient and is over ten times more expensive than the 3D-CBS.

In addition, the 3D-CBS ER/DSU unit costing $23,000 for duplicates listed on Table 6 at page 29 and described from page 149 to 170 of the proposal provides the list-mode feature of the Explorer but with higher capability to store raw data from the PET detector and at a much higher granularity for research study. Three reputable industries have provided quotes for NRE (Non-Recurring Engineering) of the 3D-CBS ER/DSU. The NRE cost is $350,000 and provides the first 7 units.

I was told on Jul. 20, 2016, by Dr. Gretchen Wood from the office of the NIH Director Francis Collins that Michael Laurer was in a meeting with NIH executives discussing who would be responsible at NIH to address analytically technical issues based on calculations and scientific evidence that my 3D-CBS is more efficient, over ten times less expensive than the Explorer and could save millions of lives with a cost-effective early cancer detection, while the Explorer funded by NIH for $15.5 million cannot and is increasing healthcare cost. She said I would hear a response from Laurer later that day or the following day; however, to date I have not received an answer. If there is no capability at NIH to solve this issue analytically, they should fund both projects and let experimental results on a sample population be the judge.
d. Several senior scientists who know the leaders in Medical Imaging and appreciate my invention have tried to bring my invention to their attention, even inviting them to a dialogue addressing scientific issues in an open forum, but they always refused.

Uwe Bratzer, the General Chairman of the 2008 IEEE-NSS-MIC Conference, agreed to organize a forum to discuss and critique my 3D-CBS invention, and asked me who he should invite as experts. I named Bill Moses, Steve DeRenzo, among others and Alberto del Guerra (http://blog.u2ec.org/wordpress/?p=1363) who was invited to participate to workshops and reviews of the 3D-CBS innovation held at the University of Pavia and at the Hospital S. Matteo in Pavia, Italy. However, they all refused.

The influential people in Medical Imaging are well known in the scientific community and there have been several in addition to those who wrote letters of endorsement of my invention who have tried to help bring my inventions to the attention of these influential scientists. For example, Chris Parkman from CERN, who was Chairman of the Industrial Exhibition of the IEEE-NSS-MIC conference in 2000 in Lyon, France, gave me a booth at the Exhibition free of charge where I could demonstrate on the computer a simulation of the 3D-Flow system made of thousands of 3D-Flow processors.

The following year he did the same, giving me a free booth at the Industrial Exhibition of the IEEE-NSS-MIC conference in San Diego, Calif., where I could setup my two Altera prototype boards with my 3D-Flow system of 8 processors implemented in two FPGAs proving the concept of my invention working in hardware. Visitors who stopped by my booth could select a desired pattern of clusters on switches; the results were displayed on LED and the oscilloscope provided the waveform of the signals with the speed of the system.

Parkman kindly told several people, including the General Chairman of the conference, to stop by my booth and strategically placed it next to the coffee booth, so many people would have a chance to see it. I also invited Bill Moses and Steve DeRenzo to stop by my booth when they were at the coffee booth, but although they agreed to, they did not stop by. The last times I spoke to Bill Moses was at the 2013 IEEE-NSS-MIC conference in Seoul, South Korea, and in 2014 IEEE-NSS-MIC conference in Seattle. On both occasions I asked him what he thought of my 3D-CBS project that I presented in Seoul. He told me that he would read my 2013 article and get back with me. Next thing I saw was his press release sent by a friend of mine regarding his $15.5 million grant from NIH which contained several of my ideas.

The late Emeritus Professor in Physics Walter Selove told me one day that he had been very fortunate to arrange a meeting with Bill Moses in Berkeley to tell him about my 3D-CBS. However, Bill Moses showed no interest.
e. Reviewers and leaders were aware of the advantages and benefits of my inventions

A few facts to help those who want to fix the rigged peer-review system. Not only were Surti (who is now one of the authors of the Explorer project) and his colleagues aware of my inventions prior to copying several of my ideas for the Explorer as they cited in 2013 in their slide 5, but this issue has been addressed for more than a decade when I wrote a book in the year 2000 and cited Surti's work and the work of his colleague Joel Karp (who I believe was his supervisor at that time) at the same University of Pennsylvania. The late Emeritus Professor in physics Walter Selove at the University of Pennsylvania who wrote a letter of endorsement to me for the 3D-CBS on Apr. 14, 1999, told me that he visited Surti's group and spoke with Joel Karp about my invention. I had a correspondence with Joel Karp (who is now one of the anchors of the Explorer project) when he was the General Chairman of the 2002 IEEE-NSS-MIC conference and all my papers were rejected. I could not convince him of the importance for the scientific community and for humanity to let me present my inventions at the conference, he repeatedly in his email confirmed the rejection of all my papers. I sent him a paper copy of my book, and he acknowledged receiving and reading it and responded on Aug. 19, 2002 with the following:

“Thank you very much for sending me your book, which I received on Friday. I did briefly look through it to get a better appreciation about your proposals for improved PET. Certainly a very large axial FOV can increase sensitivity dramatically and thereby decrease scan time and cost, while also increasing counting statistics. It's not obvious to me, however, whether the trade-off is effective between instrument cost and scan cost—certainly the instrument cost goes up significantly, even for low cost scintillators such as BGO. For LSO or GSO the cost seems prohibitive. Also, it's not immediately clear that the trade-off is effective between increase of trues and commensurate increase of scatter and randoms, especially for scintillators with poor energy resolution . . .”

However, 13 years later, Joel Karp proposed with his co-authors, including Surti, the Explorer project in articles, slide presentations, and a $15.5 million request for funding to NIH. The Explorer is using the expensive L(Y)SO that he said in his email is cost prohibitive. If fact, in the case of the Explorer, he is right as it costs 30 to 50 times the current PET and is less efficient than the 3D-CBS.
f. After crushing my inventions for more than a decade which would have been a TRUE PARADIGM CHANGE in oncology research, the same people now claim a paradigm change after copying several of my ideas to build the Explorer which cannot make a paradigm change in saving lives, cannot reduce healthcare costs and advancing research because it is less sensitive and over ten times more expensive than my 3D-CBS.

After many of the authors of the Explorer who are listed in slide 1 rejected my papers, book, and created obstacles to my inventions for more than a decade, in 2015 they took many of my ideas, even my words and claims that they had rejected years before and used them in their documents, beginning with the title of the $15.5 million proposal claiming a paradigm change (the same I claimed more than a decade before), the abstract of their $15.5 million proposal uses several of my statements such as the increase in efficiency can provide benefits in lower radiation, or higher throughput, or better signals for early detection, or a balance of all these, and even my analogy comparing the lower radiation dose to the radiation received during a transatlantic flight on the cover page of my book ISBN 0-9702897-0-7, deposited in the year 2000 at the Library of Congress Catalog-in-Publication Data Card Number: 00-191510, that I gave them and other leaders in the field when I distributed 200 copies free of charge at the 2000 IEEE-NSS-MIC conference in Lyon (France) was reported in their abstract of the request for the $15.5 million to NIH.

Despite all the articles I wrote about the 3D-CBS, my personal conversation and email exchanges with leaders of the Explorer Project decades before they announced the Explorer project, despite other scientists informing them about my 3D-CBS and 3D-Flow invention, I cannot find a citation of my prior work in Explorer articles, yet they claim in the title of their $15.5 million proposal “Explorer: changing the molecular imaging PARADIGM with a total body PET.”

It is over a decade that I spelled out in great detail the PARADIGM CHANGE in molecular imaging that a true total body PET can bring. My 3D-CBS was recognized as the first true total body PET by G. Borasi, et al. article (also cited by Simon Cherry in slide 5 of his presentation), published in the European Journal of Nuclear Medicine and Molecular Imaging (2010) 37:1629-1632. This article states that Crosetto's 3D-CBS technology is the first, true “total-body”. I supported calling it a paradigm change by detailing how it is cost-effective in accurately capturing all possible signals from the tumor markers, while the Explorer capturing only 40 TB of data per day and processing them using 4 to 6 racks of computers is not appropriate to call a paradigm change because it cannot make a significant impact on clinical studies or in reducing cancer deaths.

Here are a few of my articles and documents where I describe in detail how this paradigm change can be achieved with my 3D-CBS.
1. In 2006, I published an article [47] in World Scientific of a presentation I gave the year before at the ICATPP conference. The title of the article is “Rethinking Positron Emission Technology for Early Cancer Detection”. Excerpt from the article: “In order to use Positron Emission Technology effectively for early cancer detection, a paradigm shift is necessary. To illustrate this, I am going to compare objectives, specifications and implementations of current PET, “Type A,” and the new technology, “Type B.” Type B makes use of innovations [40], [48] and has been implemented in a device called 3D-CBS [49].” The table reporting PET of Type A and Type B details this Paradigm Change.
2. In 2008 I submitted a paper to IEEE-MIC that was rejected despite an invitation by its General Chairman, Uwe Bratzler, to submit it and his promise that in the event of rejection I would receive a scientific reason. Instead, the only reason I received from the Chairman of the MIC Conference from lines 1421 to 1431 of the document at this link was:
   1421 From: wolfgang.enghardt@oncoray.de [mailto:wolfgang.enghardt@oncoray.de]
   1422 Sent: Monday, Jul. 7, 2008 8:59 AM
   1423 To: crosetto@att.net
   1424 Subject: MIC 2008 Abstract 3073
   1425
   1426 Dear Prof. Crosetto,
   1427
   1428 We regret to inform you that your submission (#3073) “Basic Corrections
   1429 Needed for a Paradigm Change in the Direction of Cancer Research That Will
   1430 Provide a Substantial Reduction of Premature Cancer Death” has been
   1431 rejected.
      If the General Chairman of the IEEE Conference, Uwe Bratzler could not enforce professional ethical conduct from his sub-conference chairmen, this shows how powerful the leaders in the field of Medical Imaging at IEEE are in rejecting a paper on a true PARADIGM CHANGE in Medical imaging and claiming the same nine years later, . . . even without the design of a device supporting the claim.
3. In 2010, I presented a paper with 14 co-authors and approximately 1,000 cosigners at the Workshop “Physics for Health” held at CERN in Geneva, claiming a PARADIGM CHANGE in oncology research. The title of the paper is: “Progress in the domain of physics applications in life science with an invention for substantial reduction of premature cancer deaths: The need for a PARADIGM CHANGE in oncology research. The abstract was translated into ten languages, including Chinese, Japanese and Russian. Also this article was missed by the authors of the Explorer?
4. After meeting NIH Director, Elias Zerhouni at a press conference in Medical Imaging on Jan. 31, 2006, on Apr. 10, 2007, I wrote him a letter stating on page 19 that it was necessary “. . . to activate a true paradigm shift in approaching the war on cancer (by measuring the minimum abnormal metabolism targeted to early diagnosis of cancer that saves lives, rather than being limited to measure tumor dimension that does not save lives)”, which was my 3D-CBS approach.

After I received from Simon Cherry (who is one of the authors of the Explorer project) a copy of the slides of his presentation with the list in his slide 5 titled “Not a New Idea!” with a reference to my 2003 paper, I asked him why he did not reference my book, and two articles all written prior to the year 2000 (I was able to present my 2003 paper thanks to Ralph James who was the General Chairman of the IEEE-NSS-MIC conference because the years after 2000 all chairmen and reviewers rejected all my papers at IEEE-MIC, including Joel Karp who was the General Chairman in 2002).

His answer was because my 2003 paper was only 5 pages, and therefore easier to understand than the 20-page articles or the 225-page book.

I asked Simon Cherry to address the choices he made for the other references and what he believed was the innovative or key features in the articles he cited; however, Cherry never followed up. If we had had an in-depth discussion on the innovative contribution of each paper cited by Cherry, it would reveal that the breakthrough was provided by the 3D-CBS. (It is worth mentioning that the cited article by Borasi et al. is in fact an overview of several PET, including the 3D-CBS, and does not describe any other cost-effective true total body PET that can make a paradigm shift in Medical Imaging while is recognizing <<The first proposal for a truly “total body” PET system (120-160 cm AFOV) came from an Italian physicist, Crosetto[49]>>. This paper was the follow up to a presentation I gave of my 3D-CBS in 2008 to Borasi and his colleagues at the S. Maria Nuova hospital in Reggio Emilia that Borasi et al. followed with a 42-page article reporting from page 26 to 68 our discussion that was published in May 2009 in the “Notiziario di Medicina Nucleare ed Imaging Molecolare”).

Cherry did not mention an earlier design of a PET with long field of view that I discussed during a coffee break at the 2013 IEEE-NSS-MIC-RTSD conference with Benjamin Tsui who was familiar with the design because he review it, however, Tsui said the author never explained how he would implement such a device with a long field of view. None of the other designs, even those with long field of views, could claim innovations in electronics, assembly of the detector, coupling of the detector with the electronics, algorithms, etc. as the 3D-CBS can claim. Only the 3D-CBS can solve the problem of cost-effectively and accurately capturing all possible signals from tumor markers at the lowest possible cost per each valid signal captured, allowing a paradigm change in Medical Imaging capable of detecting the minimum anomalies in biological processes, enabling an effective early detection of cancer and many other diseases at a curable stage.

The above facts speak for themselves that there is a lack of fairness and professional ethical conduct in the scientific community which is essential when billions of dollars per year are at stake.

So how do funding agencies distribute taxpayer and donation money and make these scientists accountable to implement the mission of the agency? The short answer is: “The agencies do not make scientists/reviewers accountable”.

They continue to be the privileged professionals in society who apparently receive more trust from the public than judges, doctors or any other category of professionals. Looking at these facts and the damage non-accountability for scientists is causing society, the media should inform the public and a reform of the peer review system to make scientists accountable is urgently needed for the interest of everyone.
g. My consideration in the 2016IEEE-MIC 2505 Abstract/Summary to satisfy the comments/requests from previous rejections

When I prepared the 2505 abstract/summary this year I took into consideration the reviewers' comments/requests of last year reported below (full text of the rejection claims by the 2015 reviewers is available on page 245 of the proposal):

“. . . I would like to hear a discussion about this to “once and for all” settle the question raised—is this approach any good? . . . Proposes that cancer will be effectively eliminated if low cost, high efficiency PET cameras are developed. No design parameters, hardware description, performance estimates, or technical supporting material . . . No tangible material is provided that could allow to assess the pertinence of this approach. Performance estimates compared to other approaches are highly speculative. Also the link to a previous submission is not clear.”

I made sure this year the information was complete in the 300 word text abstract and two-page summary with one figure showing all components of the system with a link included in the abstract to the details whose feasibility is supported by 59 quotes from reputable industries. However, after all my papers have been rejected by IEEE-MIC for 16 years with the exception of 2003 when Ralph James was the General Chairman of the IEEE-NSS-MIC Conference and have now been rejected again this year with a comment sent by the Chairmen, which includes Surti, asking what kind of system is the <<“ultra-sensitive 3D-CBS”. I think it is not expected that the reviewer starts a literature research to find the lacking information>> after Surti explained in slides 3 and 4 what an ultra-sensitive PET is and in slide 5 provided a reference to my article, about the 3D-CBS. It is therefore evident that we have a rigged peer-review process that is damaging to taxpayers and breaches the professional ethics of scientists.

It is evident that the reviewers have not given legitimate scientific reasons to reject funding all of my proposals since my last grant of $906,000 in 1995, and all my papers and presentations at IEEE-NSS-MIC since 2000 (with the exception of 2003 when Ralph James was the General Chairman), and for closing the door to the dialogue when I proposed two workshops in 2014 to allow young and senior scientists to briefly present their work and then question each other for hours in public and not just five minutes after a presentation. Refusing the scientific dialogue in a public transparent scientific competition it is like the unfair action of not letting an athlete participate to the Olympics because has demonstrated higher skill than others who cut him/her off because otherwise will lose. However, the difference in this case is that the biggest damage and unfairness is not just to the inventor who is cut off, but advancements in science are delayed, progress and humanity are damaged for being deprived from the benefits of inventions that will provide well-being.

The excuse for rejection of my 2505 abstract because “I think it is not expected that the reviewer starts a literature research to find the lacking information” is not an appropriate reason because reviewers had all the information they needed in my 2016 IEEE-MIC 2505 abstract/summary and it is also clear that reviewers and leaders in the field knew about my inventions during the past two decades. Many have written letters of endorsement, or have worked to fix the peer-review unfairness of my papers such as the IEEE senior scientist Aaron Brill in 2000, the IEEE General Chairman Uwe Bratzler in 2008, and several others who were unsuccessful; however, until the rigged peer-review system is fixed for everyone, those in power with special personal interests or conflict of interests as in the case of Surti will take advantage of the system's loopholes and crush innovations, waste millions of taxpayer money, and deprive humanity of benefits.
2. Quantitative data & technical merits of the project/research provided in the text of the 2505 Abstract

This abstract contains extremely valuable quantitative data & technical merit of the project/research:
1. The ultra-sensitive 3D-CBS has the capability of extracting ALL valuable information from radiation (on spatial and time resolution, energy and sensitivity) at the lowest cost per valid signal captured from tumor markers
2. it reduces the radiation dose to the patient and provides valuable information to doctors on anomalies in morphological changes and biological processes.
3. enabling an effective early detection of cancer and other diseases
4. providing physicians with more accurate information to improve diagnoses, prognoses and improve monitoring of diseases
5. The basic 3D-Flow invention . . . it was proven feasible and functional in hardware using two FPGA chips in 2001 and in two modular industrialized boards in 2003, suitable to build 3D-Flow systems for detectors of any size

This abstract describes the features of the ultra-sensitive 3D-CBS Medical Imaging device based on the 3D-Flow OPRA which is capable of extracting ALL valuable information from radiation on spatial and time resolution, energy and sensitivity at the lowest cost per valid signal captured from tumor markers.

It is impossible to list all quantitative information in a 300 word abstract; however, the link (www.UnitedToEndCancer.org/doc/900.pdf) provided in the first line of the abstract or the two pages of the 2505 Abstract/Summary provided within this abstract will enable Visvikis, Surti and their reviewers, experts in the field of medical imaging, to extract quantitative data and technical merits and build Table 1. In particular, Surti who is also among the authors of the Explorer cannot ignore the clear picture in the last page of the 2505 Abstract/Summary showing how one 3D-Flow OPRA crate having nine data processing boards with 256-channels each can replace his 12 crates with 120 boards shown in slide 8 of his presentation.

All these are quantitative information that Surti, should be very familiar with as an author of the Explorer and after reading my 2505 Abstract should be able to compile the first twelve rows of Table 1.

Specifically, Explorer vs 3D-CBS in:
391,520 LYSO crystals (slide 9) vs. <3,000 BGO crystals;
1,920 electronic channels (slide 7) vs. 2,304 electronic channels;
16 channels/board (slide 8) vs. 256 channels/board;
120 electronic boards (slide 8) vs. 9 electronic boards;
12 crates (slide 8) vs. 1 crate;
capable of acquiring and processing 40 TB data each day (UC-Davis website) vs. capable of acquiring and processing >40,000 TB data each day;
the need of 40 TB data storage per day (DC-Davis website) vs. the need of 1 GB data storage per day;
4 to 6 racks containing computers to process the acquired data (UC-Davis website) vs. zero racks;
40 to 60 kW power consumption (UC-Davis website) vs. 3 to 4 kW power consumption;
The Explorer is less sensitive than the 3D-CBS;
costs 30 to 50 times that of current PET vs. 3D-CBS which is 2 to 3 times the cost of current PET;
an examination cost higher than current PET vs. an examination cost lower than current PET.

All the above are quantitative data easy to be extracted from the figure on the last page of the Abstract/Summary, and supported in great detail in the document available at the link (www.UnitedToEndCancer.org/doc/900.pdf) provided in the first line of the abstract whose construction of its components are proven feasible by 59 quotes from reputable industries. Furthermore, the 3D-Flow innovative basic concept used in the 3D-CBS (3D-Complete Body Screening) was proven functional in hardware in two modular boards each with 68×3D-Flow processors.
3. Innovation and significance of the project/research provided in the text of the 2565 Abstract

This abstract contains extremely valuable innovations and significance of the project/research:
1. The ultra-sensitive 3D-CBS (3-D Complete Body screening) Medical Imaging device based on the 3D-Flow OPRA system capable of extracting ALL valuable information from radiation at the lowest cost per valid signal captured from tumor markers, saves lives by enabling an effective early detection of cancer and other diseases, providing physicians with more accurate information to improve diagnoses, prognoses and improve monitoring of diseases with the potential to save millions of lives while reducing healthcare costs
2. it reduces radiation dose to the patient, reduces cost, and provides valuable information to doctors on anomalies in morphological changes and biological processes
3. The basic 3D-Flow invention was first acclaimed in 1992 in many letters from scientists. In 1993 it was recognized valuable by academia, industry and research centers in a formal scientific review.
4. It was proven feasible and functional in hardware using two FPGA chips in 2001 and in two modular industrialized boards in 2003, suitable to build 3D-Flow systems for detectors of any size.

This abstract describes the features of the ultra-sensitive 3D-CBS Medical Imaging device based on the 3D-Flow OPRA which because of its innovations has a very high significance in saving many lives by enabling an effective early detection of cancer and other diseases, providing physicians with more accurate information to improve diagnoses, prognoses and improve monitoring of diseases with the potential to save millions of lives while reducing healthcare costs. It provides valuable information to doctors on anomalies in morphological changes and biological processes.

The significance in saving many lives and reducing healthcare costs is provided by my 1992 basic 3D-Flow invention and all subsequent inventions in detector assembly, topology, geometry, electronics, cable assembly, coupling the detector with the electronics, algorithms, etc., which created my 2015 invention of the 3D-CBS based on the 3D-Flow OPRA offering a staggering efficiency improvement at a very competitive price compared to other PET systems/approaches such as the Explorer.

The basic conceptual invention of the 3D-Flow in 1992 has evolved into several other inventions that now allow the 3D-CBS Medical Imaging device represented on the last page of the 2505 Abstract/Summary to be built. Its significance is listed in the 300 words abstract supported in the two page summary and detailed in the document accessible at the link provided in the first line of the abstract. The 3D-Flow innovation capable of processing a frame (or data set acquired from the PET detector) for a time longer than the time interval between two consecutive frames (or data sets) has the advantage in the new 3D-Flow OPRA design to minimize distance between components, minimize power consumption, optimize algorithm executions to capture and accurately measure all possible valid signals from the tumor markers at the lowest possible cost per each valid signal captured.

To give an idea of the staggering difference in performance of the 3D-CBS based on the 3D-Flow OPRA, it has the capability to acquire and process in real time over 40,000 TB data in one day compared to 40 TB data of the Explorer.

This performance capability of the 3D-CBS enables an effective early detection of minimum anomalies in biological processes which is valuable information to the doctors that no other device can provide. This information helps doctors diagnose health problems at the earliest and most curable stage. It requires very low radiation to the patient and a low examination cost because the processing capability of the 3D-Flow OPRA allows the use of economical crystals (e.g. BGO).

The innovations of the 3D-CBS offer unprecedented advantages that can provide a great significance in a more powerful diagnostic tool for doctors to save lives and reduce costs which other approaches cannot claim because the 12 crates of electronics for data acquisition, the 6 racks of computers and the 40 TB storage capability of the Explorer cannot acquire and process 40,000 TB in one day like the 3D-CBS can. Furthermore, Joel Karp, one of the authors of the Explorer, admitted in an email (see text of the email at this link) he sent me on Aug. 19, 2002 that: “For LSO or GSO the cost seems prohibitive” (LYSO used in the Explorer has characteristics and costs similar to LSO). In fact, the LYSO crystals together with the electronics listed above consuming 60 kW (compared to the 4 kW of the 3D-CBS), makes the Explorer unsuitable to save many lives and reduce healthcare costs because it is not able to capture and process over 40,000 TB per day and because of its prohibitive cost.

The 3D-CBS innovations have solved these problems, and the last line of the first page of the 2505 Summary summarized the significance of this paper with the words: “This project plans to test 10,000 people (as detailed before) and achieve a 33% reduction of cancer deaths in 6 years and 50% in 10 years.”

The innovative merits and significance of the 3D-CBS based on the 3D-Flow system are supported by calculations, logical reasoning, scientific evidence, comparisons with other similar devices and its hardware implementation proven to be feasible in the 2505 Abstract/Summary. It is supported in great detail in the document available at the link (www.UnitedToEndCancer.org/doc/900.pdf) found on the first line of my abstract, and the construction of its components have been proven feasible by 59 quotes from reputable industries. Furthermore, the 3D-Flow innovative basic concept used in the 3D-CBS was proven functional in hardware in two modular boards each with 68×3D-Flow processors.
4. Supporting material to the quantitative data, technical merits, innovation and significance of the project/research provided in the text and figures worth a thousand words in the 2565 Summary

The first page of the 2305 Summary describes the features and advantages of the 3D-CBS (3-D Complete Body Screening), the usefulness of the new features to make a paradigm change in anatomical and molecular imaging enabling the detection and localization of minimal abnormal biological processes, providing accurate measurements, requiring minimum radiation dose safe to the patient, helping doctors to learn about the development of diseases such as cancer and other diseases at an early, highly curable stage.

Next is a list of the misconceptions that have driven the development of PET in the wrong direction of improving the spatial resolution to the detriment of sensitivity, using the traditional 16 cm Field of View (FOV) geometry with expensive crystals rather than my proposed long FOV (120 to 160 cm) with economical crystals. I have documented this wrong direction with the reference (at the bottom of the first page of the summary) to the rejection claims of ten proposals submitted to NIH in nine years. NIH reviewers also denied technological innovations in my proposals asking me to focus on improving the crystals rather than improving the electronics. These wrong directions are now being recognized as a mistake and have been reversed 15 years later as stated in the recent 2015 press release by Moses': <<We're developing the electronic interface between the detectors and the computer algorithm—and the electronics for this scanner is an order of magnitude more complicated than what's been done before.” says Moses>>

The last paragraph on the first page of the summary lays out a procedure that all researchers and leaders who want to maximize the reduction of cancer deaths and cost should be able to agree upon. This procedure sets objective results measured on a sample population that are fair to everyone who wants to achieve the goal of reducing cancer deaths and costs whether through a new drug, vaccine, medical imaging device, or healthy lifestyle promotion, etc. The procedure calls for all applicants to be responsible by requesting them to judge themselves by estimating the results they expect to attain in reducing cancer deaths and costs with their project (or combined with other existing techniques) when measured on a sample population, requesting they defend their claims with scientific arguments answering questions from colleagues and reviewers in an open, public review/forum.

The second page of the 2505 Summary in the title and subtitle summarizes the features of the 3D-CBS, its potential benefits and the detailed technical characteristics of the 3D-CBS unit with 14,400×3D-Flow processors in a volume of 16 cm cube of electronics executing up to 120 Object Pattern Recognition Algorithms Steps (OPRAS) on 64-bit data words per channel arriving in parallel from 2,304 detector channels at 20 MHz.

Next, there are six boxes posing six questions with their relative answers that address the problem to be solved, cancer, from the general problem of being the world's most deadly and costly calamity to the practical solution through a series of logical steps. The steps identify the most cost-effective item (in this case technology) that will have the greatest impact in reducing cancer deaths and cost. The questions are:
What is the most deadly and costly calamity?
Do we know what does and doesn't solve the problem?
Does data support these findings?
What is the best method for early detection?
Can we prove which method is the best?
What should we improve? What results should we expect?

For each question I provide an answer supported by data, calculations or scientific evidence. The logical reasoning continues in greater detail by answering two additional questions:
Why is extracting ALL valuable information from radiation important to improve early cancer detection?
How is ALL valuable information from radiation extracted?

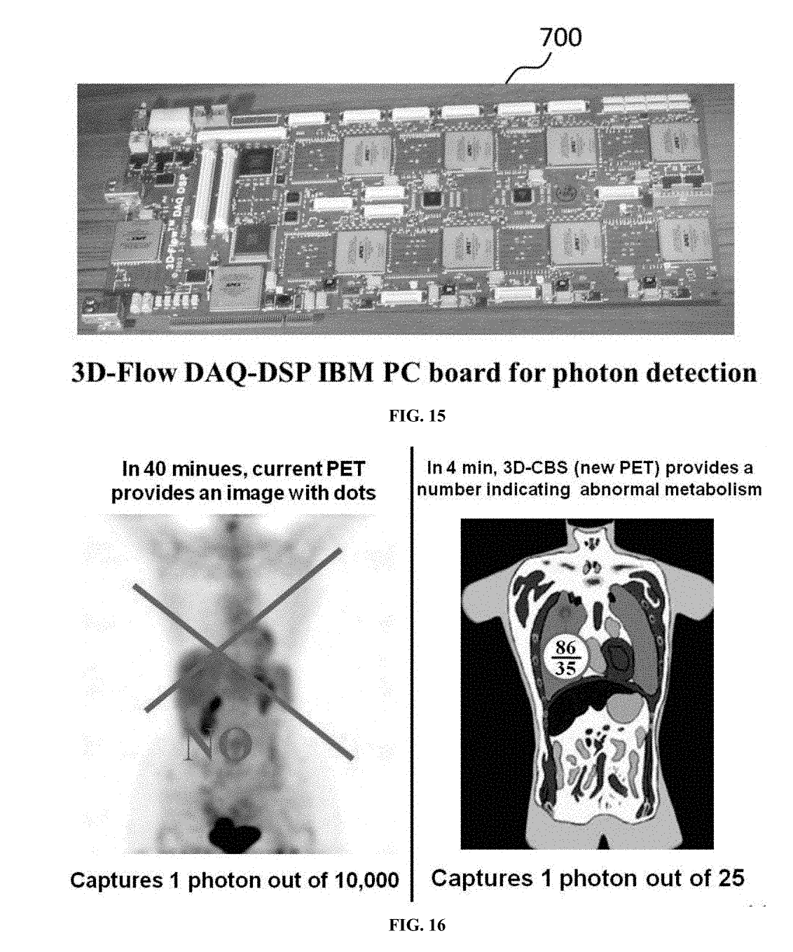

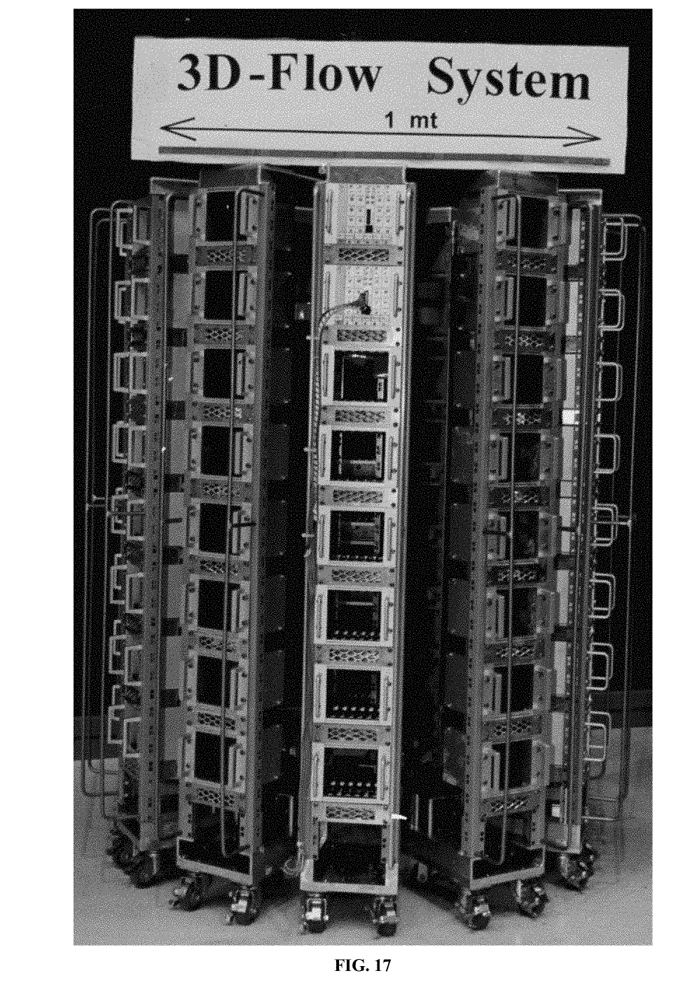

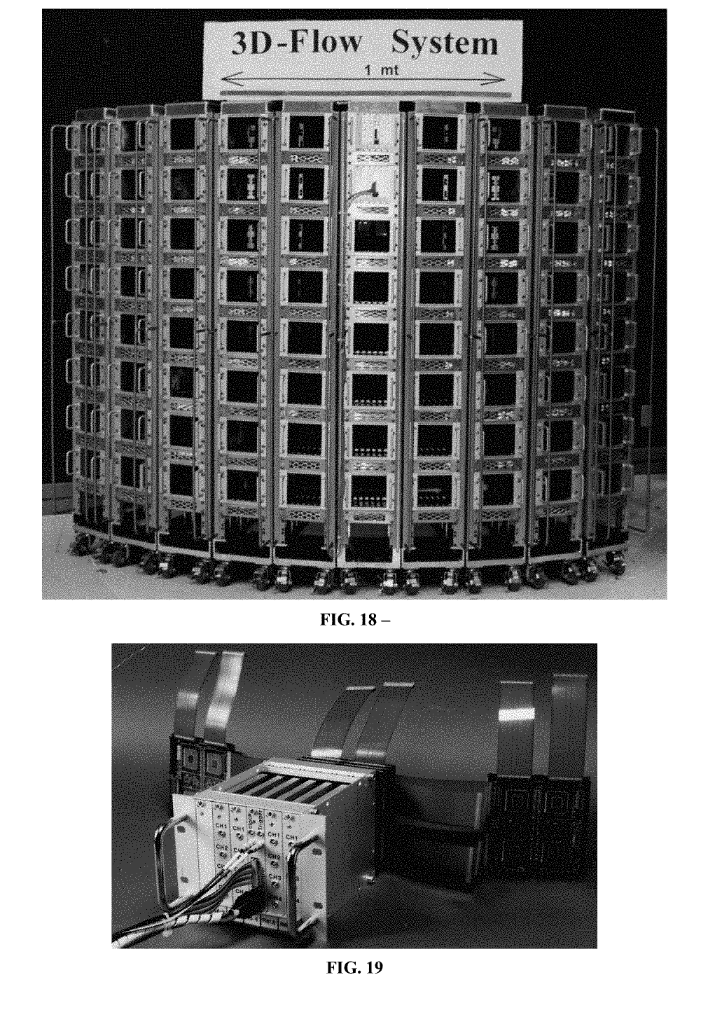

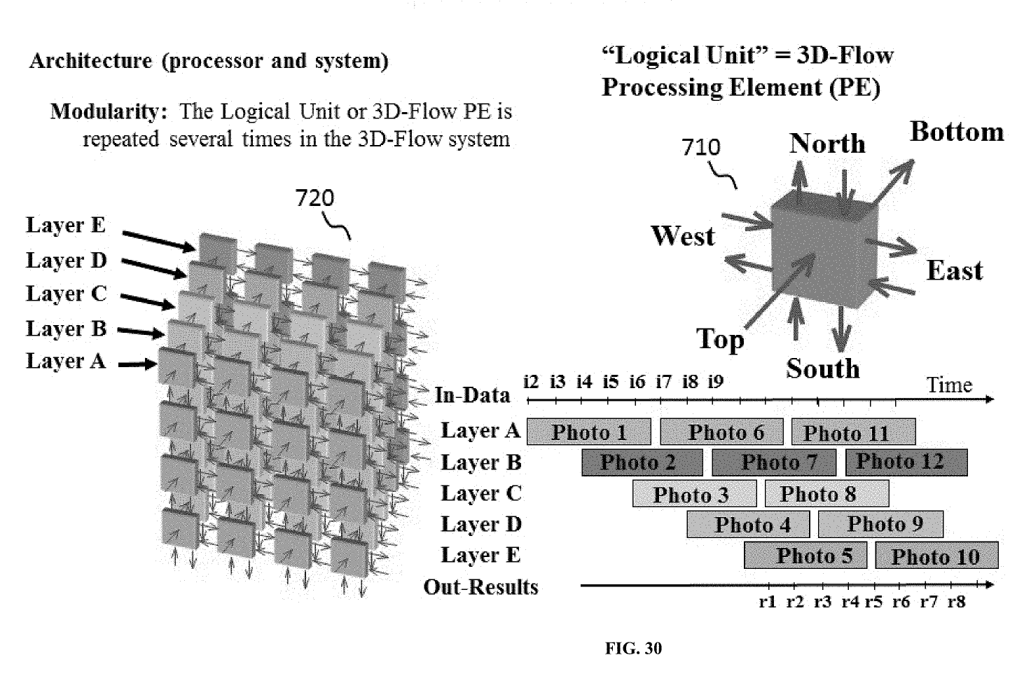

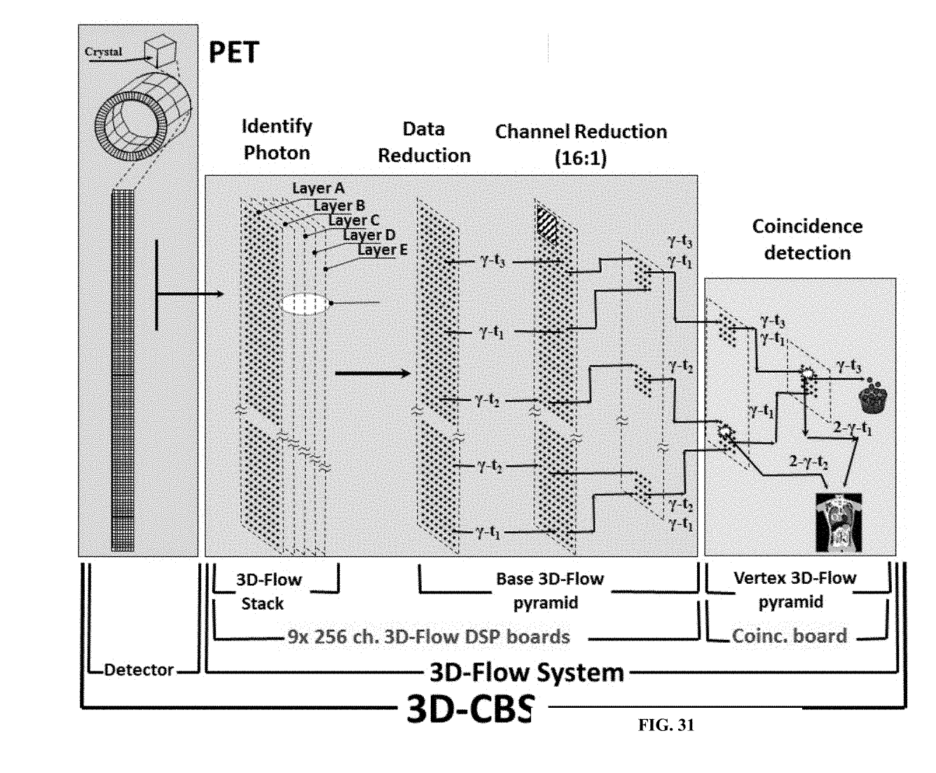

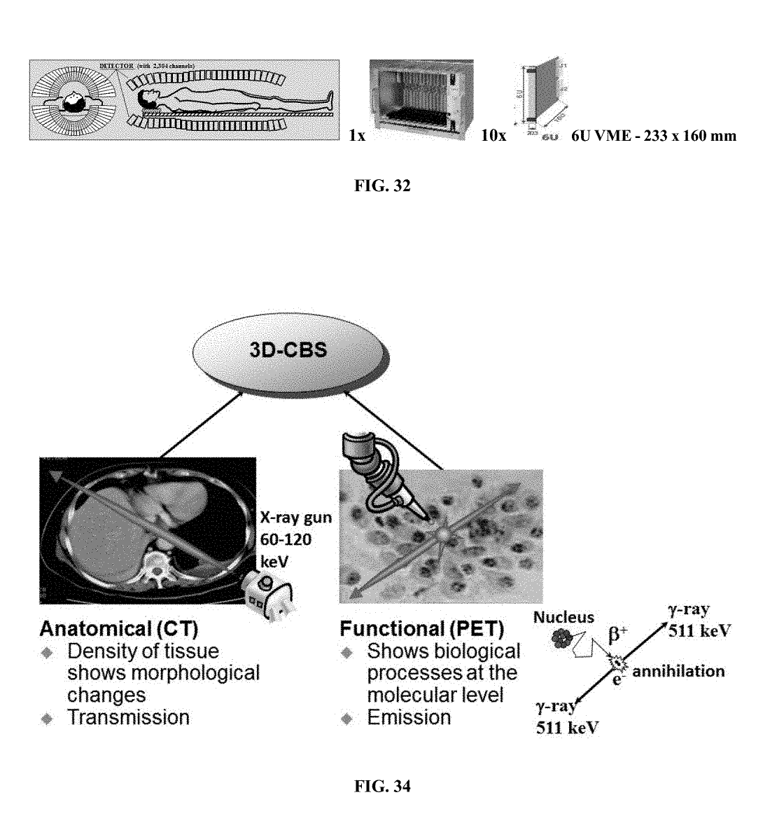

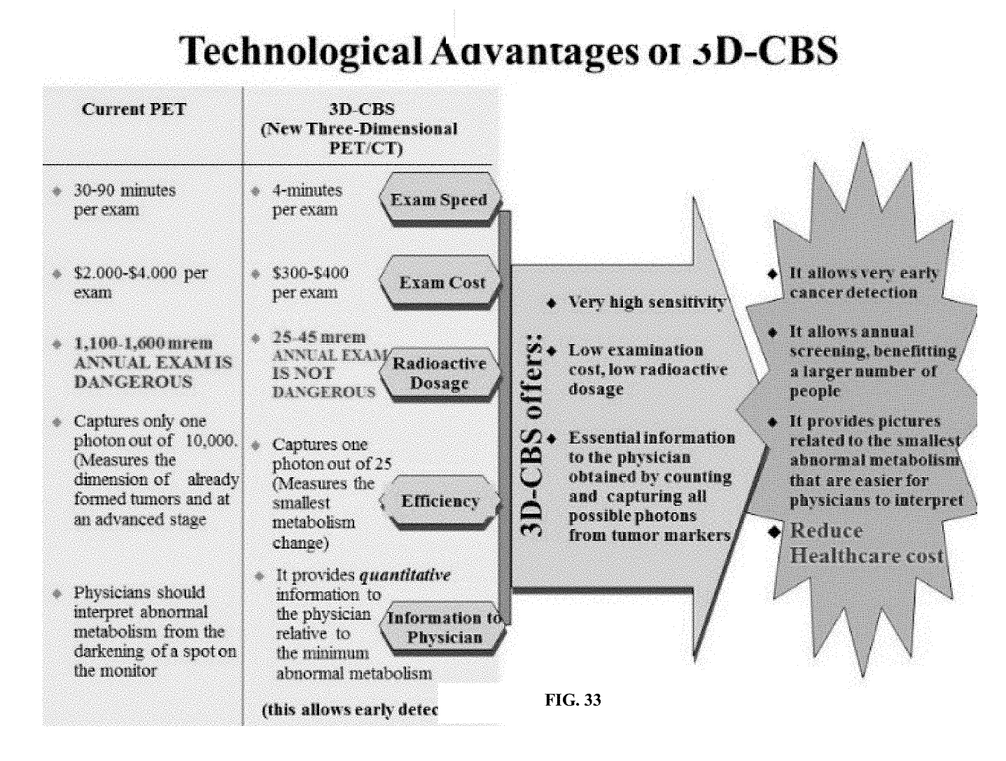

What follows is the description of FIG. 5 which worth a thousand words showing the entire 3D-CBS system with mechanical dimensions to scale:
Bottom left represents the whole 3D-CBS detector, the detector element coupled to PMT or SiPM and the type of signals generated that are sent to the bottom “FRONT-END ELECTRONICS, A/D converters” crate.
Two 400 pin-connectors carrying 256 signals per VME board through 256 Twinax cables from the “FRONT-END ELECTRONICS, A/D converters” crate to the “3D-Flow System” crate.
Each connector/cable assembly consists of two PCB boards 210 mm×37.84 mm at both ends of 128-Twinax cables grouped in 4×16 ribbon on two sides of the small board. These ribbon cables are soldered onto the small boards with traces carrying the information to the 400-pin connectors. This assembly is described in detail on page 193 of the document at the link (www.UnitedToEndCancer.org/doc/900.pdf) found on the first line of the abstract.
Central left represents the “3D-FLOW SYSTEM” crate receiving the 256-signals per board from the lower “FRONT-END ELECTRONICS, A/D converters” crate.
Center figures represent the nine VME boards, 2 cm wide housed in the “3D-FLOW SYSTEM” crate connected through 2,304 Twinax ribbon cables to the “FRONT-END ELECTRONICS, A/D converters” crate.
The description of the VME 256 channels 3D-Flow data processing board is reported on page 154 and 155 of the proposal to DOE #0000222704, responding to the 2016 solicitation DE-FOA-0001414 at the link (www.UnitedToEndCancer.org/docs/900.pdf) provided on the first line of the abstract. Two reputable industries provided the quotes for the NRE (Non-Recurring Engineering), and for a small production of 32 of these boards.
FIG. 31 shows the logical flow of the data from the detector to the 3D-Flow data processing system, through the 3D-Flow pyramid for data reduction and channel reduction. This figure has been published in several articles, starting from 2000 in FIG. 16-1 on page 172 of the book that I sent on August 2002 to Surti's colleague, Joel Karp, and he might still find the book in his laboratory.
FIG. 34 shows the two features of the 3D-CBS (anatomical and functional imaging) and FIG. 30 shows the concept of the 3D-Flow invention.
FIG. 33 shows the Technological advantages of the 3D-CBS compared to the current over 6,000 PET devices in use in hospitals, which has been published in several articles as well as the figure on the bottom right of the last page of the 2016IEEE-MIC 2505 Summary.
5. Crosetto's comments to Dimitris Visvikis and Suleman Surti responsible with their reviewers to accept/reject the 2505 paper

Dear Dimitri and Suleman,

I realize that we might have different views and approaches as to what constitutes the most valuable medical imaging device to greatly contribute to the defeat of the most deadly and costly calamity, cancer, improve healthcare and reduce costs; however, with all my heart, on behalf of taxpayers and cancer patients, I am respectfully asking you to be fair to yourselves by referring to the ethics of scientists, and to be fair to taxpayers and cancer patients by submitting yourselves to the judgement of an analytical discussion, calculations, scientific evidence and ultimately the judgement of experimental results.

Facts reported herein prove that it is not true that chairmen and reviewers were unaware of the advantages of my 3D-CBS invention based on the 3D-Flow system because I extensively presented my innovations in peer review articles, at conferences, distributing 200 copies of my technical-scientific book at the 2000 IEEE-NSS-MIC conference, talking to the joint authors of the Explorer project which includes you, Suleman Surti, who cited my work in slide 5 of your presentation.

I was surprised that Dimitri Visvikis, after answering my email on July 24 informing me that my Abstract/Summary 2505 was rejected, did not provide reasons or answer any additional emails, not even my contribution sent to him and Ms. Julie Amodeo, Director of IEEE customer relations & operations, to help him correct the “MIC Topic Description” by adding the buzz words: “early”, “detection”, “prognoses”, “biological”, “anomalies”, “tumor”, “cancer”, “isotope” to the descriptions which do not appear even once in his text. I had to disturb the President of IEEE, John Verboncoeur, to provide Visvikis' reasons for his and his reviewers' rejection of my Abstract/Summary 2505.

Now that I have proved that my Abstract/Summary 2505 contains quantitative information, does has technical merits, originality and innovation and significance in providing staggering benefits, and that the scientific community and by the authors of the competing Explorer project that copied many of my ideas knew of my inventions, it is the ethical professional duty of a scientist and only fair to the public who trust scientists, for the chairmen of the 2016 IEEE-MIC conference to provide an Abstract/Summary paper they approved that addresses technical merits, innovation and significance with a complete design from detailed quantitative data of the entire project in one figure to the innovations and its benefits as the Abstract/Summary 2505 does.

Likewise, it is time that Suleman Surti and all co-authors of the Explorer project take the courage to face a fair competition of ideas in a public scientific procedure by addressing analytically calculations and scientific evidence where we can question each other and compare in a public review and forum my 3D-CBS and 3D-Flow inventions with the Explorer approach by analyzing the description of the projects, data processing boards, components and technology data sheets.

To bring respect and to be fair to taxpayers and cancer patients who pay for the advancement in research, with all my heart, in defense of science, taxpayers and cancer patients, I respectfully invite you to reflect and resign if you cannot allow the science of medical imaging to proceed freely through fair, open, public competition.
E. Conclusion of the response to the President of IEEE

Dear John Verboncoeur,

I understand that you do not have the power or the knowledge to interfere in the operation of conference technical programs. However, you and IEEE leaders can analyze whether organizers of conferences seeking IEEE endorsement comply with IEEE's Statue and Mission statement as reported on their website: “IEEE is the world's 400,000 member largest technical professional organization dedicated to advancing technology for the benefit of humanity”.

You and IEEE leaders can inform the leaders of the conference in Strasbourg on Oct. 29-Nov. 6, 2016, that chairmen and deputies Eckhart Elsen, Susanne Kuehn, Dimitris Visvikis and Suleman Surti do not comply with the IEEE rules defined in the Statue and Mission published on IEEE's website. Facts reported in this document prove that the chairmen were familiar with my technology (they even cited my invention in slide 5 of their presentation) and the drawings in the figures of the Summaries of my papers provided information showing my 3D-Flow OPRA and 3D-CBS inventions are advancing technology for the benefit of humanity and are more advantageous in technical merits, innovation and significance with respect to other approved abstracts.

You and IEEE leaders can inform the leaders of the conference in Strasbourg to find other chairmen who can do their job by allowing me to present my breakthrough technology described in three Abstracts/Summaries 2493, 2484, 2505, replacing hundreds of crates of electronics with one crate providing staggering performance improvement at a fraction of the cost “for the benefit of humanity” that was proven feasible in 59 quotes by professionals in reputable industries who are IEEE members dedicated to advancing technology for the benefit of humanity.

You and IEEE leaders can inform the leaders of the conference in Strasbourg that IEEE is in a difficult position as it cannot break the rules stated in the Statue and Mission of the IEEE organization as published on their website.

If the leaders of the Strasbourg conference cannot find a solution that will comply with the Mission and Statue of IEEE, then the logic and compliance with the scientific integrity would call for IEEE to disassociate itself from the Strasbourg's conference organizers who are contrary to advancing technology for the benefit of humanity and remove its endorsement of the conference.

Thank you for copying your email to NPSS President-Elect Stefan Ritt for future continuity. Forgive me if I copy to other leaders in different areas who could help to resolve the problem of the independence of the peer-review process expressed by the National Academy of Science.

The above facts showing innovations suppressed causing millions of preventable deaths and billions of taxpayer dollars LOST and humanity deprived from the benefits of advancement in science clearly call for a reform of the peer-review process.

Sincerely,

Dario Crosetto

From: John Verboncoeur [mailto:johnv@egr.msu.edu]

Sent: Monday, Sep. 19, 2016 7:17 AM

To: United To End Cancer <volunteers@u2ec.org>

Cc: Ritt Stefan (PSI) <stefan.ritt@psi.ch>

Subject: Re: per Your request I am resending the 3 questions. RE: Breakthrough invention: 3D-Flow OPRA—a revolutionary electronic instrument for multiple applications: advancing science, saving lives, fighting terrorism

Dario,

Although I understand these are not the reviews you were hoping for, I do not have the power nor the knowledge to interfere in the operation of conferences technical programs. These are staffed by volunteers with significant turnover each year. Hence, we should conclude that any anomaly in their decision process should be both topically and temporally localized.

It is not feasible for a conference of this size to engage each author whose abstract is rejected in a lengthy iteration in order to try to improve the abstract to make it acceptable. You should not take criticism of your abstract as criticism of the underlying work, and indeed here two key components of the reviews point to concerns over novelty, and concerns that the style is more marketing and less scientific. These are both remedied by ensuring your abstracts are substantially different from previous versions, and by focusing on quantitative details rather than qualitative assertions. You may speak to the various technical program chairs directly at the conference to better understand the concerns and how to address them.

At this point, I am not sure there is much more I can do beyond the above recommendations. I have copied NPSS President-Elect Stefan Ritt for future continuity.

Sincerely,

John P. Verboncoeur

On Sep. 19, 2016 02:30, United To End Cancer wrote:

John,

The answers you received are not professional and not scientific.

It is like a scientists providing hundreds of pages of calculations, observation, scientific evidence that the Earth is not at the center of the Universe and the reviewers score: “Technical Merit poor, Originality & Innovation poor, Significance poor”

Would you agree that the reviewers should go over the calculation provided by the scientist and invalidate them with their calculations which provide a different result, they should refute the scientific evidence provided by the scientist to support their “poor” score evaluation otherwise the reviewers might crush a discovery?

Could you please appoint someone who represents the IEEE mission and value of the world's 400,000 members largest technical professional organization dedicated to advancing technology for the benefit of humanity who can provide professional scientific reasons?

I have provided three pages per each abstract/summary. I also provided the link www.UnitedToEndCancer.org/doc/900.pdf to 271 page supporting in detail the claims of the abstract and summary.

In my abstract/summary I am describing a 3D-Flow OPRA programmable system for Level-1 Trigger for 8,192×16-bit channels with the capability to extracts ALL valuable information from radiation using Object Pattern Real-Time Recognition Algorithms (OPRA) from 80 million events/second (radiation) @1.3 TB/second transfer rate from over a billion collisions/second, using 43,008×3D-FLOW processors @$1 each in a single VXI crate with zero dead-time costing approximately $100,000.

This 3D-Flow OPRA system in one crate has the capability to replace hundreds of crates of electronics with 4,000 data processing boards, 40 computers running 200 tasks of the Smith's CMS Level-1 Trigger.

My basic 3D-Flow invention has been endorsed by hundreds of scientists, the concept has been proven feasible and functional in hardware, providing staggering performance improvements as confirmed in a public scientific review conducted in December 1993 (see pp. 56-74) where I answered objections from other scientists. Now, my new 3D-Flow OPRA is proven to be feasible by 59 quotes from reputable industries.

Please let me know the names of the professionals appointed by you so we can address any question/concern related to the document provided in the abstract.

Looking forward to your reply

Sincerely,

Dario

From: John Verboncoeur [mailto:johnv@egr.msu.edu]

Sent: Sunday, Sep. 18, 2016 12:23 PM

To: United To End Cancer <volunteers@u2ec.org>

Subject: Re: per Your request I am resending the 3 questions. RE: Breakthrough invention: 3D-Flow OPRA—a revolutionary electronic instrument for multiple applications: advancing science, saving lives, fighting

Dario,

Here is what we have been able to obtain on the other two submissions:

The 2 of 3 reviewers suggest to reject both 2493 and 2484.

The score for 2484 is very low with 5.0 and same for 2493 with 5.0.

They are judged by different reviewers and all give:

Technical Merit poor, Originality & Innovation poor, Significance poor.

In addition for 2484 the comments are: this is pure nonsense. duplicate with #2493

And from a second reviewer: The last several sentences does a lot of damage to the credibility of this abstract.

The architecture seems like a standard parallel algorithm, with pipes that feed multiple CPU's—so I'm missing the innovation here.

For the 2016 IEEE-NSS 2493 abstract/summary: “I do not understand how 3D flow differs to various standard network topologies currently used in HEP (this is after reading a poster or two on his website).”

As you can see, there are specific concerns that can be addressed. In particular, a more convincing case is needed for identifying the innovation in your work. Note that with the new CrossCheck software being required by IEEE, abstracts get compared with all publications in the IEEE database for duplication, so that significant duplication gets flagged for manual investigation; there are of course cases where description of an apparatus is significantly the same for different projects, so this is not automatically disqualifying. One good tactic might be a single paper focusing on the innovations and comparison with existing technology, as that seems to be a recurring theme in the reviews.

Are you encountering similar challenges in submissions to other conferences? While NSS-MIC rejects hundreds of papers each year, I am not aware of others raising concerns.

Cheers,

John P. Verboncoeur

President, IEEE Nuclear and Plasma Sciences Society

Associate Dean for Research, College of Engineering

Professor, Electrical and Computer Engineering

Professor, Computational Mathematics, Science, and Engineering

Michigan State University

3410 Engineering Bldg

428 S. Shaw Lane

East Lansing, Mich. 48824-1226

517-355-5133 johnv@egr.msu.edu

From: John Verboncoeur [mailto:johnv@egr.msu.edu]

Sent: Monday, Sep. 5, 2016 2:27 PM

To: United To End Cancer <volunteers@u2ec.org>

Cc: Marie Hunter <m.m.hunter@ieee.org>; Julie Amodeo <julie.amodeo@ieee.org>

Subject: Re: per Your request I am resending the 3 questions. RE: Breakthrough invention: 3D-Flow OPRA—a revolutionary electronic instrument for multiple applications: advancing science, saving lives, fighting terrorism

Dario,

Thus far I have obtained the review summary from 2505:

“scored all categories as poor, because: The text of this “so-called” abstract might be appropriate for a newspaper. But even for such a purpose there is insufficient information for the reader. For me it is totally unclear—even after having looked at the summary, which is of ultimately bad quality—what kind of system is the “ultra-sensitive 3D-CBS”. I think it is not expected that the reviewer starts a literature research to find the lacking information.

Once again, the abstract and supporting data emphasize the difficulties in getting the project funded rather than submitting scientific material (as he's been told in the past). VERY strong reject!”

As you can see, this review does not leave much room for recovery for this conference. I think the challenge is that the reviewers are looking for more summary scientific information rather than funding concerns. I suggest looking at the accepted abstracts for the conference to see the way they present scientific information; in your case comparative performance data and a brief summary of the device itself might address the concerns.

I will keep you posted on the other reviews when I receive them.

Cheers,

John P. Verboncoeur
XV. As Galileo could not make science and justice prevail alone and the benefits of his discoveries were delayed for years after his death and he was recognized to be right by the Church only 350 years later, Crosetto cannot make science and justice prevail by himself but media and everyone need to pass on this information and stand up for the interest of their children, grandchildren and loved ones

The consequences to taxpayers and cancer patients because scientists do not take responsibility by standing up for science and for what is right are wasted money, lives lost needlessly and being deprived of benefits.

Be the change you want to see in a world of reason and respect for your children and future generations by supporting the constant consistent work of Crosetto.

During these past 25 years Crosetto has been very active in informing the decision makers about his work/inventions by submitting articles to scientific conferences and to scientific journals. He has written five books, published several documents online, sent emails to key people in the field and submitted requests for funding the NRE (Non-Recurring Engineering) to implement his 3D-Flow Processor into an ASIC (Application Specific Integrated Circuit having 4×, 16×, or 64×3D-Flow processors per ASIC) to be used as the building blocks for several other applications benefitting many fields. In particular, he made detailed designs of powerful tools (systems) for applications in physics research to discover new subatomic particles and in medical imaging to provide lifesaving 3D-CBS devices for early cancer detection.

He also submitted requests for funding to implement several other inventions/projects, some related to the 3D-Flow ASIC and others not related such as A/D, DAQ analog-to-digital and data acquisition electronic boards.

Documents show that all Crosetto's designs that he developed during the past 30 years have been thought out analytically in great detail before construction. They are both technologically superior and more cost-effectiveness than the technology available at the time of development. An example: his modular TRAM FDPP processing module that he designed, built and tested at CERN in 1989 using leading-edge technology, the very first 7 nanosecond PAL (Programmable Array Logic) where he packed components on both sides of the printed circuit board without leftover space, not even to drill a hole for a via. The FDPP is the processing node of a parallel-processing system with two memory banks and two processors on each node, a Transputer and a DSP. The Transputer handles communication between neighboring nodes while it fetches results and loads new input data onto a memory bank; the DSP number crunches data onto the other memory bank. Memory banks are swapped in less than 10 nanoseconds when both processors terminate their tasks.

His designs also include provisions for testing, monitoring functionality during operation, and features that will help technicians diagnose and repair failures due to external factors that damage the unit. An example: his modular 3D-Flow DSP IBM PC board with 68×3D-Flow processors implemented in FPGA where he provided 64 LED to visually check the functionality of the board and provide over 100 test points for testing fast signals to facilitate monitoring functionality and maintenance/repair.

He uses analytical thinking to think ahead, not only to satisfy the doubts or questions from his colleagues about the performance of his design to solve a specific problem but also how to obtain measurable, reproducible results proving his invention/project/instrument works as claimed. He achieves this by designing additional instrumentation having the capability of testing the performance of his primary invention because the ultimate judge is the experiment and not limited to the reviews of colleagues. An example: his recent design/proposal of the Level-1 Trigger system for LHC. The superiority of his 3D-Flow OPRA Level-1 Trigger system at 1.3 TB/sec, with 8,192 channels in one crate (see pp. 1-45 & 125-144 & 180-238 of the proposal to DOE #0000222704, responding to the 2016 solicitation DE-FOA-0001414) which would outperform and replace at 1/1000 the cost Smith's CERN-CMS Level-1 Trigger made of hundreds of crates of electronics, is not an unsupported claim, as its feasibility has been proven by 59 quotes from reputable industries. He subsequently designed a TER/DSU instrument costing $50,000 (see pp. 149-170 of the proposal to DOE #0000222704, responding to the 2016 solicitation DE-FOA-0001414) that allows the performance of his 3D-Flow OPRA system to be tested on a test bench by generating the functionality of the $50 billion LHC apparatus.

Had Smith would have thought how to test his claims regarding the performance of his Level-1 Trigger system on a test bench with an instrument similar to the TER/DSU event data recorder and simulator rather than focusing on preventing competing projects such as Crosetto's 3D-Flow invention from being presented at conferences or being adopted by experiments, he would not have wasted $100 million and 20 years of work by thousands of scientists but seen the failure of his design much earlier from experimental results.

Although Crosetto faced obstacles since 1992 from influential scientists who prefer power/money over reason/science, the majority of the time reason/science was the victor. However, around 1995-98 the odds began to change as several facts, documents and data will prove (see below the list of events). Among the reasons why power/money began triumphing over reason/science were the billions of dollars per year involved in the Large Hadron Collider (LHC) at CERN, the biggest and most expensive experiment in the history of the planet, and the huge amount of money spent every year on cancer research, which at NCI alone totaled $6 billion per year. As the amount of money involved began to increase, so the number of scientists who were willing to give up ethics and reason/science for power/money increased.

Still one would assume that cancer funding agencies and individual philanthropists wanting to make a difference in the world would have their own independent scientific review panel that would follow a world of reason/science, making a contribution to a project which provides the biggest bang for their buck. Instead, funding from other sources, including those who make an individual $10 donation, trust and refer to the earlier honesty of the scientific community where members followed the ethics of a scientist and therefore conclude that if Crosetto's articles are not approved by IEEE, the world's largest association of over 400,000 technical professionals dedicated to the advancement of technology for the benefit of humanity and he does not receive grants from large government funding agencies such as NIH, NCI, DOE, NSF, CPRIT, etc. the explanation must be because his invention/project is no good.

However, the reality is different. The scientific world has changed from how it used to be. The several billions of dollars per year used to fund research has affected members of the scientific community, so many are no longer these incorruptible, honest, pure members of society advertised by the media, but have now become scientists who deceive funding agencies, philanthropists, cancer and other humanitarian organizations and ultimately the laymen who trust the judgement of scientists when selecting a project.

There exists a simple, fair, logical, scientific method that cannot be refuted by honest scientists that would protect the ethics and good name of the scientific community and would greatly slash corruption if adopted: Ask each scientist to judge himself/herself before having colleagues evaluate their idea/invention/project by comparing their idea/invention/project with other prominent projects in the field claiming similar advantages/benefits. Each scientist should provide an estimate of results they would expect to attain if their project were funded and provide a plan on how to measure these results that could be applied to other proposed solutions that must be fair to all of them and to taxpayers who are paying for the research and expect to receive benefits.

For example,
in the application of physics experiments to build the TER/DSU ultra-high speed data recorder and hardware simulator so that different Level-1 Trigger projects can be tested and their performance compared. This would avoid wasting additional money on building devices that perform much less than expected or even a complete failure;
in cancer research the authors/inventors would be asked to estimate the reduction of cancer deaths and cost they expect to attain with their project (or combined with other existing techniques) and present a plan to test it on a sample population. For example, to test 10,000 people, ages 55-74 taken from a location where the mortality rate has been constant for the past 20 years. A difference or no difference in the mortality rate would quantify the success or failure of the proposed solution.

The scientific integrity of scientists who refuse to agree with their colleagues on a fair, objective methodology and rules to compare projects based on calculations and scientific evidence and then on how to measure results should be questioned. The rules should first of all protect the interests of the taxpayers before other scientists and comply with their professional ethics. If scientists cannot agree it would be like the organizers and jury of the Olympics who cannot agree on the rules and parameters to judge the athletes or where to set the finish line of the marathon. The jury would not be able to analyze objectively parameters of time or performance but the winning athlete would be determined by who endorses the athlete and how much money an athlete has for advertising their skill; those who do not have a sponsor or money to advertise their skill would not be allowed to enter the competition.

Here are provided some specific examples of events, facts, documents and data supporting the above statements and showing how the reality of the scientific world has changed from a world of reason/science to a world of power/money corrupted by the money.
1992 IEEE Moses-DeRenzo (world of power/money). In 1992 Crosetto faced the first time the difficulty of addressing scientific arguments based on calculation and scientific evidence when he was working at the Superconducting Super Collider and he communicated with two among the most influential scientists in the field, Bill Moses and Steve DeRenzo who worked at the same place at Berkeley National Laboratory. It made no difference talking to one or the other because he was receiving the same unsupported rejections and arguments that would only stand by their position of power. Moses has been for very long time in the IEEE advisory committee (Director EMC Society, Chairman Conferences Committee) and DeRenzo was considered the guru and the reference point about the characteristics of crystal detectors and he certainly is, however, Crosetto was trying to explain that experimental apparatus detecting particles are not only made of crystals but there are other components in an experimental apparatus for physics research and for PET medical imaging and scientists should be open to innovations. For example, electronics is a very important component in an experimental apparatus, as well as the detector assembly, the coupling of the detector with the electronics, etc.
1992 IEEE-CHEP92-NIM 3D-Flow invention (world of reason/science)
1993 DOE-SSC-CERN Crosetto invention endorsed by hundreds of scientists (world of reason/science)
1993 DOE-CERN Wesley Smith (world of power/money). In 1993 Crosetto faced a strong opposition from Wesley Smith who, contrary to hundreds of scientists from all over the world, even those working in competing experiments who recognized the value and endorsed Crosetto's 3D-Flow, programmable Level-1 Trigger, he stated that programmability at level-1 Trigger was not necessary.
1993 DOE-SSC; 3D-Flow-GEM (world of reason/science): Crosetto's basic invention was recognize valuable and adopted by GEM experiment, Gamma, Electron and Muon (GEM) collaboration at the Superconducting Super Collider (SSC). It was included in their detector technical design report from page 7-10 to 7-13 with references at page 7-86 of GEM-TN-93-262, SSCL-SR-1219, Jul. 31, 1993.
1993 DOE-FERMILAB-CERN-URA-Industries Crosetto's invention reviewed at FERMILAB (world of reason/science): major review of Crosetto invention. On Dec. 14, 1993, luckily the balance between a world of power and a world of reason was still in favor of the latter and those like Moses, DeRenzo, Smith and others could not stop implementing transparent scientific procedures such as the open, public, major scientific review of Crosetto 3D-Flow invention requested by the Director of the SSC and held at FERMILAB in 1993. Crosetto invention was approved, recognized valuable and he received all what was available during the closeout of the SSC laboratory. Crosetto receives a DOE grant of $150,000.
1994 IEEE Computer Society (world of reason/science) Crosetto's article “Massively Parallel-Processing System with 3D-Flow Processors. 0-81816-6322-7194, pp. 355-396.
1995-1998 DOE-Grant (world of reason/science). In 1995-98 Crosetto received a DOD grant of $100,000 and a DOE grant of $906,000, however, not sufficient to build a complete 3D-Flow system as it was endorsed in many letters from many scientists. Funding were not even sufficient to pay the NRE (Non-Recurring Engineering) of the 3D-Flow ASIC (Application Specific Integrated Circuit) with 4×, 16×, or 64×3D-Flow processors per ASIC which will be the building block to implement several applications.
1995 DOE-CERN, 3D-Flow-LHCb (world of reason/science) adopted by LHCb Letter Of Intent (LOI) 1995 adopted the 3D-Flow for the calorimeter and muon level-0 trigger
1995 IEEE Real-Time Conference, TNS 3D-Flow (world of reason/science). S. Conetti and D. Crosetto: “Implementing the Level-1 trigger algorithms for Beauty particle detector at LHC-b in real-time on a 3D-Flow system. Proceedings of the 1995 IEEE Conference on Real-Time Computer Applications in Nuclear Particle and Plasma Physics. Michigan State University, May 24-28, 1995, East Lancing. Pp. 44-48.
After 1995-1998, or around those years, the circle of friends of scientists who have control of the taxpayer and donation money by rejecting/approving articles in secret communications among themselves and approving/rejecting funding in meetings behind closed doors to split taxpayer money among themselves with no transparency and accountability to science and taxpayers, took irrefutable actions against Crosetto in rejecting articles and funding without scientific reasons, and more. They are implementing a world of power/money instead of a world of reason/science. They were able to impede any official formal, public scientific review for a world of reason similar the one held at FERMILAB in 1993, where the scientific truth for the benefit of humanity would emerge through the open, public dialogue among experts in the field.
1998 DOE-CERN Workshop Electronics for LHC, 3D-Flow (world of reason/science). Crosetto's article pp. 527-522. Cited by Eric Eisenhandler in his overview of all hardware processors at LHC at page 53.
1998 LHCb Technical Proposal 3D-Flow (world of reason/science) that change into a (world of power/money)
1999 45-page NIM article (world of reason/science) Crosetto's work was appreciated by Tom Ypsilantis from CERN, the Chief Editor of the peer-review scientific journal Nuclear Instrument and Methods in Physics Research and published in less than one month in a 45-page article
1999 IEEE-NPSS Real-Time (world of reason/science) D. Crosetto: “Real-Time system design environment for multichannel high-speed data acquisition system and pattern-recognition.” IEEE Real Time conference, Santa Fe, (N. Mex.) Jun. 14-18, 1999.
1999 IEEE-NSS article (world of reason/science) pp.329-337 D. Crosetto: “System Design and Verification Process for LHC Programmable Trigger Electronics” IEEE NSS-MIC. Seattle (Wash.) Oct. 24-30, 1999.
1999 CERN—LHCb 3D-Flow Presentation also on CERN website (world of reason/science) that change into a (world of power/money)
TURN OF EVENTS—(world of reason/science) turned into (world of power/money). Although you will see several articles and presentation by Crosetto in the following years, the ones that are important are presented and discussed at IEEE-NSS-MIC. This is the place where the players, or influential leaders in the field decide which electronics, technology, trend (e.g. focusing in PET spatial resolution or sensitivity) will be going research in physics (CERN) and in Medical Imaging.
1999 DOE-CERN Workshop Electronics for LHC, 3D-Flow (world of power/money) Wesley Smith and Peter Sharp prevented Crosetto from presenting at the 1999 Workshop on Electronics for LHC experiment at Snowmass, Colo. the results of his work from the $906,000 grant he received from DOE 1995-1998 ( ). Crosetto did not receive additional funding to translate into silicon his 3D-Flow processor, instead $50 million went to the alternative project by Smith (which has shown later to be a failure).
1999 DOE 3D-Flow Presentation On Sep. 2, 1999 Crosetto presented to DOE Office of High Energy Physics in Washington D.C. the high performance programmable, object pattern recognition Level-1 Trigger implemented with the 3D-Flow.
1999 IEEE-NSS article (world of power/money) Crosetto's paper rejected for publication in IEEE Transaction in Nuclear Science (TNS). Communications with IEEE senior reviewer Les Rogers, who was referring to the classical pipelining and to other architectures, having difficulties to understand Crosetto's invention. Only after Les Rogers expressed his view point and Crosetto pointed out the difference between his invention and Les Rogers viewpoint progress was made in understanding Crosetto's innovations. The majority of the reviewers approved the article for publication, two anonymous reviewers claimed the 3D-Flow architecture was flawed without providing any scientific reason and Crosetto's paper was never published. The 3D-Flow architecture was not flawed, it was proven feasible and functional in hardware.
1995-2003 Ten NIH $100,000 Grants rejected (world of power/money) for nine years NIH rejected all Crosetto's proposals. Crosetto used all available means provided by NIH such as appeals, talking to and exchanging emails with leaders at NIH and NCI, meeting NIH and NIBIB Directors at a press conference on Medical Imaging, requesting the help of U.S. Senator Kay Bailey Hutchison, Texas State Senator Jane Nelson, Senator John Cornyn, U.S. Congressman Michael Burgess, U.S. Congresswoman Eddie Bernice Johnson, and many others to request transparency in science by having a fair public scientific procedure similar to the one given for his basic 3D-Flow invention held on Dec. 14, 1993, at FERMILAB. The answer received from NIH executives, directors and employees to Crosetto or anyone enquiring on his behalf was that Crosetto should follow the reviewers' guidance and modify his research, approach, objectives toward improving spatial resolution (to the detriment of sensitivity), and improve crystals rather than electronics, essentially giving up on his invention. This turned out to be wrong because it has been recanted by influential leaders in the field who are now receiving $15.5 million from NIH to build the Explorer. Several authors of the Explorer are the same people who obstructed Crosetto during the past decades.
2000 IEEE CERN (world of power/money) two articles approved at the IEEE conference. D. Crosetto: “A modular VME or IBM PC based data acquisition system for multi-modality PET/CT scanners of different sizes and detector types.” Presented at the IEEE Nuclear Science Symposium and Medical Imaging Conference, Lyon, France, 2000, IEEE-2000-563. D. Crosetto: “Real-time, programmable, digital signal-processing electronics for extracting the information from a detector module for multi-modality PET/SPECT/CT scanners.” Presented at the IEEE Nuclear Science Symposium and Medical Imaging Conference, Lyon, France, 2000, IEEE-2000-567
2000 IEEE CERN 3D-CBS Book (world of reason/science). Book on the 3D-CBS distributed free of charge in 200 copies to the leaders in the field at the 2000 IEEE-NSS-MIC Conference. D. Crosetto: “400+ time improved PET efficiency for lower-dose radiation, lower cost cancer screening.” Technical-scientific book presented at the IEEE Nuclear Science Sympos. and Medical Imaging Conf., Lyon, France, 2000: ISBN 0-9702897-0-7. 2000
2000 CERN Hospital LABs—Review of Crosetto's 3D-CBS book by scientist. Public on Amazon (world of reason/science)
2000 IEEE Book review 3D-CBS (world of power/money) by DeRenzo appointed by Terry Jones stating in the year 2000 “We do not view the electronics as a problem, either in terms of performance or cost”. DeRenzo claim was reversed by the same leaders in the field in the recent 2015 press release: <<We're developing the electronic interface between the detectors and the computer aigorithm—and the electronics for this scanner is an order of magnitude more complicated than what's been done before,” says Moses>>.
2000 IEEE Book review 3D-CBS (world of power/money) by Del Guerra claims it is not necessary to increase the FOV, the sensitivity, he assumes that the radiation dose to the patient is not a problem he claims that it is not necessary to build a PET longer than 16 cm because that is the size of the largest organ in the body. See also later rejections by Alberto del Guerra (http://blog.u2ec.org/wordpress/?p=1363
2000 IEEE-CERN Crosetto's invention proven with simulation (world of reason/science) Chris Parkman from CERN, who was Chairman of the Industrial Exhibition of the IEEE-NSS-MIC conference in 2000 in Lyon, France, gave Crosetto a booth at the Exhibition free of charge where he could demonstrate on the computer a simulation of the 3D-Flow system made of thousands of 3D-Flow processors.
2001 IEEE-CERN Crosetto's invention proven in hardware (world of reason/science) At the same Industrial Exhibition of the IEEE-NSS-MIC conference in 2001 San Diego, Calif., Parkman gave a free booth to Crosetto where he could setup two Altera prototype boards with his 3D-Flow system of 8 processors implemented in two FPGAs proving the concept of his invention working in hardware. Visitors who stopped by Crosetto's booth could select a desired pattern of clusters on switches; the results were displayed on LED and the oscilloscope provided the waveform of the signals with the speed of the system.
2001 CHEP01 Japan (world of reason/science) Crosetto's article “Fast Cluster finding System for Future HEP Experiments” Presented at the Conference Computing in High Energy Physics. Tsukuba, Japan.
2001 CERN—University of Geneva (world of reason/science). D. Crosetto: “Saving lives through early cancer detection: Breaking the current PET efficiency barrier with the 3D-CBS.” Presented on May 16, 2001 at the University of Geneva, Switzerland, on May 18 at the University of Turin, Italy and on May 21, 2001 at the University hospital of Geneva.
2001 letter from U.S. Senator Kay Bailey Hutchison to NIH.
2001 letter from Texas Senator Jane Nelson to NIH. Senator Nelson raised $3 billion for cancer research, however, Crosetto's 3D-CBS was triaged out and not even reviewed
2002 IEEE Joel Karp (world of power/money) General Chairman at the 2020 IEEE-NSS-MIC Conference rejected all Crosetto's papers
2002 Siemens (world of reason/science) that change into a (world of power/money). Meeting with Siemens in 2002 stating that they have built 31 prototypes of PET and could not improve the efficiency of their PET by improving the electronics. Five years after the meeting with Crosetto Siemens announcement on their web site that they improved PET efficiency by 70% by improving the electronics.
2003 3D-CBS Dallas international review (world of reason/science) passed a major public scientific review. See the final report by the review panel.
2003, ICATPP, 3D-CBS. D. Crosetto: “Development of an Innovative Three-Dimensional Complete Body Screening Device—3D-CBS” Book: Astroparticle, Particle and Space Physics, Detectors and Medical Physics Applications. Editor: World Scientific, 2004, pp. 350-359.
2003 IEEE MIC (world of reason/science) Three articles by Crosetto were approved at the conference. D. Crosetto: “The 3-D Complete Body Screening (3D-CBS) Features and Implementation” IEEE-NSS-MIC-2003. Conference Record. M7-129. “3D-FlowTM DAQ-DSP IBM PC board for Photon Detection In PET and PET/CT. Conference Record. M7-130.
2003 NSF-DOE-NIH Grants $100,000 rejections (world of power/money). National Science Foundation (NSF) in 2002, the NIH on Aug. 1, 2002, Mar. 31, 2003, Jul. 31, 2003 and the DOE on Jan. 13, 2003 rejected funding Crosetto's $100,000 proposals to build the proof of concept in FPGA hardware or any other component of the advanced PET system such as A/D and PAQ boards. Crosetto therefore had to spend his own money to prove his invention was feasible and functional in FPGA hardware.
2004 CSS-ITALY 3D-CBS (world of power/money) The Italian Consiglio Superiore della Sanita' rejected the 3D-CBS project with no scientific reasons. Crosetto replied point-by-point to reviewers' remarks and objections, his responses were filed in the CSS dossier related to the 3D-CBS. However, two years later, Crosetto passed by the office of CSS in Rome, asked to view the dossier relative to the 3D-CBS and realized that his point-by-point responses to the reviewers were removed from the folder. The documentation provided and presented by Crosetto on Nov. 17, 2004 to the CSS, clearly demonstrates that the 3D-CBS is hundreds of times more efficient than the current PET, with a safe radiation level of less than 1 mSv. Garibaldi and other reviewers from the CSS are responsible for preventing the construction of more efficient 3D-CBS devices that could have already saved many lives. They did not accept the dialogue that would alleviate their doubts in regard to improving PET efficiency to enable effective early detection that would save lives. The disappearance of one million Euros, the disappearance of protocolled documents with Crosetto's answers to the committee from the file relative to the review of the 3D-CBS project at the office of the Minister of Health, the rejection of Crosetto's project based on the committee's statements that were later proven by calculations and experimental results from other scientists to be incorrect, calls for an investigation.
2004 NIH Crosetto. Intensive correspondence between Crosetto and leaders at NIH and NCI. Letters are available upon request.
2004 U.S. Senator Cornyn wrote a letter to NIH supporting the 3D-CBS project and inquiring why was not funded
2005 Friends from Monasterolo raised $6,500 (world of reason/science) for the 3D-CBS project in one evening. The money was used to pay the company Peteja to perform hardware tests and write drivers for the two 3D-Flow DSP, IBM PC boards each with 68×3D-Flow processors and for the development of algorithms for different applications.
2005 Rotary Alba and Friends from Monasterolo raised $64,000 donation (world of reason/science). Thanks to the initiative of Avv. Pier Mario Morra President of the Rotary Club in Alba and the Committee supporting the 3D-CBS project in Monasterolo, $64,000 were raised to support its development. The money was used to pay the company Peteja to perform hardware tests and write drivers for the two 3D-Flow DSP, IBM PC boards each with 68×3D-Flow processors and for the development of algorithms for different applications.
2005 ICATPP 3D-CBS Crosetto's article “Rethinking Positron emisssion Technology for Early Cancer Detection” (world of reason/science). Published on the Book: Astroparticle, Particle and Space Physics, Detectors and Medical Physics Applications. Editor: World Scientific, 2006, pp. 692-696.
2006 NIH Press-Conference (world of power/money) delegation to Washington to the press conference on Medical Imaging organized by NIH Director.
2007 IEEE Frey General Chairman of the IEEE-NSS-MIC (world of power/money) rejected all Crosetto's papers without providing any scientific reason.
2007 ICATPP 3D-CBS (world of reason/science) Crosetto's article: “Ignored Discovery Now Proven Capable of Saving Millions of Lives from Premature Cancer Death Demands Rethinking the Direction of Research” Book: Astroparticle, Particle and Space Physics, Detectors and Medical Physics Applications. Editor: World Scientific, pp. 624-639—2008
2008 CERN-World-Lab-Hospitals-Seminars (world of reason/science) that change into a (world of power/money) Crosetto gave several seminars at research center, CERN, World Laboratory, Hospitals, public events in Italy and Switzerland. D. Crosetto: “Logical Reasoning and Reasonable Answers Consistent with Declared Objectives for the Benefit of Mankind” International Seminars on Planetary Emergencies 40th Session, Erice, 19-24 August 2008. Editor: World Scientific 2009, pp. 531-560
2008 IEEE Bratzler (world of reason/science) that change into a (world of power/money). Uwe Bratzler, General Chairman of the IEEE-NSS-MIC Conference invited Crosetto to submit papers to the conference promising scientific reasons from his reviewers in the event Crosetto's papers were again rejected as during the previous years, All Crosetto's papers were rejected and Bratzler was unable to receive scientific reasons from his reviewers. He also kindly attempted to organize a forum where Crosetto and the leaders in the field could discuss analytically the issues based on calculations and scientific evidence. All leaders he contacted refused such open, transparent scientific procedure.
2008 3D-CBS Rome international review (world of reason/science) that change into a (world of power/money) of the 3D-CBS held at the Ordine dei Medici del Lazio in Rome, Italy. Simultaneously translate in English and broadcasted via internet streaming in two languages.
2008 UTSWMED—3D-CBS seminar by Crosetto at Southwest Medical Center in Dallas, Tex.
2008 Toronto 3D-CBS seminar by Crosetto
2008 CERN 3D-Flow and 3D-CBS seminar by Crosetto in Teleconference from CERN-CMS EVO conference room
2008 Regina Elena Hospital in Rome, Italy, 3D-CBS seminar by Crosetto
2008 CRO hospital in Aviano, Italy. 3D-CBS seminar by Crosetto
2008 1st. Tumori Hospital in Milan, Italy. 3D-CBS seminar by Crosetto
2008 S. Maria Nuova Hospital in Reggio Emilia, Italy. 3D-CBS seminar by Crosetto
2009 CPRIT Grant $4 M rejection (world of power/money) from CPRIT based on the title. Despite CPRIT CEO's promise that his proposal would receive rigorous scientific review, it was extremely disappointing as well as heartbreaking when Crosetto was notified that his proposal was not even examined. It was “triaged out” with 400 other proposals out of 800 submitted based on the Title of the proposal “3D Complete Body Screening (3D-CBS) for Early Cancer Detection Targeted to Reduce Premature Cancer Death at a Lower Cost per Life Saved Compared to Current Cost” and the 235 word abstract and significance.
2009 Nuclear Med 3D-CBS Crosetto's 42-page article. As a follow up to a presentation Crosetto gave of his 3D-CBS innovation in 2008 to Borasi and his colleagues at the S. Maria Nuova hospital in Reggio Emilia, an article/discussion between Crosetto and Borasi et al. was published from page 26 to 68 in May 2009 in the “Notiziario di Medicina Nucleare ed Imaging Molecolare”). This article reveal that the breakthrough was provided by the 3D-CBS. (The cited article by Borasi et al. is in fact an overview of several PET, including the 3D-CBS, and does not describe any other cost-effective true total body PET that can make a paradigm shift in Medical Imaging while is recognizing <<The first proposal for a truly “total body” PET system (120-160 cm AFOV) came from an Italian physicist, Crosetto [6]>>).
2009 Pavia exhibition of Crosetto's inventions lasted a week at the Sala Salome' downtown Pavia. It followed for a few days at the main building of the University of Pavia, Italy.
2010 CERN Physics for Health (world of reason/science) that change into a (world of power/money). Crosetto presented a paper with 14 co-authors and approximately 1,000 cosigners at the Workshop “Physics for Health” held at CERN in Geneva. The title of the paper is: “Progress in the domain of physics applications in life science with an invention for substantial reduction of premature cancer deaths: The need for a PARADIGM CHANGE in oncology research”. The abstract of the article was translated in 10 languages, including Japanese, Chinese and Russian.
2010 CERN Physics for Health. Del Guerra takes away the microphone from Crosetto while he is asking a question to a speaker at the Physics for Health workshop.
2010 CERN 7000 Petition Signatures (world of power/money). A delegation from the Crosetto Foundation delivered in a meeting with CERN Director General and Director of Research, over 7000 signature on paper of a petition requesting a public scientific review of Crosetto's inventions.
2011 Leonardo da Vinci Prize 3D-CBS (world of reason/science). Crosetto won the Leonardo da Vinci Prize for his 3D-CBS invention for early cancer detection.
2012 UICC-WCC world Cancer congress (world of reason/science) that change into a (world of power/money) Crosetto presentation was accepted at the World Cancer Congress on August 27-30, 2012 in Montreal, Canada. He translated his power point presentation in six languages.
2013 Crowdfunding from friends $5,300 donation (world of reason/science). Crosetto Foundation for the Reduction of Cancer Deaths pursued the crowd-funding initiative, however, checks from the Crosetto foundation were counterfeited causing the Foundation to close the account and the Foundations website and email received cyber-attacks.
2013 Cyber Attacks Crosetto Foundation website
2013 Counterfeited Checks of Crosetto Foundation
2013 Cyber Attacks Crosetto Foundation emails
2013 IEEE NSS-MIC rejection (world of power/money) also this year Crosetto's papers to IEEE-NSS-MIC have been rejected.
2013 IEEE RTSD (world of reason/science). D. Crosetto: “Breaking the Speed Barrier in Real-Time Applications to Make Advances in Particle Detection, Medical Imaging and Astrophysics.” Poster presentation R05-52 at 2013 IEEE-NSS-MIC-RTSD conference
2013 Fondazione San Paolo Grant $2,500 rejected (world of power/money) Giorgio Palestro, the referee for science at the Foundation Compagnia did not even consider a grant of approximately $2,500 that would have served to further assess the value of Crosetto's 3D-CBS invention in an international forum of the IEEE Life Science Grand Challenges Conference in Singapore on Dec. 2-3, 2013, where projects with the same objective were presented.
2014 WOKC 3D-CBS Grant $147,852 rejected (world of power/money) A survivor that was affected by cancer at childhood introduced Crosetto to WOKC and encourage him to submitted a proposal. Regretfully on Dec. 11, 2014, Crosetto receive an email from Evelyn Costolo, CEO of WOKC stating: “Thank you for your application to the WOKC Medical Grant Program. We have carefully reviewed each application and are unable to fund your request at this time.” No reasons were ever provided for the rejection.
2014 IEEE-MIC Moses (world of power/money). Crosetto meets several authors of the Explorer (Bill Moses, Simon Cherry, etc.) at the IEEE-NSS-MIC-RTSD Conference describing them the features of the 3D-CBS and asking if they believed it was more important sensitivity or spatial resolution in a PET. In 2015 the authors of the Explorer submitted a request for funding to NIH for $15.5 million, using in the abstract of their submission many ideas, features claims important to be develop, that Crosetto published in his book of the year 2000 and in subsequent articles (and they rejected for decades), even describing the same analogy of reducing the radiation in the Explore equivalent to the dose absorbed during a round trip intercontinental flight as shown in the cover page of Crosetto's 2000 book. In the year 2016, the Deputy Chairman of the IEEE-MIC conference, Suleman Surti, who is one of the authors of the Explorer rejected Crosetto's paper asking what it is an Ultra-sensitive 3D-CBS, and claiming he cannot search in the literature what is the 3D-CBS. Surti and his colleagues power point presentation of the Explorer in 2013 was titled “An Ultra-Sensitive Total Body PET Scanner for Biomedical Research” and in slide 5 titled “Not a New Idea!” cited Crosetto's article. Therefore, Surti knew the meaning of “Ultra-Sensitive PET” and was aware of Crosetto's work.
2014 IEEE-NSS the chairman takes away the microphone from Crosetto while he is asking a question to the NSS keynote speaker.
2014 IEEE-MIC, the chairman takes away the microphone from Crosetto while he is asking a question to the MIC keynote speaker.
2015 CRS-Italy 3D-CBS Grant 3,000 Euro rejected (world of power/money) submitted a proposal to the Cassa di Risparmio di Savigliano (the bank at his village where he was born) for 3000 Euros and was rejected while in the past approved grants for Crosetto's initiative on the cultural exchange between Texas and his native town in Italy.
2015 DOE Siegrist (world of reason/science) that change into a (world of power/money). Since May 5, 2015, Crosetto was solicited both verbally and in writing to formally submit a proposal of his inventions to the Department of Energy by his former supervisor at the Superconducting Super Collider, Dr. Jim Siegrist, who is now Director for the Office of High Energy Physics—HEP—at the Office of Science of the Department of Energy. During this time, Dr. Siegrist and several DOE employees told Crosetto he would need to communicate with Glenn Crawford as he is responsible for Research and Technology. However, Crawford never once answered Crosetto's technical emails, phone calls, or addressed/discussed any scientific issues with Crosetto.
2015 EU Parliament interrogation (world of power/money). Following the activity by the Crosetto Foundation for the Reduction in Cancer Deaths on May 27-28, 2015, in front of the building of the European Commission in Brussels that raised the issue in the press of the need for TRANSPARENCY in SCIENCE and in the assignment of funds for research, particularly in cancer, Hon. Alessia Mosca, a member of the European Parliament on May 29, 2015 filed a formal Priority Question to the European Commission with the request of a written answer. See the interrogation P-009063-15. It was received on Jun. 4, 2015, published on the European Union website in English on Jun. 11, 2015, with the Protocol Number P-009063/2015. See the written answer P-009063-15. Hon. Mosca commented the answer: “It doesn't seem really helpful to clarify our doubts. We will continue to following the issue.”
2016INFN 3D-Flow, 3D-CBS (a world of reason/science) that change into a (world of power/money). On Jan. 20, 2016, Crosetto gave a seminar (available in a video) of his project/proposal to the Director of the National Institute of Physics (INFN) of Turin, Amedeo Staiano and his colleagues.
   No one refuted conceptually or the feasibility of Crosetto's proposal and he satisfactorily answered the two questions:
      a) where is made the time alignment of the information arriving from different subdetectors and
      b) where are examples of 3D-Flow real-time trigger algorithms for the identification of new particles.
   Crosetto's answers to:
      a) the time alignment of trigger information arriving from several subdetectors that is packed in a single word 262,144-bit wide sent over 8,192 electronic channels every 25 nanoseconds to the 3D-Flow system is assembled in the Patch Panel Regrouping Associates Ideas (PRAI) board described in figures at pages 3, 4, 15, 19, 36, 159, 160, 200, 201 and 223 of the proposal, and
      b) example of 3D-Flow real-time algorithms to identify different particles suitable to process data arriving from different detector assembly from different experiments (e.g. CMS 3×3, Atlas 4×4, etc.) approved and recognized feasible by a majors public scientific review and endorsed by many experts that the 3D-Flow system has the capability to execute all of them and in addition can execute many algorithms that experimenters might think of in the future are reported in slides 36, 39, 40 of Crosetto's presentation on Jan. 20, 2016 at the INFN in Turin, Italy. Other examples of real-time algorithms that can be executed by the 3D-Flow are also available in several publications by Crosetto such as: at page 557, 558, 562 in 1992 World Scientific, at page 804 and 806 in 1992 CHEP, at page 48 in 1995, IEEE-RT with Sergio Conetti, at page 114 and 116 of the 2000 book, at page 3 and 6 of the 2000 IEEE-563 article, at page 93 of the 2005 book, at page 18 of the 2013 IEEE article.
   Pastrone did not keep what promised, she did not set a phone appointment, nor a time for a meeting at the University of physics in Turin between June 24 to 28. On Jun. 14, 2016, she wrote an email to Crosetto stating: “When I will have the time and opportunity to examine the documentation you sent me, if I will find it appropriate I will contact you or will ask someone to contact you.”
   Because Pastrone did not contact Crosetto to address these important issues saving taxpayer money and providing them and cancer patients great benefits in saving lives and costs, in light of the promises that Pastrone made to Crosetto to be respectful to taxpayer and cancer patients who are trusting her professional ethics and scientific integrity, it would be appropriate for her to reflect and resign in order to send the right message to those who do not respect her position and authority in doing what she admitted to Crosetto it is appropriate for a scientist to work for of a world of reason/science rather than a world of power/money.
2016 EU ENVI Meeting with the President of the ENVI, Abrami, Crosetto and Tamburini request for an audition to President Giovanni La Via, Chair of the ENVI Committee at the European Parliament who is in charge of Environment, Public Health and Food Safety. On Jun. 23, 2016, they present him their innovations benefitting humanity: effective tools to protect the environment, improve public health and food safety.
2016 NIH Laurer review rejected (world of power/money). The Director of extramural Research of the National Institute of Health (NIH), Michael Laurer, still does not want to hear Crosetto's inventions, does not want to organize a public scientific review to maximize results for taxpayers which would eliminate corruption because he believes this would not be fair to all scientists and does not give the same importance to be fair to the public. The last information that Crosetto received from the office of the Director of NIH, Dr. Collins, was that they had a meeting on Jul. 19, 2016, to find a person at NIH responsible to address scientific issues justifying the giving of $15.5 million for the Explorer project which is less efficient, it does not have the capability to save many lives, it is over ten times more expensive than the 3D-CBS device, which has instead the highest capabilities to save many lives because it can acquire and process over 40,000 TB data every day using the 3D-Flow OPRA. The office of the Director of NIH, promised that Crosetto would receive an answer late afternoon on Jul. 19, 2016, or on the following day; however, to date Crosetto has not receive an answer.
2016 DOE, 3D-Flow Grant $13.4 M Crawford's self-incriminating 8-line email (world of power/money) Jim Siegrist starting to address issues for a world of reason, however, they have changed into a world of power by Glen Crawford who was in charge to address scientific issues complying with DOE rules. Crawford's 8-line email dated May 19, 2016 and the information provided in this document reveal the path of abuse of power, stating one thing and the opposite in the next or following lines of his email, inventing and/or referring to non-existent DOE rules serving his plan of corruption, and demonstrating that if an innovation conflicts with his own plans or those of his circle of friends, it can never be funded at DOE
2016 DOE THREAT. Why didn't the DOE provide any calculations or scientific evidence to show how they came to their conclusion that Crosetto's invention to replace hundreds of crates of electronics with one crate at 1/1000 the cost and provide staggering performance improvement was not sound and feasible (which would mean that all scientists who endorsed Crosetto's inventions, the FERMILAB international review panel and engineers and industries who wrote the 59 quotes showing feasibility, are all foolish and incompetent), but instead appointed Security to threaten Crosetto into never contacting any DOE employee regarding this issue?
2016 Citigroup Grant rejected Grant $40,000 rejected (world of power/money) Crosetto's request to Citigroup for a grant of $40,000 per year (and/or a break on financial interests) until he will able to make the scientific truth prevail, benefitting humanity, clearly explained in this document with the funding agencies responsible to fund his innovations seemed legitimate and pertinent.
   It was also suggested by several people during Crosetto's seminars to ask for contributions to decision makers who make a public commitment to contribute to make a better world and who have significant budgets. These expectations from several people who attended Crosetto's seminar are legitimate because Citigroup have resources to access experts in the field of the technology of Crosetto's invention that laymen do not have. Experts appointed by Citigroup can ask question to Crosetto and assess the superiority of Crosetto's approach or pointing out another approach with higher potential to reduce cancer deaths and costs so that many people who want to contribute in a small way to a technology/project that can make a positive difference in the world. Citigroup is announcing on their website to be sensitive, attentive and supportive of projects that foster world's growth and progress. It should be legitimate to submit innovations proving to achieve Citi group's objective and expect a scientific reason accepting or refuting them.
2016 The Rockefeller Foundation Grant $2,150 rejected (world of power/money). The Rockefeller Foundation rejected a request of a mere amount of $2,150 that would help document in more detail, his inventions. His request on Jul. 27, 2016 for a $2,150 grant was justified to be within the Mission of the Rockefeller Foundation, answering to the Foundation's specific questions.
   A few hours after submitting his request for a grant for $2,150, Crosetto received the answer from Judith Rodin stating: “. . . your inquiry has been reviewed by Foundation staff. Unfortunately, at this time, we were not able to consider providing funding.” No reason was provided for the rejection.
   The Rockefeller Foundation can compare data in Crosetto's 2011 book and compare data of Table 8 between the Explorer and the 3D-CBS and determine in an analytical discussion based on calculations and scientific evidence, the best combination of a device for early cancer detection and the existing cures proven to work when cancer is detected at an early stage. They could also estimate the money this will save on the current annual cancer economic burden of over $1.4 trillion per year that could be used to solve the malnutrition problem in the world, which is stated to be an important objective on the Rockefeller Foundation website.
2016 IEEE NSS-MIC (world of power/money). The role of IEEE, the world's 400,000 member largest technical professional organization dedicated to advancing technology for the benefit of humanity to solve the inconsistencies reported in Crosetto's Response to the President of IEEE, are essential to implement IEEE Mission they stated on the their website.
2016 IEEE CERN-DOE-NIH-CPRIT-Cancer Organizations-Laymen. Crosetto prepared a 2-page summary and a trifold summarizing his inventions providing also some example of their benefits to humanity.

It is respectfully requested to investigate why innovations are suppressed, its inventor is threatened to involve security instead of providing scientific and economic REASONS why hundreds of crates of electronics that do not provide powerful tools to save lives and discover new subatomic particles are funded, knowing that the breakthrough 3D-Flow OPRA and 3D-CBS inventions, which have been recognized valuable by a formal, official majors public scientific review, endorsed by many experts in the field, proven feasible by 59 quotes from reputable industries can replace them in one chassis, providing powerful tools to save lives and to discover new subatomic particles at a fraction of the cost.

Here are the questions for which citizens of a civilized, democratic country should be receiving the answer for a world of REASON/SCIENCE:
1. Why did the U.S. Department of Energy (DOE) give $4.2 million of taxpayer money in 2016 to Wesley Smith after he wasted over $50 million building hundreds of crates with 4,000 electronic data processing board of the CMS Level-1 Trigger systems costing over $100 million which did not w ork and had to be trashed knowing that it can be replaced with one 3D-Flow OPRA crate at 1/1000 the cost with staggering performance improvements?
2. Why did the U.S. National Institutes of Health give $3,314,184 of taxpayer sisoney in 2016 (out of $15.5 million, $3,054,873 delivered in 2015) to the team of the Explorer project made of 491,520 expensive LYSO crystals, 12 crates with 120 data processing boards and 6 racks of computer processing 40 TB data acquired and stored on hard drives every day for a total power consumption of 60 kW, which cannot save many lives because it cannot acquire and process 40,000 TB data per day, knowing that the 3D-CBS device can make a paradigm change in molecular imaging because it has the capability to acquire and process over 40,000 TB data each day, using one 3D-Flow OPRA VME crate for a total power consumption of the entire system of only 4 Kw, at less than 1/10 the cost of the Explorer? Why, knowing that the 3D-CBS could have saved millions of lives with an effective early cancer detection, reduce radiation dose to less than 1/50, reduce healthcare costs and these results can be proven on a sample population, has it not been funded?
3. Why, after informing President Obama that more than one year after having proof that Glen Crawford, Director of Research and Technology at DOE is not doing his job and five months after his 8-line email incriminating himself with statements in violation of DOE rules and contradicting his own statements, does he still hold the same position and continues to waste tax payer money and be the internal arm of a circle of friends of corrupted scientists? Why, after Crosetto notified the DOE FOIA (Freedom of Information Act) agent by phone about this document published at http://blog.u2ec.org/wordpress/?P=1880 proving that Crawford ordered DOE Security to block Crosetto's messages to all DOE employees, including the Secretary of Energy, FOIA and OHA so he and his group (e.g. Helmut Marsiske) could continue to give millions of dollars to Wesley Smith and others without accountability is illegal and an abuse of power, Crosetto cannot send these facts to DOE?
4. Why, after Informing President Obama and Vice-President Joe Biden that Michael Laurer, Director of the Extramural Research at the National Institute of Health, does not want to hear Crosetto's inventions, does not want to organize a public scientific review of the 3D-CBS because he believes this would not be fair to all scientists, but does not give the same importance to be fair to the public or to serve the President and Vice-President who promise to give the public the greatest benefit for their tax-dollars, no action is taken depriving instead taxpayers from the benefits from innovations that would emerge from a fair scientific competition? Why, after proving in this document that the $15.5 million given to the Explorer project is a waste of money because the 3D-CBS can acquire and process over 40,000 TB data in one day providing an effective early cancer detection and a much more powerful research tool at less than 1/10 the cost, does Laurer not respond to hear about Crosetto's invention, organize a scientific review or consider resigning if he is being impeded from doing his job by someone, instead of complying with the ethics of a scientist and do what it is best for taxpayers?
5. Why, 3 days after Crosesto explained the reason of the great damage to the advancement in science and benefit to humanity for the rejection of his papers and providing facts proving reviewers of this year as well as previous years were either incompetent or did not tell the truth because facts and documents refute reviewers claims that they did not know about Crosetto's work, do leaders of the Nuclear Science Symposium and Medical Imaging Confernce in Strasbourg which takes place in 2 days, still not respond to let Crosetto know if he can present his inventions for the benefit of humanity at the Conference or if they will consider resigning if they are being impeded from doing their job by someone, instead of complying with the ethics of a scientist and do what it is best for taxpayers?
6. Why, 3 days after Crosetto informed the Presidents and board of Directors of IEEE, the world's 400,000 member largest technical professional organization dedicated to advancing technology for the benefit of humanity, that facts show that the organizers of the 2016IEEE-NSS-MIC-RTSD Conference in Strasbourg which takes place in 2 days, have rejected three papers regarding innovations proving to replace hundreds of crates of electronics with one crate at 1/1000 the cost and provide staggering performance improvement that has been proven feasible by 59 quotes from reputable industries, still do not respond about the logic and compliance with the scientific integrity that would call for IEEE to disassociate itself from the Strasbourg's conference organizers who are contrary to advancing technology for the benefit of humanity, and remove its endorsement of the conference.
7. Why, a) after Crosetto and two friends drove to Davos, Switzerland, to hand-deliver a letter containing Crosetto's 271-page proposal and 155-page report of 59 quotes from reputable industries to Vice-President Joe Biden who organized a forum among scientists of the Cancer Moonshot task force, b) after Crosetto sent an additional copy of the proposal to the physical address provided by Ms. Brazile who Crosetto met at an event at the University of Arlington in Texas, and c) after Mr. Charles Conner, a veteran who worked for Lyndon B. Johnson from 1959 to 1964, wrote a personal letter to Vice-President Joe Biden asking him to address the specific items in Crosetto's letter, clear out bureaucratic hurdles and have Crosetto be part of the Task Force to defeat cancer, did Mr. Conner not receive a reply, the specific items in Crosetto's letter were not addressed, and Crosetto was not included in the Task Force to defeat cancer?

161,765,000 people have died from cancer since Crosetto's invention was crushed (11,984,000 were Americans), many of them could have been saved with his 3D-CBS (3-D Complete Body Screening) technology and hundreds of millions of dollars of tax-payer money could have been saved in High Energy Physics experiments, while providing a very powerful tool to discover new subatomic particles and advance science in different fields. This needless loss of lives, waste of taxpayer money, and loss of advancement in science calls for the reform of the peer-review process to be independent from money and politics as expressed by the National Academy of Science.

What is claimed is:

1. A technology-independent, modular, scalable, ultra-high performance, low cost 3D-Flow OPRA system, implementable in different hardware form factors such as VXI, VME, VPX, ATCA, etc. for processing data signals arriving from a matrix of transducers or from an array of memory modules at several Terabytes per second per each crate, executing programmable complex Object Pattern Recognition Algorithms (OPRA) in real-time and capable of executing several functions when the desired trigger condition on the object sought is met. The innovative steps of this invention provide disruptive advantages compared to the state of the art. A 3D-Flow OPRA system for approximately 10,000 channels, sustaining an input data rated of a few Terabyte per second can be built in a VXI crate at a component and manufacturing cost of the order of $100,000. In comparison, the state of the art at CERN Research Laboratory in Geneva built a system with fewer number of channels, less performant at a cost of over one hundred million dollars.

2. The system of claim 1 comprising:
(a) A 3D-Flow OPRA processor with the capability of bidirectional communication in four directions and unidirectional communication in additional two directions. With the capability to execute uninterruptable algorithm for a time longer than the time interval between two consecutive input data
(b) A set of different electronic boards with different features in terms of number of inputs, the complexity of the OPRA algorithms can execute in real-time, the maximum speed of the input data rate it can sustain and the features provided when the desired trigger (or matching) condition is met.
(c) A set of different features such as saving data signals when the condition is met, saving data signals before and after the condition is met, taking an immediate action when the condition is met (such as in a closed-loop controlled system)

3. The system of claim 1 implemented in VXI 9U boards

4. The system of claim 1 implemented in VME 6U boards

5. A technology-independent, modular, scalable, high-speed, low cost data recorder and generator ER/DSU (Event Recorder and Detector Simulator Unit) implemented in several hardware form factors capable of reaching a transfer rate in/out of the order of a few Terabyte per second on a 9U VXI crate. A ER/DSU unit for approximately 10,000 channels, sustaining an input data rated of a few Terabyte per second can be built in a VXI crate at a component and manufacturing cost for less than $100,000. Commercially available instrumentation with less performance cost over ten times.
